(19) United States
(12) Patent Application Publication
(10) Pub. No.: US 2008/0260820 A1
(43) Pub. Date: Oct. 23, 2008

(54) Oral dosage formulations of protease-resistant polypeptides

(76) Inventors: Gilles Borrelly, Combs-la-Ville (FR); Caroline Chauchard, Viry-Chatillon (FR); Lila Drittanti, Vigneux-sur-Seine (FR); Manuel Vega, Vigneux-sur-Seine (FR)

Correspondence Address: Bell, Boyd & Lloyd LLP, 3580 Carmel Mountain Road, Suite 200, San Diego, CA, 92130, US

(21) Appl. No.: 11/788,836

(22) Filed: Apr. 19, 2007

(51) Int. Cl. A61K 38/21; A61K 31/7088; A61K 38/18; A61K 38/22; A61K 38/28; A61K 9/28; A61P 19/02; A61P 3/04; A61P 31/18; A61P 7/04; A61P 41/00; A61P 37/06; A61P 35/02; A61P 35/00; A61P 31/12; A61P 21/00; A61K 9/48; A61K 39/395; A61K 38/27; A61K 38/19; A61K 38/20

(52) U.S. Cl. 424/463; 369/44.11; 424/130.1; 424/474; 424/85.1; 424/85.2; 424/85.5; 424/85.6; 424/85.7; 424/94.6; 424/94.63; 514/12; 514/3; 514/44

(57) ABSTRACT

Oral dosage formulations of the protease-resistant therapeutic polypeptides are provided. The dosage formulations for administration per dosage per day or per week are higher than the subcutaneous dosage. Included among the oral dosage formulations are tablets and capsules, including enteric-coated forms. Methods for treatment or prevention of diseases or conditions that are treatable or preventable by the administration of such oral dosage formulations of protease-resistant polypeptides are provided.

### RELATED APPLICATIONS

This application is related to Korean application (Attorney Docket No. 17109-022KR1/932KR), Gilles Borrelly, Caroline Chauchard, Lila Drittanti and Manuel Vega, entitled “ORAL DOSAGE FORMULATIONS OF PROTEASE-RESISTANT POLYPEPTIDES,” filed on Apr. 19, 2007. This application also is related to U.S. application Ser. No. 11/176,830, to Rene Gantier, Thierry Guyon, Manuel Vega and Lila Drittanti, entitled “RATIONAL EVOLUTION OF CYTOKINES FOR HIGHER STABILITY, THE CYTOKINES AND ENCODING NUCLEIC ACID MOLECULES,” filed Jul. 6, 2005, and published as U.S. Application No. US 2006-0020116, which is a continuation of U.S. application Ser. No. 10/658,834, to Rene Gantier, Thierry Guyon, Manuel Vega and Lila Drittanti entitled “RATIONAL EVOLUTION OF CYTOKINES FOR HIGHER STABILITY, THE (sold as CYTOKINES AND ENCODING NUCLEIC ACID MOLECULES,” filed Sep. 8, 2003, and published as U.S. Application No. US-2004-0132977-A1. This application also is related to U.S. application Ser. No. 11/196,067, to Rene Gantier, Thierry Guyon, Cruz Ramos Hugo, Manuel Vega and Lila Drittanti entitled “RATIONAL DIRECTED PROTEIN EVOLUTION USING TWO-DIMENSIONAL RATIONAL MUTAGENESIS SCANNING,” filed Aug. 2, 2005, and published as U.S. Application No. US-2006-0020396-A1, which is a continuation of U.S. application Ser. No. 10/658,355, to Rene Gantier, Thierry Guyon, Cruz Ramos Hugo, Manuel Vega and Lila Drittanti, entitled “RATIONAL DIRECTED PROTEIN EVOLUTION USING TWO-DIMENSIONAL RATIONAL MUTAGENESIS SCANNING”, filed Sep. 8, 2003 and published as U.S. Application No. US 2005-0202438.

This application also is related to U.S. application Ser. No. 10/658,834, filed Sep. 8, 2003, and to published International PCT Application WO 2004/022593, to Rene Gantier, Thierry Guyon, Manuel Vega and Lila Drittanti entitled, “RATIONAL EVOLUTION OF CYTOKINES FOR HIGHER STABILITY, THE CYTOKINES AND ENCODING NUCLEIC ACID MOLECULES.” This application also is related to U.S. application Ser. No. 10/658,355, filed Sep. 8, 2003, and to International PCT Application WO 2004/022747, to Rene Gantier, Thierry Guyon, Cruz Ramos Hugo, Manuel Vega and Lila Drittanti entitled “RATIONAL DIRECTED PROTEIN EVOLUTION USING TWO-DIMENSIONAL RATIONAL MUTAGENESIS SCANNING.”

The subject matter of each of the above-referenced applications is incorporated by reference in its entirety.

### INCORPORATION BY REFERENCE OF SEQUENCE LISTING PROVIDED ON COMPACT DISCS

An electronic version on compact disc (CD-R) of the Sequence Listing is filed herewith in duplicate (labeled Copy # 1 and Copy # 2), the contents of which are incorporated by reference in their entirety. The computer-readable file on each of the aforementioned compact discs, created on Apr. 17, 2007, is identical, 3,214 kilobytes in size, and titled 932SEQ.001.txt.

### FIELD OF INVENTION

Oral dosage formulations of protease-resistant polypeptides are provided. Methods for treatment or prevention of diseases or conditions that are treatable or preventable by the administration of the protease-resistant polypeptides are provided.

### BACKGROUND

Therapeutic polypeptides have been approved for treatment of a variety of diseases and conditions including, but not limited to, proliferative diseases, such as cancer, inflammatory disease, autoimmune diseases, viral infection, bacterial infection, blood disorders, cardiovascular diseases, respiratory diseases, neural disorders, gastrointestinal disorders, and metabolic diseases, such as obesity. While oral administration is desirable, therapeutic proteins typically are administered via injection or other route that avoids the digestive tract.

Upon oral administration, these proteins are continuously digested by luminal proteases of the gastrointestinal tract resulting in little or no absorption of the therapeutic protein into the blood. Gastrointestinal degradation or other proteolytic degradation of therapeutic proteins prevents their per-oral administration. Thus, oral delivery of therapeutic proteins for clinical use is a challenge for pharmaceutical science to solve. It is desirable to have oral dosage formulations of such therapeutic polypeptides. Hence, among the objects herein, it is an object to provide oral formulations of therapeutic polypeptides.

### SUMMARY

Provided are oral dosage formulations, such as tablets and capsules, of therapeutic polypeptides. The therapeutic polypeptides are modified in their primary sequences, typically by one or two amino acid changes, to render them more resistant to proteases than absent such modification. The formulations contain an amount of the modified therapeutic polypeptide so that upon oral administration a therapeutically effective amount of the polypeptide is delivered to the bloodstream. The dosage formulations can include one or dosage unit, each of which contains the entire dosage amount or a fraction thereof. The dosage formulations generally contain an amount of therapeutic polypeptide that is higher, about or 6-200, such as about or 10-100, including, for example, 15-50, 20-30, 10-35, 10-45, typically about or 15-40 times higher (depending upon the therapeutic protein and indication treated and the formulation) for administration per day, than amounts that are administered subcutaneously for the same indication using the same therapeutic protein that does not have the modified amino acid changes that render it protease resistant. Hence if, for example, 1 mg/day is administered to a 70 kg male subcutaneously, 6 mg to 200 mg or more is administered via oral dosage formulation. The particular dosage depends upon the therapeutic polypeptide and the oral dosage formulation, and, if needed, can be empirically determined. Dosage regimens, such as number of administrations per week can vary depending upon the therapeutic protein and the indication. For oral administration, the total dosage per week for example, is greater, 10-200 times or more, than the subcutaneous weekly dosage of the same therapeutic protein that is not modified for protease resistance.

In particular, provided are dosage formulations for protease-resistant therapeutic polypeptide that contain an amount of a therapeutic protein sufficient, upon oral administration to a subject, to reach therapeutically effective levels in the bloodstream in the subject. The therapeutic protein is modified in its primary sequence of amino acids, whereby it is more protease resistant than in the absence of the modifications, and the protein can reach therapeutically effective levels in the bloodstream upon oral administration; and the amount is about or is 10-100, typically 10-50, 15-45, 15-40 times the subcutaneous dose for the same indication and for the same therapeutic protein that is not modified to be protease resistant. The dosage refers to that which is administered in a day. A greater differential, such as 10-1000, 10-600 times greater for an oral dosage compared to a subcutaneous dosage when comparing on a weekly basis. The oral dosage, however, are more convenient to administer and have greater patient compliance. Also, as shown herein, the pharmacokinetics of the oral dosages administration can be better controlled and more uniform.

The unit dosages in the oral dosage formulations of protease-resistant therapeutic polypeptides can be formulated with one or more pharmaceutically acceptable excipients. The oral dosage formulations of protease-resistant therapeutic polypeptides can contain dosage units that are coated to effect delivery of the polypeptide to the gastrointestinal tract or parts thereof. For example, oral dosage formulations or dosage unites that contain one or more dosage units that is/are enterically coated are provided.

The protease-resistant therapeutic polypeptides in the unit dosage or dosages in the oral dosage formulations of can be further modified in activity of structure, such as to include glycosylation, carboxylation, hydroxylation, hasylation, carbamylation, sulfation, phosphorylation and/or PEGylation. Included are protease-resistant therapeutic polypeptides that are hyperglycosylated. Other activities and properties of the polypeptides can be modified as well.

The protease-resistant therapeutic polypeptide can contain additional modifications. Such modifications include amino acid modifications that contribute to increased activity, altered immunogenicity, stability, thermal tolerance, protease-resistance, glycosylation, carboxylation, hydroxylation, hasylation, carbamylation, sulfation, phosphorylation, or PEGylation. Such amino acid modifications can be natural amino acids, non-natural amino acids and/or a combination of natural and non-natural amino acids.

Among the therapeutic proteins that are modified are protease-resistant variants of coagulation factors, cytokines, growth factors, hormones, hydrolases, immunoglobulins, inhibitor proteins, nuclear proteins and proteases, such as, but are not limited to,

interleukin-10, an interferon-β; an interferon alpha, interferon gamma, granulocyte colony stimulating, leukemia inhibitory factor, a growth hormone, ciliary neurotrophic factor, leptin, insulin, oncostatin M, relaxin, interleukin-6, interleukin-12, erythropoietin, granulocyte-macrophage colony stimulating factor, interleukin-2, interleukin-3, interleukin-4, interleukin-5, interleukin-13, receptor ligands, such as Flt3 ligand, monoclonal antibody, antibody fragments, cameloids, and stem cell factors. The modifications that increase protease resistance can be the only modifications in the sequence of amino acids compared to a wild-type protein. The protease-resistant therapeutic polypeptides can include additional modifications that alter properties other than protease resistance in the sequence of amino acids compared to a wild-type protein. Additional modifications include those that, upon expression in a suitable expression system, add one or more glycosylation sites to produce hyperglycosylation proteins. Modification with respect to protease resistance is compared to the absence of the modifications that result in protease resistance

Exemplary of the unmodified therapeutic proteins that can be modified to exhibit increased protease resistance are any set forth in the Sequence Listing, such as, but are not limited to, interleukin-10 (IL-10; SEQ ID NO: 809), interferon beta (IFN-0; SEQ ID NO: 147), interferon alpha-2a (IFNα-2a; SEQ ID NO: 2162), interferon alpha-2b (IFNα-2b; SEQ ID NO: 2067), interferon gamma (IFN-γ; SEQ ID NO: 661), granulocyte colony stimulating factor (G-CSF; SEQ ID NO: 47), leukemia inhibitory factor (LIF; SEQ ID NO: 1148), growth hormone (GH; SEQ ID NO: 1260), ciliary neurotrophic factor (CNTF; SEQ ID NO: 1), leptin (SEQ ID NO: 1126), oncostatin M (SEQ ID NO: 1181), interleukin-6 (IL-6; SEQ ID NO: 1080), interleukin-12 (IL-12; SEQ ID NO: 860); erythropoietin (EPO; SEQ ID NO: 1886), granulocyte-macrophage colony stimulating factor (GM-CSF; SEQ ID NO: 85), interleukin-2 (IL-2; SEQ ID NO: 946), interleukin-3 (IL-3; SEQ ID NO: 995), interleukin-4 (IL-4; SEQ ID NO: 1018), interleukin-5 (IL-5; SEQ ID NO: 1044), interleukin-13 (IL-13; SEQ ID NO: 917), Flt3 ligand (SEQ ID NO: 118) and stem cell factor (SCF; SEQ ID NO: 1215). Particular protease-resistant therapeutic polypeptides include interferon-α2a and -α2b, and human growth hormone. Modified protease-resistant therapeutic polypeptides include polypeptides whose sequences are set forth in any of SEQ ID NOS: 2-46, 48-84, 86-117, 119-146, 148-660, 662-808, 810-859, 861-916, 918-945, 947-994, 996-1017-, 1019-1043, 1045-1079, 1081-1125, 1127-1147, 1149-1180, 1182-1214, 1216-1259, 1261-1885, 1887-1981, 1983-2066, and 2176-2182.

Provided herein are oral dosage formulations of protease-resistant IFN-α polypeptides. Protease-resistant IFN-α polypeptides can be any IFN-α polypeptide that is modified in its primary sequence, typically by one or two amino acid changes, to render the polypeptide more resistant to proteases than absent such modification. Provided herein are oral dosage formulations of a protease-resistant IFN-α polypeptide, where the modification is replacement of a glutamic acid residue (E) with a glutamine residue (Q) at position 41 of a mature IFN-α polypeptide set forth in SEQ ID NO: 2067 or at corresponding position in an allelic or species variant thereof and other variants modified in other loci.

Provided herein are oral dosage formulations of protease-resistant IFN-α polypeptides formulated with one or more pharmaceutically acceptable excipients. The oral dosage formulations of protease-resistant IFN-α polypeptides can be coated or non-coated to effect delivery of the polypeptide to gastrointestinal tract or parts thereof.

Provided herein are oral dosage formulations of protease-resistant IFN-α polypeptides that are coated with an enteric coating to effect delivery of the polypeptide to lower gastrointestinal tract.

Provided herein are oral dosage formulations of protease-resistant growth hormone polypeptides. Protease-resistant growth hormone polypeptides can be any growth hormone polypeptide that is modified in its primary sequence, typically by one or two amino acid changes, to render the polypeptide more resistant to proteases than absent such modification. Provided herein are oral dosage formulations of a protease-resistant growth hormone polypeptide, where the modification is replacement of a tyrosine residue (Y) with a isoleucine residue (I) at position 42 of a mature growth hormone polypeptide set forth in SEQ ID NO: 1260 or at corresponding position in an allelic or species variant thereof. Provided herein are oral dosage formulations of a protease-resistant growth hormone polypeptide, where the modification is replacement of a tyrosine residue (Y) with a histidine residue (H) at position 42 of a mature growth hormone polypeptide set forth in SEQ ID NO: 1260 or at corresponding position in an allelic or species variant thereof.

Provided herein are oral dosage formulations of protease-resistant growth hormone polypeptides formulated with one or more pharmaceutically acceptable excipients. The oral dosage formulations of protease-resistant growth hormone polypeptides can be coated, such as enterically coated to effect delivery of the polypeptide to gastrointestinal tract or parts thereof.

Also provided are tablets and capsules that contain a protease-resistant therapeutic polypeptide. The tablets and capsules are dosage units. Dosage formulations containing one or more, such as 2, 3, 4, 5, 6, 7, 8, 9, 10 or more of the tablets and capsules are provided.

Also provided are tablets and capsules that contain about or 10-100 μg, such as 3, 5 or 10 μg-50 μg, 3, 5 or 10 μg-30 μg, 3, 5 or 10 μg-40 μg, 3, 5 or 10 μg-45 μg, such as 10-50 μg of an interferon-α (IFN-α). Exemplary tablets or capsules contain 20, 30, 40 or 50 μg of the INF-α, such as IFN-α2a or -α2b. The IFN-α2 is protease resistant by virtue of modifications in its primary sequence of amino acids. The IFN-α can be IFN-α2a or IFN-α2b. The tablet or capsule can include further therapeutic or active agents, such as a second therapeutic agent for the same indication as the interferon. Exemplary second therapeutic agents include, but are not limited to, nucleoside analog, such as ribavirin, an L-nucleoside, a type II interferon receptor antagonist, a tumor necrosis factor (TNF) antagonist, thymosin-α, a SAPK inhibitor, amantidine, an NS3 inhibitor, an NS5B inhibitor and an alpha-glucosidase inhibitor. The IFN-α, such the IFN-α2b, can further include additional modifications, whereby it is hyperglycosylated upon expression. The amount is corrected to reflect the additional mass due to hyperglycosylation or other modification. The tablet or capsule of can be enterically coated. Exemplary of the modified IFN-α2b is the IFN-2a, such as IFN-α2b that contains a modification that is or corresponds to E41Q of SEQ ID NO: 2067, such as the polypeptide whose sequence set forth in SEQ ID NO: 1995 or allelic and species variants thereof.

Also provided are tablets and capsules that contain protease-resistant therapeutic polypeptides, such as human growth hormone. The tablet or capsule containing the modified hGH can contain, for example, 1-, 3- or 5-50 mg, 10-50 mg, 10-40 mg, 15-35 mg, 1-40 mg, 1-45 mg, 5-30 mg, 10-20 mg or 3-30 mg of human growth hormone, wherein the human growth hormone is protease resistant. The tablet or capsule can contain additional therapeutic or active agents, such as second therapeutic agent for the same indication as the growth hormone, such as, for example a gonadotropin releasing hormone (GNRH) analog, insulin-like growth factor 1, (rhIGF-1), a growth hormone-releasing peptide (GRP), a free fatty acid regulation agent, leutenizing hormone releasing hormone, and an anabolic steroid. Exemplary tablets or capsules contain 3 mg, 12 mg, 24 mg or 30 mg of the protease-resistant growth hormone polypeptide. The tablets and capsules can be formulated to be enterically coated.

The human growth hormone can contain additional modifications that alter other functional properties and/or activities, such as addition of glycosylation sites, such that upon expression in a suitable system, the polypeptide is hyperglycosylated. Exemplary of protease-resistant therapeutic polypeptides is a human growth hormone contains a modification that is or corresponds to Y42I of SEQ ID NO: 1260 and/or allelic or species variants thereof, such as a human growth hormone whose sequence of amino acids is set forth in SEQ ID NO: 1318.

Provided herein are dosage units, such as tablets and capsules, and oral dosage formulations containing one or more dosage units of protease-resistant growth hormone polypeptides or IFN-α polypeptide. The dosage units can be coated with or formulated form an enteric coating so that the tablet or capsule has increased resistance to digestion or degradation in the digestive tract. For example, provided are tablets and capsules that contain about or 1-100 gm, 1-50 mg, 10-50 mg, 10-40 mg, 15-35 mg, 1-40 mg, 1-45 mg, 5-30 mg, 10-20 mg or 3-30 mg of human growth hormone. The human growth hormone is protease resistant by virtue of modifications of the primary sequence of amino acids. The tablets and capsules optionally are enterically coated.

Provided are tablets and capsules that contain about or 10, 20, 30, 40, 50, 60, 70, 75, 80, 90 100, 125 μg or more of IFN-α. IFN-α polypeptide is protease resistant by virtue of modifications of the primary sequence of amino acids. The tablets and capsules optionally are enterically coated.

Use of the dosage formulations and dosage unites and/or the tablets and capsules for treatment of a disease or condition are provided. Also provided are uses of a modified therapeutic protein for treatment of a disease, where the therapeutic protein is modified in its primary sequence of amino acids, whereby it is more protease resistant than in the absence of the modifications, and the protein can reach therapeutically effective levels in the bloodstream upon oral administration; and the amount of therapeutic protein in the formulation 15-40 times the subcutaneous dose for the same indication and for the same therapeutic protein that is not modified to be protease resistant.

Uses of a modified therapeutic protein for formulation of a medicament for treatment of a disease are provided. The protease-resistant therapeutic polypeptide is modified in its primary sequence of amino acids, whereby it is more protease resistant than in the absence of the modifications; the polypeptide can reach therapeutically effective levels in the bloodstream upon oral administration; and the amount of therapeutic protein in the formulation 15-40 times the subcutaneous dose for the same indication and for the same therapeutic protein that is not modified to be protease resistant. Any modified protease-resistant therapeutic polypeptide can be employed, including, for example, treatment of a disease or condition for which treatment by administration of IFN-α or growth hormone is indicated, where the protein is an IFN-α for treatment where IFN-α is indicated; and the protein is growth hormone for treatment where growth hormone is indicated.

Also provided are methods of treatment of a disease or condition by administering a dosage formulation or tablet or capsule provided herein. The dosage formulation can contain one or a plurality of dosage units and all are administered in a single day. Any suitable regimen can be followed, such as one in which a dosage formulation is administered one, two, three, four, five, six or seven times or more a week, or one in which a dosage formulation is administered one, two, three for four times a day, every day, 2, 3, 4, 5, 6 or 7 times a week.

### BRIEF DESCRIPTION OF THE FIGURES

FIG. 1(A) illustrates the process by which native or wild-type therapeutic polypeptides degraded by gastrointestinal proteases of the intestinal lumen, resulting in little or no absorption of the polypeptide into the blood. The graphs above each figure illustrate the amount of active polypeptide in the blood.

FIG. 1(B) illustrates the process by which protease-resistant polypeptides are resistant to gastrointestinal proteases of the intestinal lumen, resulting in sustained absorption of the polypeptide into the blood. The graphs above each figure illustrate the amount of active polypeptide in the blood.

FIG. 2 depicts the effects of native hGH and hGH(Y42I) on body weight following administration of liquid formulations of the proteins either orally (oral) or subcutaneously (SC) in a hypophesectomized rat model of growth hormone deficiency.

### DETAILED DESCRIPTION

Outline
A. Definitions
B. Gastrointestinal Absorption and Detection of Protease-resistant Polypeptides
C. Oral Dosage Formulations
   1. Oral Dosage Units
   2. Preparation of the Oral Dosage Formulation and Oral Dosage Units
      a. Delivery Control
         i. Enteric coatings
         ii. Multiple Coatings and Sub-coatings
      b. Other Delivery Additives and Carriers
D. Protease-resistant Polypeptides for Oral Dosage Formulations
   1. Protease Resistant Polypeptides
   2. Cytokines
      a. Interferon/Interleukin-10 Family
      b. Long-chain Cytokine Family
      c. Short-chain Cytokine Family
   3. Other Exemplary Polypeptides
      a. Blood Factors
      b. Insulin
      c. Relaxin
      d. Antibodies
      e. Growth factors
      f. Soluble receptors
      g. Chemokines
      h. Angiogenic agents
      i. Neuroactive peptides
      j. Additional proteins
E. Oral Dosage Formulations of IFN-α Protease-resistant Polypeptides
   1. Protease-resistant IFN-α Polypeptides
      a. Further Modifications of Protease-resistant IFN-α Polypeptides
   2. Expression of Protease-resistant IFN-α Polypeptides
   3. Oral formulations and Unit Dosage Forms
F. Oral Dosage Formulations of Growth Hormone Protease-resistant Polypeptides
   1. Protease-resistant Growth Hormone Polypeptides
      a. Other Modifications of Protease-resistant Growth Hormone Polypeptides
   2. Expression of Nucleic Acid Encoding Protease-resistant Growth Hormone Polypeptides
   3. Oral formulations and Unit Dosage Forms
G. Methods Of Treatment And Uses Of The Oral Dosage Formulations
   1. Treatment Methods Using IFN-α
      a. Fibrotic Disorders
         i. Idiopathic Pulmonary Fibrosis
         ii. Liver Fibrosis
         iii. Renal Fibrosis
      b. Cancer
      c. Viral Infections
         i. Hepatitis Virus Infection
   2. Treatment Methods Using Growth Hormone
      a. Growth deficiencies
      b. Cachexia
      c. Anti-aging
      d. Renal osteodystrophy
      e. Cystic fibrosis
      f. Other conditions
H. Combination Therapies
I. Articles of Manufacture and Kits
J. Examples

### A. DEFINITIONS

Unless defined otherwise, all technical and scientific terms used herein have the same meaning as is commonly understood by one of skill in the art to which the invention(s) belong. All patents, patent applications, published applications and publications, GenBank sequences, websites and other published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it understood that such identifiers can change and particular information on the internet can come and go, but equivalent information can be found by searching the internet or other sources. Reference thereto evidences the availability and public dissemination of such information.

As used herein, dosage regimen refers to the number of oral dosage formulations administered per time period, such as a day or week.

As used herein, an oral dosage formulation refers a formulation containing one or more oral dosage units of a protease-resistant therapeutic polypeptide formulated for oral delivery, where the oral dosage formulation containing the one or more oral dosage units administered in a single dose provide an amount of the protease-resistant therapeutic polypeptide sufficient to achieve an therapeutically effective amount of the polypeptide in the blood via absorption through the gastrointestinal tract. Accordingly, an oral dosage formulation refers to the amount of the protease-resistant therapeutic polypeptide that is administered per dose and that achieves a therapeutically effective level in the bloodstream.

As used herein, an “oral dosage unit” contains a full or fractional therapeutically effect amount of a protease resistant polypeptide. Hence, an oral dosage formulation can include one or more oral dosage units. Thus, a pill, tablet, or capsule, containing the protease-resistant polypeptide is exemplary of an oral dosage unit. The pill, tablet or capsule can contain the protease-resistant polypeptide, for example, in the form of a lyophilized powder, granules, particles, gel or solution. It also can include one or more inactive ingredients, such as pharmaceutically-acceptable excipients (e.g., suspending agents, surfactants, disintegrants, binders, diluents, lubricants, stabilizers, antioxidants, osmotic agents, colorants, plasticizers and coatings) that are used to manufacture and deliver active pharmaceutical agents, such as a protease-resistant polypeptide. Such pills, tablets, or capsules can be coated for enteric delivery or non-coated. Capsules can contain enteric-coated beads or enteric-coated granules of the protease-resistant polypeptide.

As used herein, “therapeutically effective amount administered” or “therapeutically effective dose,” refers to an amount of an agent, compound, material, in a dosage formulation that is at least sufficient to produce a therapeutic effect in a subject. Typically, the amount is high enough to reach a therapeutically effective amount in the blood. The therapeutically effective amount in the blood to be achieved is known for many of the therapeutic polypeptides employed in the methods and dosage formulations effective dosages have been established for the unmodified proteins. For the modified proteins the dosages can be selected to achieve the same effect. The therapeutically effective amount of a protease-resistant polypeptide for use for treatment will vary with the particular condition being treated, the age and physical condition of the patient being treated, the severity of the condition, the duration of the treatment, the nature of concurrent therapy, the particular protease-resistant polypeptide being employed, the particular pharmaceutically-acceptable excipients and/or factors within the knowledge and expertise of the attending physician.

As used herein, a therapeutically effective amount in the blood is an amount at least sufficient to produce a therapeutic effect in a subject.

As used herein, a “protease-resistant polypeptide” is a protein that contains one or more modifications in its primary sequence of amino acids compared to a native or wild-type polypeptide and exhibits increased resistance to proteolysis compared to the native or wild-type polypeptide without the one or more amino acid modifications. Hence a protease-resistant polypeptide, such as a protease-resistant therapeutic polypeptide, is a polypeptide that, by virtue of changes in the primary sequence of amino acids, typically only one, two or three changes, can be formulated for oral administration, and upon administration, enter the bloodstream. Generally such polypeptides are resistant to proteases present in the digestive tract, but, because of the relatedness of proteases, can exhibit or be selected to be resistant to proteases in the blood. Such increase can be manifested as a concomitant increase in serum half-life. By increased protease resistance is meant an increase as assessed by in vitro or in vivo assays, including those exemplified herein, and is compared to the polypeptide absent the amino acid sequence changes. Any increase in resistance is contemplated as long as the resulting polypeptide can be administered orally so that it is absorbed into the blood in therapeutically effective amounts.

Increased resistance to proteases can be assessed by testing for activity following exposure to particular proteases present in the gastrointestinal tract and/or serum. Typically the increase in protease resistance is at least about 1%, 2%, 3%, 4%, 5%, 6%, 7%, 8%, 9%, 10%, 20%, 30%, 40%, 50%, 60%, 70%, 80%, 90%, 91%, 92%, 93%, 94%, 95%, 96%, 97%, 98%, 99%, 100%, 200%, 300%, 400%, 500%, or more compared to the same polypeptide, absent the changes in amino acid sequence that confer the resistance. In other embodiments, the resistance to proteases of the variant polypeptides for use in oral dosage formulations provided herein is increased by an amount of at least, 2 time, 3 times, 4 times, 5 times, 6 times, 7 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times, 900 times, 1000 times, or more, compared to the same polypeptide, absent the changes in amino acid sequence that confer the resistance.

As used herein, “resistance to proteolysis” refers to any amount of decreased cleavage of polypeptide by a proteolytic agent, such as a protease. This can be achieved by modifying particular amino acid residues in a polypeptide that are susceptible to cleavage by a particular protease to render the polypeptide less susceptible to cleavage compared to cleavage of the polypeptide without the modification, by the same protease under the same conditions. A modified polypeptide that exhibits increased resistance to proteolysis exhibits, for example, 1%, 2%, 3%, 4%, 5%, 6%, 7%, 8%, 9%, 10%, 20%, 30%, 40%, 50%, 60%, 70%, 80%, 90%, 91%, 92%, 93%, 94%, 95%, 96%, 97%, 98%, 99%, 100%, 200%, 300%, 400%, 500%, or more resistance to proteolysis compared to the same polypeptide, absent the amino acid modification(s).

As used herein, native or wild-type therapeutic protein refers to a protein that has not been modified to be protease resistant. Such proteins do not reach therapeutically effective levels for any indication when administered orally.

As used herein, “proteases,” “proteinases” or “peptidases” are interchangeably used to refer to enzymes that catalyze the hydrolysis of covalent peptidic bonds. Proteases include, for example, serine proteases and matrix metalloproteinases. Serine proteases or serine endopeptidases constitute a class of peptidases, which are characterized by the presence of a serine residue in the active center of the enzyme. Serine proteases participate in a wide range of functions in the body, including blood clotting, inflammation as well as digestion in prokaryotes and eukaryotes. The mechanism of cleavage by “serine proteases,” is based on nucleophilic attack of a targeted peptidic bond by a serine. Cysteine, threonine or water molecules associated with aspartate or metals also can play this role. Aligned side chains of serine, histidine and aspartate form a catalytic triad common to most serine proteases. The active site of serine proteases is shaped as a cleft where the polypeptide substrate binds. Amino acid residues are labeled from N to C termini of a polypeptide substrate (Pi, . . . , P3, P2, P1, P1′, P2′, P3′, . . . , Pj). The respective binding sub-sites are labeled (Si, . . . , S3, S2, S1, S1′, S2′, S3′, . . . , Sj). The cleavage is catalyzed between P1 and P1′. Proteases of the lower gastrointestinal tract are provided by the pancreas, which secretes bile into the gastrointestinal tract via the duodenum. Such proteases include, for example, trypsin, chymotrypsin, elastase, and carboxypeptidase.

As used herein term “pharmaceutically-acceptable excipients” includes any physiologically inert, pharmacologically inactive material known to one skilled in the art, which is compatible with the physical and chemical characteristics of the particular protease-resistant polypeptide selected for use. Pharmaceutically-acceptable excipients include, but are not limited to, polymers, resins, plasticizers, fillers, lubricants, solvents, co-solvents, buffer systems, surfactants, preservatives, sweetening agents, flavoring agents, pharmaceutical grade dyes or pigments, and viscosity agents. All or part of the pharmaceutically-acceptable excipients contained in the pharmaceutically compositions described herein can be part of the enteric coating.

As used herein, the term “enteric-coated oral dosage formulation” or “enteric-coated oral dosage form” relates to an oral dosage formulation containing a modified therapeutic protein that has an enteric coating to effect the release of the protease-resistant polypeptide in the lower intestinal tract. The enteric coating prevents early digestion or degradation of the tablet, capsule or other oral dosage form. The enteric coated oral dosage formulations, include, for example, a compressed tablet (coated or uncoated) containing granules, particles, which are themselves coated or uncoated, or a lyophilized powder of the protease-resistant polypeptide. The enteric coated oral dosage formulation can be a gelatin capsule (coated or uncoated) containing beads granules, particles, which are themselves coated or uncoated, or a lyophilized powder of the protease-resistant polypeptide.

As used herein, the term “enteric-coating” relates to a mixture of pharmaceutically-acceptable excipients which is applied to, combined with, mixed with or otherwise added to the protease-resistant polypeptide. The coating can be applied to a compressed tablet, a gelatin capsule, and/or the beads, granules, particles, or a lyophilized powder of the protease-resistant polypeptide, which are encapsulated into starch or gelatin capsules or compressed into tablets.

Accordingly, an enteric coating can be applied to a compressed tablet which contains granules, particles, or a lyophilized powder of the protease-resistant-polypeptide; however, in the event the granules or particles are themselves enterically-coated before being compressed into a tablet, then the enteric coating of the compressed tablet itself is optional. The enteric coating also can applied to the beads or small particles' of the protease-resistant-polypeptide, which can be encapsulated into a starch or gelatin capsule. The capsule can then be coated with an enteric coating, if desired. Because of their enteric coating, these oral dosage formulations will prohibit the undesirable delivery of the protease-resistant polypeptide to the mucosal and epithelial tissues of the upper gastrointestinal tract, especially the mouth, pharynx and esophagus. The coating also achieves the delivery of the active to the lower gastrointestinal tract at a point which can be manipulated by one skilled in the art by choosing the excipients which make up the coating, its type, and/or its thickness.

As used herein, a “therapeutic polypeptide” refers to any polypeptide that is administered for treatment of an animal, including a human. Such polypeptides can be prepared by any methods, and hence, include, but are not limited to, a recombinantly produced polypeptides, synthetically produced polypeptides, therapeutic polypeptides extracted from cells or tissues and other sources. As isolated from any sources or as produced, mature therapeutic polypeptides can be heterogeneous in length. Heterogeneity of therapeutic polypeptides can differ depending on the source of the therapeutic polypeptides. Hence reference to therapeutic polypeptides refers to the heterogeneous population as produced or isolated. When a homogeneous preparation is intended, it will be so-stated. References to therapeutic polypeptides herein are to their monomeric, dimeric or other multimeric forms, as appropriate.

Human therapeutic polypeptides include allelic variant isoforms, synthetic molecules produced from encoding nucleic acid molecules, proteins isolated from human tissue and cells, synthetic proteins, and modified forms thereof. Exemplary unmodified mature human therapeutic polypeptides include, but are not limited to, unmodified and native (i.e., wild-type) therapeutic polypeptides and the unmodified and native precursor therapeutic polypeptides that include a signal peptide and/or propeptide, and polymorphic native therapeutic polypeptides. Other exemplary human therapeutic polypeptides are those that are truncated at the N- or C-terminus.

Reference to therapeutic polypeptides also includes allelic or species variants of therapeutic polypeptides, and truncated forms or fragments thereof and forms that contain modifications in addition to those that increase protease resistance. Therapeutic polypeptides include homologous polypeptides from different species including, but not limited to animals, including humans and non-human species, such as other mammals. As with human therapeutic polypeptides, non-human therapeutic polypeptides also include heterogeneous lengths or fragments or portions of therapeutic polypeptides that are of sufficient length or include appropriate regions to retain at least one activity of full-length mature polypeptide.

Non-human therapeutic polypeptides include therapeutic polypeptides, allelic variant isoforms, synthetic molecules prepared from nucleic acids, protein isolated from non-human tissue and cells, and modified forms thereof. Therapeutic polypeptides of non-human origin include, but are not limited to, bovine, ovine, porcine, equine, murine, leporine, canine, feline, avian and other primate, such as chimpanzee and macaque, therapeutic polypeptides.

As used herein, “treating” a subject with a disease or condition means that the subject's symptoms are partially or totally alleviated, or remain static following treatment. Hence treatment encompasses prophylaxis, therapy and/or cure. Prophylaxis refers to prevention of a potential disease or condition and/or a prevention of worsening of symptoms or progression of a disease or condition. Prevention of a disease or condition encompasses alleviation or elimination of one or more risk factors for development of the disease or condition. Treatment also encompasses any pharmaceutical use of a modified therapeutic polypeptide and oral compositions provided herein.

As used herein, “patient” or “subject” to be treated includes humans or non-human animals. Mammals include primates, such as humans, chimpanzees, a gorillas and monkeys; domesticated animals, such as dogs, horses, cats, pigs, goats, cows; and rodents such as mice, rats, hamsters and gerbils.

As used herein, the term “gastrointestinal tract” relates to the alimentary canal, i.e., the musculo-membranous tube about thirty feet in length in a human subject, for example, extending from the mouth to the anus. As used herein, the term “upper gastrointestinal tract”, means the buccal cavity, the pharynx, the esophagus, and the stomach.

As used herein, the term “lower gastrointestinal tract” means the small intestine, and the large intestine.

As used herein, the term “buccal cavity” means the mouth or oral cavity and is lined with a mucous membrane which is continuous with the integument of the lips and with the mucous lining of the pharynx.

As used herein, the term “pharynx” relates to that part of the upper gastrointestinal tract which is placed behind the nose, mouth and larynx. The pharynx is a mucomembraneous tube about 4 inches in length and it is contiguous anteriorly with the mouth and posteriorly with the esophagus and is composed of a mucous coat, a fibrous coat, and a muscular coat.

As used herein, the term “esophagus” is a muscular canal about nine inches long extending from the pharynx to the stomach. The esophagus has three coats: an internal mucous coat surrounding the lumen, a middle areolar coat, and an external muscular coat.

As used herein, the term “stomach” means the part of the gastrointestinal tract between the esophagus and the small intestine.

As used herein, the term “small intestine” a means that part of the lower gastrointestinal tract that includes the duodenum, the jejunum, and the ileum, i.e., the portion of the intestinal tract just distal to the duodenal sphincter of the fundus of the stomach and proximal to the large intestine.

As used herein, the term “large intestine” includes the part of the lower gastrointestinal tract just distal to the small intestine, beginning with the cecum, including the ascending colon, the transverse colon, the descending colon, the sigmoid colon, and the rectum.

As used herein, “oromucosal” refers to refers to the mucosa lining the oral and/or nasopharyngeal cavities.

As used herein, the term “delayed-release” refers to a delivery of a protease-resistant polypeptide, which is effected by formulating the protease-resistant polypeptide in a pharmaceutical composition so that the release will be accomplished at some generally predictable location in the lower intestinal tract more distal to that which would have been accomplished if there had been no alteration in the delivery of the protease-resistant polypeptide. An exemplary method for effecting the delayed-release of the active ingredient involves coating (or otherwise encapsulating) the active ingredient with a substance which is not absorbed, or otherwise broken down, by the gastrointestinal fluids to release the active ingredient until a specific desired point in the intestinal tract is reached. An exemplary type of delayed-release formulation for use herein is achieved by coating the tablet, capsule, or particles, granules, or beads of active ingredient with a substance which is pH-dependent, i.e., broken down at a pH which is generally present in the small intestine, but not broken down at a pH which is generally present in the mouth, pharynx, esophagus or stomach. However, if it is desired to effect the topical delivery via the oral administration of a pharmaceutical composition containing the protease-resistant polypeptide to only the large intestine, or to the entire length of the intestinal tract beginning with the small intestine, then the selection of the coating material and/or the method of coating or otherwise combining the protease-resistant polypeptide with the selected coating material or other pharmaceutically-acceptable excipients can be varied or altered as is described herein or by any method known to one skilled in the art.

As used herein, AIDS wasting and other forms of cachexia are metabolic disorders that cause the body to consume vital muscle and organ tissue (lean body mass) for energy instead of primarily relying on the body's fat supplies. Subjects with AIDS wasting typically experience a loss of 5-10% or more of lean body mass, which includes muscle tissue, body organs, blood cells and lymphatic fluids

As used herein, the term “sustained-release” means the type of release mechanism designed to effect the delivery of the active ingredient over an extended period of time, as contrasted to the delivery of a delayed-release type dose. An exemplary sustained-release type method involves the coating of granules of the protease-resistant polypeptide with a pH-independent coating, chosen from the group including, but not limited to ethylcellulose, hydroxypropylmethylcellulose, methylcellulose, hydroxymethylcellulose, hydroxyethylcellulose, and sodium carboxymethylcellulose. Various sustained-release dosage forms can be fashioned by one skilled in the art to achieve the delivery of the protease-resistant polypeptide to the small intestine and the large intestine, to only the small intestine, or to only the large intestine, depending upon the choice of the various coating materials, and/or coating thickness.

As used herein, a therapeutic polypeptide dimer refers to a combination of two monomeric therapeutic polypeptides having the same or a different number of amino acids and/or different sequence of amino acids. Typically, dimeric forms of the polypeptide include those that contain two monomers linked via non-covalent interactions, including hydrophobic interactions, hydrogen bonds, van der Waals and other such interactions. Such dimers can form spontaneously when expressed and typically form spontaneously, such as, for example, as occurs using the methods of protein production described herein. Dimers also can be produced as fusion proteins, such as in the form of a single chain dimeric therapeutic polypeptide due to direct or indirect linkage of the same or different monomers. For purposes herein, the first monomer of a dimer is designated “A” and the second monomer of the dimer is designated “B.” Helices in each monomer are numbered, such that the helices in a polypeptide with six helices are designated A1-A6 for monomer A and B1-B6 for monomer B.

As used herein, an allelic variant or allelic variation references a polypeptide encoded by a gene that differs from a reference form of a gene (i.e., is encoded by an allele) among a population. Typically, the reference form of the gene encodes a native form and/or predominant form of a polypeptide from a population or single reference member of a species. Typically, allelic variants have at least 80%, 90%, 95% or greater amino acid identity with a native and/or predominant form from the same species.

As used herein, species variants refer to variants of the same polypeptide between and among species. Generally, interspecies allelic variants have at least about 60%, 70%, 80%, 85%, 90% or 95% identity or greater with a native and/or predominant form from another species, including 96%, 97%, 98%, 99% or greater identity with a native and/or predominant form of a polypeptide.

As used herein, “native therapeutic polypeptide” or “wild-type therapeutic polypeptide” refers to a therapeutic polypeptide encoded by a naturally occurring gene that is present in an organism in nature, such as in an animal, including a human or other mammal. Included among native therapeutic polypeptides are the encoded precursor polypeptide, fragments thereof, and processed forms thereof, such as a mature form lacking the signal peptide as well as any pre- or post-translationally processed or modified form thereof. For example, humans express therapeutic polypeptides. Exemplary of a native human therapeutic polypeptide is a precursor therapeutic polypeptide containing the signal peptide as well as a mature therapeutic polypeptide lacking the signal peptide. Also included among native therapeutic polypeptides are those that are post-translationally modified, including those that are proteolytically processed or that include other post-translational modifications such as, for example, glycosylation. Other animals, such as mammals, express native therapeutic polypeptides, and include, but are not limited to, hamsters, mice, rats, rabbits, birds, cows, horses, pigs, cats, dogs, monkeys, orangutans, baboons, chimpanzees, macaques, gibbons, and gorillas. As noted above, in nature, the polypeptides occur as a heterogeneous mixture that contains polypeptides of varying lengths and epigenetic modification, such as differences in glycosylation patterns.

As used herein, a “portion or fragment of a therapeutic polypeptide” or “an active portion” refers to any portion of a human or non-human therapeutic polypeptide that exhibits one or more activities of the full-length polypeptide.

As used herein, an “activity” of a therapeutic polypeptide refers to any activity exhibited by the therapeutic polypeptide. Such activities can be tested in vitro and/or in vivo. Activity can be any level of percentage of activity of the polypeptide, including but not limited to, 1% of the activity, 2%, 3%, 4%, 5%, 10%, 20%, 30%, 40%, 50%, 60%, 70%, 80%, 90%, 95% or more of functional activity compared to the full-length polypeptide. For example, percentage of activity of the polypeptide also includes 96%, 97%, 98%, 99%, 100%, 200%, 300%, 400%, 500%, or more of functional activity compared to the full-length polypeptide. Activities can be measured in vitro or in vivo using recognized assays. The results of such assays that indicate that a polypeptide exhibits an activity can be correlated to activity of the polypeptide in vivo, which can be referred to as biological activity.

As used herein, “EC50” refers to the effective concentration of a therapeutic polypeptide necessary to give one-half of a maximum response. For purposes herein, the response measured is any activity of a therapeutic polypeptide, such as but not limited to, antiviral activity or proliferation activity.

As used herein, “half-life” refers to the time required for a measured parameter, such as the potency, activity or effective concentration of a polypeptide or molecule to fall to half of its original level, such as half of its original potency, activity or effective concentration at time zero. Thus, the parameter, such as potency, activity, or effective concentration of a polypeptide molecule is generally measured over time. For purposes herein, half-life can be measured in vitro or in vivo. For example, the half-life of a therapeutic polypeptide or a modified therapeutic polypeptide can be measured in vitro by assessing its activity following incubation over time under certain conditions, such as for example, following exposure to proteases. In another example, the half-life of a therapeutic polypeptide or a modified therapeutic polypeptide can be measured in vivo following administration (e.g., intravenous, subcutaneous, intraduodenal, oral) of the polypeptide to a human or other animal, followed by sampling of the blood over time to determine the remaining effective concentration and/or activity of the polypeptide in the blood sample.

As used herein, a “property” of a therapeutic polypeptide refers to any property exhibited by such polypeptide. Such properties include, but are not limited to, protein stability, resistance to proteolysis, conformational stability, thermal tolerance, and tolerance to pH conditions. Changes in properties can alter an “activity” of the polypeptide.

As used herein, “serum stability” refers to protein stability in serum.

As used herein, a “directed evolution method” refers to methods that “adapt” either proteins, including natural proteins, synthetic proteins or protein domains to have changed properties, such as the ability to act in different or existing natural or artificial chemical or biological environments and/or to elicit new functions and/or to increase or decrease a given activity, and/or to modulate a given feature. Exemplary directed evolution methods include, among others, rational directed evolution methods described in U.S. application Ser. No. 10/022,249; and U.S. Published Application No. US-2004/0132977-A1.

As used herein, “two dimensional rational mutagenesis scanning (2-D scanning)” refers to the processes in which two dimensions of a particular protein sequence are scanned: (1) one dimension is to identify specific amino acid residues along the protein sequence to replace with different amino acids, referred to as is-HIT target positions, and (2) the second dimension is the amino acid type selected for replacing the particular is-HIT target, referred to as the replacing amino acid.

As used herein, “variant,” “therapeutic polypeptide variant,” “modified therapeutic polypeptide” and “modified therapeutic protein” refers to a therapeutic polypeptide that has one or more mutations compared to an unmodified therapeutic polypeptide. The one or more mutations can be one or more amino acid replacements, insertions or deletions and any combination thereof. Typically, a modified therapeutic polypeptide has one or more modifications in primary sequence compared to an unmodified therapeutic polypeptide. For example, a modified therapeutic polypeptide provided herein can have 1, 2, 3, 2, 5, 6, 7, 8, 9, 10, 11, 12, 13, 14, 15, 16, 17, 18, 19, 20 or more mutations compared to an unmodified therapeutic polypeptide. Modified therapeutic polypeptides provided herein include the specified or recited modification, but can be produced as heterogeneous mixtures and/or can be produced with a variety of lengths. Any length polypeptide is contemplated as long as the resulting polypeptide exhibits at least one therapeutic polypeptide activity.

As used herein, a “single amino acid replacement” refers to the replacement of one amino acid by another amino acid. The replacement can be by a natural amino acid or non-natural amino acids. When one amino acid is replaced by another amino acid in a protein, the total number of amino acids in the protein is unchanged.

As used herein, corresponding residues refer to residues compared among or between to polypeptides that are allelic or species variants or other isoforms. One of skill in the art can readily identify residues that correspond between or among such polypeptides. For example, by aligning the sequences of such polypeptides, one of skill in the art can identify corresponding residues, using conserved and identical amino acid residues as guides. In other instances, corresponding regions can be identified. One skilled in the art also can employ conserved amino acid residues as guides to find corresponding amino acid residues between and among human and non-human sequences.

As used herein, the phrase “structural homology” refers to the degree of coincidence in space between two or more protein backbones. Protein backbones that adopt the same protein structure, fold and show similarity upon three-dimensional structural superposition in space can be considered structurally homologous. Structural homology is not based on sequence homology, but rather on three-dimensional homology. Two amino acids in two different proteins that are homologous based on structural homology between those proteins do not necessarily need to be in sequence-based homologous regions. For example, protein backbones that have a root mean squared (RMS) deviation of less than 3.5, 3.0, 2.5, 2.0, 1.7 or 1.5 angstroms at a given space position or defined region between each other can be considered to be structurally homologous in that region and are referred to herein as having a “high coincidence” between their backbones. It is contemplated herein that substantially equivalent (e.g., “structurally related”) amino acid positions that are located on two or more different protein sequences that share a certain degree of structural homology have comparable functional tasks; also referred to herein as “structurally homologous loci.” These two amino acids are considered to be “structurally similar” or “structurally related” with each other, even if their precise primary linear positions on the sequences of amino acids, when these sequences are aligned, do not match with each other. Amino acids that are “structurally related” can be far away from each other in the primary protein sequences, when these sequences are aligned following the rules of classical sequence homology.

As used herein, “at a position corresponding to” refers to a position of interest (i.e., base number or residue number) in a nucleic acid molecule or protein relative to the position in another reference nucleic acid molecule or protein. The position of interest to the position in another reference protein can be in, for example, a precursor protein, an allelic variant, a heterologous protein, an amino acid sequence from the same protein of another species (i.e., species variant), etc. Corresponding positions can be determined by comparing and aligning sequences to maximize the number of matching nucleotides or residues, for example, such that identity between the sequences is greater than 95%, such as than 96%, 97%, 98%, 99% and higher. The position of interest is then given the number assigned in the reference nucleic acid molecule.

As used herein, the terms “homology” and “identity”” are used interchangeably, but homology for proteins can include conservative amino acid changes. In general to identify corresponding positions the sequences of amino acids are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carillo et al. (1988) SIAM J Applied Math 48:1073).

As use herein, “sequence identity” refers to the number of identical amino acids (or nucleotide bases) in a comparison between a test and a reference polypeptide or polynucleotide. Homologous polypeptides refer to a pre-determined number of identical or homologous amino acid residues. Homology includes conservative amino acid substitutions as well identical residues. Sequence identity can be determined by standard alignment algorithm programs used with default gap penalties established by each supplier. Homologous nucleic acid molecules refer to a pre-determined number of identical or homologous nucleotides. Homology includes substitutions that do not change the encoded amino acid (i.e., “silent substitutions”) as well identical residues. Substantially homologous nucleic acid molecules hybridize typically at moderate stringency or at high stringency all along the length of the nucleic acid or along at least about 70%, 80%, 85%, 90%, 91%, 92%, 93%, 94%, 95%, 96%, 97%, 98%, or 99% of the full-length nucleic acid molecule of interest. Also contemplated are nucleic acid molecules that contain degenerate codons in place of codons in the hybridizing nucleic acid molecule. (For determination of homology of proteins, conservative amino acids can be aligned as well as identical amino acids; in this case, percentage identity and percentage homology vary). Whether any two nucleic acid molecules have nucleotide sequences (or any two polypeptides have amino acid sequences) that are at least 70%, 80%, 85%, 90%, 91%, 92%, 93%, 94%, 95%, 96%, 97%, 98% or 99% “identical” can be determined using known computer algorithms such as the “FAST A” program, using for example, the default parameters as in Pearson et al. Proc. Natl. Acad. Sci. USA 85: 2444 (1988) (other programs include the GCG program package (Devereux, J., et al., Nucleic Acids Research 12(I): 387 (1984)), BLASTP, BLASTN, FASTA (Atschul, S. F., et al., J. Molec. Biol. 215:403 (1990); Guide to Huge Computers, Martin J. Bishop, ed., Academic Press, San Diego (1994), and Carillo et al. SIAM J Applied Math 48: 1073 (1988)). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.)). Percent homology or identity of proteins and/or nucleic acid molecules can be determined, for example, by comparing sequence information using a GAP computer program (e.g., Needleman et al. J. Mol. Biol. 48: 443 (1970), as revised by Smith and Waterman (Adv. Appl. Math. 2: 482 (1981)). Briefly, a GAP program defines similarity as the number of aligned symbols (i.e., nucleotides or amino acids) which are similar, divided by the total number of symbols in the shorter of the two sequences. Default parameters for the GAP program can include: (1) a unary comparison matrix (containing a value of 1 for identities and 0 for non identities) and the weighted comparison matrix of Gribskov et al. Nucl. Acids Res. 14: 6745 (1986), as described by Schwartz and Dayhoff, eds., Atlas of Protein Sequence and Structure, National Biomedical Research Foundation, pp. 353-358 (1979); (2) a penalty of 3.0 for each gap and an additional 0.10 penalty for each symbol in each gap; and (3) no penalty for end gaps.

Therefore, as used herein, the term “identity” represents a comparison between a test and a reference polypeptide or polynucleotide. In one non-limiting example, “at least 90% identical to” refers to percent identities from 90 to 100% relative to the reference polypeptides. Identity at a level of 90% or more is indicative of the fact that, assuming for exemplification purposes a test and reference polynucleotide length of 100 amino acids are compared, no more than 10% (i.e., 10 out of 100) of amino acids in the test polypeptide differs from that of the reference polypeptides. Similar comparisons can be made between a test and reference polynucleotides. Such differences can be represented as point mutations randomly distributed over the entire length of an amino acid sequence or they can be clustered in one or more locations of varying length up to the maximum allowable, e.g., 10/100 amino acid difference (approximately 90% identity). Differences are defined as nucleic acid or amino acid substitutions, insertions or deletions. At the level of homologies or identities above about 85-90%, the result should be independent of the program and gap parameters set; such high levels of identity can be assessed readily, often without relying on software.

As used herein, the phrase “sequence-related proteins” refers to proteins that have at least 50%, at least 60%, at least 70%, at least 80%, at least 90%, at least 91%, at least 92%, at least 93%, at least 94%, at least 95%, at least 96%, at least 97%, at least 98% or at least 99% amino acid sequence identity or homology with each other.

As used herein, families of non-related proteins or “sequence-non-related proteins” refer to proteins having less than 50%, less than 40%, less than 30%, less than 20% amino acid identity, or homology with each other.

As used herein, it also is understood that the terms “substantially identical” or “similar” varies with the context as understood by those skilled in the relevant art.

As used herein, “a naked polypeptide chain” refers to a polypeptide that is not post-translationally modified or otherwise chemically modified, but contains only covalently linked amino acids.

As used herein, the amino acids that occur in the various sequences of amino acids provided herein are identified according to their known, three-letter or one-letter abbreviations (Table 1). The nucleotides which occur in the various nucleic acid fragments are designated with the standard single-letter designations used routinely in the art.

As used herein, an “amino acid” is an organic compound containing an amino group and a carboxylic acid group. A polypeptide contains two or more amino acids. For purposes herein, amino acids include the twenty naturally-occurring amino acids, non-natural amino acids, and amino acid analogs (e.g., amino acids wherein the α-carbon has a side chain).

As used herein, the abbreviations for any protective groups, amino acids and other compounds are, unless indicated otherwise, in accord with their common usage, recognized abbreviations, or the IUPAC-IUB Commission on Biochemical Nomenclature (1972) Biochem. 11: 1726). Each naturally occurring L-amino acid is identified by the standard three letter code (or single letter code) or the standard three letter code (or single letter code) with the “L-;” the “D-” indicates that the stereoisomeric form of the amino acid is D.

As used herein, “amino acid residue” refers to an amino acid formed upon chemical digestion (hydrolysis) of a polypeptide at its peptide linkages. The amino acid residues described herein are presumed to be in the “L” isomeric form. Residues in the “D” isomeric form, which are so designated, can be substituted for any L-amino acid residue as long as the desired functional property is retained by the polypeptide. “NH₂” refers to the free amino group present at the amino terminus of a polypeptide. “COOH” refers to the free carboxy group present at the carboxyl terminus of a polypeptide. In keeping with standard polypeptide nomenclature described in J. Biol. Chem., 243: 3552-3559 (1969), and adopted 37 C.F.R. §§ 1.821-1.822, abbreviations for amino acid residues are shown in Table 1:

TABLE 1
Table of Correspondence

SYMBOL

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| Z | Glx | Glu and/or Gln |
| W | Trp | tryptophan |
| R | Arg | arginine |
| D | Asp | aspartic acid |
| N | Asn | asparagine |
| B | Asx | Asn and/or Asp |
| C | Cys | cysteine |
| X | Xaa | Unknown or other |

It should be noted that all amino acid residue sequences represented herein by formulae have a left to right orientation in the conventional direction of amino-terminus to carboxyl-terminus. In addition, the phrase “amino acid residue” is broadly defined to include the amino acids listed in the Table of Correspondence (Table 1) and modified and unusual amino acids, such as those referred to in 37 C.F.R. §§ 1.821-1.822, and incorporated herein by reference. Furthermore, it should be noted that a dash at the beginning or end of an amino acid residue sequence indicates a peptide bond to a further sequence of one or more amino acid residues, to an amino-terminal group such as NH₂ or to a carboxyl-terminal group such as COOH.

As used herein, “naturally occurring amino acids” refer to the 20 L-amino acids that occur in polypeptides.

As used herein, the term “non-natural amino acid” refers to an organic compound that has a structure similar to a natural amino acid but has been modified structurally to mimic the structure and reactivity of a natural amino acid. Non-naturally occurring amino acids thus include, for example, amino acids or analogs of amino acids other than the 20 naturally occurring amino acids and include, but are not limited to, the D-isostereomers of amino acids. Exemplary non-natural amino acids are described herein and are known to those of skill in the art.

As used herein, “purified” preparations prepared from 1 cells or hosts or other sources refers to at least a purity of a cell extracts containing the indicated DNA or protein including a crude extract of the DNA or protein of interest. For example, in the case of a protein, a purified preparation can be obtained following an individual technique or a series of preparative or biochemical techniques, and the DNA or protein of interest can be present at various degrees of purity in these preparations. The procedures can include, but are not limited to, ammonium sulfate fractionation, gel filtration, ion exchange chromatography, affinity chromatography, density gradient centrifugation, and electrophoresis.

As used herein, a preparation of DNA or protein that is “substantially pure” or “isolated” refers to a preparation substantially free from naturally-occurring materials with which such DNA or protein is normally associated in nature and generally contains 5% or less of the other contaminants.

As used herein, a composition refers to any mixture of two or more products or compounds (e.g., agents, modulators, regulators, etc.). A composition includes a solution, a suspension, liquid, powder, a paste, aqueous, non-aqueous formulations or any combination thereof.

As used herein, a combination refers to any association between two or more items. Items of a combination for administration to a subject can be administered separately or together, used simultaneously or sequentially, or packaged together or packaged separately,

As used herein, an “article of manufacture” is a packaged composition. As used throughout this application, the term is intended to encompass pharmaceutical compositions of modified polypeptides and/or nucleic acids as described herein contained in articles of packaging optionally containing instructions for administration, particularly as an oral dosage formulation.

As used herein, a “kit” refers to a combination of a modified polypeptides or nucleic acid molecules as described herein provided in pharmaceutical compositions and another item for a purpose including, but not limited to, administration, diagnosis, and assessment of an activity or property of the polypeptides described herein. Kits, optionally, include instructions for use.

### B. GASTROINTESTINAL ABSORPTION AND DETECTION OF PROTEASE-RESISTANT POLYPEPTIDES

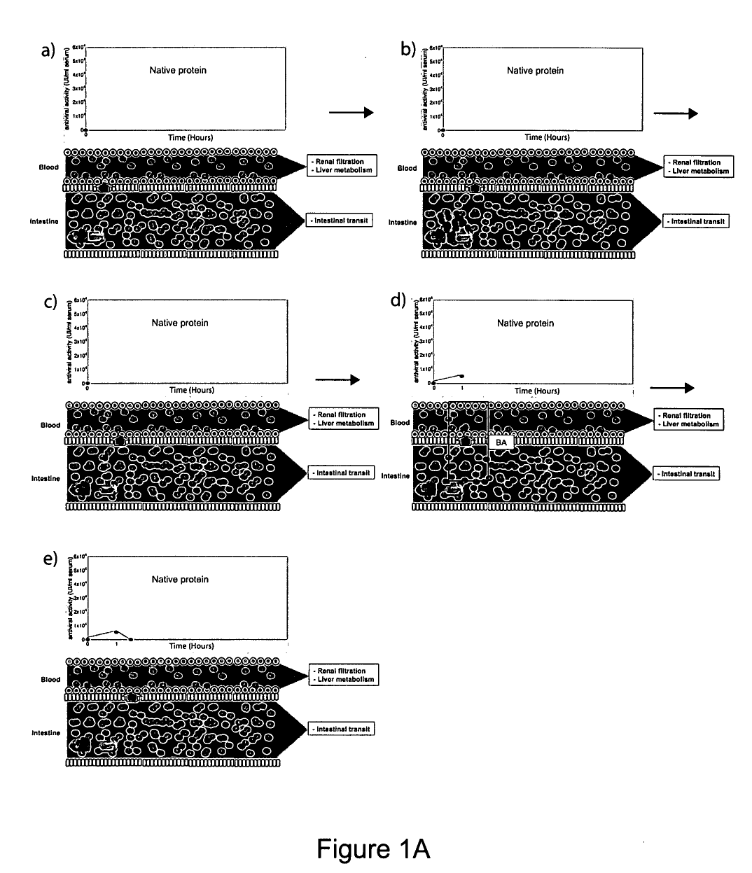

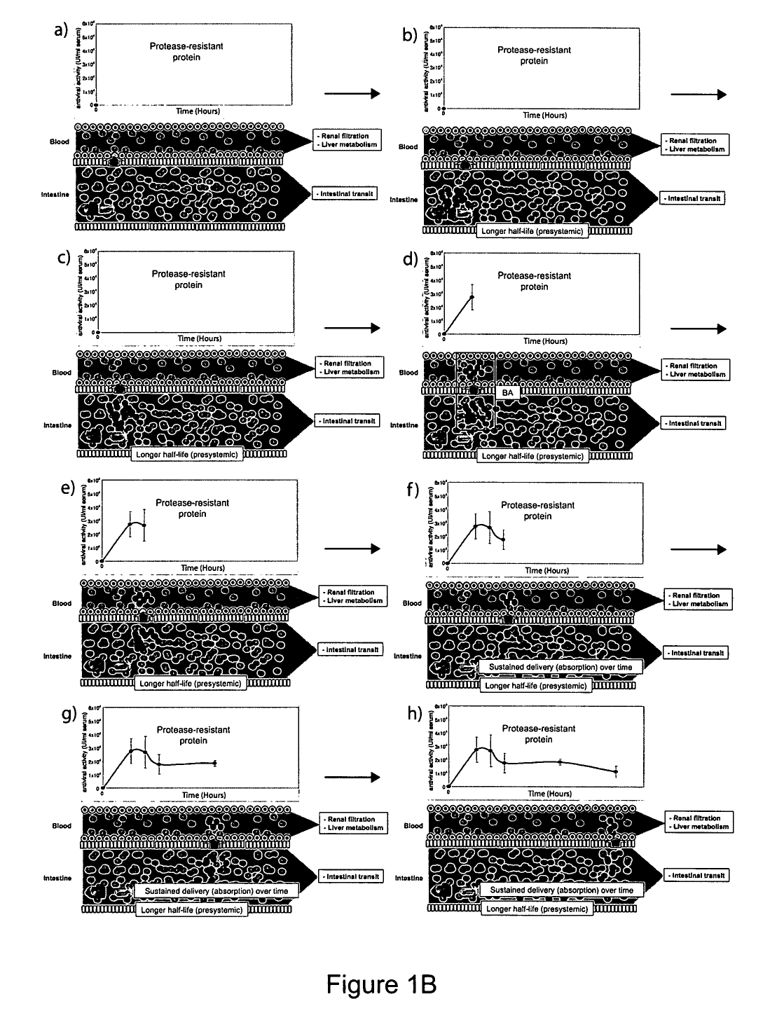

As shown herein, for therapeutic proteins that are not modified to be protease resistant by virtue of changes in their primary sequence, no amount of protein can be administered via oral administration that achieves delivery of therapeutically effective amounts of native, or wild-type, polypeptides to the bloodstream. This results from degradation of the polypeptides in the gastrointestinal tract by gastrointestinal proteases (FIG. 1A). The native polypeptides are susceptible to rapid degradation by proteases present in the lower intestinal tract (FIG. 1A(a-c)). Therefore, no therapeutic protein is available for adsorption from the gastrointestinal tract due to proteolytic degradation. Although native polypeptides can be formulated with coatings for enteric delivery this is not sufficient to effect resistance to proteases in the gastrointestinal tract to allow for release into the bloodstream. Once the enteric coating is dissolved and the native polypeptides are released into the lumen of the gastrointestinal tract, high levels of proteolytic degradation of the polypeptides prevent efficient absorption of the polypeptides from the intestine into the blood (FIG. 1A(d-e)). Hence, therapeutically effective amounts of the therapeutic polypeptide in the bloodstream cannot be achieved by oral delivery of the native polypeptide. The modified therapeutic proteins used in the oral dosage formulations and dosage units provided herein are more resistant to proteases in the bloodstream than therapeutic proteins not so-modified.

In contrast, as shown herein, protease-resistant polypeptides that are resistant to degradation by proteases, such as gastrointestinal proteases, are available for absorption and uptake into the blood (FIG. 1B). When released into the lumen of the gastrointestinal tract, the protease-resistance peptides are present in high concentrations due do their resistance to intestinal proteases (FIG. 1B(a-c)). As a result, therapeutically effective amounts of the proteases can be absorbed from the intestine into the bloodstream (FIG. 1B(d-e)). Additionally, resistance to proteolytic degradation in the digestive tract and in the bloodstream allows for sustained uptake of the protease-resistant therapeutic polypeptides. Hence, the protease-resistant polypeptides can be absorbed and maintained at therapeutically effective concentrations in the bloodstream over longer periods of time (FIG. 1B(f-h)). Thus, the protease-resistant polypeptides can be administered orally. Provided herein are dosage formulations that provide formulations of the protease-resistant polypeptides. Typically such formulations are tablets or capsules. Advantageously, they include enteric coatings that protect against the pH conditions of the stomach and allow for efficient delivery of the polypeptides to the lower digestive tract.

The absorption into the bloodstream of protease resistant polypeptides contained in the oral dosage formulations provided herein can be assessed following oral administration. Typically, following administration of the dosage formulation orally, blood is sampled 0.2, 0.5, 1, 2, 3, 4, 5, 6, 12, 24, or 48 hours following administration and the amount of the polypeptide present in the blood is assessed. The assessment of levels of protein over time permits analysis of the sustained presence of the polypeptide in the blood. If desired, the half-life of the polypeptide in the blood also can be determined.

In one example, the blood can be assayed for the presence of the polypeptide using routine protein screening assays. Such screening assays include, but are not limited to, Coomassie Blue Staining, Silver staining, Western Blot, and ELISA. In another example, the blood can be assayed for the presence of the polypeptide using functional activity assays. For example, anti-viral, anti-proliferation, chemotactic, coagulation, and other such activity assays can be used depending on the protein to be assayed. As exemplified in the examples herein, the absorption and activity of a protease resistant interferon-alpha (IFN-α) polypeptide in the bloodstream following oral administration is assessed in an anti-viral activity assay. In another example, the absorption and activity of a protease resistant growth hormone (GH) in the bloodstream following oral administration is assessed in a proliferation assay. Typically, such functional assays correlate with the amount of active polypeptide present in the bloodstream and can be an accurate indicator of polypeptides that retain activity and thereby are therapeutically active.

Generally, oral administration of the dosage formulations provided herein permits absorption of the protease resistant polypeptide at a therapeutically effective amount. The amount of polypeptide present in the blood that is at a therapeutically effective level can be empirically determined, and will differ based on the particular therapeutic polypeptide administered and the disease or disorder to be treated. In some cases, the therapeutically effective level of a polypeptide in the bloodstream is known based on standard treatments using the approved drugs of the same parent polypeptide. In other cases, such a therapeutic effective level can be empirically determined. For example, the therapeutically effective amount can be deduced from various in vitro and in vivo activity assays. Hence, the oral dosage formulation required to effect absorption of the polypeptide into the blood at levels sufficient to elicit an activity can be determined. For example, the Examples herein exemplify the assessment of oral dosage formulations required to effect absorption of the polypeptide into the blood such that the amount of polypeptide in the blood is sufficient for an activity (i.e. anti-viral or proliferation). In another example, animal models of a disease or condition can be used in order to assess the oral dosage formulation required to have an ameliorating or therapeutic effect on a disease or condition. For example, the Examples show a study assessing the effects of oral administration of dosage formulations of growth hormone in hypophysectomized rats. Hypophysectomized rat have been used as a model to study the effects of growth hormone deficiency on bone. Thus the use of such an animal model permits assessment of the oral dosage formulation required for absorption into the bloodstream of a therapeutic amount of polypeptide required to overcome such deficiency. Typically, such results in animal models can be extrapolated to humans. Thus, by virtue of their resistance to proteases in the digestive tract, particularly proteases in the esophagus, stomach and intestine, such dosage formulations containing protease resistant therapeutic proteins permit sustained release and absorption of the proteins into the blood stream following oral administration at amounts that are therapeutically effective. The advantage of an oral dosage formulation of a therapeutic protein over an injectable protein is that it could lead to increased patient compliance and savings related to medical assistance and patient care, devices for injection, manufacturing and purification of the protein and storage and transportation of products. For example, one of the drawbacks to injectables is the frequency of injection required, which is deterrent to patient compliance. While the development of some therapeutic proteins has focused on modifying polypeptides in order to decrease the frequency of administration (i.e. by pegylation, glycosylation, albumation, conjugating to Fc), a decreased frequency of administration is not a prerequisite for orally administered proteins, particularly those in the forms of tablets and capsules that can be easily and efficiently administered simply by swallowing (i.e. orally taking) the dosage formulation for release in the gastrointestinal tract. In some dosing regimes, however, the dosage formulations can be administered at a decreased frequency if desired.

Also, protease resistant polypeptides contained in the dosage formulations provided herein are modified with only a few amino acid changes (in many cases only a single change) in the primary sequence of the polypeptide. The mutations in the primary sequence themselves render the protease resistant to polypeptides. Further, because the polypeptide only contains a few changes, it can retain its activity to levels equal to, or in some cases greater than, the activity of the same polypeptide absent the mutations. This has advantages for several reasons. First, this means that the dosage formulation required to achieve a therapeutic effect is not limited by any change (i.e. decrease) of activity of the polypeptide. This is a problem with many therapeutic polypeptides, such as, for example, pegylated polypeptides, where the modification to the polypeptide renders the protein less active. Thus, to achieve a therapeutic effect, such therapeutic polypeptides must be administered at a higher dose.

Second, a change to only the primary sequence of the polypeptide means that there is no other processing requirements or requirements for mixing the therapeutic protein with other compounds in the formulation to effect protease resistance or absorption of the polypeptide into the bloodstream. Accordingly, the manufacturing of the dosage formulations is simplified. For example, as exemplified herein in the examples, tablets containing only the lyophilized protein can be manufactured for oral administration, i.e. the protease resistant proteins are orally available per se. As noted, it also is advantageous to include the dosage formulation in an enteric coating in order to further increase resistance of the polypeptides to protease, particularly upon route to the gastrointestinal tract upon oral administration.

### C. ORAL DOSAGE FORMULATIONS

Protease-resistant therapeutic polypeptides provided herein are administered orally in an amount such that therapeutically effective amount of the therapeutic polypeptide is delivered to the bloodstream. The amount is effective the treatment of a disease, disorder or condition, and depends upon the indication treated. The therapeutically effective amounts of the protease-resistant therapeutic polypeptides are provided dosage formulation, which contain one or a plurality of unit dosages. A unit dosage is a fractional amount up to a full amount of therapeutically effective protein. Unit dosages contain any suitable form of a protease-resistant therapeutic polypeptides, and optionally contain pharmaceutically acceptable carriers and coatings, such as enteric coatings.

The amount of protease-resistant therapeutic polypeptide in a dosage formulation generally are higher, generally about or 6-200 times, such as 10-100 times, including, for example, 15-50 times, 20-30 times, typically 15-40 times higher (depending upon the therapeutic protein and indication treated and the formulation) per day, than amounts that in a dosage formulation of a therapeutic protein, that is not modified to be protease resistant, for subcutaneous administration for the same indication. Dosage regimens for oral administration can differ from those for subcutaneous administration. For example, therapeutic proteins administered subcutaneously several times a week, can be administered daily in an oral regimen. Hence, total dosages in a particular time period, such as a week, can be substantially higher, such as 10-500 times more for oral administration compared to a the total amount administered in a week subcutaneously.

The amount of a protease-resistant polypeptide depends on absorption, inactivation and excretion rates of the active compound, the dosage schedule, and amount administered as well as other factors known to those of skill in the art. Its amount in a dosage unit also can vary but must be an amount that is suitable for oral ingestion. As described further herein, dosages can be determined empirically using dosages known in the art for administration of unmodified therapeutic polypeptides, and comparisons of properties and activities (e.g., protease resistance and activities) of the protease-resistant polypeptide compared to the unmodified and/or native polypeptide. As provided herein, oral dosage formulations of protease-resistant therapeutic polypeptide are higher, typically 10-600-fold, generally 10-100, 10-50, 15-40, 15-30, 20-40 fold, than a comparable dosage formulation of the therapeutic polypeptide not modified for protease resistance.

Any therapeutic protein can be modified to be protease resistant and provided in for oral administration as described herein. Exemplary oral dosage formulations, and dosage units therefor, for exemplary protease-resistant polypeptides, such as interferon-α (E41Q) (SEQ ID NO: 1995) and growth hormone (Y42I) (SEQ ID NO: 1318) are provided herein (see, e.g., the Examples below). Exemplary dosage units and oral dosage formulations for interferon-α (E41Q) are presented in Example 3 and Example 4. Exemplary dosage units and oral dosage formulations for growth hormone (Y42I) are presented in Example 22.

1. Oral Dosage Units

Typically, the oral dosage formulations are provided as unit dosages, such as tablets and/or capsules that contain a single dosage amount or a fraction thereof. The oral dosage formulations are provided as dosage formulations that contain a sufficient amount of the therapeutic protein as a daily dosage to delivery a therapeutically effective amount to the bloodstream upon oral administration. The dosage formulations can contain one or a plurality of dosage units.

Oral dosage formulations of protease-resistant therapeutic proteins are provided. The formulations provide an amount of each protein that is sufficient to achieve a therapeutically effective level in the bloodstream upon administration. The therapeutically effective level depends upon the protein and indication treated. The formulations typically contain unit dosages, such as tablets and/or capsules, and can contain one or a plurality thereof.

Dosage units provided herein, include a protease-resistant polypeptide formulated alone or in combination with appropriate additives or excipients. For oral administration, the dosage units provided herein containing a protease-resistant polypeptide can take the form of, for example, tablets, pills, or capsules prepared by conventional means with pharmaceutically acceptable excipients. Compositions of protease-resistant polypeptides for oral administration can be formulated with additional factors, such as one or more therapeutic agents, useful in the disease or disorder to be treated. Such combinations of therapeutic agents for use in the compositions provided are described elsewhere herein. The pharmaceutical compositions provided herein can be formulated for single dosage (direct) administration or for dilution or other modification. The amount of the polypeptide in the dosage unit or oral dosage formulations is effective for delivery of an amount, upon administration, that is effective for the intended treatment. The compositions can be formulated for single dosage administration or for multiple dosage administration. The dosage unit formulations provided herein include delayed-release formulations and sustained-release formulations.

2. Preparation of the Oral Dosage Formulation and Oral Dosage Units

Oral dosage formulations of protease-resistant polypeptides provided herein can be formulated in any conventional manner by mixing a selected amount of the polypeptide with one or more physiologically acceptable carriers or excipients. To formulate a composition, the weight fraction of a compound or mixture thereof is dissolved, suspended, dispersed, or otherwise mixed in a selected vehicle in a predetermined amount. The amount depends upon the number of dosage units to be administered per day or week and upon the therapeutic protein and condition treated. Selection of the carrier or excipient is within the skill of the administering professional and can depend upon a number of parameters. These include, for example, the form for oral administration (e.g., tablet, pill or capsule) and the disorder to be treated. Pharmaceutical carriers or vehicles suitable for administration of the compounds provided herein include any such carriers known to those skilled in the art to be suitable for oral administration. A variety of pharmaceutically acceptable excipients are known in the art (see, e.g., A. Gennaro (2000) “Remington: The Science and Practice of Pharmacy,” 2nd edition, Lippincott, Williams, & Wilkins; Pharmaceutical Dosage Forms and Drug Delivery Systems (1999) H. C. Ansel et al., eds., 7th ed., Lippincott, Williams, & Wilkins; and Handbook of Pharmaceutical Excipients (2000) A. H. Kibbe et al., eds., 3rd ed. Amer. Pharmaceutical Assoc., which each describe exemplary excipients). Hence, exemplary additives and excipients that can be used in oral dosage formulations provided include, but are not limited to, binding agents (e.g., pregelatinized maize starch, potato starch, acacia, polyvinylpyrrolidone, magnesium aluminum silicate, pectins, alginates, gelatin, gum such as gum arabic, gum tragacanth, guar gum, or a cellulose derivative such as ethyl cellulose, methyl cellulose, hydroxypropyl methylcellulose, hydroxypropylcellulose, or carboxymethyl cellulose); fillers (e.g., lactose, microcrystalline cellulose or calcium hydrogen phosphate); lubricants (e.g., magnesium stearate, calcium sulfate, talc or silica); disintegrants (e.g., potato starch, sodium starch glycolate, sodium carboxymethyl starch, agar, alginic acid and the sodium salt thereof, croscarmelose, crospovidone, clays, and ion exchange resins.); wetting agents (e.g., sodium lauryl sulphate); and diluents, buffering agents, solvents, co-solvents, surfactants, preservatives, pharmaceutical-grade dyes or pigments, viscosity agents, polymers, resins, plasticizers, and flavoring agents.

The protease-resistant therapeutic polypeptide is provided in solid or other suitable form, such as powder, crystal or gel, and mixed with an excipient(s). Procedures for preparation of the lyophilized powder, particles, and granules of the protease-resistant polypeptide for formulating an oral composition, such as a compressed tablet or pill, or for filling capsules are known, and/or can be modified to produce compositions with desired properties, including but not limited to, particle size, tackiness, yield, and dimer formation of the polypeptide. Such procedures can include alteration of lyophilization buffer conditions, such as adjustment of pH, salt content, sugar content and choice (e.g., mannitol or sucrose) or addition of other stabilization agents, such as for example, L-arginine. Manipulations of such conditions are within the knowledge of one of skill in the art. Exemplary procedures for modifying such conditions are presented herein in the Examples below and can used to modify the properties to resulting lyophilized protease-resistant polypeptide including, for example, particle size, tackiness, yield, and dimer formation of the polypeptide.

Further, moisture content of the oral composition can be varied with the addition or removal of one or more drying steps in the production of the lyophilized powder, particles, or granules of the protease-resistant polypeptide or in the preparation of the compressed tablet or pill.

In addition to the above mentioned variations, in order to obtain the desired release pattern, the excipients also can be varied, as long as they do not affect the activity of the particular protease-resistant polypeptide selected.

a. Delivery Control

As described herein, the site or the rate of delivery of the protease-resistant polypeptides in the intestinal tract can be controlled by one skilled in the art, by manipulating any one or more of the following: the type of the coating, and the concomitant desirable thickness and permeability, or swelling properties, of the coating; the time-dependent conditions of the coating itself or within the coated tablet, capsule, particle, bead, or granule; the particle size of the granulated protease-resistant polypeptide or dissolution properties of tablet formulation, such as the packing density of the tablet or thickness of the tablet; and the pH-dependent conditions of the coating itself and/or within the coated tablet, capsule, particle, bead, or granule. In particular, the solubility, acidity, and susceptibility to hydrolysis of the different protease-resistant polypeptides, and the properties of the lyophilized powder can be used as guidelines for the proper choice. In addition, suitable pH-conditions might be established within the coated tablets, particles, granules, or beads by adding a suitable buffer to the active ingredient in accordance with the desired release pattern. Further, some protease-resistant polypeptides are moisture sensitive and perform better if delivered in particular forms, such a tablet dosage form.

The dosage units in the oral dosage formulations provided herein permit reliable delivery of the therapeutic protein to the upper or lower intestinal tract, or any part thereof. If delivery to the lower intestinal tract only is desired, oral dosage formulations described here can effect such delivery, thereby prohibiting the exposure of protease-resistant polypeptide in the mucosal and epithelial tissues of the mouth, pharynx, and/or esophagus and inhibiting its release in the stomach. The oral dosage formulations render the protease-resistant polypeptide readily available for absorption from the lower gastrointestinal tract. Accordingly, oral dosage formulations suitable for use herein can be enteric-coated delayed-release formulations or enteric-coated sustained-release formulations.

Exemplary methods suitable for use in coating a compressed tablet containing a protease-resistant-polypeptide which will effect the delivery of the polypeptide to the small intestine are provided herein in the Examples. The Examples provided herein describe oral dosage formulations for exemplary protease-resistant polypeptides, such as interferon-α (E41Q) (SEQ ID NO: 1995) and growth hormone (Y42I) (SEQ ID NO: 1318). Exemplary oral dosage formulations for interferon-α (E41Q) are presented in Example 3 and Example 4. Exemplary oral dosage formulations for growth hormone (Y42I) are presented in Example 22.

i. Enteric Coatings

Enteric-coated oral dosage formulations of protease-resistant polypeptides can help effect delivery to the lower intestine (e.g., to the small intestine) of a human or other mammal. The enteric-coated oral dosage formulations contain an amount of a protease-resistant polypeptide and pharmaceutically-acceptable excipients. The enteric-coating of the tablet or capsule not soluble in the fluids of the mouth, the pharynx, the esophagus, or the stomach and thereby prohibits the release of the protease-resistant polypeptide until the lower is intestine is reached, e.g., the small intestine. In addition to coated tablets and capsules, oral dosage formulations can take other forms, such as a gelatin capsule that contains beads or small particles of the protease-resistant polypeptide which have themselves been enterically coated.

Enteric coatings are typically made with suitable pharmaceutical excipients including, but not limited to, Eudragit® L, Eudragit® L-100, Eudragit® S, Eudragit® S-100, Eudragit® L 30 D-55, Eudragit® 100-55, hydroxypropyl methylcellulose acetate succinate (HPMCAS), hydroxypropyl methyl cellulose phthalate (HPMCP), cellulose acetate phthalate (CAP), polyvinyl phthalic acetate (PVPA), cellulose acetate trimellatate, polyethylene glycol 400-8000, triacetin, dibutyl phthalate, acetylated monoglycerides, shellac, triethyl citrate, talc, and iron oxide.

An exemplary oral dosage formulation of a protease-resistant polypeptide is an enteric-coated compressed tablet, which is formulated from granules, particles, or a lyophilized powder of the protease-resistant polypeptide. Tablets can be made combining, mixing or otherwise adding the protease-resistant polypeptide to suitable pharmaceutical excipients as exemplified above. That mixture is then compressed into a tablet utilizing various tableting techniques available to those skilled in the art. The compressed tablet is then coated with an enteric-coating material, utilizing methods well known in the art, including numerous spraying techniques available to those skilled in the art.

An exemplary oral dosage formulation which effects delivery to the small intestine contains a protease-resistant polypeptide and has a pH dependent enteric coating material made from a partly methyl esterified methacrylic acid polymer. The solid oral dosage formulation can be in the form of an enteric coated compressed tablet made of granules, particles, or a lyophilized powder of the protease-resistant polypeptide. In a particular example the protease-resistant polypeptide is produced in the form of a lyophilized powder.

Any enteric coating which is insoluble at a pH below 5.5 (i.e., that generally found in the mouth, pharynx, esophagus and stomach), but soluble at pH 5.5 or above (i.e., that present in the small intestine and the large intestine) can be used in the oral dosage formulations provided. Accordingly, when it is desired to effect the delivery of the protease-resistant polypeptide to the small intestine, any enteric coating is suitable, which is wholly- or partially-insoluble at a pH below 5.5 and soluble at pH 5.5 or above.

The partly methyl esterified methacrylic acid polymer can be applied to the compressed tablet, the gelatin capsule and/or the beads, particles or granules of the protease-resistant polypeptide in a sufficient thickness so that the entire coating does not dissolve in gastrointestinal fluids at a pH below 5.5, but does dissolve at a pH of 5.5 or above. The dissolution or disintegration of the excipient coating generally does not occur until the entry of the coated dosage form into the small intestine. In particular, there is substantially no release of the protease-resistant polypeptide upstream of the duodenum.

Any anionic polymer exhibiting the requisite pH-dependent solubility profile can be used as an enteric coating in the practice of the present methods to achieve delivery of the protease-resistant polypeptide to the lower intestine. The coating chosen must be compatible with the particular protease-resistant polypeptide selected. Exemplary polymers for use in the dosage forms provided are anionic carboxylic polymers. In a particular example, the polymers are acrylic polymers, such as partly methyl-esterified methacrylic acid polymers, in which the ratio of anionic free carboxyl groups to ester groups is about 1:1.

An exemplary methacrylic acid-methyl methacrylate copolymer, which is suitable for use in coating the oral dosage formulations and/or the granules, particles or beads of the protease resistant-polypeptide, which can be employed in the method of treatment described herein, either alone or in combination with other coatings, is Eudragit L®, particularly Eudragit® L-100, manufactured by Rohm Pharma GmbH, Weiterstadt, West Germany. In Eudragit® L-100, the ratio of free carboxyl groups to ester groups is approximately 1:1. Further, the copolymer is known to be insoluble in gastrointestinal fluids having a pH below 6.0, generally 1.5-6.0, i.e., that generally present in the fluid of upper gastrointestinal tract (e.g., stomach <pH 5.5) and duodenum (e.g., pH 5.5-6.0), but readily soluble at pH above 6.0, i.e., that generally present in the fluid of the lower gastrointestinal tract, particularly in the jejunum of the small intestine.

Other formulations of Eudragit® L include Eudragit® L 30 D-55 which allow delivery the protease-resistant polypeptide at pH>5.5. Such coating can be used to effect delivery of the protease-resistant polypeptide in the duodenum, prior to entry into the jejunum of the small intestine.

Another exemplary methacrylic acid-methyl methacrylate copolymer, which is suitable for use in coating the oral dosage formulations and/or the granules, particles or beads of the protease resistant-polypeptide, which can be employed in the method of treatment described herein, either alone or in combination with other coatings, is Eudragit® S-100, manufactured by Rohm Pharma GmbH, Weiterstadt, West Germany. Eudragit® S differs from Eudragit® L-100 only insofar as the ratio of free carboxyl groups to ester groups is approximately 1:2. Eudragit® S-100 is also, like Eudragit® L-100, is insoluble at pH below 5.5, generally 1.5-5.5, such as that present in gastric juice, but, unlike Eudragit® L-30-D, is poorly soluble in gastrointestinal fluids having a pH of 5.5-7.0, such as that present in upper small intestinal juice. The copolymer is soluble at pH 7.0 and above, i.e., that generally present in the lower small intestine (i.e., ileum) and colon.

Eudragit® S-100 can be used alone as a coating which would provide delivery of the protease-resistant polypeptide at the ileum via a delayed-release mechanism. In addition, Eudragit® S-100, being poorly soluble in intestinal juice below pH 7.0, can be used in combination with Eudragit® L coatings such as Eudragit® L-100 or Eudragit® L 30 D-55, in order to effect a delayed-release, composition that can be formulated to deliver the active ingredient at various segments of the intestinal tract; the more Eudragit® L used, the more proximal release and delivery begins and the more Eudragit® S used, the more distal release and delivery begins.

The coating can, contain a plasticizer and possibly other coating excipients such as coloring agents, talc, and/or magnesium stearate, many of which are well known in the coating art. In particular, anionic carboxylic acrylic polymers can contain 10-25% by weight of a plasticizer, e.g., dibutyl phthalate, polyethylene glycol, triethyl citrate, triacetin, glyceryltriacetate, acetyltriethylcitrate, dibutyl sebacate, diethylphthalate, polyethylene glycol having a molecular weight in the range of 200 to 8000, glycerol, castor oil, copolymers of propylene oxide and ethylene oxide, or mixtures thereof. Conventional coating techniques such as spray or pan coating can be employed to apply the coating. As described herein, the coating thickness must be sufficient to ensure that the oral dosage formulation remains intact until the desired site of topical delivery in the lower intestinal tract is reached.

As described herein, the solid oral dosage formulation can be in the form of a coated compressed tablet which contains granules, particles, or a lyophilized powder of the protease-resistant polypeptide or of a gelatin capsule, coated or uncoated, which contains beads of the protease-resistant polypeptide, which themselves are enteric coated.

In an exemplary coating method described herein utilizing methylacrylate copolymers, when the desired site of delivery is the small intestine, a coating thickness of between 20 and 100 microns usually is required. In a particular example, the coating thickness is between 30 and 75 microns. In another particular example, the coating thickness is between 30 and 50 microns. The thickness of coating required on the tablets or capsules will depend upon the dissolution profile of the particular coating materials and possibly also upon the dissolution profile of the enteric coating on the capsule. It is well within the ability of one of skill in the art to determine by standard testing procedure the optimum thickness of a particular coating required for a particular dosage form.

ii. Multiple Coatings and Sub-Coatings

In another non-limiting example of an oral dosage formulation, protease-resistant polypeptides can be formulated with one or more pharmaceutical excipients and coated with an enteric coating (see, e.g., U.S. Pat. No. 6,346,269). For example, a solution containing a solvent, a protease-resistant polypeptide, and a stabilizer is coated onto a core containing pharmaceutically acceptable excipients, to form an active agent-coated core; a sub-coating layer is applied to the active agent-coated core, which is then coated with an enteric coating layer. The core generally includes the protease-resistant polypeptide and one or more pharmaceutically acceptable additives or excipients as described herein. The sub-coating layer can contain one or more of an adhesive, a plasticizer, and an anti-tackiness agent. Suitable anti-tackiness agents include, but are not limited to, talc, stearic acid, stearate, sodium stearyl fumarate, glyceryl behenate, kaolin and aerosil. Exemplary adhesives include polyvinyl pyrrolidone (PVP), gelatin, hydroxyethyl cellulose (HEC), hydroxypropyl cellulose (HPC), hydroxypropyl methyl cellulose (HPMC), vinyl acetate (VA), polyvinyl alcohol (PVA), methyl cellulose (MC), ethyl cellulose (EC), hydroxypropyl methyl cellulose phthalate (HPMCP), cellulose acetate phthalates (CAP), xanthan gum, alginic acid, salts of alginic acid, Eudragit®, copolymer of methyl acrylic acidlmethyl methacrylate with polyvinyl acetate phthalate (PVAP). Suitable plasticizers include glycerin, polyethylene glycol, triethyl citrate, tributyl citrate, propanyl triacetate and castor oil. Exemplary enteric soluble coating materials include hydroxypropyl methylcellulose acetate succinate (HPMCAS), hydroxypropyl methyl cellulose phthalate (HPMCP), cellulose acetate phthalate (CAP), polyvinyl phthalic acetate (PVPA), Eudragit® and shellac.

b. Other Delivery Additives and Carriers

In other non-limiting examples of an oral dosage formulation, protease-resistant polypeptides can be formulated together with any of the following: microgranules (see, e.g., U.S. Pat. No. 6,458,398); biodegradable macromers (see, e.g., U.S. Pat. No. 6,703,037); biodegradable hydrogels (see, e.g., Graham and McNeill (1989) Biomaterials 5:27-36); biodegradable particulate vectors (see, e.g., U.S. Pat. No. 5,736,371); bioabsorbable lactone polymers (see, e.g., U.S. Pat. No. 5,631,O15); slow release protein polymers (see, e.g., U.S. Pat. No. 6,699,504; Pelias Technologies, Inc.); a poly(lactide-co-glycolide/polyethylene glycol block copolymer (see, e.g., U.S. Pat. No. 6,630,155; Atrix Laboratories, Inc.); a composition containing a biocompatible polymer and particles of metal cation-stabilized agent dispersed within the polymer (see, e.g., U.S. Pat. No. 6,379,701; Alkermes Controlled Therapeutics, Inc.); and microspheres (see, e.g., U.S. Pat. No. 6,303,148; Octoplus, B.V.).

In other non-limiting examples of an oral dosage formulation, protease-resistant polypeptides can be formulated together with any of the following: a carrier such as, for example, EmisphereQ (Emisphere Technologies, Inc.); TIMERx, a hydrophilic matrix combining xanthan and locust bean gums which, in the presence of dextrose, form a strong binder gel in water (Penwest); Geminex™ (Penwest); Procise™ (Claxo SmithKline); SAVIT™ (Mistral Pharma Inc.); RingCap™ (Alza Corp.); Smartrix® (Smartrix Technologies, Inc.); SQZgel™ (MacroMed, Inc.); Geomatrix™ (Skye Pharma, Inc.); Oros® Tri-layer (Alza Corporation).

In other non-limiting examples of an oral dosage formulation, protease-resistant polypeptides can be formulated as described in U.S. Pat. No. 6,296,842 (Alkermes Controlled Therapeutics, Inc.); and U.S. Pat. No. 6,187,330 (Scios, Inc.).

In other non-limiting examples of an oral dosage formulation, protease-resistant polypeptides can contain an intestinal absorption enhancing agent. Suitable intestinal absorption enhancers include, but are not limited to, calcium chelators (e.g., citrate, ethylenediamine tetracetic acid); surfactants (e.g., sodium dodecyl sulfate, bile salts, palmitoylcamitine, and sodium salts of fatty acids); and toxins (e.g., zonula occludens toxin).

The modified polypeptides for use in the oral dosage formulations provided herein exhibit increased resistance to proteolysis in the gastrointestinal tract. Thus, preparations for oral administration can be suitably formulated without the use of protease inhibitors, such as a Bowman-Birk inhibitor, a conjugated Bowman-Birk inhibitor, aprotinin and camostat. Such compounds, however, are not excluded from use in the compositions provided.

### D. PROTEASE-RESISTANT POLYPEPTIDES FOR ORAL DOSAGE FORMULATIONS

1. Protease Resistant Polypeptides

Protease-resistant polypeptides for use in the oral dosage formulations provided herein include any polypeptide that has increased resistance to one or more proteases compared to the native form of the polypeptide due to replacement of one or more amino acids in the primary amino acid sequence of the polypeptide. Protease-resistant polypeptides for use in the oral dosage formulations provided herein include therapeutic polypeptides. Such polypeptides include, but are not limited to, protease-resistant forms of polypeptides selected from exemplary protein families, such as, but not limited to, coagulation factors, cytokines, growth factors, hormones, hydrolases, immunoglobulins, inhibitor proteins, nuclear proteins, and proteases. The proteases to which the proteins are resistant or more resistant that prior to modification include those are those present in the bloodstream.

Numerous such polypeptides are known and described (see, e.g., U.S. Patent Application Publication Nos. 2004/0132977-A1, US 2005/0202438-A1, US 2006/0251619-A1, US 2006/0094655-A1; U.S. Provisional Application Nos. 60/787,208, 60/861,615; International PCT Application Publication Nos. WO2006/020580, WO 2004/022593, WO 2004/022747, WO2006048777; and International PCT Application No. IB2006/002034).

Exemplary protease-resistant polypeptides for use in the oral dosage formulations provided herein are provided in SEQ ID NOS: 2-46, 48-84, 86-117, 119-146, 148-660, 662-808, 810-859, 861-916, 918-945, 947-994, 996-1017-, 1019-1043, 1045-1079, 1081-1125, 1127-1147, 1149-1180, 1182-1214, 1216-1259, 1261-1885, 1887-1981, 1983-2066, and 2176-2182 as well allelic and species variants thereof. For exemplification purposes, the Examples, below, detail studies using oral formulations interferon-α (IFN-α) and growth hormone (hGH). The Examples show how dosages can be selected for a particular therapeutic protein, and also demonstrate the effectiveness of oral tablet and capsule formulations of IFN-α and hGH.

Exemplary protease-resistant polypeptides for use in the oral dosage formulations provided include species and allelic variants of the polypeptides exemplified herein. Modifications for protease resistance in the primary sequence of amino acids with respect to an exemplary polypeptide provided herein also can be made at corresponding positions in species or allelic variants of the polypeptide. Such modified species and allelic variants also can be used in the oral dosage formulations provided.

Modifications for protease resistance in the primary sequence of amino acids at corresponding amino acid positions can be made in polypeptides that are species or allelic variants or homologous to or are members of the same protein family as the polypeptides exemplified herein. For example, by virtue of the knowledge of the 3-dimensional structural amino acid positions within exemplary protease-resistant mutants provided herein that confer higher resistance to a challenge with either proteases or blood lysate or serum, while maintaining or improving the requisite biological activity, the corresponding structurally related (e.g., structurally similar) amino acid residues on a variety of other polypeptides can be identified. Such modified homologous polypeptides or modified protein family members also can be used in the oral dosage formulations provided (see, e.g., U.S. application Ser. No. 10/658,834, which describes such methods).

Numerous methods are well known in the art for identifying structurally related amino acid positions with 3-dimensionally structurally homologous proteins. Exemplary methods include, but are not limited to: CATH (Class, Architecture, Topology and Homologous superfamily) which is a hierarchical classification of protein domain structures based on four different levels (Orengo et al., Structure, 5(8):1093-1108 (1997)); CE (Combinatorial Extension of the optimal path), which is a method that calculates pairwise structure alignments (Shindyalov et al., Protein Engineering, 11(9):739-747 (1998)); FSSP (Fold classification based on Structure-Structure alignment of Proteins), which is a database based on the complete comparison of all 3-dimensional protein structures that reside in the Protein Data Bank (PDB) (Holm et al., Science, 273:595-602 (1996)); SCOP® (Structural Classification of Proteins) Database, which provides a descriptive database based on the structural and evolutionary relationships between all proteins whose structure is known (Murzin et al., J. Mol. Biol., 247:536-540 (1995)); and VAST (Vector Alignment Search Tool) Database, which compares newly determined 3-dimensional protein structure coordinates to those found in the MMDB/PDB database (Gibrat et al., Current Opinion in Structural Biology, 6:377-385 (1995)).

In one example, publicly available programs, such as the program referred to as TOP (Lu, G., J. Appl. Cryst., 33:176-189 (2000)) can be used for protein structure comparison. This program runs two steps for each protein structure comparison. In the first step topology of secondary structure in the two structures is compared. The program uses two points to represent each secondary structure element (alpha helices or beta strands) then systematically searches all the possible super-positions of these elements in 3-dimensional space (defined as the root mean square deviation—rmsd, the angle between the two lines formed by the two points and the line-line distance). The program searches to determine whether additional secondary structure elements can fit by the same superposition operation. If secondary structures that can fit each other exceed a given number, the programidentifies the two structures as similar. The program gives as an output a comparison score called “Structural Diversity” that considers the distance between matched α-carbon atoms and the number of matched residues. The lower the “Structural Diversity” score, the more the two structures are similar.

Among the protease-resistant polypeptides for use in oral dosage formulations provided herein are polypeptides modified to increase stability to conditions amendable to oral delivery, which includes administration to gastrointestinal tract, but also resistance to proteases in the bloodstream. Such modifications can include those that result in a detectable increase in protein-half life under one or more conditions such as exposure to saliva, exposure to proteases in the gastrointestinal tract, and exposure to particular pH conditions, such as the low pH of the stomach and/or pH conditions in the intestine. Modifications can include resistance to one or more proteases including pepsin, trypsin, chymotrypsin, elastase, aminopeptidase, gelatinase B, gelatinase A, α-chymotrypsin, carboxypeptidase, endoproteinase Arg-C, endoproteinase Asp-N, endoproteinase Glu-C, endoproteinase Lys-C, and trypsin, luminal pepsin, microvillar endopeptidase, dipeptidyl peptidase, enteropeptidase, hydrolase, NS3, elastase, factor Xa, Granzyme B, thrombin, trypsin, plasmin, urokinase, tPA and PSA. Modifications also can include increasing overall stability to potentially denaturing or conformation-altering conditions such as thermal tolerance, and tolerance to mixing and aeration (e.g., chewing).

Protease-resistant polypeptides can be generated or identified or produced by any method known in the art including, but not limited to, directed evolution methods such as 2D- and 3D-scanning mutagenesis methods for protein rational evolution (see, e.g., U.S. Publication Nos. US 2005/0202438 A1 and US-2004/0132977-A1 and published International applications WO 2004/022593 and WO 2004/022747). Modification of polypeptides for suitability for oral delivery can include removal of proteolytic digestion sites and/or increasing the overall stability of the protein structure. Such polypeptide variants exhibit increased protease-resistance compared to an unmodified and/or native polypeptide in one or more conditions for oral delivery, which include exposure to proteases.

In one example, the protease resistance of the variant polypeptides for use in oral dosage formulations provided herein is assessed by measuring protein concentration or residual activity in the presence of one or more proteases such as pepsin, trypsin, chymotrypsin, elastase, aminopeptidase, gelatinase B, gelatinase A, α-chymotrypsin, carboxypeptidase, endoproteinase Arg-C, endoproteinase Asp-N, endoproteinase Glu-C, endoproteinase Lys-C, and trypsin, luminal pepsin, microvillar endopeptidase, dipeptidyl peptidase, enteropeptidase, hydrolase, NS3, elastase, factor Xa, Granzyme B, thrombin, trypsin, plasmin, urokinase, tPA and PSA. The variant polypeptide can be mixed with one or more proteases and then assessed for activity and/or protein structure after a suitable reaction time. Assessment of residual activity also can include exposure to increased temperature, such as the body temperature of a subject; exposure to gastric juices and/or simulated gastric juices; exposure to particular pH conditions and/or a combination of two or more conditions. Following exposure to one or more conditions, activity and/or assessment of protein structure can be used to assess the stability and/or resistance to proteolytic digestion of the variant polypeptide in comparison to an appropriate control (e.g., an unmodified and/or native polypeptide).

Protease-resistant forms of therapeutic polypeptides can be administered to a subject for the prevention or treatment of any disease or condition for which the native form of the polypeptide has been used for the prevention or treatment of such disease(s) or condition(s).

Modified polypeptides for use in the oral dosage formulations provided also include protease-resistant polypeptides that have been further modified by glycosylation, carboxylation, hydroxylation, hasylation, carbamylation, sulfation, phosphorylation, and/or PEGylation.

Modified polypeptides for use in the oral dosage formulations provided also include protease-resistant polypeptides that have been further modified by one or more amino acid modifications that contribute to, for example, increased activity, altered immunogenicity, stability, thermal tolerance, protease-resistance, glycosylation, carboxylation, hydroxylation, hasylation, carbamylation, sulfation, phosphorylation, and/or PEGylation. Such amino acid modifications can be natural amino acids, non-natural amino acids or a combination of natural and non-natural amino acids. The protease-resistant polypeptides for use in the oral dosage formulations provided also can be conjugated or expressed as a fusion protein with one or more proteins or peptides, such as albumin.

2. Cytokines

Examples of protease-resistant polypeptides for use in the oral dosage formulations provided herein include polypeptides of the cytokine superfamily. Modified cytokine polypeptides for use in the oral dosage formulations provided contain one or more amino acid replacements at one or more target positions and exhibit greater resistance to proteolysis compared to the cytokine protein without the one or more amino acid replacements.

Cytokines all share a common helix bundle fold, which is used to structurally define the 4-helical cytokine superfamily in the structural classification of the protein database SCOP© (Structural Classification of Proteins; see, e.g., Murzin et al., J. Mol. Biol., 247:536-540, 1995 and “scop.mrc-lmb.cam.ac.uk/scop/”). This superfamily includes three different families: 1) the interferons/interleukin-10 protein family; 2) the long-chain cytokine family; and 3) the short-chain cytokine family. Although the degree of similarity among the underlying amino acid sequences of these cytokines does not appear high, their corresponding 3-dimensional structures present a high level of similarity. Effectively, the best structural similarity is obtained between two 3-dimensional protein structures of the same family in the 4-helical cytokine superfamily.

An exemplary distinct feature of cytokines from the interferons/interleukin-10 family is an additional (fifth) helix. This family includes, for example, interleukin-10 (IL-10; SEQ ID NO: 809), interferon beta (IFN-β; SEQ ID NO: 147), interferon alpha-2a (IFNα-2a; SEQ ID NO: 2162), interferon alpha-2b (IFNα-2b; SEQ ID NO: 2067), and interferon gamma (IFN-γ; SEQ ID NO: 661).

The long-chain cytokine protein family includes, among others, granulocyte colony stimulating factor (G-CSF; SEQ ID NO: 47), leukemia inhibitory factor (LIF; SEQ ID NO: 1148), growth hormone (hGH; SEQ ID NO: 1260), ciliary neurotrophic factor (CNTF; SEQ ID NO: 1), leptin (SEQ ID NO: 1126), oncostatin M (SEQ ID NO: 1181), interleukin-6 (IL-6; SEQ ID NO: 1080) and interleukin-12 (IL-12; SEQ ID NO: 860).

The short-chain cytokine protein family includes, among others, erythropoietin (EPO; SEQ ID NO: 1886), granulocyte-macrophage colony stimulating factor (GM-CSF; SEQ ID NO: 85), interleukin-2 (IL-2; SEQ ID NO: 946), interleukin-3 (IL-3; SEQ ID NO: 995), interleukin-4 (IL-4; SEQ ID NO: 1018), interleukin-5 (IL-5; SEQ ID NO: 1044), interleukin-13 (IL-13; SEQ ID NO: 917), Flt3 ligand (SEQ ID NO: 118) and stem cell factor (SCF; SEQ ID NO: 1215).

Modified cytokines for use in the oral dosage formulations provided include allelic and species variants of such cytokines containing corresponding modifications for protease resistance.

a. Interferon/Interleukin-10 Family

As set forth above, provided herein are oral dosage formulations that contain modified cytokines that contain one or more amino acid replacements at one or more target positions in polypeptides of the interferon/interleukin-10 family.

In a particular example of an oral dosage formulation, the protease-resistance polypeptide is a modified interferon, such as interferon-α (IFN-α). Exemplary IFN-α protease-resistant polypeptides include one or more amino acid modifications in an IFN-α2b polypeptide, where the one or more amino acid replacements lead to greater resistance to proteases. Such modifications include, but are not limited to, modifications shown in Table 2, where the replacements are made compared to the sequence of amino acids set forth in SEQ ID NO: 2067. In reference to such mutants, the first amino acid (one-letter abbreviation) corresponds to the amino acid that is replaced, the number corresponds to position in the IFN-α2b polypeptide sequence with reference to SEQ ID NO: 2067, and the second amino acid (one-letter abbreviation) corresponds to the amino acid selected that replaces the first amino acid at that position. In Table 2, the sequence identifier (SEQ ID NO.) for the accompanying Sequence Listing is in parenthesis next to each substitution. Such exemplary polypeptides can be used in the oral dosage formulations provided herein.

TABLE 2
Interferon-α (IFN-α) Modifications to Increase Resistance to Proteolysis

| P4A (1983) | L3V (1984) | R12H (1985) | R13H (1986) | M16V (1987) |
|---|---|---|---|---|
| M16I (1988) | R22H (1989) | F27I (1990) | F27V (1991) | L30I (1992) |
| K31Q (1993) | R33H (1994) | E41Q (1995) | E41H (1996) | E58Q (1997) |
| E58H (1998) | K70T (1999) | E78Q (2000) | E78H (2001) | Y89I (2002) |
| E107Q (2003) | E107H (2004) | P109A (2005) | L110V (2006) | M111I (2007) |
| E113Q (2008) | E113H (2009) | L117V (2010) | L117I (2011) | K121Q (2012) |
| K121T (2013) | R125H (2014) | R125Q (2015) | L128V (2016) | L128I (2017) |
| K131Q (2018) | K131T (2019) | E132Q (2020) | E132H (2021) | K133Q (2022) |
| K133T (2023) | K134Q (2024) | Y135H (2025) | Y135I (2026) | P137A (2027) |
| M148V (2028) | M148I (2029) | R149H (2030) | R149Q (2031) | E159Q (2032) |
| E159H (2033) | P4S (2034) | M111V (2035) | Y89H (2036) | L3I (2037) |
| R12Q (2038) | R13Q (2039) | R22Q (2040) | R23H (2041) | R23Q (2042) |
| L30V (2043) | K31T (2044) | R33Q (2045) | K49Q (2046) | K49T (2047) |
| K70Q (2048) | K83Q (2049) | K83T (2050) | E96Q (2051) | E96H (2052) |
| P109S (2053) | L110I (2054) | R120H (2055) | R120Q (2056) | K134T (2057) |
| P137S (2058) | L161V (2059) | L161I (2060) | R162H (2061) | R162Q (2062) |
| K164Q (2063) | K164T (2064) | E165Q (2065) | E165H (2066) | |
| N45D | D94G | G102R | A139G | |
| E41H/Y89H/ | E58Q/F27V | R125H/M111V | E159H/Y89H | K121Q/P109A (2180) |
| N45D (2176) | (2177) | (2178) | (2179) | |
| K121Q/P109A/ | E78H/R33H/ | | | |
| K133Q/ | E58H/L110V | | | |
| G102R (2181) | (2182) | | | |

In addition to IFNα, protease-resistant polypeptides of this family that can be used in the oral dosage formulations include, but are not limited to, interleukin-10 (IL-10; SEQ ID NO: 809), interferon beta (IFN-β; SEQ ID NO: 147), and interferon gamma (IFN-γ; SEQ ID NO: 661) polypeptides. Exemplary protease-resistant polypeptides include one or more amino acid modifications in such polypeptides, where the one or more amino acid replacements lead to greater resistance to proteases. Such modifications include, but are not limited to, modifications shown in Tables 3-5, where the replacements are made compared to the sequence of amino acids set forth in SEQ ID NOS: 809, 147, or 661, respectively. In reference to such mutants, the first amino acid (one-letter abbreviation) corresponds to the amino acid that is replaced, the number corresponds to position in the IL-10, IFN-β or IFN-γ polypeptide sequence with reference to SEQ ID NO: 809, 147, or 661, respectively and the second amino acid (one-letter abbreviation) corresponds to the amino acid selected that replaces the first amino acid at that position. In the tables, the sequence identifier (SEQ ID NO.) for the accompanying Sequence Listing is in parenthesis next to each substitution. Such exemplary polypeptides can be used in the oral dosage formulations provided herein.

TABLE 3
Interleukin-10 (IL-10) Modifications to Increase Resistance to Proteolysis

| | | | | |
|---|---|---|---|---|
| K49N (810) | K49Q (811) | E50N (812) | E50Q (813) | E50H (814) |
| L52I (815) | L52V (816) | L53I (817) | L53V (818) | E54N (819) |
| E54Q (820) | E54H (821) | D55N (822) | D55Q (823) | F56I (824) |
| F56V (825) | K57N (826) | K57Q (827) | Y59H (828) | Y59I (829) |
| L60I (830) | L60V (831) | E67N (832) | E67Q (833) | E67H (834) |
| M68I (835) | M68V (836) | F71I (837) | F71V (838) | Y72H (839) |
| Y72I (840) | E74N (841) | E74Q (842) | E74H (843) | E75N (844) |
| E75Q (845) | E75H (846) | P78A (847) | P78S (848) | E81N (849) |
| E81Q (850) | E81H (851) | D84N (852) | D84Q (853) | P85A (854) |
| P85S (855) | D86N (856) | D86Q (857) | K88N (858) | K88Q (859) |

TABLE 4
Interferon-β (IFN- β) Modifications to Increase Resistance to Proteolysis

| | | | | |
|---|---|---|---|---|
| M1I (148) | M1V (149) | M1T (150) | M1Q (151) | M1A (152) |
| Y3H (153) | Y3I (154) | L5I (155) | L5V (156) | L5T (157) |
| L5Q (158) | L5H (159) | L5A (160) | L6I (161) | L6V (162) |
| F8I (163) | F8V (164) | L9I (165) | L9V (166) | L9T (167) |
| L9Q (168) | L9H (169) | L9A (170) | R11H (171) | R11Q (172) |
| F15I (173) | F15V (174) | K19N (175) | K19Q (176) | K19T (177) |
| K19S (178) | K19H (179) | L20I (180) | L20V (181) | L21I (182) |
| L21V (183) | W22S (184) | W22H (185) | L24I (186) | L24V (187) |
| N25H (188) | N25Q (189) | N25S (190) | R27H (191) | R27Q (192) |
| L28I (193) | L28V (194) | L28T (195) | L28Q (196) | L28H (197) |
| L28A (198) | E29N (199) | E29Q (200) | E29H (201) | Y30H (202) |
| Y30I (203) | L32I (204) | L32V (205) | L32T (206) | L32Q (207) |
| L32H (208) | L32A (209) | K33N (210) | K33Q (211) | K33T (212) |
| K33S (213) | K33H (214) | D34N (215) | D34Q (216) | R35H (217) |
| R35Q (218) | M36I (219) | M36V (220) | M36T (221) | M36Q (222) |
| M36A (223) | F38I (224) | F38V (225) | D39N (226) | D39Q (227) |
| D39H (228) | D39G (229) | P41A (230) | P41S (231) | E42N (232) |
| E42Q (233) | E42H (234) | E43N (235) | E43Q (236) | E43H (237) |
| K45N (238) | K45Q (239) | K45T (240) | K45S (241) | K45H (242) |
| L47I (243) | L47V (244) | L47T (245) | L47Q (246) | L47H (247) |
| L47A (248) | F50I (249) | F50V (250) | K52N (251) | K52Q (252) |
| K52T (253) | K52S (254) | K52H (255) | E53N (256) | E53Q (257) |
| E53H (258) | D54N (259) | D54Q (260) | L57I (261) | L57V (262) |
| Y60H (263) | Y60I (264) | E61N (265) | E61Q (266) | E61H (267) |
| M62I (268) | M62V (269) | L63I (270) | L63V (271) | F67I (272) |
| F67V (273) | F70I (274) | F70V (275) | R71H (276) | R71Q (277) |
| D73N (278) | D73Q (279) | D73H (280) | D73G (281) | W79S (282) |
| W79H (283) | E81N (284) | E81Q (285) | E81H (286) | E85N (287) |
| E85Q (288) | E85H (289) | L87I (290) | L87V (291) | L88I (292) |
| L88V (293) | Y92H (294) | Y92I (295) | L98I (296) | L98V (297) |
| K99N (298) | K99Q (299) | K99T (300) | K99S (301) | K99H (302) |
| L102I (303) | L102V (304) | E103N (305) | E103Q (306) | E103H (307) |
| E104N (308) | E104Q (309) | E104H (310) | K105N (311) | K105Q (312) |
| K105T (313) | K105S (314) | K105H (315) | L106I (316) | L106V (317) |
| E107N (318) | E107Q (319) | E107H (320) | K108N (321) | K108Q (322) |
| K108T (323) | K108S (324) | K108H (325) | E109N (326) | E109Q (327) |
| E109H (328) | D110N (329) | D110Q (330) | D110H (331) | D110G (332) |
| F111I (333) | F111V (334) | R113H (335) | R113Q (336) | K115N (337) |
| K115Q (338) | L116I (339) | L116V (340) | L116T (341) | L116Q (342) |
| L116H (343) | L116A (344) | M117I (345) | M117V (346) | L120I (347) |
| L120V (348) | L120T (349) | L120Q (350) | L120H (351) | L120A (352) |
| L122I (353) | L122V (354) | K123N (355) | K123Q (356) | K123T (357) |
| K123S (358) | K123H (359) | R124H (360) | R124Q (361) | Y125H (362) |
| Y125I (363) | Y126H (364) | Y126I (365) | R128H (366) | R128Q (367) |
| L130I (368) | L130V (369) | L130T (370) | L130Q (371) | L130H (372) |
| L130A (373) | Y132H (374) | Y132I (375) | L133I (376) | L133V (377) |
| K134N (378) | K134Q (379) | K134T (380) | K134S (381) | K134H (382) |
| K136N (383) | K136Q (384) | K136T (385) | K136S (386) | K136H (387) |
| E137N (388) | E137Q (389) | E137H (390) | Y138H (391) | Y138I (392) |
| W143S (393) | W143H (394) | R147H (395) | R147Q (396) | E149N (397) |
| E149Q (398) | E149H (399) | L151I (400) | L151V (401) | R152H (402) |
| R152Q (403) | F154I (404) | F154V (405) | Y155H (406) | Y155I (407) |
| F156I (408) | F156V (409) | R159H (410) | R159Q (411) | L160I (412) |
| L160V (413) | Y163H (414) | Y163I (415) | L164I (416) | L164V (417) |
| R165H (418) | R165Q (419) | M1D (420) | M1E (421) | M1K (422) |
| M1N (423) | M1R (424) | M1S (425) | L5D (426) | L5E (427) |
| L5K (428) | L5R (429) | L5N (430) | L5S (431) | L6D (432) |
| L6E (433) | L6K (434) | L6N (435) | L6Q (436) | L6R (437) |
| L6S (438) | L6T (439) | F8D (440) | F8E (441) | F8K (442) |
| F8R (443) | L9D (444) | L9E (445) | L9K (446) | L9N (447) |
| L9R (448) | L9S (449) | Q10D (450) | Q10E (451) | Q10K (452) |
| Q10N (453) | Q10R (454) | Q10S (455) | Q10T (456) | S12D (457) |
| S12E (458) | S12K (459) | S12R (460) | S13D (461) | S13E (462) |
| S13K (463) | S13N (464) | S13Q (465) | S13R (466) | S13T (467) |
| N14D (468) | N14E (469) | N14K (470) | N14Q (471) | N14R (472) |
| N14S (473) | N14T (474) | F15D (475) | F15E (476) | F15K (477) |
| F15R (478) | Q16D (479) | Q16E (480) | Q16K (481) | Q16N (482) |
| Q16R (483) | Q16S (484) | Q16T (485) | C17D (486) | C17E (487) |
| C17K (488) | C17N (489) | C17Q (490) | C17R (491) | C17S (492) |
| C17T (493) | L20N (494) | L20Q (495) | L20R (496) | L20S (497) |
| L20T (498) | L20D (499) | L20E (500) | L20K (501) | W22D (502) |
| W22E (503) | W22K (504) | W22R (505) | Q23D (506) | Q23E (507) |
| Q23K (508) | Q23R (509) | L24D (510) | L24E (511) | L24K (512) |
| L24R (513) | G78D (514) | G78E (515) | G78K (516) | G78R (517) |
| W79D (518) | W79E (519) | W79K (520) | W79R (521) | N80D (522) |
| N80E (523) | N80K (524) | N80R (525) | T82D (526) | T82E (527) |
| T82K (528) | T82R (529) | I83D (530) | I83E (531) | I83K (532) |
| I83R (533) | I83N (534) | I83Q (535) | I83S (536) | I83T (537) |
| N86D (538) | N86E (539) | N86K (540) | N86R (541) | N86Q (542) |
| N86S (543) | N86T (544) | L87D (545) | L87E (546) | L87K (547) |
| L87R (548) | L87N (549) | L87Q (550) | L87S (551) | L87T (552) |
| A89D (553) | A89E (554) | A89K (555) | A89R (556) | N90D (557) |
| N90E (558) | N90K (559) | N90Q (560) | N90R (561) | N90S (562) |
| N90T (563) | V91D (564) | V91E (565) | V91K (566) | V91N (567) |
| V91Q (568) | V91R (569) | V91S (570) | V91T (571) | Q94D (572) |
| Q94E (573) | Q94K (574) | Q94N (575) | Q94R (576) | Q94S (577) |
| Q94T (578) | I95D (579) | I95E (580) | I95K (581) | I95N (582) |
| I95Q (583) | I95R (584) | I95S (585) | I95T (586) | H97D (587) |
| H97E (588) | H97K (589) | H97N (590) | H97Q (591) | H97R (592) |
| H97S (593) | H97T (594) | L98D (595) | L98E (596) | L98K (597) |
| L98N (598) | L98Q (599) | L98R (600) | L98S (601) | L98T (602) |
| V101D (603) | V101E (604) | V101K (605) | V101N (606) | V101Q (607) |
| V101R (608) | V101S (609) | V101T (610) | M1C (611) | L6C (612) |
| Q10C (613) | S13C (614) | Q16C (615) | N90C (616) | V91C (617) |
| Q94C (618) | H97C (619) | L98C (620) | V101C (621) | L5D/L6E (622) |
| L5E/Q10D | L5Q/M36I | L6E/L47I (625) | L5E/K108S | L5E/L6E (627) |
| (623) | (624) | | (626) | |
| L5D/Q10D | L5N/M36I (629) | L6Q/L47I (630) | L5D/K108S | L5N/L6E (632) |
| (628) | | | (631) | |
| L5Q/Q10D | L6E/M36I (634) | L5E/N86Q | L5Q/K108S | L5Q/L6E (637) |
| (633) | | (635) | (636) | |
| L5N/Q10D | L6Q/M36I | L5D/N86Q | L5N/K108S | L5D/L6Q (642) |
| (638) | (639) | (640) | (641) | |
| L6E/Q10D | L5E/L47I (644) | L5Q/N86Q | L6E/K108S | L5E/L6Q (647) |
| (643) | | (645) | (646) | |
| L6Q/Q10D | L5D/L47I (649) | L5N/N86Q | L6Q/K108S | L5N/L6Q (652) |
| (648) | | (650) | (651) | |
| L5E/M36I (653) | L5Q/L47I (654) | L6E/N86Q | L5Q/L6Q (656) | L5D/M36I (657) |
| | | (655) | | |
| L5N/L47I (658) | L6Q/N86Q | L6E/K108S | L6H (1887) | L6A (1888) |
| | (659) | (660) | | |
| L20H (1889) | L20A (1890) | L21T (1891) | L21Q (1892) | L21H (1893) |
| L21A (1894) | L24T (1895) | L24Q (1896) | L24H (1897) | L24A (1898) |
| D34G (1899) | D54G (1900) | L57T (1901) | L57Q (1902) | L57H (1903) |
| L57A (1904) | M62T (1905) | M62Q (1906) | M62A (1907) | L63T (1908) |
| L63Q (1909) | L63H (1910) | L63A (1911) | L87H (1912) | L87A (1913) |
| L88T (1914) | L88Q (1915) | L88H (1916) | L88A (1917) | L98H (1918) |
| L98A (1919) | L102T (1920) | L102Q (1921) | L102H (1922) | L102A (1923) |
| L106T (1924) | L106Q (1925) | L106H (1926) | L106A (1927) | K115S (1928) |
| K115H (1929) | K155N (1930) | M117T (1931) | M117Q (1932) | M117A (1933) |
| L122T (1934) | L122Q (1935) | L122H (1936) | L122A (1937) | L133T (1938) |
| L133Q (1939) | L133H (1940) | L133A (1941) | L151T (1942) | L151Q (1943) |
| L151H (1944) | L151A (1945) | L160T (1946) | L160Q (1947) | L160H (1948) |
| L160A (1949) | L164T (1950) | L164Q (1951) | L164H (1952) | L164A (1953) |
| Q18H (1954) | Q18S (1955) | Q18T (1956) | Q18N (1957) | Q23H (1958) |
| Q23S (1959) | Q23T (1960) | Q23N (1961) | Q48H (1962) | Q48S (1963) |
| Q48T (1964) | Q48N (1965) | Q49H (1966) | Q49S (1967) | Q49T (1968) |
| Q49N (1969) | Q51H (1970) | Q51S (1971) | Q51T (1972) | Q51N (1973) |
| Q64H (1974) | Q64S (1975) | Q64T (1976) | Q64N (1977) | Q72H (1978) |
| Q72S (1979) | Q72T (1980) | Q72N (1981) | | |

TABLE 5
Interferon-y (IFN-y) Modifications to Increase Resistance to Proteolysis

| | | | | |
|---|---|---|---|---|
| Y2H (662) | Y2I (663) | D5N (664) | D5Q (665) | P6A (666) |
| P6S (667) | Y7H (668) | Y7I (669) | K9N (670) | K9Q (671) |
| E10N (672) | E10Q (673) | E10H (674) | E12N (675) | E12Q (676) |
| E12H (677) | L14I (678) | L14V (679) | K15N (680) | K15Q (681) |
| K16N (682) | K16Q (683) | Y17H (684) | Y17I (685) | F18I (686) |
| F18V (687) | D24N (688) | D24Q (689) | D27N (690) | D27Q (691) |
| N28Q (692) | N28S (693) | L31I (694) | L31V (695) | F32I (696) |
| F32V (697) | L33I (698) | L33V (699) | L36I (700) | L36V (701) |
| K37N (702) | K37Q (703) | W39S (704) | W39H (705) | K40N (706) |
| K40Q (707) | E41N (708) | E41Q (709) | E41H (710) | E42N (711) |
| E42Q (712) | E42H (713) | D44N (714) | D44Q (715) | R45H (716) |
| R45Q (717) | F57I (718) | F57V (719) | K58N (720) | K58Q (721) |
| L59I (722) | L59V (723) | F60I (724) | F60V (725) | K61N (726) |
| K61Q (727) | F63I (728) | F63V (729) | K64N (730) | K64Q (731) |
| D65N (732) | D65Q (733) | D66N (734) | D66Q (735) | K71N (736) |
| K71Q (737) | E74N (738) | E74Q (739) | E74H (740) | K77N (741) |
| K77Q (742) | E78N (743) | E78Q (744) | E78H (745) | D79N (746) |
| D79Q (747) | K83N (748) | K83Q (749) | F84I (750) | F84V (751) |
| K89N (752) | K89Q (753) | K90N (754) | K90Q (755) | K91N (756) |
| K91Q (757) | R92H (758) | R92Q (759) | D93N (760) | D93Q (761) |
| D94N (762) | D94Q (763) | F95I (764) | F95V (765) | E96N (766) |
| E96Q (767) | E96H (768) | K97N (769) | K97Q (770) | L98I (771) |
| L98V (772) | Y101H (773) | Y101I (774) | D105N (775) | D105Q (776) |
| L106I (777) | L106V (778) | E115N (779) | E115Q (780) | E115H (781) |
| E122N (782) | E122Q (783) | E122H (784) | L123I (785) | L123V (786) |
| P125A (787) | P125S (788) | K128N (789) | K128Q (790) | K131N (791) |
| K131Q (792) | R132H (793) | R132Q (794) | K133N (795) | K133Q (796) |
| R134H (797) | R134Q (798) | M137I (799) | M137V (800) | L138I (801) |
| L138V (802) | F139I (803) | F139V (804) | R142H (805) | R142Q (806) |
| R143H (807) | R143Q (808) | | | |

b. Long-Chain Cytokine Family

As set forth above, provided herein are oral dosage formulations containing modified cytokines that contain one or more amino acid replacements at one or more target positions in polypeptides of the long-chain cytokine family, including, but not limited to, granulocyte colony stimulating factor (G-CSF; SEQ ID NO: 47), leukemia inhibitory factor (LIF; SEQ ID NO: 1148), growth hormone (hGH; SEQ ID NO: 1260), ciliary neurotrophic factor (CNTF; SEQ ID NO: 1), teptin (SEQ ID NO: 1126), oncostatin. M (SEQ ID NO: 1181), interleukin-6 (IL-6; SEQ ID NO: 1080) and interleukin-12 (IL-12; SEQ ID NO: 860). Exemplary protease-resistant polypeptides include one or more amino acid modifications in such polypeptides, where the one or more amino acid replacements lead to greater resistance to proteases. Such modifications include, but are not limited to, modifications shown in Tables 6-13, where the replacements are made compared to the sequence of amino acids set forth in SEQ ID NOS: 47, 1148, 1260, 1, 1126, 1181, 1080, or 860, respectively. In reference to such mutants, the first amino acid (one-letter abbreviation) corresponds to the amino acid that is replaced, the number corresponds to position in the G-CSF, LIF, hGH, CNTF, leptin, oncostatin M, IL-6, or IL-12 polypeptide sequence with reference to SEQ ID NOS: 47, 1148, 1260, 1, 1126, 1181, 1080, or 860, respectively, and the second amino acid (one-letter abbreviation) corresponds to the amino acid selected that replaces the first amino acid at that position. In the tables, the sequence identifier (SEQ ID NO.) for the accompanying Sequence Listing is in parenthesis next to each substitution. Such exemplary polypeptides can be used in the oral dosage formulations provided herein.

TABLE 6
G-CSF Modifications to Increase Resistance to Proteolysis

| | | | | |
|---|---|---|---|---|
| E38N (48) | E38Q (49) | E38H (50) | E41N (51) | E41Q (52) |
| E41H (53) | E45N (54) | E45Q (55) | E45H (56) | M46I (57) |
| M46V (58) | D48N (59) | D48Q (60) | L49I (61) | L49V (62) |
| E51N (63) | E51Q (64) | E51H (65) | E60N (66) | E60Q (67) |
| E60H (68) | K63N (69) | K63Q (70) | P92A (71) | P92S (72) |
| E93N (73) | E93Q (74) | E93H (75) | F119I (76) | F119V (77) |
| D120N (78) | D120Q (79) | E123N (80) | E123Q (81) | E123H (82) |
| P124A (83) | P124S (84) | | | |

TABLE 7
Leukocyte Inhibitory Factor (LIF)
Modifications to Increase Resistance to Proteolysis

| P69A | P69S (1150) | F70I (1151) | F70V (1152) | R85H (1153) |
|---|---|---|---|---|
| (1149) | | | | |
| R85Q | R99H (1155) | R99Q (1156) | K102N (1157) | K102Q (1158) |
| (1154) | | | | |
| L104I | L104V | P106A (1161) | P106S (1162) | L109I (1163) |
| (1159) | (1160) | | | |
| L109V | Y137H | Y137I (1166) | D143N (1167) | D143Q (1168) |
| (1164) | (1165) | | | |
| Y146H | Y146I | P148A (1171) | P148S (1172) | D149N (1173) |
| (1169) | (1170) | | | |
| D149Q | K153N | K153Q (1176) | D154N (1177) | D154Q (1178) |
| (1174) | (1175) | | | |
| F156I | F156V | | | |
| (1179) | (1180) | | | |

TABLE 8
Growth Hormone (GH) Modifications to Increase Resistance to Proteolysis

| F1I/P2S | F1I/P2A | F1V/P2S | F1V/P2A | P5A (1265) |
|---|---|---|---|---|
| (1261) | (1262) | (1263) | (1264) | |
| P5S | L6I (1267) | L6V (1268) | R8H (1269) | R8Q (1270) |
| (1266) | | | | |
| L9I | L9V (1272) | F10I (1273) | F10V (1274) | D11N (1275) |
| (1271) | | | | |
| D11Q | M14I (1277) | M14V (1278) | L15I (1279) | L15V (1280) |
| (1276) | | | | |
| R16H | R16Q (1282) | R19H (1283) | R19Q (1284) | L20I (1285) |
| (1281) | | | | |
| L20V | L23I (1287) | L23V (1288) | F25I (1289) | F25V (1290) |
| (1286) | | | | |
| D26N | D26Q (1292) | Y28H (1293) | Y28I (1294) | E30N (1295) |
| (1291) | | | | |
| E30Q | E30H (1297) | F31I (1298) | F31V (1299) | E32N (1300) |
| (1296) | | | | |
| E32Q | E32H (1302) | E33N (1303) | E33Q (1304) | E33H (1305) |
| (1301) | | | | |
| Y35H | Y35I (1307) | P37A (1308) | P37S (1309) | K38N (1310) |
| (1306) | | | | |
| K38Q | E39N (1312) | E39Q (1313) | E39H (1314) | K41N (1315) |
| (1311) | | | | |
| K41Q | Y42H (1317) | Y42I (1318) | F44I (1319) | F44V (1320) |
| (1316) | | | | |
| L45I | L45V (1322) | P48A (1323) | P48S (1324) | L52I (1325) |
| (1321) | | | | |
| L52V | F54I (1327) | F54V (1328) | E56N (1329) | E56Q (1330) |
| (1326) | | | | |
| E56H | P59A (1332) | P59S (1333) | P61A (1334) | P61S (1335) |
| (1331) | | | | |
| R64H | R64Q (1337) | E65N (1338) | E65Q (1339) | E65H (1340) |
| (1336) | | | | |
| E66N | E66Q (1342) | E66H (1343) | K70N (1344) | K70Q (1345) |
| (1341) | | | | |
| L73I | L73V (1347) | E74N (1348) | E74Q (1349) | E74H (1350) |
| (1346) | | | | |
| L75I | L75V (1352) | L76I (1353) | L76V (1354) | R77H (1355) |
| (1351) | | | | |
| R77Q | L80I (1357) | L80V (1358) | L81I (1359) | L81V (1360) |
| (1356) | | | | |
| L82I | L82V (1362) | W86S (1363) | W86H (1364) | L87I (1365) |
| (1361) | | | | |
| L87V | E88N (1367) | E88Q (1368) | E88H (1369) | P89A (1370) |
| (1366) | | | | |
| P89S | F92I (1372) | F92V (1373) | L93I (1374) | L93V (1375) |
| (1371) | | | | |
| R94H | R94Q (1377) | F97I (1378) | F97V (1379) | L101I (1380) |
| (1376) | | | | |
| L101V | Y103H | Y103I (1383) | D107N (1384) | D107Q (1385) |
| (1381) | (1382) | | | |
| Y111H | Y111I | D112N (1388) | D112Q (1389) | L113I (1390) |
| (1386) | (1387) | | | |
| L113V | L114I (1392) | L114V (1393) | K115N (1394) | K115Q (1395) |
| (1391) | | | | |
| D116N | D116Q | L117I (1398) | L117V (1399) | E118N (1400) |
| (1396) | (1397) | | | |
| E118Q | E118H | E119N (1403) | E119Q (1404) | E119H (1405) |
| (1401) | (1402) | | | |
| L124I | L124V | M125I (1408) | M125V (1409) | R127H (1410) |
| (1406) | (1407) | | | |
| R127Q | L128I (1412) | L128V (1413) | E129N (1414) | E129Q (1415) |
| (1411) | | | | |
| E129H | D130N | D130Q (1418) | P133A (1419) | P133S (1420) |
| (1416) | (1417) | | | |
| R134H | R134Q | F139I (1423) | F139V (1424) | K140N (1425) |
| (1421) | (1422) | | | |
| K140Q | Y143H | Y143I (1428) | K145N (1429) | K145Q (1430) |
| (1426) | (1427) | | | |
| F146I | F146V | D147N (1433) | D147Q (1434) | D153N (1435) |
| (1431) | (1432) | | | |
| D153Q | D154N | D154Q (1438) | L156I (1439) | L156V (1440) |
| (1436) | (1437) | | | |
| L157I | L157V | K158N (1443) | K158Q (1444) | Y160H (1445) |
| (1441) | (1442) | | | |
| Y160I | L162I (1447) | L162V (1448) | L163I (1449) | L163V (1450) |
| (1446) | | | | |
| Y164H | Y164I | F166I (1453) | F166V (1454) | R167H (1455) |
| (1451) | (1452) | | | |
| R167Q | K168N | K168Q (1458) | D169N (1459) | D169Q (1460) |
| (1456) | (1457) | | | |
| M170I | M170V | D171N (1463) | D171Q (1464) | K172N (1465) |
| (1461) | (1462) | | | |
| K172Q | E174N | E174Q (1468) | E174H (1469) | F176I (1470) |
| (1466) | (1467) | | | |
| F176V | L177I (1472) | L177V (1473) | R178H (1474) | R178Q (1475) |
| (1471) | | | | |
| R183H | R183Q | E186N (1478) | E186Q (1479) | E186H (1480) |
| (1476) | (1477) | | | |
| F191I | F191V | | | |
| (1481) | (1482) | | | |

TABLE 9
Ciliary Neurotrophic Factor (CTNF)
Modifications to Increase Resistance to Proteolysis

| | | | | |
|---|---|---|---|---|
| D62N (2) | D62Q (3) | W64S (4) | W64H (5) | E66N (6) |
| E66Q (7) | E66H (8) | L67I (9) | L67V (10) | L86I (11) |
| L86V (12) | R89H (13) | R89Q (14) | E92N (15) | E92Q (16) |
| E92H (17) | P100A (18) | P100S (19) | E102N (20) | E102Q (21) |
| E102H (22) | D104N (23) | D104Q (24) | E131N (25) | E131Q (26) |
| E131H (27) | Y132H (28) | Y132I (29) | K133N (30) | K133Q (31) |
| P135A (32) | P135S (33) | R136H (34) | R136Q (35) | E138N (36) |
| E138Q (37) | E138H (38) | D140N (39) | D140Q (40) | P143A (41) |
| P143S (42) | D148N (43) | D148Q (44) | L151I (45) | L151V (46) |

TABLE 10
Leptin Modifications to Increase Resistance to Proteolysis

| P43A | P43S (1128) | L49I (1129) | L49V (1130) | P99A (1131) |
|---|---|---|---|---|
| (1127) | | | | |
| P99S | W100S | W100H (1134) | L104I (1135) | L104V (1136) |
| (1132) | (1133) | | | |
| E105N | E105Q | E105H (1139) | L107I (1140) | L107V (1141) |
| (1137) | (1138) | | | |
| D108N | D108Q | D141N (1144) | D141Q (1145) | L142I (1146) |
| (1142) | (1143) | | | |
| L142V | | | | |
| (1147) | | | | |

TABLE 11
Oncostatin M Modifications to Increase Resistance to Proteolysis

| E59N (1182) | E59Q (1183) | E59H (1184) | E60N (1185) | E60Q (1186) |
|---|---|---|---|---|
| E60H (1187) | R63H (1188) | R63Q (1189) | L65I (1190) | L65V (1191) |
| R84H (1192) | R84Q (1193) | D87N (1194) | D87Q (1195) | E89N (1196) |
| E89Q (1197) | E89H (1198) | R91H (1199) | R91Q (1200) | K94N (1201) |
| K94Q (1202) | D97N (1203) | D97Q (1204) | E99N (1205) | E99Q (1206) |
| E99H (1207) | R100H | R100Q | L103I (1210) | L103V (1211) |
| | (1208) | (1209) | | |
| E106N | E106Q | E106H | | |
| (1212) | (1213) | (1214) | | |

TABLE 12
Interleukin-6 (IL-6) Modifications to Increase Resistance to Proteolysis

| P64A | P64S (1082) | K65N (1083) | K65Q (1084) | M66I (1085) |
|---|---|---|---|---|
| (1081) | | | | |
| M66V | E68N (1087) | E68Q (1088) | E68H (1089) | K69N (1090) |
| (1086) | | | | |
| K69Q | F73I (1092) | F73V (1093) | F77I (1094) | F77V (1095) |
| (1091) | | | | |
| E92N | E92Q (1097) | E92H (1098) | E98N (1099) | E98Q (1100) |
| (1096) | | | | |
| E98H | R103H | R103Q (1103) | E105N (1104) | E105Q (1105) |
| (1101) | (1102) | | | |
| E105H | E108N | E108Q (1108) | E108H (1109) | D133N (1110) |
| (1106) | (1107) | | | |
| D133Q | P138A | P138S (1113) | D139N (1114) | D139Q (1115) |
| (1111) | (1112) | | | |
| P140A | P140S | K149N (1118) | K149Q (1119) | W156S (1120) |
| (1116) | (1117) | | | |
| W156H | R178H | R178Q (1123) | R181H (1124) | R181Q (1125) |
| (1121) | (1122) | | | |

TABLE 13
Interleukin-12 (IL-12) Modifications to Increase Resistance to Proteolysis

| K56N | K56Q | E61N | E61Q | E61H | L66I | L66V | E67N |
|---|---|---|---|---|---|---|---|
| (861) | (862) | (863) | (864) | (865) | (866) | (867) | (868) |
| E67Q | E67H | L68I | L68V | K70N | K70Q | E72N | E72Q |
| (869) | (870) | (871) | (872) | (873) | (874) | (875) | (876) |
| E72H | L75I | L75V | R78H | R78Q | E79N | E79Q | E79H |
| (877) | (878) | (879) | (880) | (881) | (882) | (883) | (884) |
| F82I | F82V | L89I | L89V | R92H | R92Q | K93N | K93Q |
| (885) | (886) | (887) | (888) | (889) | (890) | (891) | (892) |
| E107N | E107Q | E107H | K110N | K110Q | M111I | M111V | E115N |
| (893) | (894) | (895) | (896) | (897) | (898) | (899) | (900) |
| E115Q | E115H | K117N | K117Q | L124I | L124V | M125I | M125V |
| (901) | (902) | (903) | (904) | (905) | (906) | (907) | (908) |
| P127A | P127S | K128N | K128Q | R129H | R129Q | R189H | R189Q |
| (909) | (910) | (911) | (912) | (913) | (914) | (915) | (916) |

c. Short-Chain Cytokine Family

As set forth above, provided herein are oral dosage formulations containing modified cytokines that contain one or more amino acid replacements at one or more target positions in polypeptides of the short-chain cytokine protein family, including, but not limited to, erythropoietin (EPO; SEQ ID NO: 1886), granulocyte-macrophage colony stimulating factor (GM-CSF; SEQ ID NO: 85), interleukin-2 (IL-2; SEQ ID NO: 946), interleukin-3 (IL-3; SEQ ID NO: 995), interleukin-4 (IL-4; SEQ ID NO: 1018), interleukin-5 (IL-5; SEQ ID NO: 1044), interleukin-13 (IL-13; SEQ ID NO: 917), Flt3 ligand (SEQ ID NO: 118) and stem cell factor (SCF; SEQ ID NO: 1215). Exemplary protease-resistant polypeptides include one or more amino acid modifications in such polypeptides, where the one or more amino acid replacements lead to greater resistance to proteases. Such modifications include, but are not limited to, modifications shown in Tables 14-22, where the replacements are made compared to the sequence of amino acids set forth in SEQ ID NOS: 1886, 85, 946, 995, 1018, 1044, 917, 118, or 1215, respectively. In reference to such mutants, the first amino acid (one-letter abbreviation) corresponds to the amino acid that is replaced, the number corresponds to position in the EPO, GM-CSF, IL-2, IL-3, IL-4, IL-5, IL-13, Flt3 ligand, or SCF polypeptide sequence with reference to SEQ ID NOS: 1886, 85, 946, 995, 1018, 1044, 917, 118, or 1215, respectively, and the second amino acid (one-letter abbreviation) corresponds to the amino acid selected that replaces the first amino acid at that position. In the tables, the sequence identifier (SEQ ID NO.) for the accompanying Sequence Listing is in parenthesis next to each substitution. Such exemplary polypeptides can be used in the oral dosage formulations provided herein.

TABLE 14
Erythropoietin (EPO) Modifications to Increase Resistance to Proteolysis

| P2S | P2A (1484) | P3S (1485) | P3A (1486) | R4H (1487) |
|---|---|---|---|---|
| (1483) | | | | |
| R4Q | L5I (1489) | L5V (1490) | C7S (1491) | C7V (1492) |
| (1488) | | | | |
| C7A | C7I (1494) | C7T (1495) | D8Q (1496) | D8H (1497) |
| (1493) | | | | |
| D8N | R10H (1499) | R10Q (1500) | L12V (1501) | L12I (1502) |
| (1498) | | | | |
| E13Q | E13H (1504) | E13N (1505) | R14H (1506) | R14Q (1507) |
| (1503) | | | | |
| Y15H | Y15I (1509) | Y15K (1510) | Y15R (1511) | L16I (1512) |
| (1508) | | | | |
| L16V | L16E (1514) | L16D (1515) | L16K (1516) | L16R (1517) |
| (1513) | | | | |
| L17I | L17V (1519) | L17E (1520) | L17D (1521) | L17K (1522) |
| (1518) | | | | |
| L17R | E18Q (1524) | E18H (1525) | E18N (1526) | E18K (1527) |
| (1523) | | | | |
| E18R | K20Q (1529) | K20T (1530) | K20N (1531) | E21Q (1532) |
| (1528) | | | | |
| E21H | E21N (1534) | E21K (1535) | E21R (1536) | E23Q (1537) |
| (1533) | | | | |
| E23H | E23N (1539) | E23K (1540) | E23R (1541) | I25K (1542) |
| (1538) | | | | |
| I25R | T26E (1544) | T26D (1545) | T26K (1546) | T26R (1547) |
| (1543) | | | | |
| T27E | T27D (1549) | T27K (1550) | T27R (1551) | G28E (1552) |
| (1548) | | | | |
| G28D | G28K (1554) | G28R (1555) | C29S (1556) | C29V (1557) |
| (1553) | | | | |
| C29A | C29I (1559) | C29T (1560) | C29K (1561) | C29R (1562) |
| (1558) | | | | |
| A30E | A30D (1564) | A30K (1565) | A30R (1566) | E31Q (1567) |
| (1563) | | | | |
| E31H | E31N (1569) | H32K (1570) | H32R (1571) | C33K (1572) |
| (1568) | | | | |
| C33R | S34K (1574) | S34R (1575) | L35V (1576) | L35K (1577) |
| (1573) | | | | |
| L35R | L35I (1579) | N36E (1580) | N36D (1581) | N36K (1582) |
| (1578) | | | | |
| N36R | E37Q (1584) | E37H (1585) | E37N (1586) | E37K (1587) |
| (1583) | | | | |
| E37R | I39K (1589) | I39R (1590) | T40E (1591) | T40D (1592) |
| (1588) | | | | |
| T40K | T40R (1594) | P42S (1595) | P42A (1596) | P42K (1597) |
| (1593) | | | | |
| P42R | D43Q (1599) | D43H (1600) | D43N (1601) | K45Q (1602) |
| (1598) | | | | |
| K45T | K45N (1604) | F48I (1605) | F48V (1606) | Y49H (1607) |
| (1603) | | | | |
| Y49I | W51S | W51H (1610) | K52Q (1611) | K52T (1612) |
| (1608) | (1609) | | | |
| K52N | R53H (1614) | R53Q (1615) | M54V (1616) | M54I (1617) |
| (1613) | | | | |
| E55Q | E55H (1619) | E55N (1620) | V61K (1621) | V61R (1622) |
| (1618) | | | | |
| E62Q | E62H (1624) | E62N (1625) | E62K (1626) | E62R (1627) |
| (1623) | | | | |
| W64S | W64H | W64K (1630) | W64R (1631) | Q65K (1632) |
| (1628) | (1629) | | | |
| Q65R | L67I (1634) | L67V (1635) | A68K (1636) | A68R (1637) |
| (1633) | | | | |
| L69V | L69I (1639) | L69K (1640) | L69R (1641) | L70I (1642) |
| (1638) | | | | |
| L70V | S71K (1644) | S71R (1645) | E72Q (1646) | E72H (1647) |
| (1643) | | | | |
| E72N | E72K (1649) | E72R (1650) | V74K (1651) | V74R (1652) |
| (1648) | | | | |
| L75V | L75I (1654) | L75E (1655) | L75D (1656) | L75K (1657) |
| (1653) | | | | |
| L75R | R76H (1659) | R76Q (1660) | Q78E (1661) | Q78D (1662) |
| (1658) | | | | |
| Q78K | Q78R (1664) | A79K (1665) | A79R (1666) | L80V (1667) |
| (1663) | | | | |
| L80I | L80K (1669) | L80R (1670) | L81I (1671) | L81V (1672) |
| (1668) | | | | |
| V82E | V82D (1674) | V82K (1675) | V82R (1676) | S84E (1677) |
| (1673) | | | | |
| S84D | S84K (1679) | S84R (1680) | S85D (1681) | S85K (1682) |
| (1678) | | | | |
| S85R | Q86E (1684) | Q86D (1685) | Q86K (1686) | Q86R (1687) |
| (1683) | | | | |
| P87S | P87A (1689) | P87K (1690) | P87R (1691) | W88S (1692) |
| (1688) | | | | |
| W88H | W88E | W88D (1695) | W88K (1696) | W88R (1697) |
| (1693) | (1694) | | | |
| E89Q | E89H (1699) | E89N (1700) | E89K (1701) | E89R (1702) |
| (1698) | | | | |
| P90S | P90A (1704) | P90K (1705) | P90R (1706) | L91I (1707) |
| (1703) | | | | |
| L91V | L91K (1709) | L91R (1710) | Q92E (1711) | Q92D (1712) |
| (1708) | | | | |
| Q92K | Q92R (1714) | L93V (1715) | L93I (1716) | L93E (1717) |
| (1713) | | | | |
| L93D | L93K (1719) | L93R (1720) | H94K (1721) | H94R (1722) |
| (1718) | | | | |
| V95K | V95R (1724) | D96Q (1725) | D96H (1726) | D96N (1727) |
| (1723) | | | | |
| K97Q | K97T (1729) | K97N (1730) | L102V (1731) | L102I (1732) |
| (1728) | | | | |
| R103H | R103Q | L105I (1735) | L105V (1736) | L108I (1737) |
| (1733) | (1734) | | | |
| L108V | L109I (1739) | L109V (1740) | R110H (1741) | R110Q (1742) |
| (1738) | | | | |
| L112V | L112I (1744) | K116Q (1745) | K116T (1746) | K116N (1747) |
| (1743) | | | | |
| E117Q | E117H | E117N (1750) | A118E (1751) | A118D (1752) |
| (1748) | (1749) | | | |
| A118K | A118R | I119E (1755) | I119D (1756) | I119K (1757) |
| (1753) | (1754) | | | |
| I119R | S120E | S120D (1760) | S120K (1761) | S120R (1762) |
| (1758) | (1759) | | | |
| P121S | P121A | P121E (1765) | P121D (1766) | P121K (1767) |
| (1763) | (1764) | | | |
| P121R | P122S | P122A (1770) | P122E (1771) | P122D (1772) |
| (1768) | (1769) | | | |
| P122K | P122R | D123Q (1775) | D123H (1776) | D123N (1777) |
| (1773) | (1774) | | | |
| D123E | D123K | D123R (1780) | A124E (1781) | A124D (1782) |
| (1778) | (1779) | | | |
| A124K | A124R | A125E (1785) | A125D (1786) | A125K (1787) |
| (1783) | (1784) | | | |
| A125R | S126E | S126D (1790) | S126K (1791) | S126R (1792) |
| (1788) | (1789) | | | |
| A127E | A127D | A127K (1795) | A127R (1796) | A128E (1797) |
| (1793) | (1794) | | | |
| A128D | A128K | A128R (1800) | P129S (1801) | P129A (1802) |
| (1798) | (1799) | | | |
| P129K | P129R | P129E (1805) | P129D (1806) | L130V (1807) |
| (1803) | (1804) | | | |
| L130I | L130K | L130R (1810) | L130E (1811) | L130D (1812) |
| (1808) | (1809) | | | |
| R131H | R131Q | T132K (1815) | T132R (1816) | I133K (1817) |
| (1813) | (1814) | | | |
| I133R | I133E | I133D (1820) | T134K (1821) | T134R (1822) |
| (1818) | (1819) | | | |
| T134E | T134D | D136Q (1825) | D136H (1826) | D136K (1827) |
| (1823) | (1824) | | | |
| D136R | T137K | T137R (1830) | T137E (1831) | T137D (1832) |
| (1828) | (1829) | | | |
| F138I | F138V | R139H (1835) | R139Q (1836) | K140N (1837) |
| (1833) | (1834) | | | |
| K140Q | L141I (1839) | L141V (1840) | L141K (1841) | L141R (1842) |
| (1838) | | | | |
| F142I | F142V | F142K (1845) | F142R (1846) | R143H (1847) |
| (1843) | (1844) | | | |
| D136N | R143Q | V144K (1850) | V144R (1851) | Y145H (1852) |
| (1848) | (1849) | | | |
| Y145I | Y145K | Y145R (1855) | S146K (1856) | S146R (1857) |
| (1853) | (1854) | | | |
| F148I | F148V | L149I (1860) | L149V (1861) | R150H (1862) |
| (1858) | (1859) | | | |
| R150Q | K152Q | K152T (1865) | K152N (1866) | L153I (1867) |
| (1863) | (1864) | | | |
| L153V | K154Q | K154T (1870) | K154N (1871) | L155V (1872) |
| (1868) | (1869) | | | |
| L155I | Y156H | Y156I (1875) | E159Q (1876) | E159H (1877) |
| (1873) | (1874) | | | |
| E159N | R162H | R162Q (1880) | D165N (1881) | D165Q (1882) |
| (1878) | (1879) | | | |
| D165H | R166H | R166Q (1885) | | |
| (1883) | (1884) | | | |

TABLE 15
GM-CSF Modifications to Increase Resistance to Proteolysis

| | | | | |
|---|---|---|---|---|
| W61S (86) | W61H (87) | P63A (88) | P63S (89) | P68A (90) |
| P68S (91) | L72I (92) | L72V (93) | F86I (94) | F86V (95) |
| E96N (96) | E96Q (97) | E96H (98) | P100A (99) | P100S (100) |
| E101N (101) | E101Q (102) | E101H (103) | P131A (104) | P131S (105) |
| L133I (106) | L133V (107) | P135A (108) | P135S (109) | F147I (110) |
| F147V (111) | R169H (112) | R169Q (113) | R172H (114) | R172Q (115) |
| P177A (116) | P177S (117) | | | |

TABLE 16
Interleukin-2 (IL-2) Modifications to Increase Resistance to Proteolysis

| | | | | |
|---|---|---|---|---|
| K43N (947) | K43Q (948) | Y45H (949) | Y45I (950) | K48N (951) |
| K48Q (952) | K49N (953) | K49Q (954) | E52N (955) | E52Q (956) |
| E52H (957) | L53I (958) | L53V (959) | E60N (960) | E60Q (961) |
| E60H (962) | E61N (963) | E61Q (964) | E61H (965) | P65A (966) |
| P65S (967) | E67N (968) | E67Q (969) | E67H (970) | E68N (971) |
| E68Q (972) | E68H (973) | L72I (974) | L72V (975) | E100N (976) |
| E100Q (977) | E100H (978) | F103I (979) | F103V (980) | M104I (981) |
| M104V (982) | E106N (983) | E106Q (984) | E106H (985) | Y107H (986) |
| Y107I (987) | D109N (988) | D109Q (989) | E110N (990) | E110Q (991) |
| E110H (992) | L132I (993) | L132V (994) | | |

TABLE 17
Interleukin-3 (IL-3) Modifications to Increase Resistance to Proteolysis

| F37I (996) | F37V (997) | E43N (998) | E43Q (999) | E43H (1000) |
|---|---|---|---|---|
| D46N (1001) | D46Q (1002) | E59N (1003) | E59Q (1004) | E59H (1005) |
| R63H (1006) | R63Q (1007) | K66N (1008) | K66Q (1009) | P96A (1010) |
| P96S | K100N | K100Q | D101N | D101Q |
| (1011) | (1012) | (1013) | (1014) | (1015) |
| D103N (1016) | D103Q | | | |
| | (1017) | | | |

TABLE 18
Interleukin-4 (IL-4) Modifications to Increase Resistance to Proteolysis

| E26N (1019) | E26Q (1020) | E26H (1021) | K37N (1022) | K37Q |
|---|---|---|---|---|
| | | | | (1023) |
| R53H (1024) | R53Q (1025) | E60N (1026) | E60Q (1027) | E60H |
| | | | | (1028) |
| K61N (1029) | K61Q (1030) | R64H (1031) | R64Q (1032) | L66I |
| | | | | (1033) |
| L66V (1034) | P100A (1035) | P100S (1036) | K102N | K102Q |
| | | | (1037) | (1038) |
| E103N (1039) | E103Q (1040) | E103H (1041) | K126N | K126Q |
| | | | (1042) | (1043) |

TABLE 19
Interleukin-5 (IL-5) Modifications to Increase Resistance to Proteolysis

| R32H (1045) | R32Q (1046) | P34A (1047) | P34S (1048) | K39N |
|---|---|---|---|---|
| | | | | (1049) |
| K39Q (1050) | E46N (1051) | E46Q (1052) | E46H (1053) | E47N |
| | | | | (1054) |
| E47Q (1055) | E47H (1056) | E56N (1057) | E56Q (1058) | E56H |
| | | | | (1059) |
| K84N (1060) | K84Q (1061) | K85N (1062) | K85Q (1063) | E88N |
| | | | | (1064) |
| E88Q (1065) | E88H (1066) | E89N (1067) | E89Q (1068) | E89H |
| | | | | (1069) |
| R90H (1070) | R90Q (1071) | E102N (1072) | E102Q | E102H |
| | | | (1073) | (1074) |
| E110N (1075) | E110Q (1076) | E110H (1077) | W111S | W111H |
| | | | (1078) | (1079) |

TABLE 20
Interleukin-13 (IL-13) Modifications to Increase Resistance to Proteolysis

| | | | | |
|---|---|---|---|---|
| M32I (918) | M32V (919) | W34S (920) | W34H (921) | L38I (922) |
| L38V (923) | E48N (924) | E48Q (925) | E48H (926) | F79I (927) |
| F79V (928) | L82I (929) | L82V (930) | R85H (931) | R85Q (932) |
| D86N (933) | D86Q (934) | K88N (935) | K88Q (936) | R107H (937) |
| R107Q (938) | E108N (939) | E108Q (940) | E108H (941) | R110H (942) |
| R110Q (943) | F111I (944) | F111V (945) | | |

TABLE 21
Flt 3 Ligand Modifications to Increase Resistance to Proteolysis

| | | | | |
|---|---|---|---|---|
| D3N (119) | D3Q (120) | D40N (121) | D40Q (122) | E42N (123) |
| E42Q (124) | E42H (125) | L43I (126) | L43V (127) | R55H (128) |
| R55Q (129) | E58N (130) | E58Q (131) | E58H (132) | R59H (133) |
| R59Q (134) | K61N (135) | K61Q (136) | P89A (137) | P89S (138) |
| P90A (139) | P90S (140) | P91A (141) | P91S (142) | R95H (143) |
| R95Q (144) | F96I (145) | F96V (146) | | |

TABLE 22
Stem Cell Factor (SCF) Modifications to Increase Resistance to
Proteolysis

| M27I (1216) | M27V (1217) | K31N (1218) | K31Q (1219) | P34A |
|---|---|---|---|---|
| | | | | (1220) |
| P34S (1221) | D37N (1222) | D37Q (1223) | D54N (1224) | D54Q |
| | | | | (1225) |
| D58N (1226) | D58Q (1227) | D61N (1228) | D61Q (1229) | K62N |
| | | | | (1230) |
| K62Q (1231) | F63I (1232) | F63V (1233) | K96N (1234) | K96Q |
| | | | | (1235) |
| L98I (1236) | L98V (1237) | K99N (1238) | K99Q (1239) | K100N |
| | | | | (1240) |
| K100Q (1241) | F102I (1242) | F102V (1243) | K103N | K103Q |
| | | | (1244) | (1245) |
| E106N (1246) | E106Q (1247) | E106H (1248) | P107A | P107S |
| | | | (1249) | (1250) |
| R108H (1251) | R108Q (1252) | L109I (1253) | L109V | E134N |
| | | | (1254) | (1255) |
| E134Q (1256) | E134H (1257) | D137N (1258) | D137Q | |
| | | | (1259) | |

3. Other Exemplary Polypeptides

Additional protease-resistant polypeptides of the proteins exemplified below can be used in the oral dosage formulations provided herein. Protease resistant polypeptides can be produced by modifying therapeutic proteins using any method known in the art to generate, including, but not limited to, the methods described in US Pat. Pub. Nos. US 2004/0132977 and US 2005/0202438. Exemplary of these polypeptides that can be modified for protease resistance for use in the oral dosage formulations provided are proteins of pharmacologic and/or therapeutic interest, such as those described above. Oral dosage formulations of protease-resistant polypeptides provided herein can include protease-resistant polypeptides that belong to families of therapeutic polypeptides including, but not limited to, blood factors, insulin, relaxin, antibodies, growth factors, soluble receptors, chemokines, angiogenic agents, and neuroactive peptides some of which also include polypeptides already mentioned above. The modified cytokines for use in the oral dosage formulations provided include allelic and species variants of such polypeptides containing corresponding modifications for protease resistance.

a. Blood Factors

Oral dosage formulations of protease-resistant polypeptides provided herein can include a protease-resistant polypeptide that is a blood factor. Exemplary of blood factors include, but are not limited to, tissue plasminogen activator (tPA) (GenBank Accession No. P00750; SEQ ID NO: 2122), Factor VII (GenBank Accession No. P08709; SEQ ID NO: 2123), Factor VIII (GenBank Accession No. P00451; SEQ ID NO: 2124), Factor IX (GenBank Accession No. P00740; SEQ ID NO: 2125), β-globin (GenBank Accession No. P68871; SEQ ID NO: 2126), and hemoglobin. Blood factor polypeptides can be modified using any method known in the art to generate a protease-resistant polypeptide, including, but not limited to, the methods described in US Pat. Pub. Nos. US 2004/0132977 and US 2005/0202438. Such protease-resistant blood factor polypeptides can be prepared as an oral dosage formulation using the methods provided herein.

b. Insulin

Oral dosage formulations of protease-resistant polypeptides can include a protease-resistant polypeptide that is an insulin. Exemplary of insulins include, but are not limited to, human insulin (GenBank Accession No. P01308; SEQ ID NO: 2127) and the preproinsulin, proinsulin and the insulin forms disclosed in U.S. Pat. Nos. 4,992,417; 4,992,418; 5,474,978, 5,514,646, 5,504,188, 5,547,929, 5,650,486, 5,693,609, 5,700,662, 5,747,642, 5,922,675, 5,952,297, 6,034,054, and 6,211,144, and published PCT applications WO 00/121197; WO 09/010,645; and WO 90/12814. Insulin polypeptides also include insulin derivatives and analogs, including, but not limited to, superactive insulin analogs, monomeric insulins, and hepatospecific insulin analogs. Various forms of insulin analogs include, but are not limited to, insulin lispro (Humalog®), insulin lispro mixed with insulin lispro protamine (sold as Humalog® 50/50™, Humalog®75/25™), insulin isophane (sold as Humulin®, Humulin® N, Humulin®N Pen, NPH Iletin® II Pork, Insulin Purified NPH Pork, Novolin® N, Novolin® N Innolet, Novolin® N PenFill), insuline regular (sold as Humulin® R, Iletin® II Regular Pork, Insulin Purified Regular Pork, Novolin® R, Velosulin® BR), insulin isophane mixed with insulin regular (sold as Humulin®50/50™, Humulin®70/30™, Humulin® 70/30Pen™, Novolin® 70/30, Novolin® 70/30 Innolet), insulin zinc (sold as Lente®, Humulin® L, Iletin® Lente, Insulin Lente Pork, Novolin® L) and insulin glargine (Lantus®).

The insulin polypeptides used to generate protease-resistant polypeptides for use in the methods herein can include amino acid substitutions at one or more amino acid positions in the B-chain of insulin (set forth in SEQ ID NO: 2128, which corresponds to amino acid positions 25-54 in SEQ ID NO:2127), or can be glycosylated or acylated. For example, an insulin polypeptide can be is derivatized with a C6-C2 fatty acid (e.g., myristic, pentadecylic, palmitic, heptadecylic, or stearic acid) at an α- or ε-amino group of a glycine, phenylalanine, or lysine residue (U.S. Pat. No. 5,922,675). Exemplary human insulin analogs and derivatives include, but are not limited to, insulin polypeptides wherein the amino acid in position 28 of the B-chain (i.e. B28, corresponding to amino acid position 28 of the sequence set forth in SEQ ID NO:2128) is Asp, Lys, Leu, Val or Ala and the amino acid residue in position 29 of the B-chain (i.e. B29) is Lys or Pro; insulin polypeptides in which amino acid position B28 is Asp and position B29 is Lys or Pro; insulin polypeptides in which position B28 is Lys, and position B29 is Lys or Pro; insulin polypeptides in which position B28 is Asp (i.e. Asp^{B28}); des(B26-B30) human insulin; des(B28-B30) human insulin; des(B27) human insulin; des(B30) human insulin; insulin polypeptides containing Lys^{B28} and Pro^{B29}; B29-N^{ε}-myristoyl-des(B30) human insulin; B29-N^{ε}-palmitoyl-des(B30) human insulin; B29-N^{ε}-myristoyl human insulin; B29-N^{ε}-palmitoyl human insulin; B28-N^{ε}-myristoyl Lys^{B28} Pro^{B29} human insulin; B28-N N^{ε}-palmitoyl Lys^{B28} Pro^{B29} human insulin; B30-N^{ε}-myristoyl-Thr^{B29} Lys^{B30} human insulin; B30-N^{ε}-palmitoyl-Thr^{B29} Lys^{B30} human insulin; B29-N^{ε}-(N-palmitoyl-glutamyl)-des(B30) human insulin; B29-N^{ε}-(N-lithocholyl-glutamyl)-des(B30) human insulin; B29-N^{ε}-(-carboxyheptadecanoyl)-des(B30) human insulin; and B29-N^{ε}-(G-carboxyheptadecanoyl) human insulin.

Insulin polypeptides, including insulin variants, analogs and derivatives, can be modified using any method known in the art to generate a protease-resistant polypeptide, including, but not limited to, the methods described in US Pat. Pub. Nos. US 2004/0132977 and US 2005/0202438. Such protease-resistant insulin polypeptides can be prepared as an oral dosage formulation using the methods provided herein.

c. Relaxin

Oral dosage formulations of protease-resistant polypeptides provided herein can include a protease-resistant polypeptide that is a relaxin. The relaxin polypeptide can be a naturally-occurring relaxin or a synthetic relaxin. Naturally-occurring relaxin can be of human, murine (i.e., rat or mouse), porcine or other mammalian species origin. Human relaxin polypeptides include, but are not limited to, H1 relaxin (GenBank Accession No. P01137; SEQ ID NO: 2155), H2 relaxin (GenBank Accession No. P01137; SEQ ID NO: 2156), H3 relaxin (GenBank Accession No. P01137; SEQ ID NO: 2157), and recombinant human relaxin (rhRLX). Relaxin polypeptides include, but are not limited to, preprorelaxin polypeptides, which are the precursor polypeptides and contain a signal sequence; prorelaxin polypeptides, in which the signal sequence has been removed and the B and A chains are linked by the connecting (C) chain; and the mature relaxin polypeptide, in which the C-chain has been cleaved and the A and B chains are connected by disulphide bonds. The relaxin polypeptides also can contain A and B chains having N- and/or C-terminal truncations. For example, in H2 relaxin, the A chain can be varied from containing amino acids A1-A24 to A10-A24 (corresponding to amino acid positions 162-185 and 171-185, respectively, of the sequence set forth in SEQ ID NO:2156), and the B chain can be varied from containing amino acids B-1-B33 to B10-B22 (corresponding to amino acid positions 25-58 and 35-47, respectively, of the sequence set forth in SEQ ID NO:2156); and in H1 relaxin, the A chain can be varied from containing amino acids A1-A24 to A10-A24 (corresponding to amino acid positions 162-185 and 171-185, respectively, of the sequence set forth in SEQ ID NO:2155) and the B chain can be varied from containing amino acids B1-32 to B10-22 (corresponding to amino acid positions 26-57 and 35-47, respectively, of the sequence set forth in SEQ ID NO:2155) (see e.g., U.S. Pat. No. 5,179,195).

Relaxin polypeptides that can be modified to be protease-resistant for use in the methods provided herein include relaxin analogs and variants including, but not limited to, relaxin polypeptides disclosed in U.S. Pat. No. 5,811,395, and U.S. Pat. No. 6,200,953. Other relaxins include those formulated as described in U.S. Pat. No. 5,945,402. Relaxin analogs and variants can include polypeptides having a replacement of one or more amino acids in the B and/or A chains with a different amino acid, including a D-form of a natural amino-acid. For example, the relaxin polypeptide can contain a substitution of the methionine at B24 (corresponding to amino acid position 49 of the sequences set forth in SEQ ID NOS:2155-2157) with norleucine (Nle), valine (Val), alanine (Ala), glycine (Gly), serine (Ser), or homoserine (Homo Ser). Other relaxin polypeptides can include amino acid substitutions at the B/C and C/A junctions of prorelaxin, or can contain a non-naturally occurring C peptide, such as described in U.S. Pat. No. 5,759,807.

Relaxin polypeptides can be modified using any method known in the art to generate a protease-resistant polypeptide, including, but not limited to, the methods described in US Pat. Pub. Nos. US 2004/0132977 and US 2005/0202438. Such relaxin growth factor polypeptides can be prepared as an oral dosage formulation using the methods provided herein.

d. Antibodies

Oral dosage formulations of protease-resistant polypeptides can include a protease-resistant polypeptide that is an antibody. Exemplary of antibodies include, but are not limited to, antibodies of various isotypes (e.g., IgG1, IgG3 and IgG4), monoclonal antibodies produced by any method known in the art, humanized antibodies, chimeric antibodies, single-chain antibodies, antibody fragments such as Fv, F(ab′) 2, Fab′, Fab and Facb, and any antibody or fragment thereof that is capable of binding an antigen. Exemplary antibodies include antibodies that are specific for a cell surface receptor and that function as antagonists to the receptor, including, but not limited to, antibodies to transforming growth factor (TGF)-β receptor, antibodies to tumor necrosis factor (TNF)-α receptor, antibodies to vascular endothelial growth factor (VEGF) receptor (see, e.g., U.S. Pat. Nos. 6,617,160, 6,448,077, and 6,365,157) and antibodies to epidermal growth factor receptor; antibodies specific for receptor ligands, including, but not limited to, antibodies to TGF-β, antibodies to TNF-α, and antibodies to VEGF; antibodies specific for a tumor-associated antigen; antibodies specific for CD20; antibodies specific for epidermal growth factor receptor-2; antibodies specific for the receptor binding domain of IgE; antibodies specific for adhesion molecules, such as the α subunit (CD11a) of LFA-1 and antibodies specific for α4β7. Antibody polypeptides can be modified using any method known in the art to generate a protease-resistant polypeptide, including, but not limited to, the methods described in US Pat. Pub. Nos. US 2004/0132977 and US 2005/0202438. Such protease-resistant antibody polypeptides can be prepared as an oral dosage formulation using the methods provided herein.

e. Growth Factors

Oral dosage formulations of protease-resistant polypeptides can include a protease-resistant polypeptide that is a growth factors. Exemplary of growth factors include, but are not limited to, keratinocyte growth factor (GenBank Accession No. P21781; SEQ ID NO: 2129), acidic fibroblast growth factor (GenBank Accession No. P05230; SEQ ID NO: 2130), stem cell factor (GenBank Accession No. P21583; SEQ ID NO: 2131), basic fibroblast growth factor (GenBank Accession No. P09038; SEQ ID NO: 2132), hepatocyte growth factor (GenBank Accession No. P14210; SEQ ID NO: 2133), insulin-like growth factor 1A (GenBank Accession No. P01343; SEQ ID NO: 2134), insulin-like growth factor 1B (GenBank Accession No. P05019; SEQ ID NO: 2135), and any fragments thereof. Also contemplated are fusion proteins containing a growth factor or fragment thereof. Growth factor polypeptides can be modified using any method known in the art to generate a protease-resistant polypeptide, including, but not limited to, the methods described in US Pat. Pub. Nos. US 2004/0132977 and US 2005/0202438. Such protease-resistant growth factor polypeptides can be prepared as an oral dosage formulation using the methods provided herein.

f. Soluble Receptors

Oral dosage formulations of protease-resistant polypeptides can include a protease-resistant polypeptide that is a soluble receptor. Exemplary of soluble receptors include, but are not limited to, a TNF-α-binding soluble receptor, a soluble VEGF receptor, soluble interleukin receptor, soluble IL-1 receptor, a soluble type II IL-1 receptor, a soluble γδT cell receptor, soluble EGFR or other ErbB receptor and any active portion, such as ligand-binding fragment, of a soluble receptor. Soluble receptors bind a ligand that, under normal physiological conditions, binds to a membrane-bound or cell surface receptor which can lead to various activation and/or signaling events. Thus, a soluble receptor is one that can function as a receptor antagonist, by binding the ligand and preventing activation and/or signaling via the membrane-bound or cell surface receptor.

The amino acid sequences of various soluble receptors are known in the art and can be used to generate protease-resistant polypeptides for use in the methods provided herein. For example, amino acid sequences of soluble VEGF receptors are known in the databases (GenBank Accession No. AAC50060; SEQ ID NO: 2136, and GenBank Accession No. NP_{—}002010; SEQ ID NO: 2137) and described in U.S. Pat. Nos. 6,383,486, 6,375,929, and 6,100,071; soluble IL-4 receptors are described U.S. Pat. No. 5,599,905; and soluble IL-1 receptors are described in U.S. Patent Publication No. 20040023869.

Soluble receptor polypeptides can be modified using any method known in the art to generate a protease-resistant polypeptide, including, but not limited to, the methods described in US Pat. Pub. Nos. US 2004/0132977 and US 2005/0202438. Such protease-resistant soluble receptor polypeptides can be prepared as an oral dosage formulation using the methods provided herein.

g. Chemokines

Oral dosage formulations of protease-resistant polypeptides can include a protease-resistant polypeptide that is a chemokine. Exemplary of chemokines include, but are not limited to, IP-10 (GenBank Accession No. P02778; SEQ ID NO: 2138), MIG (GenBank Accession No. Q07325; SEQ ID NO: 2139), Groa (GenBank Accession No. P09341; SEQ ID NO: 2140), RANTES (GenBank Accession No. P13501; SEQ ID NO: 2124), MIP-1α (GenBank Accession No. P10147; SEQ ID NO: 2142), MIP-11 (GenBank Accession No. P13236; SEQ ID NO: 2143), MCP-1 (GenBank Accession No. P13500; SEQ ID NO: 2144), PF-4 (GenBank Accession No. P02776; SEQ ID NO: 2145), and any other chemokine, fragment thereof, and fusion protein containing such. The amino acid sequences of chemokines and variants thereof are known in the art, and can be used to generate protease-resistant polypeptides for use in the methods provided herein. For example, amino acid sequences of IP-10 are disclosed in U.S. Pat. Nos. 6,491,906, 5,935,567, 6,153,600, 5,728,377, and 5,994,292; amino acid sequences of MIG are disclosed in U.S. Pat. No. 6,491,906, and Farber et al., (1993) Biochem. Biophys. Res. Comm. 192(1):223-230; and amino acid sequences of RANTES are disclosed in U.S. Pat. Nos. 6,709,649, 6,168,784, and 5,965,697.

Chemokine polypeptides can be modified using any method known in the art to generate a protease-resistant polypeptide, including, but not limited to, the methods described in US Pat. Pub. Nos. US 2004/0132977 and US 2005/0202438. Such protease-resistant chemokine polypeptides can be prepared as an oral dosage formulation using the methods provided herein.

h. Angiogenic Agents

Oral dosage formulations of protease-resistant polypeptides can include a protease-resistant polypeptide that is an angiogenic agent. Exemplary of angiogenic agents include, include, but are not limited to, VEGF polypeptides, including VEGF₁₂₁ (QenBank Accession No. AAF19659; SEQ ID NO: 2152 and GenBank Accession No. ABO26344; SEQ ID NO: 2153), VEGF₁₆₅ (GenBank Accession No. P15692-4; SEQ ID NO: 2154), VEGF-A (GenBank Accession No. P15692; SEQ ID NO: 2146), VEGF-B (GenBank Accession No. P49765; SEQ ID NO: 2147), VEGF-C (GenBank Accession No. P49767; SEQ ID NO: 2148), and VEGF-D (GenBank Accession No. O43915; SEQ ID NO: 2149), transforming growth factor-beta (GenBank Accession No. P01137; SEQ ID NO: 2150), basic fibroblast growth factor (GenBank Accession No. P09038; SEQ ID NO: 2132) angiogenin (GenBank Accession No. P03950; SEQ ID NO: 2151) and gliomas-derived growth factor. The amino acid sequences of angiogenic agents and variants thereof are known in the art and can be used to generate protease-resistant polypeptides for use in the methods provided herein. For example, amino acid sequences of VEGF polypeptides are disclosed in U.S. Pat. Nos. 5,194,596, 5,332,671, 5,240,848, 6,475,796, 6,485,942, and 6,057,428; amino acid sequences of VEGF-C (also known as VEGF-2) polypeptides are disclosed in U.S. Pat. Nos. 5,726,152 and 6,608,182; and amino acid sequences of glioma-derived growth factors having angiogenic activity are disclosed in U.S. Pat. Nos. 5,338,840 and 5,532,343. Angiogenic agent polypeptides can be modified using any method known in the art to generate a protease-resistant polypeptide, including, but not limited to, the methods described in US Pat. Pub. Nos. US 2004/0132977 and US 2005/0202438. Such protease-resistant angiogenic agent polypeptides can be prepared as an oral dosage formulation using the methods provided herein

i. Neuroactive Peptides

Oral dosage formulations of protease-resistant polypeptides can include a protease-resistant polypeptide that is a neuroactive peptide. Exemplary of neuroactive peptides include, but are not limited to, nerve growth factor, bradykinin, cholecystokinin, gastin, secretin, oxytocin, gonadotropin-releasing hormone, beta-endorphin, enkephalin, substance P, somatostatin, prolactin, galanin, growth hormone-releasing hormone, bombesin, dynorphin, neurotensin, motilin, thyrotropin, neuropeptide Y, luteinizing hormone, calcitonin, insulin, glucagons, vasopressin, angiotens in II, thyrotropin-releasing hormone, vasoactive intestinal peptide, and sleep peptide. Neuroactive peptide polypeptides can be modified using any method known in the art to generate a protease-resistant polypeptide, including, but not limited to, the methods described in US Pat. Pub. Nos. US 2004/0132977 and US 2005/0202438. Such protease-resistant neuroactive peptide polypeptides can be prepared as an oral dosage formulation using the methods provided herein.

j. Additional Proteins

Additional exemplary polypeptides for use in the oral dosage formulations provided in the methods herein include other proteins of pharmacologic interest including, but not limited to, thrombolytic agents, atrial natriuretic peptides, bone morphogenic protein, thrombopoletin, glial fibrillary acidic protein, follicle stimulating hormone, alpha-1 antitrypsin, leukemia inhibitory factor, transforming growth factor, insulin-like growth factor, a luteinizing hormone, macrophage activating factor, tumor necrosis factor, a neutrophil chemotactic factor, a nerve growth factor, a tissue inhibitor of metalloproteinases, a vasoactive intestinal peptide, angiotropin, fibrin, hirudin, and leukemia inhibitory factor.

### E. ORAL DOSAGE FORMULATIONS OF IFN-α PROTEASE-RESISTANT POLYPEPTIDES

Oral dosage formulations for oral administration of amounts of protease-resistant polypeptide to achieve therapeutically effective amounts in the bloodstream. Exemplary of such polypeptides is interferon-α (IFN-α). Such protease-resistant polypeptides can be used in the treatment of any disease or condition for which a native or wild-type or other mutant form of the polypeptide, such as IFN-α, is administered. Native or wild-type and other forms of IFN-α are administered for the treatment of a variety of diseases or conditions, some of which are described below, including chronic hepatitis C viral infections, chronic hepatitis B viral infection, condylomata acuminate, and cancers such as hairy cell leukemia, malignant lymphoma, follicular lymphoma, and AIDS-related Karposi sarcoma. Treatment of these diseases or conditions with other forms of IFN-α, however, is by subcutaneous and intravenous routes non-resistant polypeptides do not reach therapeutic levels in the bloodstream when administered orally. As shown in the Examples provided below, little or no native IFN-α (i.e. IFN-α that has not been modified to be protease resistant) is absorbed into the bloodstream following oral administration, even when the native IFN-α polypeptide formulation is delivered to the lower intestinal tract via an enteric coating (see e.g., Examples 9 and 11 for studies done in both rats and primates). By contrast, the oral dosage formulations provided herein containing protease-resistant IFN-α polypeptides can be absorbed into the bloodstream when administered orally.

As shown in the Examples, oral delivery of a protease-resistant IFN-α polypeptide, IFN-α(E41Q), via various forms of oral administration, including enterically-coated tablets and capsules, intraduodenal delivery and liquid gavage, results in high levels of absorption of IFN-α polypeptide into the bloodstream. Furthermore, administration of oral dosage formulations of a protease-resistant IFN-α polypeptide can achieve levels of IFN-α in the blood comparable to the levels observed for subcutaneous administration (see e.g., Table 69). Hence, the oral formulations of protease-resistant IFN-α polypeptides provided herein provide a way to achieve therapeutically effective amounts of IFN-α by oral administration.

1. Protease-Resistant IFN-α Polypeptides

Exemplary IFN-α protease-resistant polypeptides include one or more amino acid modifications in the primary amino acid sequence of an IFN-α polypeptide, such as an IFN-α2b polypeptide (SEQ ID NO: 2067), where the one or more amino acid replacements lead to greater resistance to proteases, including but not limited to proteases of the gastrointestinal tract. Such modifications include, but are not limited to, modifications shown in Table 2, where the replacements are made compared to the sequence of amino acids set forth in SEQ ID NO: 2067. Such modifications can be made in an IFN-α2b polypeptide (SEQ ID NO: 2067) or any allelic or species variant thereof.

The oral dosage formulations provided of IFN-α protease-resistant polypeptides also can contain allelic variants and structural homologues of IFNα-2b that are modified by one or more amino acid replacements in the primary amino acid sequence of the polypeptide that lead to greater resistance to proteases, including but not limited to proteases of the gastrointestinal tract. Such allelic variants and structural homologues of IFNα-2b include, but are not limited to, IFNα-2a (SEQ ID NO: 2162), IFNα-c (SEQ ID NO: 2163), IFNα-2c (SEQ ID NO: 2164), IFNα-d (SEQ ID NO: 2165), IFNα-5 (SEQ ID NO: 2166), IFNα-6 (SEQ ID NO: 2167), IFNα-4 (SEQ ID NO: 2168), IFNα-4-b (SEQ ID NO: 2169), IFNα-I (SEQ ID NO: 2170), IFNα-J (SEQ ID NO: 2171), IFNα-H (SEQ ID NO: 2172), IFNα-F (SEQ ID NO: 2173), IFNα-8 (SEQ ID NO: 2174) and IFNα-consensus cytokine (SEQ ID NO: 2175), and other consensus cytokines disclosed, for example in U.S. Pat. Nos. 4,695,623 and 4,897,471 (e.g., IFN-con1 is the consensus interferon agent in the InfergenB alfacon-1 product (interferon alfacon-1, InterMune, Inc., Brisbane, Calif.)).

Accordingly, the modified IFNα cytokines for use in the oral dosage formulations provided are those with one or more amino acid replacements at one or more amino acid positions in either IFNα-2a, IFNα-c, IFNα-2c, IFNα-d, IFNα-5, IFNα-6, IFNα-4, IFNα-4b, IFNα-I, IFNα-J, IFNα-H, IFNα-F, IFNα-8, or IFNα-consensus cytokine corresponding to a structurally-related modified amino acid position within the 3-dimensional structure of the IFNα-2b modified proteins for protease-resistance, such as for example, IFNα-2b modifications presented in Table 2.

In a particular example of a protease-resistant IFN-α that can be used in the oral dosage formulations provided, the modification in an IFN-α polypeptide is replacement of a glutamic acid residue (E) with a glutamine residue (Q). Exemplary of such replacement is replacement of a glutamic acid residue (E) with a glutamine residue (Q) at position 41 in a mature IFN-α (e.g., a mature IFN-α is set forth in SEQ ID NO: 2067) or allelic or species variants thereof. Further, an exemplary protease-resistance polypeptide is an IFN-α2b mature polypeptide, where the glutamic acid residue (E) at position 41 is replaced with a glutamine residue (Q) (referred to herein as IFN-α2b(E41Q); SEQ ID NO: 1995). An exemplary mature human IFN-α2b(E41Q) polypeptide that can be used in the oral dosage formulations provided herein has the following sequence of amino acids:

(SEQ ID NO: 1995)

CDLPQTHSLGSRRTLMLLAQMRRISLFSCLKDRHDFGFPQQEFGNQFQKA
ETIPVLHEMIQQIFNLFSTKDSSAAWDETLLDKFYTELYQQLNDLEACVI
QGVGVTETPLMKEDSILAVRKYFQRITLYLKEKKYSPCAWEVVRAEIMRS
FSLSTNLQESLRSKE.

a. Further Modifications of Protease-Resistant IFN-α Polypeptides

Protease-resistant IFN-α polypeptides for use in the oral dosage formulations provided also include protease-resistant IFN-α polypeptides that have been further modified by glycosylation, carboxylation, hydroxylation, hasylation, carbamylation, sulfation, phosphorylation, and/or PEGylation. Protease-resistant IFN-α polypeptides for use in the oral dosage formulations provided also include protease-resistant IFN-α polypeptides that have been further modified by one or more amino acid modifications that contribute to, for example, increased activity (e.g., increased antiviral activity), altered immunogenicity, stability, thermal tolerance, protease-resistance, glycosylation, carboxylation, hydroxylation, hasylation, carbamylation, sulfation, phosphorylation, and/or PEGylation. Such amino acid modifications can be natural amino acids, non-natural amino acids or a combination of natural and non-natural amino acids. The protease-resistant IFN-α polypeptides for use in the oral dosage formulations provided also can be conjugated or expressed as a fusion protein with one or more proteins or peptides, such as albumin. In addition, protein modifications also can include modification to facilitate the detection, purification, and assay development of an IFN-α polypeptide, such as for example, modification of a polypeptide with a Sulfo-NHS-LC-biotin for covalent attachment to a primary amine on a protein, or other similar modification for florescent, non-isotopic, or radioactive labels. Generally, the modification results in increased stability without losing at least one activity, such as antiviral activity (i.e., retains at least one activity as defined herein) of an unmodified IFN-α polypeptide.

Modifications to decrease immunogenicity of the IFN-α polypeptide typically involve replacement of at least one amino acid residue resulting in a substantial reduction in activity of or elimination of one or more T cell epitopes from the protein, i.e., deimmunization of the polypeptide. One or more amino acid modifications at particular positions within any of the MHC class II ligands can result in a deimmunized IFN-α polypeptide with a reduced immunogenic when administered as a therapeutic to a host, such as for example, a human host. Exemplary modifications to decrease the immunogenicity of the IFN-α polypeptide include, for example, mutations disclosed in U.S. Patent Application Publication No. US2006/0062761 A1, such as but not limited to, amino acid modifications at one or more positions corresponding to any of the following positions: I24P, L26P, F27S, F38E, I63T, F64A, F64C, F64D, F64E, F64G, F64H, F64K, F64M, F64N, F64P, F64Q, F64R, F64S, F64T, F64W, F64Y, N65A, N65C, N65D, N65E, N65F, N65G, N65H, N65I, N65K, N65L, N65M, N65P, N65Q, N65R, N65S, N65T, N65V, N65W, N65YL66A, L66C, L66D, L66E, L66G, L66H, L66K, L66N, L66P, L66Q, L66R, L66S, L66T, L66M, L66V, L66W, L66Y, F67A, F67C, F67D, F67E, F67G, F67H, F67K, F67N, F67P, F67Q, F67R, F67S, F67T, F67M, F67V, F67W, F67Y, S68A, S68F, S68G, S68H, S68I, S68M, S68P, S68T, S68V, S68W, S68Y, K70A, K70C, K70H, K70P, D71F, D71H, D71I, D71L, D71P, D71T, D71V, D71W, D71Y, S72D, S72H, S72P, S72T, S72W, S72Y, S73A, S73C, S73G, S73P, S73T, A74D, A74P, A74Q, A74R, A74S, A74T, A75C, A75D, A75E, A75G, A75H, A75K, A75N, A75P, A75Q, A75R, A75S, A75T, A75W, A75, A75, AW76A, W76C, W76D, W76E, W76G, W76H, W76K, W76N, W76P, W76Q, W76R, W76S, W76T, W76M, W76, WE78A, E78C, E78G, F84D, F84E, FY85S, Y89E, Y89N, Y89D, V103E, L110G, L110S, M111T, M111S, M111E, K112A, K112C, K112G, K112P, D114P, S115A, S115C, S115D, S115F, S115G, S115H, S115I, S115P, S115W, S115Y, I116A, I116C, I116D, I116E, I116G, I116H, I116K, I116N, I116P, I116Q, I116R, I116S, I116T, I116W, I116Y, L117A, L117C, L117D, L117E, L117G, L117H, L117K, L117N, L117P, L117Q, L117R, L117S, L117T, L117M, L117W, L117Y, V118D, V118E, V118H, V1118K, V118N, V118P, V118Q, V118R, V118S, V118T, V118, V118, V118, V118, V119A, V119C, V119G, V119P, V119T, R120P, Y122A, Y122C, Y122D, Y122E, Y122G, Y122H, Y122K, Y122N, Y122P, Y122Q, Y122R, Y122S, Y122T, Y122, F123A, F123C, F123D, F123E, F123G, F123H, F123K, F123N, F123P, F123Q, F123R, F123S, F123T, F123, Q124A, Q124C, Q124G, Q124H, Q1241, Q124M, Q124P, Q124T, Q124V, Q124W, Q124Y, Q124, Q124, Q124, R125P, R125T, R125W, R125Y, I126A, I126C, I126D, I126E, I126G, I126H, I126K, I126N, I126P, I126Q, I126R, I126S, I126T, I126M, T127P, T127W, T127Y, L128A, L128C, L128G, L128P, L128Q, L128R, L128S, L128T, Y129D, Y129E, Y129H, Y129K, Y129P, Y129Q, Y129R, Y129S, Y129T, Y129M, and L153S of a mature IFN-α polypeptide set forth in SEQ ID NO: 2067 or an allelic or species variant thereof.

The degree of glycosylation of IFN-α polypeptides for use in the oral dosage formulations provided can be altered in order to achieve 1) reduced immunogenicity; 2) less frequent administration of the protein; 3) increased protein stability such as increased serum half-life; and 4) reduction in adverse side effects such as inflammation. The further glycosylation of an IFN-α polypeptide confers one or more advantages including increased serum half-life; reduced immunogenicity; increased functional in vivo half-life; reduced degradation by gastrointestinal tract conditions such as gastrointestinal tract proteases; and increased rate of absorption by gut epithelial cells. An increased rate of absorption by gut epithelial cells and reduced degradation by gastrointestinal tract conditions is important for enteral oral formulations of an IFN-α polypeptide.

A hyperglycosylated IFN-α polypeptide for use in the oral dosage formulations provided can include O-linked glycosylation, N-linked glycosylation, and/or a combination thereof. In some examples, a hyperglycosylated IFN-α polypeptide can include 1, 2, 3, 4, 5, or more carbohydrate moieties, each linked to different glycosylation sites. The glycosylation site can be a native or non-native glycosylation site. In other examples, the hyperglycosylated polypeptide can be glycosylated at a single non-native glycosylation site. In other examples, the hyperglycosylated polypeptide can be glycosylated at more than one non-native glycosylation site, for examples, the hyperglycosylated IFN-α polypeptide can be glycosylated at 1, 2, 3, 4, 5, or more non-native glycosylation sites. Glycosylation sites in an IFN-α polypeptide can be created, altered, eliminated, or rearranged. For example, native glycosylation sites can be modified to prevent glycosylation or enhance or decrease glycosylation, while other positions in the IFN-α polypeptide can be modified to create new glycosylation sites or enhance or decrease glycosylation of existing sites. In some examples, the carbohydrate content of the IFN-α polypeptide can be modified. For example, the number position, bond strength, structure and composition of the carbohydrate linkages (i.e., structure of the carbohydrate based on the nature of the glycosidic linkages or branches of the carbohydrate) of carbohydrate moieties added to the IFN-α polypeptide can be altered.

Changes in the carbohydrate content of the glycosylated IFN-α polypeptides for use in the oral dosage formulations provided, including sialic acid content, can be generated by any method known in the art including but not limited to modification of the primary sequence of the IFN-α polypeptide, enzymatic or chemical modification, production in different host cells, or modified host cells, to produce differences in the glycosylation pattern, and purification methods to enrich IFN-α polypeptides with specific glycosylation profiles. Additionally, growth conditions (e.g., media composition) in which host cells express the modified IFN-α polypeptides can be altered to provide changes in glycosylation, in particular, sialic acid content.

Any amino acid modification that contributes to altered glycosylation of the IFN-α polypeptide can be combined with one or more modifications that increase protease-resistance of the IFN-α polypeptide for use in the oral dosage formulations provided. Exemplary amino acid modifications for modification of an IFN-α glycosylation site, for attachment of a carbohydrate moiety, are described in, for example U.S. Patent Publication No. 2004/0132977-A1 and International PCT Application Publication Nos. WO2006/020580 and WO 2004/022593, and include exemplary modifications corresponding to D2N/P4S, D2N/P4T, L3N/Q5S, L3N/Q5T, P4N/T6S, P4N/T6T, Q5N/H7S, Q5N/H7T, T6N/S8S, T6N/S8T, H7N/L9S, H7N/L9T, S8N/G10S, S8N/G10T, L9N/S11S, L9N/S11T, M21N/K23S, M21N/K23T, R22N/I24S, R22N/I24T, R23N/S25S, R23N/S25T, I24N/L26S, I24N/L26T, S25N/F27S, S25N/F27T, L26N/S28S, L26N/S28T, S28N/L30S, S28N/L30T, L30N/D32S, L30N/D32T, K31N/R33S, K31N/R33T, D32N/H34S, D32N/H34T, R33N/D35S, R33N/D35T, H34N/F36S, H34N/F36T, D35N/G37S, D35N/G37T, F36N/F38S, F36N/F38T, G37N/P39S, G37N/P39T, F38N/Q40S, F38N/Q40T, P39N/E41S, P39N/E41T, Q40N/E42S, Q40N/E42T, E41N/F43S, E41N/F43T, E42N/G44S, E42N/G44T, F43N/N45S, F43N/N45T, G44N/Q46S, G44N/Q46T, N45N/F47S, N45N/F47T, Q46N/Q48S, Q46N/Q48T, F47N/K49S, F47N/K49T, Q48N/A50S, Q48N/A50T, K49N/E51S, K49N/E51T, A50N/T52S, A50N/T52T, S68N/K70S, S68N/K70T, K70N/S72S, K70N/S72T, A75N/D77S, A75N/D77T, D77N/T79S, D77N/T79T, I100N/G102S, I100N/G102T, Q101N/V103S, Q101N/V103T, G102N/G104S, G102N/G104T, V103N/V105S, V103N/V105T, G104N/T106S, G104N/T106T, V105N/E107S, V105N/E107T, T106N/T108S, T106N/T108T, E107N/P109S, E107N/P109T, T108N/I110S, T108N/I110T, K134N/S136S, K134N/S136T, S154N/N156S, S154N/N156T, T155N/L157S, T155N/L157T, N156N/Q158S, N156N/Q158T, L157N/E159S, L157N/E159T, Q158N/S160S, Q158N/S160T, E159N/L161S, E159N/L161T, S160N/R162S, S160N/R162T, L161N/S163S, L161N/S163T, R162N/K164S, R162N/K164T, S163N/E165S, S163N/E165T, D71N, D77N, T106N, D71N/D77N, D71N/T106N, D77N/T106N and D71N/D77N/T106N of a mature IFN-α polypeptide set forth in SEQ ID NO: 2067 or an allelic or species variant thereof.

2. Expression of Protease-Resistant IFN-α Polypeptides

Protease-resistant IFN-α polypeptides for the use in the oral formulations provided can be generated by any method known in the art, including, for example introduction of nucleic acid molecules encoding the protease-resistant IFN-α polypeptide, such as IFN-α2b(E41Q), into a host cell or host animal and expression from nucleic acid molecules encoding the protease-resistant IFN-α polypeptide. Expression hosts include E. coli, yeast, plants (e.g., algae), insect cells, mammalian cells, including cell lines (e.g., Chinese hamster ovary (CHO) cells, Baby hamster kidney (BHK) cells, VERO, HT1080, MDCK, W138, Balb/3T3, HeLa, MT2, mouse NSO (non-secreting) and other myeloma cell lines, hybridoma and heterohybridoma cell lines, lymphocytes, RPMI 1788 cells, fibroblasts, Sp2/0, COS, NIH3T3, HEK293, 293S, 2B8, EBNA-1, and HKB cells (see e.g. U.S. Pat. Nos. 5,618,698, 6,777,205)) and transgenic animals (e.g., production in serum, urine, milk and eggs). Expression hosts can differ in their protein production levels as well as the types of post-translational modifications that are present on the expressed proteins. The choice of expression host can be made based on these and other factors, such as regulatory and safety considerations, production costs and the need and methods for purification.

3. Oral Formulations and Unit Dosage Forms

Protease-resistant IFN-α polypeptides, such as IFN-α2b(E41Q) polypeptides, can be formulated for oral administration as described herein. For example, protease-resistant IFN-α polypeptides can be formulated with one or more pharmaceutically acceptable excipients and lyophilized to generate a powder that can be used to fill a capsule or compressed into a tablet. Such tablets or capsules containing protease-resistant IFN-α polypeptide can optionally include an enteric coating to effect the delivery of the polypeptide to the lower intestinal tract.

Exemplary oral dosage formulations of a protease-resistant IFN-α2b(E41Q) polypeptide are provided in the Examples below. Exemplary steps for the production of the oral dosage formulations of a protease-resistant IFN-α2b(E41Q) polypeptide are provided in the Examples, such as, for the production and purification of the IFN-α2b(E41Q) polypeptide (see e.g., Examples 2, 3, and 4), and production and purification of the lyophilized protein (Example 5).

The oral dosage formulations containing a protease-resistant IFN-α polypeptide, such as IFN-α2b(E41Q) polypeptide, contain one or a plurality of unit dosage forms. Exemplary unit dosage forms include tablets or capsules containing an amount of a protease-resistant IFN-α polypeptide that is a full or a fraction of dosage to be administered for the treatment of a disease or condition. The amount of polypeptide contained in a tablet or a capsule is dependent on various factors, including the desired unit dosage for the disease or condition to be treated, the maximum size of the tablet of capsule, the properties of the pharmaceutical excipients, and the properties of the lyophilized polypeptide. Typically, the unit dosage form contains about 0.001-100 mg of the polypeptide. Exemplary processes for the production of unit dosage forms containing an IFN-α2b(E41Q) polypeptide, including enterically-coated unit dosage forms containing an IFN-α2b(E41Q) polypeptide are provided in the Examples below (see e.g., Example 6). Exemplary unit dosage forms of IFN-α2b(E41Q) are described in Example 6 below, and include, for example, 40 μg, 200 μg and 450 μg of the IFN-α2b(E41Q) polypeptide in a 150 mg compressed tablet. Such unit dosage forms are exemplary and are not intended to limit the amount of a protease-resistant IFN-α polypeptide that can formulated into unit dosage form, such as a tablet or a capsule.

For treatment, the oral dosage formulation can be administered daily, two or more times per day, 6 times per week, 5 times per week, 4 times per week, 3 times per week, 2 times per week, or once per week. Typically, the oral dosage formulation is administered daily.

Oral dosage formulations of protease-resistant IFN-α polypeptides, generally are higher, generally about or 6-400 times, such as 10-100 times, including, for example, 15-50 times, 20-30 times, typically 15-40 times higher (depending upon the therapeutic protein and indication treated and the formulation) per day, than amounts that are administered subcutaneously for the same indication using an unmodified therapeutic protein in the same individual.

For the treatment of HCV, native or other IFN-α polypeptides are administered subcutaneously at a dosage of 12 μg per dose per 70 kg human, three times a week to provide a weekly dosage of 36 μg per week. In an exemplary oral dosage formulation for IFN-α2b(E41Q) where the disease to be treated is an HCV viral infection, exemplary dosage formulations provide herein are selected generally in the range from generally about or 6-200 times, such as 10-100 times, including, for example, 15-50 times, 20-30 times, typically 15-40 times higher than the subcutaneous dose of native IFN-α peptides. For example, the protease resistant IFN-α polypeptide can be administered in a dosage generally about 70-2400 μg, such as 120-1200 μg, including, for example, 180-600 μg, 240-360 μg, typically 180-480 μg. The dosage formulations are provided as dosage unit capsules or tablets contain 6, 12, 18, 24, 32, 40, 50, 60 μg or more per dosage unit. Multiple dosage units typically are administered as an oral dosage formulation.

Such oral formulations can be administered daily for the treatment of HCV infection to provide a weekly dosage of generally about 500-16800 μg, such as 840-8400 μg, including, for example, 1260-4200 μg, 1680-2520 μg, typically 1260-3360 μg. Alternatively, such oral formulations can be administered more or less frequently. For example, oral dosage formulations for the treatment of HCV can be administered 3 times a week to provide a weekly dosage of about of generally about 210-7200 μg, such as 360-3600 μg, including, for example, 540-1800 μg, 720-1080 μg, typically 540-1440 μg.

Provided are dosage units that contain 10 μg, 20 μg, 30 μg, 40 μg, 50 μg, 60 μg, 70 μg, 80 μg, 90 μg, 100 μg, 120 μg, 150 μg, 300 μg and more modified IFN-α2b. Such dosage units can be administered one at time or a plurality at a time. For example, for treatment of HCV infection treatment is effected by daily administration of 30 μg-950 μg/day or about or 80 μg to 6600 μg/week. For example, 80 μg-2200 μg dosage units can be administered daily or three times a week for as long as treatment is indicated.

### F. ORAL DOSAGE FORMULATIONS OF GROWTH HORMONE PROTEASE-RESISTANT POLYPEPTIDES

As mentioned above, oral dosage formulations provided can contain a protease-resistance polypeptide that is a modified growth hormone, such a human growth hormone (hGH). Such protease-resistant polypeptides can be used in the treatment of any disease or condition for which a native or wild-type form of the hGH polypeptide is administered. Native or wild-type forms of hGH are administered for the treatment of a variety of diseases or conditions, some of which are described below, including pediatric and adult growth deficiency disorders (including but not limited to Turner's syndrome, intrauterine growth retardation, idiopathic short stature, Prader Willi syndrome, Thalassaemia), AIDS wasting, aging, impaired immune function of HIV-infected subjects, catabolic illnesses (including those associated with respiratory failure and burn injuries), recovery from surgery, congestive cardiomyopathy, liver transplantation, liver regeneration after hepatectomy, chronic renal failure, renal osteodystrophy, osteoporosis, achondroplasia/hypochondroplasia, skeletal dysplasia, chronic inflammatory or nutritional disorders (such as Crohn's disease), short bowel syndrome, juvenile chronic arthritis, cystic fibrosis, male infertility, X-linked hypophosphatemic rickets, Down's syndrome, Spina bifida, Noonan Syndrome, obesity, impaired muscle strength and fibromyalgia. Treatment of these diseases or conditions with native hGH, however, is limited to subcutaneous and intravenous routes since degradation of the native polypeptide in the gastrointestinal tract when administered via the oral route prevents absorption of the therapeutic polypeptide into the bloodstream. As shown in the Examples provided below, little or no native hGH is absorbed into the bloodstream following oral administration (see e.g., Example 27). By contrast, the oral dosage formulations provided herein containing protease-resistant hGH polypeptides can be absorbed into the bloodstream when administered orally.

As shown in the Examples, oral delivery of protease-resistant hGH polypeptides, hGH(Y42I) and hGH(Y42H), via various forms of oral administration, including capsules or liquid gavage, results in high levels of absorption of hGH polypeptides into the bloodstream. The absorbed protease-resistant hGH polypeptides exhibit growth hormone activity comparable to native hGH administered subcutaneously (see e.g., Example 28). Furthermore, administration of oral dosage formulations of protease-resistant hGH polypeptides can achieve levels of hGH in the blood comparable to the levels observed for subcutaneous administration (see e.g., Examples 26 and 27). Hence, the oral formulations of protease-resistant hGH polypeptides provided herein provide a way to achieve therapeutically effective amounts of hGH by oral administration.

1. Protease-Resistant Growth Hormone Polypeptides

Exemplary hGH protease-resistant polypeptides include one or more amino acid modifications in the primary amino acid sequence of a growth hormone polypeptide, such as a human growth hormone polypeptide (SEQ ID NO: 1260), where the one or more amino acid replacements lead to greater resistance to proteases, including but not limited to proteases of the gastrointestinal tract. Such modifications include, but are not limited to, modifications shown in Table 8, where the replacements are made compared to the sequence of amino acids set forth in SEQ ID NO: 1260. The modifications can be made in hGH polypeptide (SEQ ID NO: 1260) or any allelic or species variant thereof.

In a particular example of a protease-resistant hGH polypeptide that can be used in the oral dosage formulations provided, the modification in an hGH polypeptide is replacement of a tyrosine residue (Y) with an isoleucine residue (I) or a histidine residue (H). Exemplary of such replacement is replacement of a tyrosine residue (Y) with an isoleucine residue (I) at position 42 in a mature hGH (e.g., SEQ ID NO: 1318) or allelic or sequence variants thereof. Another example of such replacement is replacement of a tyrosine residue (Y) with an isoleucine residue (H) at position 42 in a mature hGH (e.g., SEQ ID NO: 1317) or allelic or sequence variants thereof.

An exemplary mature hGH polypeptide that can be used in the oral dosage formulations provided herein is hGH(Y42I) polypeptide and has the following sequence of amino acids:

(SEQ ID NO: 1995)

CDLPQTHSLGSRRTLMLLAQMRRISLFSCLKDRHDFGFPQQEFGNQFQKA
ETIPVLHEMIQQIFNLFSTKDSSAAWDETLLDKFYTELYQQLNDLEACVI
QGVGVTETPLMKEDSILAVRKYFQRITLYLKEKKYSPCAWEVVRAEIMRS
FSLSTNLQESLRSKE.

An exemplary mature hGH polypeptide that can be used in the oral dosage formulations provided herein is hGH(Y42H) polypeptide and has the following sequence of amino acids:

(SEQ ID NO: 1317)

FPTIPLSRLFDNAMLRAHRLHQLAFDTYQEFEEAYIPKEQKHSFLQNPQT
SLCFSESIPTPSNREETQQKSNLELLRISLLLIQSWLEPVQFLRSVFANS
LVYGASDSNVYDLLKDLEEGIQTLMGRLEDGSPRTGQIFKQTYSKFDTNS
HNDDALLKNYGLLYCFRKDMDKVETFLRIVQCRSVEGSCGF.

a. Other Modifications of Protease-Resistant Growth Hormone Polypeptides

Protease-resistant hGH polypeptides for use in the oral dosage formulations provided also include protease-resistant hGH polypeptides that have been further modified by glycosylation, carboxylation, hydroxylation, hasylation, carbamylation, sulfation, phosphorylation, and/or PEGylation. Protease-resistant hGH polypeptides for use in the oral dosage formulations provided also include protease-resistant hGH polypeptides that have been further modified by one or more amino acid modifications that contribute to, for example, increased activity (e.g., increased cell proliferation activity), altered immunogenicity, stability, thermal tolerance, protease-resistance, glycosylation, carboxylation, hydroxylation, hasylation, carbamylation, sulfation, phosphorylation, and/or PEGylation. Such amino acid modifications can be natural amino acids, non-natural amino acids or a combination of natural and non-natural amino acids. The protease-resistant hGH polypeptides for use in the oral dosage formulations provided also can be conjugated or expressed as a fusion protein with one or more proteins or peptides, such as albumin. In addition, protein modifications also can include modification to facilitate the detection; purification, and assay development of an hGH polypeptide, such as for example, modification of a polypeptide with a Sulfo-NHS-LC-biotin for covalent attachment to a primary amine on a protein, or other similar modification for florescent, non-isotopic, or radioactive labels. Generally, the modification results in increased stability without losing at least one activity, such as cell proliferation activity (i.e., retains at least one activity as defined herein) of an unmodified hGH polypeptide.

Others have provided hGH molecules including modified hGH and schemes for its recombinant production, purification and therapeutic use [EP 0107890, U.S. Pat. No. 4,517,181, EP 0105759; U.S. Pat. No. 4,703,035; U.S. Pat. No. 4,658,021; EP0022242; EP0001929; EP0001939; U.S. Pat. No. 4,342,832; U.S. Pat. No. 4,601,980; U.S. Pat. No. 4,604,359; U.S. Pat. No. 4,634,677; U.S. Pat. No. 4,898,830; U.S. Pat. No. 5,424,119; U.S. Pat. No. 4,366,246; U.S. Pat. No. 4,425,437; U.S. Pat. No. 4,431,739; U.S. Pat. No. 4,563,424; U.S. Pat. No. 4,571,421; EP 0131843; EP 0319049; U.S. Pat. No. 4,831,120; U.S. Pat. No. 4,871,835; U.S. Pat. No. 4,997,916; U.S. Pat. No. 5,612,315; U.S. Pat. No. 5,633,352; U.S. Pat. No. 5,618,697; U.S. Pat. No. 5,635,604; EP 0127658; EP 0217814; U.S. Pat. No. 5,898,030; EP0804223; U.S. Pat. No. 7,153,943; U.S. Pat. No. 6,451,561; Lowman H. B. & Wells J. A. (1993) J. Mol. Biol. 243: 564-578]. Such modified hGH proteins can be combined with one or modifications for increased protease resistance to be used in the oral dosage forms provided.

Modifications to decrease immunogenicity of the hGH polypeptide typically involve replacement of at least one amino acid residue resulting in a substantial reduction in activity of or elimination of one or more T cell epitopes from the protein, i.e., deimmunization of the polypeptide. One or more amino acid modifications at particular positions within any of the MHC class II ligands can result in a deimmunized hGH polypeptide with a reduced immunogenic when administered as a therapeutic to a host, such as for example, a human host. Exemplary modifications to decrease the immunogenicity of the hGH polypeptide include, for example, mutations disclosed in U.S. Patent Application Publication No. US2005/0020494-A1.

The degree of glycosylation of hGH polypeptides for use in the oral dosage formulations provided can be altered in order to achieve 1) reduced immunogenicity; 2) less frequent administration of the protein; 3) increased protein stability such as increased serum half-life; and 4) reduction in adverse side effects such as inflammation. The further glycosylation of an hGH polypeptide confers one or more advantages including increased serum half-life; reduced immunogenicity; increased functional in vivo half-life; reduced degradation by gastrointestinal tract conditions such as gastrointestinal tract proteases; and increased rate of absorption by gut epithelial cells. An increased rate of absorption by gut epithelial cells and reduced degradation by gastrointestinal tract conditions is important for enteral oral formulations of an hGH polypeptide.

A hyperglycosylated hGH polypeptide for use in the oral dosage formulations provided can include O-linked glycosylation, N-linked glycosylation, and/or a combination thereof. In some examples, a hyperglycosylated hGH polypeptide can include 1, 2, 3, 4, 5, or more carbohydrate moieties, each linked to different glycosylation sites. The glycosylation site can be a native or non-native glycosylation site. In other examples, the hyperglycosylated polypeptide can be glycosylated at a single non-native glycosylation site. In other examples, the hyperglycosylated polypeptide can be glycosylated at more than one non-native glycosylation site, for examples, the hyperglycosylated hGH polypeptide can be glycosylated at 1, 2, 3, 4, 5, or more non-native glycosylation sites. Glycosylation sites in an hGH polypeptide can be created, altered, eliminated, or rearranged. For example, native glycosylation sites can be modified to prevent glycosylation or enhance or decrease glycosylation, while other positions in the hGH polypeptide can be modified to create new glycosylation sites or enhance or decrease glycosylation of existing sites. In some examples, the carbohydrate content of the hGH polypeptide can be modified. For example, the number position, bond strength, structure and composition of the carbohydrate linkages (i.e., structure of the carbohydrate based on the nature of the glycosidic linkages or branches of the carbohydrate) of carbohydrate moieties added to the hGH polypeptide can be altered.

Changes in the carbohydrate content of the glycosylated hGH polypeptides for use in the oral dosage formulations provided, including sialic acid content, can be generated by any method known in the art including but not limited to modification of the primary sequence of the hGH polypeptide, enzymatic or chemical modification, production in different host cells, or modified host cells, to produce differences in the glycosylation pattern, and purification methods to enrich hGH polypeptides with specific glycosylation profiles. Additionally, growth conditions (e.g., media composition) in which host cells express the modified hGH polypeptides can be altered to provide changes in glycosylation, in particular, sialic acid content.

Any amino acid modification that contributes to altered glycosylation of the hGH polypeptide can be combined with one or more modifications that increase protease-resistance of the hGH polypeptide for use in the oral dosage formulations provided. Exemplary amino acid modifications for modification of an hGH glycosylation site, for attachment of a carbohydrate moiety, are described in, for example, U.S. Patent Publication No. US2005/0250678 and International PCT Application Publication Nos. WO2006/121569 and WO2002/055532.

2. Expression of Nucleic Acid Encoding Protease-Resistant Growth Hormone Polypeptides

Protease-resistant hGH polypeptides for the use in the dosage units and oral formulations provided here can be produced by any method know in the art, including, for example introduction of nucleic acid molecules encoding the protease-resistant hGH polypeptide, such as hGH(Y42I) or hGH(Y42H), into a host cell or host animal and expression from nucleic acid molecules encoding the protease-resistant hGH polypeptide. Expression hosts include E. coli, yeast, plants (e.g., algae), insect cells, mammalian cells, including cell lines (e.g., Chinese hamster ovary (CHO) cells, Baby hamster kidney (BHK) cells, VERO, HT1080, MDCK, W138, Balb/3T3, HeLa, MT2, mouse NSO (non-secreting) and other myeloma cell lines, hybridoma and heterohybridoma cell lines, lymphocytes, RPMI 1788 cells, fibroblasts, Sp2/0, COS, NIH3T3, HEK293, 293S, 2B8, EBNA-1, and HKB cells (see e.g. U.S. Pat. Nos. 5,618,698, 6,777,205)) and transgenic animals (e.g., production in serum, urine, milk and eggs). Expression hosts can differ in their protein production levels as well as the types of post-translational modifications that are present on the expressed proteins. The choice of expression host can be made based on these and other factors, such as regulatory and safety considerations, production costs and the need and methods for purification.

3. Oral Formulations and Unit Dosage Forms

Protease-resistant hGH polypeptides, such as hGH(Y42I) and hGH(Y42H) polypeptides, can be formulated for oral administration as described herein. Tablets and/or capsules containing the modified hGH polypeptides are provided. For example, protease-resistant hGH polypeptides can be formulated with one or more pharmaceutically acceptable excipients and lyophilized to generate a powder that can be used to fill a capsule or compressed into a tablet. Such tablets or capsules containing a protease-resistant hGH polypeptides optionally include an enteric coating to effect the delivery of the polypeptide to the lower intestinal tract.

Exemplary unit oral dosage formulations of a protease-resistant hGH(Y42I) polypeptide are provided in the Examples below. Exemplary steps for the production of the oral dosage formulations of a protease-resistant hGH(Y42I) polypeptide are provided in the Examples, such as, for the production and purification of the hGH(Y42I) polypeptide (see e.g., Examples 21), and production and purification of the lyophilized protein (Example 22).

The oral dosage formulations containing a protease-resistant hGH polypeptide, such as hGH(Y42I) or hGH(Y42H) polypeptide, contain one or a plurality of unit dosage forms. Exemplary unit dosage forms include tablets or capsules containing an amount of a protease-resistant IFN-α polypeptide that is a full or a fraction of dosage to be administered for the treatment of a disease or condition. The amount of polypeptide contained in a tablet or a capsule is dependent on various factors, including the desired unit dosage for the disease or condition to be treated, the maximum size of the tablet of capsule, the properties of the pharmaceutical excipients, and the properties of the lyophilized polypeptide. Typically, the unit dosage form contains about 0.001-100 mg, such as 3, 4, 5, 6, 7, 8, 9, 10, 15, 20, 25, 30, 40, 50, 60, 70, 75, 80, 85, 90, 95 or 100 mg of the polypeptide. Higher dosage units can contain more, such as 125 mg or 150 mg. Oral dosage formulations for administration per day or per week can contain a plurality of dosage units.

Exemplary processes for the production of unit dosage forms containing an hGH(Y42I) or hGH(Y42H) polypeptide, including enterically-coated unit dosage forms containing an hGH(Y42I) or hGH(Y42H) polypeptide are provided in the Examples below (see e.g., Examples 24, 25). Any suitable process based thereon can be sued. Exemplary unit dosage forms of hGH(Y42I) are described in Example 25 below, and include, for example, 3 mg 12 mg and 24 mg of the hGH(Y42I) polypeptide in a 400 mg compressed tablet. Such unit dosage forms are exemplary and are not intended to limit the amount of a protease-resistant IFN-α polypeptide that can formulated into unit dosage form, such as a tablet or a capsule.

For treatment, the oral dosage formulation can be administered daily, two or more times per day, 6 times per week, 5 times per week, 4 times per week, 3 times per week, 2 times per week or once per week or any regimen determined empirically or by a physician for a particular patient or subject. Typically, the oral dosage formulation is administered daily.

Oral dosage formulations of protease-resistant hGH polypeptides, generally are higher, generally about or 6-400 times, such as 10-100 times, including, for example, 15-50 times, 20-30 times, typically 15-40 times higher (depending upon the therapeutic protein and indication treated and the formulation) per day, than amounts that are administered subcutaneously for the same indication using an unmodified therapeutic protein in the same individual. Dosage units contain 0.1, 0.5, 2, 3, 4, 5, 6, 7, 8, 9, 10 mg or higher amounts of the modified hGH polypeptide. Hence provided are dosage units containing the noted amounts, as well as 15 mg, 20 mg, 30 mg, 50 mg, 75 mg, 100 mg and higher amounts. Dosage formulations can contain one or a plurality of dosage units.

For the treatment of a growth hormone deficiency in adults, native hGH peptides are normally administered subcutaneously at a dosage of at our about 0.00625 mg/kg/day for one month followed by a maintenance dose of at or about 0.0125 mg/kg/day. Accordingly, for a 70 kg human, the dosage of native hGH is 0.4375 mg per day for 1 month followed by a maintenance dosage of 0.9375 mg per day. In an exemplary oral dosage formulation for hGH(Y42I), where the disease to be treated is a growth hormone deficiency, exemplary dosage formulations range generally from about 2-90 mg, such as 4-45 mg, including, for example, 6-25 mg, 8-15 mg, and typically 6-18 mg per day. Such dosages can be administered continuously or can be administered for an initial time, such as 1 month dose regimen, followed by a lower maintenance dose, such as, for example about 5-190 mg, such as 9-95 mg, including, for example, 14-47 mg, 18-28 mg, and typically 14-38 mg per day. Such oral formulations can be administered more or less frequently than once per day. The amounts and regimens depend upon the condition treated, the subject and the physician's discretion.

### G. METHODS OF TREATMENT AND USES OF THE ORAL DOSAGE FORMULATIONS

Oral dosage formulations of protease-resistant therapeutic polypeptides provided herein can be used in methods of treating any disease, disorder, or condition for which a corresponding native therapeutic polypeptide is administered. The oral dosage formulation of a protease-resistant polypeptide or dosage units thereof is/are administered orally to a patient in an amount that can effect an increase in the blood concentration of the polypeptide to an amount that is therapeutically effective for the treatment of the disease, disorder, or condition. Typically, the amount of a protease-resistant polypeptide in a composition for oral administration is greater than the amount of a protease-resistant polypeptide that is administered by methods, such as intravenous or subcutaneous injection. The amount of a protease-resistant polypeptide in a composition for oral administration can be determined empirically with respect to the amount of a protease-resistant or native polypeptide that is administered by methods, such as intravenous or subcutaneous injection. In some examples, the amount of a protease-resistant polypeptide in a composition for oral administration is generally about or 6-400 times, such as 10-100 times, including, for example, 15-50 times, 20-30 times, typically 15-40 times higher (depending upon the therapeutic protein and indication treated and the formulation) per day, than amounts that are administered subcutaneously for the same indication using an unmodified therapeutic protein in the same individual. The concentration of a protease-resistant polypeptide in a formulation for oral administration is effective for the treatment of the disease, disorder, or condition in a patient when administered orally.

Additionally, because variant polypeptides used in the oral dosage formulations provided herein exhibit increased protein stability, there is more flexibility in the administration of pharmaceutical compositions than their native polypeptide counterparts. Typically, orally ingested polypeptides are administered in the morning before eating (i.e., before digestive enzymes are activated). The modified polypeptides provided herein exhibit protease resistance to digestive enzymes and can offer the ability to administer oral pharmaceutical compositions containing a protease-resistant polypeptide at other periods during the day and under conditions when digestive enzymes are present and active.

1. Treatment Methods Using IFN-α

In one example, the oral dosage formulation of a protease-resistant polypeptide is administered to a patient who has a disease, disorder, or condition that can be treated by administration of an interferon, such as an IFN-α polypeptide. Typically, oral dosage formulations of protease-resistant IFN-α polypeptides provided herein are administered orally using an amount of IFN-α that is generally about or 6-400 times, such as 10-100 times, including, for example, 15-50 times, 20-30 times, typically 15-40 times higher (depending upon the therapeutic protein and indication treated and the formulation) per day, than amounts that are administered subcutaneously for the same indication using an unmodified therapeutic protein in the same individual, Native IFN-α has been administered for the treatment of a hepatitis C virus (HCV) infection at a dosage of 2 million units (or 15 micrograms or 8.0×10⁻¹⁰ mol) per day three times per week for 48 weeks by subcutaneous injection. Oral dosage formulations of protease-resistant IFN-α polypeptides provided herein are administered orally using an amount of IFN-α which is greater than 2 million units (or 15 micrograms or 8.0×10⁻¹⁰ mol). Typically, the oral dosage formulations of protease-resistant IFN-α polypeptides provided herein are administered orally using an amount of a protease IFN-α in an oral dosage formulation that is generally about or 6-400 times, such as 10-100 times, including, for example, 15-50 times, 20-30 times, typically 15-40 times higher per day, than amounts that are administered subcutaneously for treatment of HCV using an unmodified therapeutic protein in the same individual. Exemplary dosages of a protease-resistant IFN-α polypeptide for oral delivery can thus be determined empirically based on dosages of IFN-α polypeptides administered by other methods, such as subcutaneous or intravenous methods.

In one non-limiting example, oral dosage formulations of protease-resistant IFN-α polypeptides provided herein contain a protease-resistant IFN-α polypeptide, that contains one or more single amino acid replacements at one or more target positions, such as for example, E41 of SEQ ID NO: 2067. Replacement of amino acids at target positions alters or destroys a native protease cleaving site. An exemplary replacement to generate a protease-resistant IFN-α polypeptide includes, but is not limited to, E41Q (SEQ ID NO: 1995).

The oral dosage formulations of protease-resistant IFN-α polypeptides provided herein can be used for treatment of any condition for which unmodified IFN-α is employed. This section provides exemplary uses of protease-resistant IFN-α polypeptides and administration methods. These described therapies are exemplary and do not limit the applications of oral dosage formulations of protease-resistant IFN-α.

The oral dosage formulations of protease-resistant IFN-α polypeptides provided herein are intended for use in various therapeutic as well as diagnostic methods in which IFN-α is used for treatment. Such methods include, but are not limited to, methods of treatment of physiological and medical conditions described and listed below. By virtue of their improved stability, protease-resistant IFN-α polypeptides provided herein exhibit improvement in the corresponding in vivo activities and therapeutic effects.

Exemplary human diseases and/or disorders for which oral dosage formulations of protease-resistant IFN-α polypeptides can be administered include, but are not limited to, cancers and tumors, infectious diseases, venereal diseases, immunologically related diseases and/or autoimmune diseases and disorders, cardiovascular diseases, metabolic diseases, central nervous system diseases, and disorders connected with chemotherapy treatments.

Exemplary cancers and tumors that can be treated with an oral dosage formulation of a protease-resistant IFN-α polypeptide provided herein include carcinomas containing metastasizing renal carcinomas, melanomas, lymphomas containing follicular lymphomas and cutaneous T cell lymphoma, leukemias containing hairy-cell leukemia, chronic lymphocytic leukemia and chronic myeloid leukemia, cancers of the liver, neck, head and kidneys, multiple myelomas, carcinoid tumors and tumors that appear following an immune deficiency, such as Kaposi's sarcoma in the case of AIDS.

Exemplary infectious diseases that can be treated with an oral dosage formulation of a protease-resistant IFN-α polypeptide provided herein include viral infections, such as chronic hepatitis B and C and HIV/AIDS, infectious pneumonias and venereal diseases, such as genital warts.

Exemplary immunologically and auto-immunologically related diseases that can be treated with an oral dosage formulation of a protease-resistant IFN-α polypeptide provided herein include the rejection of tissue or organ grafts, allergies, asthma, psoriasis, rheumatoid arthritis, multiple sclerosis, Crohn's disease and ulcerative colitis.

Exemplary metabolic diseases that can be treated with an oral dosage formulation of a protease-resistant IFN-α polypeptide provided herein include such non-immune associated diseases as obesity.

Exemplary inflammatory diseases that can be treated with an oral formulation of a protease-resistant IFN-α polypeptide provided herein include such inflammatory diseases as Behcet's disease.

Exemplary diseases of the central nervous system that can be treated with an oral dosage formulation of a protease-resistant IFN-α polypeptide provided herein include Alzheimer's disease, Parkinson's disease, schizophrenia and depression.

Exemplary diseases and disorders that can be treated with an oral dosage formulation of a protease-resistant IFN-α polypeptide provided herein also include healing of wounds, anemia in dialyzed patient, and osteoporosis.

An oral dosage formulation of a protease-resistant IFN-α polypeptide is useful for treating various IFN-α-mediated disorders, including viral infections, fibrotic disorders, and proliferative disorders. Thus, methods of treating viral infections, fibrotic disorders, and/or proliferative disorders are provided herein. Such methods generally involve administering to an individual in need thereof an effective amount of an oral dosage formulation of a protease-resistant IFN-α polypeptide, wherein the subject protease-resistant IFN-α polypeptide is prepared as described above, e.g., as an enterically coated tablet, pill, or capsule. In some examples, a treatment method further includes administering at least one additional therapeutic agent for treating a viral disorder, a fibrotic disorder or a proliferative disorder. In some examples, a treatment method further includes administering a side effect management agent, to treat a side effect induced by a therapeutic agent.

a. Fibrotic Disorders

Provided herein are methods for treating a fibrotic disorder in an individual having a fibrotic disorder. The method generally involves administering to an individual in need thereof an effective amount of a protease-resistant IFN-α polypeptide in an oral dosage formulation provided, wherein the subject protease-resistant IFN-α polypeptide is prepared as described above, e.g., as an enterically coated tablet, pill, or capsule. The methods provide for treatment of fibrotic diseases, including those affecting the lung such as idiopathic pulmonary fibrosis, pulmonary fibrosis from a known etiology, liver fibrosis or cirrhosis, cardiac fibrosis, and renal fibrosis. The etiology can be due to any acute or chronic insult including toxic, metabolic, genetic and infectious agents.

Fibrosis is generally characterized by the pathologic or excessive accumulation of collagenous connective tissue. Fibrotic disorders include, but are not limited to, collagen disease, interstitial lung disease, human fibrotic lung disease (e.g., obliterative bronchiolitis, idiopathic pulmonary fibrosis, pulmonary fibrosis from a known etiology, tumor stroma in lung disease, systemic sclerosis affecting the lungs, Hermansky-Pudlak syndrome, coal worker's pneumoconiosis, asbestosis, silicosis, chronic pulmonary hypertension, AIDS-associated pulmonary hypertension, and sarcoidosis), fibrotic vascular disease, arterial sclerosis, atherosclerosis, varicose veins, coronary infarcts, cerebral infarcts, myocardial fibrosis, musculoskeletal fibrosis, post-surgical adhesions, human kidney disease (e.g., nephritic syndrome, Alport's syndrome, HIV-associated nephropathy, polycystic kidney disease, Fabry's disease, diabetic nephropathy, chronic glomerulonephritis, and nephritis associated with systemic lupus), cutis keloid formation, progressive systemic sclerosis (PSS), primary sclerosing cholangitis (PSC), liver fibrosis, liver cirrhosis, renal fibrosis, pulmonary fibrosis, cystic fibrosis, chronic graft versus host disease, scleroderma (local and systemic), Grave's opthalmopathy, diabetic retinopathy, glaucoma, Peyronie's disease, penis fibrosis, urethrostenosis after the test using a cystoscope, inner accretion after surgery, scarring, myelofibrosis, idiopathic retroperitoneal fibrosis, peritoneal fibrosis from a known etiology, drug-induced ergotism, fibrosis incident to benign or malignant cancer, fibrosis incident to microbial infection (e.g., viral, bacterial, parasitic, and fungal), Alzheimer's disease, fibrosis incident to inflammatory bowel disease (including stricture formation in Crohn's disease and microscopic colitis), fibrosis induced by chemical or environmental insult (e.g., cancer chemotherapy, pesticides, and radiation (e.g., cancer radiotherapy)).

In some examples, an effective amount of a protease-resistant IFN-α polypeptide is an amount that, when administered to an individual having a fibrotic disorder, is effective to reduce fibrosis or reduce the rate of progression of fibrosis by at least about 10%, at least about 15%, at least about 20%, at least about 25%, at least about 30%, at least about 35%, at least about 40%, at least about 45%, or at least about 50%, or more, compared with the degree of fibrosis in the individual prior to treatment or compared to the rate of progression of fibrosis that would have been experienced by the patient in the absence of treatment.

In some examples, an effective amount of a protease-resistant IFN-α polypeptide is an amount that, when administered to an individual having a fibrotic disorder, is effective to increase, or to reduce the rate of deterioration of, at least one function of the organ affected by fibrosis (e.g., lung, liver, or kidney) by at least about 10%, at least about 15%, at least about 20%, at least about 25%, at least about 30%, at least about 35%, at least about 40%, at least about 45%, or at least about 50%, or more, compared to the basal level of organ function in the individual prior to treatment or compared to the rate of deterioration in organ function that would have been experienced by the individual in the absence of treatment.

Methods of measuring the extent of fibrosis in a given organ, and methods of measuring the function of any given organ, are well known in the art.

i. Idiopathic Pulmonary Fibrosis

Methods of treating idiopathic pulmonary fibrosis (IPF) are provided. The methods generally involve administering to an individual having IPF an effective amount of a protease-resistant IFN-α polypeptide in an oral dosage formulation provided.

In some examples, a diagnosis of IPF is confirmed by the finding of usual interstitial pneumonia (UIP) on histopathological evaluation of lung tissue obtained by surgical biopsy. The criteria for a diagnosis of IPF are known. Ryu et al. (1998) Mayo Clin. Proc. 73:1085-1101.

In other examples, a diagnosis of IPF is a definite or probable IPF made by high resolution computer tomography (HRCT). In a diagnosis by HRCT, the presence of the following characteristics is noted: (1) presence of reticular abnormality and/or traction bronchiectasis with basal and peripheral predominance; (2) presence of honeycombing with basal and peripheral predominance; and (3) absence of atypical features such as micronodules, peribronchovascular nodules, consolidation, isolated (non-honeycomb) cysts, ground glass attenuation (or, if present, is less extensive than reticular opacity), and mediastinal adenopathy (or, if present, is not extensive enough to be visible on chest x-ray). A diagnosis of definite IPF is made if characteristics (1), (2), and (3) are met. A diagnosis of probable IPF is made if characteristics (1) and (3) are met.

In some examples, an “effective amount” of a protease-resistant IFN-α polypeptide is a dosage that is effective to decrease disease progression by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or more, compared with a placebo control or an untreated control.

Disease progression is the occurrence of one or more of the following: (1) a decrease in predicted FVC of 10% or more; (2) an increase in A-a gradient of 5 mm Hg or more; (3) a decrease of 15% of more in single breath DLc. Whether disease progression has occurred is determined by measuring one or more of these parameters on two consecutive occasions 4 to 14 weeks apart, and comparing the value to baseline.

Thus, for example, where an untreated or placebo-treated individual exhibits a 50% decrease in FVC over a period of time, an individual administered with an effective amount of a protease-resistant IFN-α polypeptide exhibits a decrease in FVC of 45%, about 42%, about 40%, about 37%, about 35%, about 32%, about 30%, or less, over the same time period.

In some examples, an “effective amount” of a protease-resistant IFN-α polypeptide is a dosage that is effective to increase progression-free survival time, e.g., the time from baseline (e.g., a time point from 1 day to 28 days before beginning of treatment) to death or disease progression is increased by at least about 10%, at least about 20%, at least about 25%, at least about 30%, at least about 40%, at least about 50%, at least about 60%, at least about 70%, at least about 80%, at least about 90%, at least about 2-fold, at least about 3-fold, at least about 4-fold, at least about 5-fold, or more, compared a placebo-treated or an untreated control individual. Thus, for example, in some examples an effective amount of a subject a protease-resistant IFN-α polypeptide is a dosage that is effective to increase the progression-free survival time by at least about 1 week, at least about 2 weeks, at least about 3 weeks, at least about 4 weeks, at least about 2 months, at least about 3 months, at least about 4 months, at least about 5 months, at least about 6 months, at least about 8 months, at least about 10 months, at least about 12 months, at least about 18 months, at least about 2 years, at least about 3 years, or longer, compared to a placebo-treated or untreated control.

In some examples, an effective amount of a protease-resistant IFN-α polypeptide is a dosage that is effective to increase at least one parameter of lung function, e.g., an effective amount of a protease-resistant IFN-α polypeptide increases at least one parameter of lung function by at least about 10%, at least about 20%, at least about 25%, at least about 30%, at least about 40%, at least about 50%, at least about 60%, at least about 70%, at least about 80%, at least about 90%, at least about 2-fold, at least about 3-fold, at least about 4-fold, at least about 5-fold, or more, compared to an untreated individual or a placebo-treated control individual. In some of these examples, a determination of whether a parameter of lung function is increased is made by comparing the baseline value with the value at any time point after the beginning of treatment, e.g., 48 weeks after the beginning of treatment, or between two time points, e.g., about 4 to about 14 weeks apart, after the beginning of treatment.

In some examples, an effective amount of a protease-resistant IFN-α polypeptide is a dosage that is effective to increase the FVC by at least about 10% at least about 20%, at least about 25%, at least about 30%, at least about 40%, at least about 50%, at least about 60%, at least about 70%, at least about 80%, at least about 90%, at least about 2-fold, at least about 3-fold, at least about 4-fold, at least about 5-fold, or more compared to baseline on two consecutive occasions 4 to 14 weeks apart.

In some of these examples, an effective amount of a protease-resistant IFN-α polypeptide is a dosage that results in a decrease in alveolar:arterial (A-a) gradient of at least about 5 mm Hg, at least about 7 mm Hg, at least about 10 mm Hg, at least about 12 mm Hg, at least about 15 mm Hg, or more, compared to baseline.

In some of these examples, an effective amount of a protease-resistant IFN-α polypeptide is a dosage that increases the single breath DL_{co} by at least about 15%, at least about 20%, at least about 30%, at least about 40%, at least about 50%, at least about 60%, at least about 70%, at least about 80%, at least about 90%, at least about 2-fold, at least about 3-fold, at least about 4-fold, at least about 5-fold, or more, compared to baseline. DL_{co} is the lung diffusing capacity for carbon monoxide, and is expressed as mL CO/mm Hg/second.

Parameters of lung function include, but are not limited to, forced vital capacity (FVC); forced expiratory volume (FEV₁); total lung capacity; partial pressure of arterial oxygen at rest; partial pressure of arterial oxygen at maximal exertion. Lung function can be measured using any known method, including, but not limited to spirometry.

ii. Liver Fibrosis

Methods are provided for treating liver fibrosis, including reducing clinical liver fibrosis, reducing the likelihood that liver fibrosis will occur, and reducing a parameter associated with liver fibrosis. The methods generally involve administering an effective amount of a protease-resistant IFN-α polypeptide in an oral dosage formulation provided to an individual in need thereof. Of particular interest in many examples is treatment of humans.

Liver fibrosis is a precursor to the complications associated with liver cirrhosis, such as portal hypertension, progressive liver insufficiency, and hepatocellular carcinoma. A reduction in liver fibrosis thus reduces the incidence of such complications. Accordingly, methods are provided for reducing the likelihood that an individual will develop complications associated with cirrhosis of the liver.

The present methods generally involve administering a therapeutically effective amount of a protease-resistant IFN-α polypeptide in an oral dosage formulation. In some examples, an “effective amount” of a protease-resistant IFN-α polypeptide is an amount that is effective in reducing liver fibrosis or reduce the rate of progression of liver fibrosis; and/or that is effective in reducing the likelihood that an individual will develop liver fibrosis; and/or that is effective in reducing a parameter associated with liver fibrosis; and/or that is effective in reducing a disorder associated with cirrhosis of the liver.

Methods are provided for treatment of liver fibrosis in an individual by administering to a subject an amount of a protease-resistant IFN-α polypeptide that is effective for prophylaxis or therapy of liver fibrosis in the individual, e.g., increasing the probability of survival, reducing the risk of death, ameliorating the disease burden or slowing the progression of disease in the individual.

Whether treatment with a protease-resistant IFN-α polypeptide is effective in reducing liver fibrosis is determined by any of a number of well-established techniques for measuring liver fibrosis and liver function. Whether liver fibrosis is reduced is determined by analyzing a liver biopsy sample. An analysis of a liver biopsy includes assessments of two major components: necroinflammation assessed by “grade” as a measure of the severity and ongoing disease activity, and the lesions of fibrosis and parenchymal or vascular remodeling as assessed by “stage” as being reflective of long-term disease progression. See, e.g., Brunt (2000) Hepatol. 31:241-246; and METAVIR (1994) Hepatology 20:15-20. Based on analysis of the liver biopsy, a score is assigned. A number of standardized scoring systems exist which provide a quantitative assessment of the degree and severity of fibrosis. These include the METAVIR, Knodell, Scheuer, Ludwig, and Ishak scoring systems.

The METAVIR scoring system is based on an analysis of various features of a liver biopsy, including fibrosis (portal fibrosis, centrilobular fibrosis, and cirrhosis); necrosis (piecemeal and lobular necrosis, acidophilic retraction, and ballooning degeneration); inflammation (portal tract inflammation, portal lymphoid aggregates, and distribution of portal inflammation); bile duct changes; and the Knodell index (scores of periportal necrosis, lobular necrosis, portal inflammation, fibrosis, and overall disease activity). The definitions of each stage in the METAVIR system are as follows: score: 0, no fibrosis; score: 1, stellate enlargement of portal tract but without septa formation; score: 2, enlargement of portal tract with rare septa formation; score: 3, numerous septa without cirrhosis; and score: 4, cirrhosis.

Knodell's scoring system, also called the Hepatitis Activity Index, classifies specimens based on scores in four categories of histologic features: I. Periportal and/or bridging necrosis; II. Intralobular degeneration and focal necrosis; III. Portal inflammation; and IV. Fibrosis. In the Knodell staging system, scores are as follows: score: 0, no fibrosis; score: 1, mild fibrosis (fibrous portal expansion); score: 2, moderate fibrosis; score: 3, severe fibrosis (bridging fibrosis); and score: 4, cirrhosis. The higher the score, the more severe the liver tissue damage. Knodell (1981) Hepatol. 1:431.

In the Scheuer scoring system scores are as follows: score: 0, no fibrosis; score: 1, enlarged, fibrotic portal tracts; score: 2, periportal or portal-portal septa, but intact architecture; score: 3, fibrosis with architectural distortion, but no obvious cirrhosis; score: 4, probable or definite cirrhosis. Scheuer (1991) J. Hepatol. 13:372.

The Ishak scoring system is described in Ishak (1995) J. Hepatol. 22:696-699. Stage 0, No fibrosis; Stage 1, Fibrous expansion of some portal areas, with or without short fibrous septa; stage 2, Fibrous expansion of most portal areas, with or without short fibrous septa; stage 3, Fibrous expansion of most portal areas with occasional portal to portal (P-P) bridging; stage 4, Fibrous expansion of portal areas with marked bridging (P-P) as well as portal-central (P-C); stage 5, Marked bridging (P-P and/or P-C) with occasional nodules (incomplete cirrhosis); stage 6, Cirrhosis, probable or definite. The benefit of anti-fibrotic therapy also can be measured and assessed by using the Child-Pugh scoring system is a multicomponent point system based upon abnormalities in serum bilirubin level, serum albumin level, prothrombin time, the presence and severity of ascites, and the presence and severity of encephalopathy. Based upon the presence and severity of abnormality of these parameters, patients can be placed in one of three categories of increasing severity of clinical disease: A, B, or C.

In some examples, a therapeutically effective amount of a protease-resistant IFN-α polypeptide is an amount that effects a change of one unit or more in the fibrosis stage based on pre- and post-therapy liver biopsies. In particular examples, a therapeutically effective amount of a protease-resistant IFN-α polypeptide reduces liver fibrosis by at least one unit in the METAVIR, the Knodell, the Scheuer, the Ludwig, or the Ishak scoring system.

Secondary, or indirect, indices of liver function also can be used to evaluate the efficacy of treatment with a protease-resistant IFN-α polypeptide. Morphometric computerized semi-automated assessment of the quantitative degree of liver fibrosis based upon specific staining of collagen and/or serum markers of liver fibrosis also can be measured as an indication of the efficacy of a subject treatment method. Secondary indices of liver function include, but are not limited to, serum transaminase levels, prothrombin time, bilirubin, platelet count, portal pressure, albumin level, and assessment of the Child-Pugh score.

In another example, an effective amount of a protease-resistant IFN-α polypeptide is an amount that is effective to increase an index of liver function by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the index of liver function in an untreated individual, or in a placebo-treated individual. Those skilled in the art can readily measure such indices of liver function, using standard assay methods, many of which are commercially available, and are used routinely in clinical settings.

Serum markers of liver fibrosis also can be measured as an indication of the efficacy of a subject treatment method. Serum markers of liver fibrosis include, but are not limited to, hyaluronate, N-terminal procollagen III peptide, 7S domain of type IV collagen, C-terminal procollagen I peptide, and laminin. Additional biochemical markers of liver fibrosis include .alpha.-2-macroglobulin, haptoglobin, gamma globulin, apolipoprotein A, and gamma glutamyl transpeptidase.

In another example, a therapeutically effective amount of a protease-resistant IFN-α polypeptide is an amount that is effective to reduce a serum level of a marker of liver fibrosis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the level of the marker in an untreated individual, or in a placebo-treated individual. Those skilled in the art can readily measure such serum markers of liver fibrosis, using standard assay methods, many of which are commercially available, and are used routinely in clinical settings. Methods of measuring serum markers include immunological-based methods, e.g., enzyme-linked immunosorbent assays (ELISA), radioimmunoassays, using antibody specific for a given serum marker.

Quantitative tests of functional liver reserve also can be used to assess the efficacy of treatment with a protease-resistant IFN-α polypeptide. These include: indocyanine green clearance (ICG), galactose elimination capacity (GEC), aminopyrine breath test (ABT), antipyrine clearance, monoethylglycine-xylidide (MEG-X) clearance, and caffeine clearance.

As used herein, a “complication associated with cirrhosis of the liver” refers to a disorder that is a sequelae of decompensated liver disease, i.e., or occurs subsequently to and as a result of development of liver fibrosis, and includes, but it not limited to, development of ascites, variceal bleeding, portal hypertension, jaundice, progressive liver insufficiency, encephalopathy, hepatocellular carcinoma, liver failure requiring liver transplantation, and liver-related mortality.

In another example, a therapeutically effective amount of a protease-resistant IFN-α polypeptide is an amount that is effective in reducing the incidence (e.g., the likelihood that an individual will develop) of a disorder associated with cirrhosis of the liver by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to an untreated individual, or in a placebo-treated individual.

Whether therapy with a protease-resistant IFN-α polypeptide is effective in reducing the incidence of a disorder associated with cirrhosis of the liver can readily be determined by those skilled in the art.

Reduction in liver fibrosis increases liver function. Thus, methods are provided for increasing liver function, generally involving administering a therapeutically effective amount of a protease-resistant IFN-α polypeptide. Liver functions include, but are not limited to, synthesis of proteins such as serum proteins (e.g., albumin, clotting factors, alkaline phosphatase, aminotransferases (e.g., alanine transaminase, aspartate transaminase), 5′-nucleosidase, .gamma.-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splanchnic and portal hemodynamics.

Whether a liver function is increased is readily ascertainable by those skilled in the art, using well-established tests of liver function. Thus, synthesis of markers of liver function such as albumin, alkaline phosphatase, alanine transaminase, aspartate transaminase and bilirubin, can be assessed by measuring the level of these markers in the serum, using standard immunological and enzymatic assays. Splanchnic circulation and portal hemodynamics can be measured by portal wedge pressure and/or resistance using standard methods. Metabolic functions can be measured by measuring the level of ammonia in the serum.

Whether serum proteins normally secreted by the liver are in the normal range can be determined by measuring the levels of such proteins, using standard immunological and enzymatic assays. Those skilled in the art know the normal ranges for such serum proteins. The following are non-limiting examples. The normal range of alanine transaminase is from about 7 to about 56 units per liter of serum. The normal range of aspartate transaminase is from about 5 to about 40 units per liter of serum. Bilirubin is measured using standard assays. Normal bilirubin levels are usually less than about 1.2 mg/dL. Serum albumin levels are measured using standard assays. Normal levels of serum albumin are in the range of from about 35 to about 55 g/L. Prolongation of prothrombin time is measured using standard assays. Normal prothrombin time is less than about 4 seconds longer than control.

In another example, a therapeutically effective amount of a protease-resistant IFN-α polypeptide is an amount that is effective to increase liver function by at least about 10%, at least about 20%, at least about 30%, at least about 40%, at least about 50%, at least about 60%, at least about 70%, at least about 80%, or more. For example, a therapeutically effective amount of a protease-resistant IFN-α polypeptide is an amount that is effective to reduce an elevated level of a serum marker of liver function by at least about 10%, at least about 20%, at least about 30%, at least about 40%, at least about 50%, at least about 60%, at least about 70%, at least about 80%, or more, or to reduce the level of the serum marker of liver function to within a normal range. A therapeutically effective amount of a protease-resistant IFN-α polypeptide also is an amount effective to increase a reduced level of a serum marker of liver function by at least about 10%, at least about 20%, at least about 30%, at least about 40%, at least about 50%, at least about 60%, at least about 70%, at least about 80%, or more, or to increase the level of the serum marker of liver function to within a normal range.

iii. Renal Fibrosis

The present methods are provided for treating renal fibrosis. The methods generally involve administering to an individual having renal fibrosis, or at risk of having renal fibrosis, an effective amount of a protease-resistant IFN-α polypeptide in an oral dosage formulation. In some examples, an “effective amount” of a protease-resistant IFN-α polypeptide that is effective in reducing renal fibrosis; and/or that is effective in reducing the likelihood that an individual will develop renal fibrosis; and/or that is effective in reducing a parameter associated with renal fibrosis; and/or that is effective in reducing a disorder associated with fibrosis of the kidney.

In one example, an effective amount of a protease-resistant IFN-α polypeptide is an amount that is sufficient to reduce renal fibrosis, or reduce the rate of progression of renal fibrosis, by at least about 10%, at least about 15%, at least about 20%, at least about 25%, at least about 30%, at least about 35%, at least about 40%, at least about 45%, at least about 50%, compared to the degree of renal fibrosis in the individual prior to treatment, or compared to the rate of progression of renal fibrosis that would have been experienced by the patient in the absence of treatment.

Whether fibrosis is reduced in the kidney is determined using any known method. For example, histochemical analysis of kidney biopsy samples for the extent of extracellular matrix (ECM) deposition and/or fibrosis is performed. Other methods are known in the art. See, e.g., Masseroli et al. (1998) Lab. Invest. 78:511-522; U.S. Pat. No. 6,214,542.

In some examples, an effective amount of a protease-resistant IFN-α polypeptide is an amount that is effective to increase kidney function by at least about 10%, at least about 15%, at least about 20%, at least about 25%, at least about 30%, at least about 35%, at least about 40%, at least about 45%, at least about 50%, compared to the basal level of kidney function in the individual prior to treatment.

In some examples, an effective amount of a protease-resistant IFN-α polypeptide is an amount that is effective to slow the decline in kidney function by at least about 10%, at least about 15%, at least about 20%, at least about 25%, at least about 30%, at least about 35%, at least about 40%, at least about 45%, at least about 50%, compared to the decline in kidney function that would occur in the absence of treatment.

Kidney function can be measured using any known assay, including, but not limited to, plasma creatinine level (where normal levels are generally in a range of from about 0.6 to about 1.2 mg/dL); creatinine clearance (where the normal range for creatinine clearance is from about 97 to about 137 mL/minute in men, and from about 88 to about 128 mL/minute in women); the glomerular filtration rate (either calculated or obtained from inulin clearance or other methods), blood urea nitrogen (where the normal range is from about 7 to about 20 mg/dL); and urine protein levels.

b. Cancer

Methods are provided for treating a proliferative disorder (e.g., cancer), the method generally involving administering to an individual in need thereof an effective amount of a protease-resistant IFN-α polypeptide in an oral dosage formulation provided, wherein the protease-resistant IFN-α polypeptide is prepared as described above.

The methods are effective to reduce the growth rate of a tumor by at least about 5%, at least about 10%, at least about 20%, at least about 25%, at least about 50%, at least about 75%, at least about 85%, or at least about 90%, up to total inhibition of growth of the tumor, when compared to a suitable control. Thus, in these examples, an “effective amount” of a protease-resistant IFN-α polypeptide is an amount that is sufficient to reduce tumor growth rate by at least about 5%, at least about 10%, at least about 20%, at least about 25%, at least about 50%, at least about 75%, at least about 85%, or at least about 90%, up to total inhibition of tumor growth, when compared to a suitable control. In an experimental animal system, a suitable control can be a genetically identical animal not treated with oral dosage formulation of the protease-resistant IFN-α polypeptide. In non-experimental systems, a suitable control can be the tumor load present before administering the dosage form of the protease-resistant IFN-α polypeptide. Other suitable controls can be a placebo control.

Whether growth of a tumor is inhibited can be determined using any known method, including, but not limited to, a proliferation assay wherein the number of cells in an in vitro cell culture is measured after a period of time, where the cells are-cultured in the presence or the absence of the composition and a ³H-thymidine uptake assay.

The methods are useful for treating a wide variety of cancers, including carcinomas, sarcomas, leukemias, and lymphomas.

Carcinomas that can be treated using a subject method include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelieal carcinoma, and nasopharyngeal carcinoma, etc.

Sarcomas that can be treated using a subject method include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

Other solid tumors that can be treated using a subject method include, but are not limited to, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

Leukemias that can be treated using a subject method include, but are not limited to, a) chronic myeloproliferative syndromes (neoplastic disorders of multipotential hematopoietic stem cells); b) acute myelogenous leukemias (neoplastic transformation of a multipotential hematopoietic stem cell or a hematopoietic cell of restricted lineage potential; c) chronic lymphocytic leukemias (CLL; clonal proliferation of immunologically immature and functionally incompetent small lymphocytes), including B-cell CLL, T-cell CLL prolymphocytic leukemia, and hairy cell leukemia; and d) acute lymphoblastic leukemias (characterized by accumulation of lymphoblasts). Lymphomas that can be treated using a subject method include, but are not limited to, B-cell lymphomas (e.g., Burkitt's lymphoma) and Hodgkin's lymphoma.

c. Viral Infections

Methods are provided for treating a virus infection, and methods of reducing viral load, or reducing the time to viral clearance, or reducing morbidity or mortality in the clinical outcomes, in patients suffering from a virus infection. Methods are provided for reducing the risk that an individual will develop a pathological viral infection that has clinical sequelae. The methods generally involve administering a therapeutically effective amount of a protease-resistant IFN-α polypeptide for the treatment of a virus infection, wherein the protease-resistant IFN-α polypeptide is prepared as described above.

In some examples, the IFN-α polypeptide can be administered prophylactically. Where a subject treatment method is prophylactic, the methods reduce the risk that an individual will develop pathological infection with a virus. An effective amount of a protease-resistant IFN-α polypeptide in an oral dosage formulation is an amount that reduces the risk or reducing the probability that an individual will develop a pathological infection with a virus. For example, an effective amount reduces the risk that an individual will develop a pathological infection by at least about 10%, at least about 20%, at least about 25%, at least about 30%, at least about 35%, at least about 40%, at least about 50%, at least about 60%, at least about 70%, at least about 80%, at least about 90%, or more, compared to the risk of developing a pathological infection with the virus in the absence of treatment with an oral dosage formulation of a protease-resistant IFN-α polypeptide.

In some examples, an effective amount of a protease-resistant IFN-α polypeptide in an oral dosage formulation is an amount that reduces viral load by at least about 10%, at least about 20%, at least about 25%, at least about 30%, at least about 35%, at least about 40%, at least about 50%, at least about 60%, at least about 70%, at least about 80%, at least about 90%, or more, compared to the viral load in the absence of treatment with an oral dosage formulation of a protease-resistant IFN-α polypeptide.

In some examples, an effective amount of a protease-resistant IFN-α polypeptide in an oral dosage formulation is an amount that that reduces the time to viral clearance, by at least about 10%, at least about 20%, at least about 25%, at least about 30%, at least about 35%, at least about 40%, at least about 50%, at least about 60%, at least about 70%, at least about 80%, at least about 90%, or more, compared to the time to viral clearance in the absence of treatment.

In some examples, an effective amount of a protease-resistant IFN-α polypeptide in an oral dosage formulation is an amount that reduces morbidity or mortality due to a virus infection by at least about 10%, at least about 20%, at least about 25%, at least about 30%, at least about 35%, at least about 40%, at least about 50%, at least about 60%, at least about 70%, at least about 80%, at least about 90%, or more, compared to the morbidity or mortality in the absence of treatment.

Whether a subject treatment method is effective in reducing the risk of a pathological virus infection, reducing viral load, reducing time to viral clearance, or reducing morbidity or mortality due to a virus infection is readily determined by those skilled in the art. Viral load is readily measured by measuring the titer or level of virus in serum. The number of virus in the serum can be determined using any known assay, including, e.g., a quantitative polymerase chain reaction assay using oligonucleotide primers specific for the virus being assayed. Whether morbidity is reduced can be determined by measuring any symptom associated with a virus infection, including, e.g., fever, respiratory symptoms (e.g., cough, ease or difficulty of breathing).

In some examples, methods are provided for reducing viral load, and/or reducing the time to viral clearance, and/or reducing morbidity or mortality in an individual who has been exposed to a virus (e.g., an individual who has come into contact with an individual infected with a virus), the method involving administering an effective amount of a protease-resistant IFN-α polypeptide in an oral dosage formulation. In these examples, therapy is begun from about 1 hour to about 14 days following exposure, e.g., from about 1 hour to about 24 hours, from about 24 hours to about 48 hours, from about 48 hours to about 3 days, from about 3 days to about 4 days, from about 4 days to about 7 days, from about 7 days to about 10 days, or from about 10 days to about 14 days following exposure to the virus.

In some examples, methods are provided for reducing the risk that an individual who has been exposed to a virus (e.g., an individual who has come into contact with an individual infected with a virus) will develop a pathological virus infection with clinical sequelae, the method involving administering an effective amount of a protease-resistant IFN-α polypeptide in an oral dosage formulation. In these examples, therapy is begun from about 1 hour to about 35 days following exposure, e.g., from about 1 hour to about 24 hours, from about 24 hours to about 48 hours, from about 48 hours to about 3 days, from about 3 days to about 4 days, from about 4 days to about 7 days, from about 7 days to about 10 days, from about 10 days to about 14 days, from about 14 days to about 21 days, or from about 21 days to about 35 days following exposure to the virus.

In some examples, methods are provided for reducing viral load, and/or reducing the time to viral clearance, and/or reducing morbidity or mortality in an individual who may or may not have been infected with a virus, and who has been exposed to a virus. In some of these examples, the methods involve administering an effective amount of a protease-resistant IFN-α polypeptide in an oral dosage formulation within 24 hours of exposure to the virus.

In some examples, methods are provided for reducing viral load, and/or reducing the time to viral clearance, and/or reducing morbidity or mortality in an individual who has not been infected with a virus, and who has been exposed to a virus. In some of these examples, the methods involve administering an effective amount of a protease-resistant IFN-α polypeptide in an oral dosage formulation within 48 hours of exposure to the virus. In other examples, the methods involve administering a protease-resistant IFN-α polypeptide in an oral dosage formulation more than 48 hours after exposure to the virus, e.g., from 72 hours to about 35 days, e.g., 72 hours, 4 days, 5 days, 6 days, or 7 days after exposure, or from about 7 days to about 10 days, from about 10 days to about 14 days, from about 14 days to about 17 days, from about 17 days to about 21 days, from about 21 days to about 25 days, from about 25 days to about 30 days, or from about 30 days to about 35 days after exposure to the virus.

In some examples, methods are provided for reducing the risk that an individual who has been exposed to a virus will develop a pathological virus infection with clinical sequelae. In some of these examples, the methods involve administering an effective amount of a protease-resistant IFN-α polypeptide in an oral dosage formulation within 24 hours of exposure to the virus.

In some examples, methods are provided for reducing the risk that an individual who has been exposed to a virus (e.g., an individual who has come into contact with an individual infected with a virus) will develop a pathological viral infection with clinical sequelae. In some of these examples, the methods involve administering an effective amount of a protease-resistant IFN-α polypeptide in an oral dosage formulation within 48 hours of exposure to the virus.

i. Hepatitis Virus Infection

HCV is exemplary of viral infections treated by IFN-α administration. Methods are provided for treating a hepatitis virus infection. In particular examples, methods are provided for treating a hepatitis C virus (HCV) infection; methods of reducing the incidence of complications associated with HCV and cirrhosis of the liver; and methods of reducing viral load, or reducing the time to viral clearance, or reducing morbidity or mortality in the clinical outcomes, in patients suffering from HCV infection. The methods generally involve administering to the individual an effective amount of a protease-resistant IFN-α polypeptide in an oral dosage formulation.

The methods decrease viral load in the individual, and to achieve a sustained viral response. Optionally, the subject method further provides administering to the individual an effective amount of a nucleoside analog, such as ribavirin, levovirin, isatoribine and viramidine. Of particular interest in many examples is treatment of humans.

Whether a method is effective in treating an HCV infection can be determined, for example, by measuring viral load, or by measuring a parameter associated with HCV infection, including, but not limited to, liver fibrosis, elevations in serum transaminase levels, and necroinflammatory activity in the liver. Indicators of liver fibrosis are discussed in detail below.

Viral load can be measured by measuring the titer or level of virus in serum. These methods include, but are not limited to, a quantitative polymerase chain reaction (PCR) and a branched DNA (bDNA) test. Quantitative assays for measuring the viral load (titer) of HCV RNA have been developed. Many such assays are available commercially, including a quantitative reverse transcription PCR (RT-PCR) (Amplicor HCV Monitor™, Roche Molecular Systems, New Jersey); and a branched DNA (deoxyribonucleic acid) signal amplification assay (Quantiplex™ HCV RNA Assay (bDNA), Chiron Corp., Emeryville, Calif.). See, e.g., Gretch et al. (1995) Ann. Intern. Med. 123:321-329. Also of interest is a nucleic acid test (NAT), developed by Qen-Probe Inc. (San Diego) and Chiron Corporation, and sold by Chiron Corporation under the trade name Procleix®, which NAT simultaneously tests for the presence of HIV-1 and HCV. See, e.g., Vargo et al. (2002) Transfusion 42:876-885.

In general, an effective amount of a protease-resistant IFN-α polypeptide in an oral dosage from is an amount that is effective to reduce viral load to undetectable levels, e.g., to less than about 5000, less than about 1000, less than about 500, or less than about 200 genome copies/mL serum. In some examples, an effective amount of a subject agent is an amount that is effective to reduce viral load to less than 100 genome copies/mL serum. In many examples, the methods provided achieve a sustained viral response, e.g., the viral load is reduced to undetectable levels for a period of at least about one month, at least about two months, at least about three months, at least about four months, at least about five months, or at least about six months following cessation of treatment.

Whether a subject method is effective in treating an HCV infection can be determined by measuring a parameter associated with HCV infection, such as liver fibrosis. Methods of determining the extent of liver fibrosis are discussed in detail below. In some examples, the level of a serum marker of liver fibrosis indicates the degree of liver fibrosis.

As one non-limiting example, levels of serum alanine aminotransferase (ALT) are measured, using standard assays. In general, an ALT level of less than about 45 international units is considered normal. In some examples, an effective amount of a protease-resistant IFN-α polypeptide in an oral dosage formulation for treatment of an HCV infection is an amount effective to reduce ALT levels to less than about 45 U/ml serum.

2. Treatment Methods Using Growth Hormone

In one example, the oral dosage formulation of a protease-resistant polypeptide is administered to a patient who has a disease, disorder, or condition that can be treated by administration of a growth hormone (GH) polypeptide. Typically, formulations of protease-resistant GH polypeptides provided herein are administered orally using an amount of a GH that is generally about or 6-400 times, such as 10-100 times, including, for example, 15-50 times, 20-30 times, typically 15-40 times higher per day, than amounts that are administered subcutaneously for the same indication that that can be prevented or treated by administration an unmodified therapeutic protein in the same individual.

Native GH has been administered for the treatment of a growth hormone deficiency at a dosage of about 0.004-0.016 mg/kg/day by subcutaneous injection. Oral dosage formulations of protease-resistant growth hormone polypeptides provided herein are administered orally using an amount of growth hormone that is greater than the dosage of growth hormone for subcutaneous administration. Typically, the oral dosage formulations of protease-resistant growth hormone polypeptides provided herein are administered orally using an amount of growth hormone that is generally about or 6-400 times, such as 10-100 times, including, for example, 15-50 times, 20-30 times, typically 15-40 times higher per day, than amounts that are administered subcutaneously for treatment of a growth hormone deficiency using an unmodified therapeutic protein in the same individual.

The oral dosage formulations of protease-resistant GH polypeptides provided herein can be used for treatment of any condition for which unmodified GH is employed. This section provides exemplary uses of modified GH polypeptides and administration methods. These described therapies are exemplary and do not limit the applications of GH.

The oral dosage formulations of protease-resistant GH polypeptides provided herein are intended for use in various therapeutic, lifestyle related, as well as diagnostic methods in which GH is used for treatment. Such methods include, but are not limited to, methods of treatment of physiological and medical conditions described and listed below. By virtue of their improved stability, protease-resistant GH polypeptides provided herein exhibit improvement in the corresponding in vivo activities and therapeutic effects.

In particular, the oral dosage formulations of protease-resistant GH polypeptides are intended for use in therapeutic methods in which the natural protein has been used for treatment. Treatment of disorders can include, but are not limited to, growth deficiency disorders (including but not limited to Turner's syndrome, intrauterine growth retardation, idiopathic short stature, Prader Willi syndrome, Thalassaemia), AIDS wasting, aging, impaired immune function of HIV-infected subjects, catabolic illnesses (including those associated with respiratory failure and burn injuries), recovery from surgery, congestive cardiomyopathy, liver transplantation, liver regeneration after hepatectomy, chronic renal failure, renal osteodystrophy, osteoporosis, achondroplasia/hypochondroplasia, skeletal dysplasia, chronic inflammatory or nutritional disorders (such as Crohn's disease), short bowel syndrome, juvenile chronic arthritis, cystic fibrosis, male infertility, X-linked hypophosphatemic rickets, Down's syndrome, Spina bifida, Noonan Syndrome, obesity, impaired muscle strength and fibromyalgia. The oral dosage formulations of protease-resistant GH polypeptides also can be administered in combination with other therapies including other biologics and small molecule compounds.

In particular, the oral formulations of protease-resistant GH polypeptides also are intended for use in lifestyle related methods in which the natural protein has been used for treatment. Applications related to life-style can include, but are not limited to anti-aging, sarcopenia (age-related muscle wasting), improvement of sex drive and/or libido, improvement of body chemistry and/or metabolism, increase in muscle strength and/or mass. The oral formulations of protease-resistant GH polypeptides also can be administered in combination with other therapies including other biologics and small molecule compounds.

Treatment of diseases and conditions oral dosage formulations of protease-resistant GH polypeptides can be effected by oral administration using suitable oral dosage formulations as described herein. If necessary, a particular dosage and duration and treatment protocol can be empirically determined or extrapolated based on dosages and regimens employed for other modes of GH administration, such as subcutaneous or intravenous injections. For example, exemplary doses of recombinant and native GH polypeptides can be used as a starting point to determine appropriate dosages. Dosages provided herein for treatments and therapies with GH and recombinant forms are exemplary dosages. Such exemplary dosages, however, can provide guidance in selecting dosing regimes for oral dosage formulation of protease-resistant GH polypeptides. Since the mutant GH polypeptides provided herein exhibit increased stability, dosages and administration regimens can differ from those for the unmodified growth hormones. Particular dosages and regimens can be empirically determined.

Dosage levels are apparent to one of skill in the art and can be determined based on a variety of factors, such as body weight of the individual, general health, age, the activity of the specific compound employed, sex, diet, time of administration, rate of excretion, drug combination, the severity and course of the disease or condition, and the subject's disposition to the disease/condition and the judgment of the treating physician. The amount of active ingredient that can be combined with the carrier materials to produce a single dosage form with vary depending upon the subject treated and the particular mode of administration.

Upon improvement of a subject's condition, a maintenance dose of a compound or composition provided herein can be administered, if necessary; and the dosage, the dosage form, or frequency of administration, or a combination thereof, can be varied. In some cases, the subject can require intermittent treatment on a long-term basis upon any recurrence of disease symptoms.

a. Growth Deficiencies

The growth of an infant into an adult is a complex process involving a number of genes and hormones, as well as nutrition, diet, exercise, and rest. Growth hormone is central to growth and development, and is the principal hormone governing height in an individual. Growth hormone deficiency is a disease often caused by a problem in the pituitary gland or the hypothalamus in the brain. Growth hormone deficiency can result either when GH is not present in the pituitary gland in adequate amounts or when GH is present in adequate amounts but the hormone required to release it (GHRH) is lacking. Growth retardation is a medical condition in which the normal growth of children is slowed down or stopped, due to a deficiency in the growth hormone (GH) system.

There are two different types of GH deficiencies in children: congenital and acquired.

Congenital growth hormone deficiencies arise from problems with the pituitary gland or hypothalamus while the fetus is growing in the womb, whereas acquired growth hormone deficiencies occur when the area around the pituitary and hypothalamus is damaged in some way. In some instances, growth hormone deficiencies may not have an identifiable cause (“idiopathic”)). Such instances include Prader-Willi Syndrome (a congenital disorder that features GH deficiency and short stature), Turner's Syndrome (a genetic defect that is manifested only in girls and characterized by short stature), chronic renal insufficiency (kidney malfunction, which can often cause growth retardation in children), and Thalassaemia (an inherited condition characterized by imbalance in the synthesis of hemoglobin causing severe anemia and malformed red blood cells that can cause reduced GH secretion and short stature).

Growth deficiencies not only affect children, but also can be a significant problem for adults. GH deficiency in adults is a specific clinical syndrome with numerous physiological consequences, including, but not limited to: changes in body composition, including central obesity; lipids in the blood; muscle strength; bone composition; exercise capacity and energy; cardiovascular risk; and psychological well-being (e.g., social isolation and depression). Additionally, studies indicate that subjects with hypo-pituitarism have an increased risk of mortality from cardiovascular disease, possibly attributable to their GH deficiency. GH deficiency in adults can result from a pituitary or peri-pituitary tumor, or as a direct result of the surgery/radiation used to manage these conditions. Less commonly, GH deficiency in adults arises from a deficiency acquired in childhood.

Recombinant GH, used as therapeutic treatment for growth deficiency supplements and/or replaces GH the body should normally produce. Treatments for adults and children can include systemic administration of GH. For example, GH can be administered alone or in combination with, prior to, intermittently with, or subsequent to other treating agents. Modes of administration include, but are not limited to, GH injection.

The oral dosage formulations of protease-resistant GH polypeptides described herein can be used in growth-deficiency therapies. The protease-resistant GHs herein exhibit increased protease resistance to proteases of the gastrointestinal tract, thereby improving therapeutic efficacy of a pharmaceutical composition. Thus, oral dosage formulations of protease-resistant GH polypeptides can be used to deliver longer lasting, more stable growth-deficiency therapies. Examples of therapeutic improvements using oral dosage formulations of protease-resistant GH polypeptides include, for example, but are not limited to, lower dosages, fewer and/or less frequent administrations, decreased side effects and increased therapeutic effects.

Dosages and regimens of oral dosage formulations protease-resistant GH polypeptides provided herein can be empirically determined. Dosages for unmodified GH can be used as guidance for determining dosages for modified GH. Factors such as the level of activity and half-life of a modified GH in comparison to an unmodified GH can be used in making such determinations.

Among the goals of GH therapy for the treatment of growth deficiency is long-term replacement of GH to physiologic levels comparable to healthy persons of same sex and similar age. Dosing regimens of GH can depend upon a number of factors including, but not limited to, age of subject; pubertal status; subject tolerance and incidence of adverse effects; and the source of the GH, whether recombinant or natural. For example, the potency of recombinant GH is about one-third the potency of pituitary GH. Particular doses and dosing regimes can be determined empirically. Exemplary doses of unmodified pituitary GH can be 0.1 mg/kg/wk (0.3 IU/kg/wk); exemplary doses of recombinant unmodified GH can be 0.18 to 0.3 mg/kg/wk for children with GHD (MacGillivray et al. Pediatrics 102: 527-530 (1998)). The average dose of GH given to children with growth hormone deficiency is 0.3 mg/kg/week divided daily doses given by subcutaneous injections. Other exemplary doses in treatment of GH deficient disorders in children include 0.35 mg/kg/week for children with chronic renal insufficiency; 0.375 mg/kg/week for children with Turner's syndrome; 0.3 mg/kg/week for children with idiopathic short stature; and 0.7 mg/kg/week for children with intrauterine growth retardation (see, for example, Vance et al. The New England Journal of Medicine 341(6): 1206-1216 (1999)).

The starting dose of GH in adults is typically 0.01-0.03 mg/kg/wk by subcutaneous injection. The maximal daily dose for subjects up to 35 years of age is typically 0.18 mg/kg/wk and 0.09 mg/kg/wk for older subjects. In various treatment studies on growth hormone-replacement, the dose of unmodified growth hormone has ranged from about 0.04 to about 0.18 mg/kg/wk (see, Vance et al. The New England Journal of Medicine 341(6): 1206-1216 (1999)). Others recommend (see e.g., the Growth Hormone Research Society recommendations) a starting dose of 105-210 mg/week, regardless of body weight (see, e.g., Consensus guidelines for the diagnosis and treatment of adults with growth hormone deficiency: Summary statement of the Growth Hormone Research Society Workshop on Adults Growth Hormone Deficiency. J. Clin. Endocrinol. Metab. 83: 379-381 (1998)).

b. Cachexia

In spite of the anti-retroviral therapy, which extends the lives of people with HIV, AIDS wasting is one of the principal causes of ill health in people with HIV/AIDS. Estimates of the prevalence of AIDS wasting range from 4-30% of HIV infected individuals. Studies have demonstrated that AIDS wasting, when left untreated, is directly correlated with mortality. Similar manifestations of wasting are observed in connection with other diseases, e.g., cancers.

Dosages and regimens of oral dosage formulations protease-resistant GH polypeptides provided herein can be empirically determined. Dosages for unmodified GH can be used as guidance for determining dosages for modified GH. Factors such as the level of activity and half-life of the modified GH in comparison to the unmodified GH can be used in making such determinations. The modified GHs provided herein have increased protease resistance to proteases of the gastrointestinal tract and in turn deliver longer lasting, more stable therapeutic effects in the treatment of AIDS wasting. Recombinant GH received approval from the US FDA in 1996 for the treatment of AIDS wasting. The recommended initial adult dosage of unmodified growth hormone therapy used in the treatment of cachexia is not more than 0.04 mg/kg/week divided into six or seven subcutaneous injections. The dose can be increased at four- to eight-week intervals according to individual subject requirements up to a maximum of 0.08 mg/kg/week, depending upon subject tolerance of treatment (see package insert for Serostim®). These exemplary dosages can be used as guidance in determination of dosing regimes for modified GH polypeptides, along with additional determinations of properties and activities of modified GH compared with an unmodified form.

c. Anti-Aging

Aging is associated with a decline in gonadotropins, thyroid-stimulating hormone and pituitary function; often termed “somatopause.” Thus, as a pituitary hormone, GH also has been reported to decline with age beginning in the third decade (see, for example Corpas et al. Endocr Rev 14: 20-39 (1993)). Decreased GH is associated with many of the changes seen with aging including increasing fat, decreasing muscle mass, and decreasing bone mass. Growth hormone replacement in growth hormone-deficient older individuals can improve quality of life, enhance bone and muscle mass, and reduce cardiovascular risk.

Dosages and dosing regimens of oral dosage formulations protease-resistant GH polypeptides provided herein can be empirically determined. The initial therapeutic dose of modified GH provided herein can be determined using the guidance of the recommended initial adult dosage approved for unmodified growth hormone and then titrated according to the improved therapeutic effect resulting from the modified GH. Exemplary subcutaneous dosing of recombinant hGH therapy in male subjects aged 61 to 81 can be 0.03 mg/kg of body weight, injected three times a week in the morning; the interval between injections being either one or two days (2.6 IU per milligram of hormone) (see, for example, Rudman et al., The New England Journal of Medicine 323(1): 1-6 (1990)). Exemplary subcutaneous dosing of recombinant hGH therapy in female subjects can be 0.025 mg/kg body weight/day (Bonello et al. J. Am. Geriatr. Soc. 44(9): 1038-42 (1996)). These doses can be used along with comparisons of properties of the modified and unmodified GH polypeptides to determine dosages for the modified GH. For example, the modified GHs provided herein have increased protease resistance to proteases of the gastrointestinal tract and, in turn, deliver longer lasting, more stable anti-aging therapeutic effects. Typical subcutaneous dosages for anti-aging range from about 12-24 IU per week, depending upon the individual, age, sex and other parameters. Precise dosages can be determined empirically and adjusted based on parameters, such as measurement of levels of IGF-1. Oral dosage formulations provided should contain at least as much, typically, 15-40 times as much as a daily subcutaneous dosage for anti-aging treatment.

d. Renal Osteodystrophy

Renal osteodystrophy, which includes a variety of skeletal disorders ranging from high turnover to low turnover lesions, both leading to reduced bone mineral density and higher fracture incidences, is common in subjects with chronic renal failure. Clinical trials have shown positive effects of recombinant human GH therapy as a treatment for improving bone turnover and bone mineral density in growth hormone-deficient subjects as well as subjects with chronic renal disease on hemodialysis (see, for example, Kotzmann et al. Journal of Nephrology 17(1): 87-94 (2004)). GH, as well as IGF-1, have marked effects on bone metabolism and bone mineral density. GH can stimulate chondrocyte growth and function as well as increase, directly or indirectly, bone turnover by stimulating osteoblasts and osteoclasts and inducing collagen synthesis, thereby enhancing long bone growth.

Dosages and dosing regimens of oral dosage formulations protease-resistant GH polypeptides provided herein can be empirically determined. The initial therapeutic dose of modified GH provided herein can be determined using the guidance of the recommended initial adult dosage approved for unmodified growth hormone and then titrated according to the improved therapeutic effect resulting from the modified GH. Exemplary dosing of adult subjects with chronic renal failure on hemodialysis can be 0.125 IU/kg (40.5 μg/kg) of GH injected subcutaneously 3 times per week after each dialysis session during the first 4 weeks of treatment and 0.25 IU/kg (81 μg/kg) thereafter. The length and dosage of GH treatment can vary according to subject tolerance (see Kotzmann et al. Journal of Nephrology 17(1): 87-94 (2004)). Comparisons of properties and activities of modified GH compared to unmodified GH can be used to determine alternate dosages and dosing regimes.

e. Cystic Fibrosis

Subjects, in particular children, with cystic fibrosis have problems with poor linear growth, inadequate weight gain, and protein catabolism. Multiple studies have demonstrated improved height and weight in children treated with GH. Other studies have shown that GH treatment results in improved forced vital capacity, improved exercise tolerance and bone accumulation. Still others have found GH treatment improves clinical status as measured by decreased hospitalizations and courses of intravenous antibiotics (see, for example, Hardin D. S., Eur. J. Endocrinol. 151(Suppl 1): S81-85 (2004)).

Dosages and dosing regimens of the oral dosage formulations protease-resistant GH polypeptides provided herein can be determined empirically. For example, guidance of dosages and dosing from unmodified GH and comparison of properties and activities of modified GH with unmodified GH can be used in the determination. Exemplary dosing of pediatric subjects with cystic fibrosis includes, but is not limited to, daily subcutaneous GH injections amounting to 0.3 mg/kg/wk (see Hardin D. S., Eur. J. Endocrinol. 151(Suppl 1): S81-85 (2004)). The initial therapeutic dose of modified GH provided herein can be the recommended initial adult dose for treatments using unmodified growth hormone and thereafter titrated according to the longer lasting and improved therapeutic effects of the modified GH provided herein. The modified GHs provided herein have increased protease resistance to proteases of the gastrointestinal tract. Such modified GH can deliver longer lasting, more stable therapeutic effect in the treatment of subjects with cystic fibrosis and can allow for lower dosing and less frequent dosages.

f. Other Conditions

A number of other physiological or pathological conditions are potential targets for GH therapy. These physiological or pathological conditions include, but are not limited to, stress, decreased energy, decreased physical power, catabolic illnesses including, for example, those associated with respiratory failure and burn injuries (see for example, Hart et al. Ann. Surg. 233(6): 827-34 (2001)), recovery from surgery (see for example, Yeo et al. Growth Horm. IGF Res. 13(6): 361-70 (2003)), congestive cardiomyopathy (see for example, Adamapolous et al. Eur. Heart J. 24(24): 2186-96 (2003)), liver transplantation or liver regeneration after hepatectomy (see for example Luo et al. World J Gastroenterol. 10(9): 1292-6 (2004)), chronic inflammatory or nutritional disorders such as short bowel syndrome, Crohn's disease (see for example, Slonim et al. N. Engl. J. Med. 342(22): 1633-7 (2000)), juvenile chronic arthritis (see for example, Saha et al. J Rheumatol. 1(7): 1413-7 (2004)), male fertility disorders (see for example Ovesen et al. Fertil Steril. 66(2): 292-8 (1996)), and other disorders such as impaired immune function of HIV-infected subjects, osteoporosis, achondroplasia/hypochondroplasia, skeletal dysplasia, X-linked hypophosphatemic rickets (see for example, Seikaly et al. Pediatrics. 100(5): 879-84 (1997)), Noonan Syndrome (see for example, Noordam et al. Acta Paediatr. 90(8): 889-94 (2001)), obesity, Down's syndrome, Spina bifida, and fibromyalgia (see for example, Bennet et al. Am. J. Med. 104(3): 227-31 (1998)).

### H. COMBINATION THERAPIES

Any of the oral dosage formulations of protease-resistant polypeptides, described herein can be administered in combination with, prior to, intermittently with, or subsequent to, other therapeutic agents, therapies, or procedures including, but not limited to, other biologics, small molecule compounds and surgery. The protease-resistant therapeutics can be formulated with other active agents, particularly others for treatment of the same condition.

For any disease or condition, including all those exemplified above, for which one or more therapeutic polypeptides is indicated or has been used and for which other therapeutic agents and treatments are available, protease-resistant therapeutic polypeptides can be used in combination therewith. Hence, the oral dosage formulations of protease-resistant therapeutic polypeptides provided herein similarly can be used. Therapeutic agents, such as biologics or molecules, used in combination with the oral dosage forms provided herein can be formulated together (e.g., in the same tablet or capsule) or separately. For combinations formulated together in the same tablet or capsule, the combinations can be formulated for simultaneous release of both active ingredients or for release of active components at different time points following oral administration. Such combinations can be formulated with combinations of various coatings to effect release of the active ingredients a one or more time points. Combinations of the dosage formulations provided herein with dosage formulations or compositions containing other agents are provided. Such combinations can be provided in kits, optionally packaged with other items, such as instructions for use, syringes, vials and other items.

The biologics for the combinations can include one or more forms of the same polypeptide (e.g., native or modified, for example, by further amino acid modifications, glycosylation, pegylation, or albumation) or allelic or species variants thereof. Furthermore, combination therapies of the oral dosage formulations provided also include administration of the same polypeptide by more than one route. For example, combination therapies can include administration of the oral dosage formulation of the protease-resistant polypeptide in addition to administration of a dosage formulation of the protease-resistant polypeptide or unmodified, native, or other modified form (e.g., hyperglycosylated or pegylated) polypeptide by other mode of administration (e.g., subcutaneous intravenous or oromucosal)).

Exemplary combination therapies of oral dosage formulations of protease-resistant IFN-α polypeptides for the treatment of viral infections, such as HCV infection, include but are not limited to treatment with nucleoside analogs such as ribavirin and viramidine (prodrug of ribavirin), L-nucleosides such as levovirin, Type II interferon receptor agonists (e.g., IFN-γ), TNF antagonists (e.g., etanercept, infliximab, or adalimumab), thymosin-α, SAPK inhibitors (e.g., pirfenidone or pirfenidone analogs), amantidine, NS3 inhibitors, NS5B inhibitors, alpha-glucosidase inhibitors, or a combination thereof.

Exemplary combination therapies of oral dosage formulations provided of protease-resistant IFN-α polypeptides for the treatment of cancer include but are not limited to cytokines, chemokines, growth factors, photosensitizing agents, toxins, anti-cancer antibiotics, chemotherapeutic compounds, radionuclides, angiogenesis inhibitors, signaling modulators, anti-metabolites, anti-cancer vaccines, anti-cancer oligopeptides, mitosis inhibitor proteins, antimitotic oligopeptides, anti-cancer antibodies, immunotherapeutic agents, hyperthermia or hyperthermia therapy, radiation therapy or a combination thereof.

Exemplary combination therapies of oral dosage formulations provided of protease-resistant IFN-α polypeptides for the treatment of fibrotic disorders include combination treatment with one or more anti-fibrotic agents, including, but not limited to, a SAPK inhibitor (e.g., pirfenidone or a pirfenidone analog), TNF antagonists (e.g., etanercept, infliximab, or adalimumab), TGF-β antagonists (e.g., GLEEVEC), and endothelin receptor antagonists (e.g., TRACLEER).

Exemplary combination therapies of oral dosage formulations provided of protease-resistant growth hormone polypeptides for the treatment of growth hormone deficiencies, or diseases or conditions for which growth hormone is administered, include combination treatment with one or more therapeutic agents, including, but not limited to, gonadotropin releasing hormone (GNRH) analogs, insulin-like growth factor 1, (rhIGF-1), growth hormone-releasing peptides (GRP), Acipimox® or other free fatty acid regulation agents, leutenizing hormone releasing hormone, anabolic steroids, such as oxandrolone and testosterone, letrozole, and cyproterone acetate.

### I. ARTICLES OF MANUFACTURE AND KITS

Oral dosage formulations of protease-resistant polypeptides can be packaged as articles of manufacture containing packaging material, a pharmaceutical composition which is effective for treating a disease or disorder, and a label that indicates that the protease-resistant polypeptide is to be used for treating the disease or disorder. A wide array of oral dosage formulations of the protease-resistant polypeptides is contemplated as are a variety of treatments for any disease or disorder that can be prevented or treated by administration of the polypeptide.

The oral dosage formulation, if desired, can be provided as a package, in a kit or dispenser device, that can contain one or more unit dosage forms containing the protease-resistant polypeptide. The package, for example, can contain a metal or plastic foil, such as a blister pack. The pack or dispenser device can be accompanied by instructions for administration. The compositions containing the protease-resistant polypeptides can be packaged as articles of manufacture containing packaging material, an oral dosage formulation provided herein, and a label that indicates the disorder for which the oral dosage formulation is provided.

The articles of manufacture provided herein contain packaging materials. Packaging materials for use in packaging pharmaceutical products are well known to those of skill in the art. See, for example, U.S. Pat. Nos. 5,323,907, 5,052,558 and 5,033,352, each of which is incorporated herein in its entirety. Examples of pharmaceutical packaging materials for oral dosage formulations include, but are not limited to, blister packs, a blister card, a box, a foil packet, bottles, tubes, bags, vials, containers and any packaging material or a combination thereof, suitable for a selected oral dosage formulation, mode of administration, and treatment. The kits also can be designed in a manner such that they are tamper resistant or designed to indicate if tampering has occurred. Optionally, a kit can contain the oral dosage formulation provided herein in combination with another pharmaceutical composition.

The oral dosage formulations of protease-resistant polypeptides can be provided as kits. Kits can include an oral pharmaceutical composition described herein and an item for administration. For example an oral dosage formulation of a therapeutic polypeptide can be supplied with a device for administration, such as a dosage cup. The kit can, optionally, include a notice or printed instructions for application including dosages, dosing regimens and instructions for modes of administration. Such printed instructions also can be in a form prescribed by a governmental agency regulating the manufacture, use, or sale of pharmaceuticals or biological products, which notice reflects approval by the agency of the manufacture, use, or sale for human administration to treat a condition that can be treated by administration of the protease-resistant polypeptide. In some examples, the kit optionally contains printed matter or other medium, such as a CD, such as instruction for use of the dosage form to treat a condition or disease. Kits also can include an oral pharmaceutical composition described herein and an item for diagnosis. For example, such kits can include an item for measuring the concentration, amount or activity of a protease-resistant therapeutic polypeptide or a therapeutic polypeptide regulated system of a subject.

### J. EXAMPLES

The following examples are included for illustrative purposes only and are not intended to limit the scope of the embodiments provided herein. The results of pharmacokinetic (PK) studies presented herein illustrate exemplary model systems for studying the PK profiles of the oral dosage formulations provide. As such, the dosages employed are substantially higher and do not necessarily represent a linear relationship for the selection of dosages for treatment of humans, but can be used as a guide for the empiric determination of therapeutically effective doses for treatment of humans based on the disease or condition to be treated.

### Example 1

### Assessment of Protease Resistance of Candidate LEAD Polypeptides

### I. Methods

A. Resistance to Proteolysis

The following protocol to assess resistance to proteases was used for all proteins tested, unless otherwise indicated.

Mutants were treated with proteases in order to identify resistant molecules. The relative resistance of the mutant proteins compared to the native protein against enzymatic cleavage was determined by exposure to a mixture of proteases (containing 1.5 pg of each of the following proteases (1% wt/wt, Sigma): α-chymotrypsin, carboxypeptidase, endoproteinase Arg-C, endoproteinase Asp-N, endoproteinase Glu-C, endoproteinase Lys-C, and trypsin) at 25° C. for a set time period between 30 minutes to either 24 or 48 hours depending on the experiment. At the end of the incubation time, 10 μl of anti-protease complete medium containing mini EDTA free tablets, Roche (one tablet was dissolved in 10 ml of DMEM and then diluted to 1/1000) was added to each reaction in order to inhibit protease activity. Treated samples were then used to determine residual activity such as anti-viral or proliferative activity as set forth below in part B and C.

For hGH, a 3% mixture of endoproteinase AspN, endoproteinase GluC, alpha-chymotrypsin, and trypsin was used. The proteins were incubated with the proteases at 25° C. for a set time period between 30 minutes to 120 minutes. Residual activity was determined as described above.

For erythropoietin, the relative resistance of the mutant proteins compared to the native protein against enzymatic cleavage was determined by exposure to a 1.5% protease mixture (wt/wt) containing each of the following proteases: α-chymotrypsin, Endoproteinase GluC and trypsin (Sigma) or by exposure to a 3% protease mixture (wt/wt) containing each of the following proteases, α-chymotrypsin, Endoproteinase GluC and trypsin (Sigma). The protease reaction was processed for a period of 0.5 to 2 hours as described above and residual activity was determined.

B. Anti-Viral Activity

Residual activity of Interferon-alpha (i.e., IFN-α-2b), Interferon-gamma (IFN-γ) and Interferon-beat (IFN-β) variant polypeptides were assessed in an anti-viral assay. Anti-viral activity can be measured by cytopathic effects (CPE). Anti-viral activity of variant polypeptides was determined by the capacity of the cytokine to protect HeLa cells against EMC (mouse encephalomyocarditis) virus-induced cytopathic effects. The day before, HeLa cells (2×10⁵ cells/ml) were seeded in flat-bottomed 96-well plates containing 100 μl/well of Dulbecco's MEM-Glutamax-sodium pyruvate medium supplemented with 5% SVF and 0.2% of gentamicin. Cells were grown at 37° C. in an atmosphere of 5% CO₂ for 24 hours.

Two-fold serial dilutions of variant polypeptide samples preincubated with protease (as described in part A above) were made with MEM complete media in 96-deep well plates. Twenty-four (24) hours after seeding the cells, the medium was aspirated from each well and 100 μl of diluted samples were added to HeLa cells. Each sample dilution was assessed in triplicate. The last two rows of the plates were filled with 100 μl of medium without added sample in order to serve as controls for cells with and without virus. After 24 hours of growth, a 1/1000 EMC virus dilution solution was placed in each well, except for the cell control row. Plates were returned to the CO₂ incubator for 40-48 hours. The medium was discarded, and the cells were washed twice with 100 μl of 1×PBS and stained for 1 hour with 80-100 μl of staining solution (trypan blue or ethanol-formamide-methyl blue mixture) to determine the proportion of intact cells. Plates were washed in a distilled water bath and the cell-bound dye was extracted using 80-100 μl of ethylene-glycol mono-ethyl-ether (Sigma). The absorbance of the dye was measured using an ELISA plate reader (Spectramax; Molecular devices) at 660 nm.

C. Cell Proliferation Activity

1. Nb2-11 Cells

Residual activity of Growth Hormone (i.e. hGH) variants were tested by assessing their effects on the proliferation of rat lymphoblast Nb2-11 cells. Nb2-11 cells were cultured in Fisher medium supplemented with 10% of SVF and 10% of equine serum (ES). Twenty four (24) hours before the proliferation assay, the cells were centrifuged and washed with phosphate buffered saline (PBS). The cells were then cultured in Fisher medium supplemented only with 10% of ES at a density of 0.5-0.8×10⁶ cells/ml. After 24 hours of culture the cells were seeded in 96-well plates at 4×10⁴ cells/well and treated with three-fold serial dilution of pre-treated native human growth hormone (hGH) or mutants between 6000 pg/ml and 0.3 pg/ml in triplicate. National Institute for Biological Standards and Control (NIBSC) hGH was used as an internal control for each proliferation assay.

After 48 hours of treatment with either native or mutant hGH (pre-treated with proteases) the proliferation of Nb2-11 cells was measured. Twenty microliters (20 μl) of Cell Titer 96 AQ (Promega) per well was added and cells incubated for 1 hour at 37° C. The conversion of tetrazolium MTS into a soluble formazan was measured. Samples were read in a Spectramax reader (Molecular Device) at 490 nm.

2. TF-1 Cells

Residual activity of erythropoietin (EPO) variants was tested by assessing their effects on the proliferation of human erythroleukemia cells (TF-1 cell line). TF-1 cell line was maintained in RPMI 1640 medium (Invitrogen) supplemented with 10% FCS, 2 mM L-glutamine and 2 ng/ml of human recombinant GM-CSF at 37° C. in a humid atmosphere with a composition of 7% CO₂/95% air in T175 (175 cm2) polystyrene tissue culture flask and split two times per week. Twenty four (24) hours before use in proliferation assays, cells were washed two times in ice cold PBS and re-suspended for 16 hours in GM-CSF free RPMI medium supplemented with 2 mM glutamine and 10% FCS.

TF-1 cells were plated into 96-well plates at 4×10⁴ cells per well in 70 μl of GM-CSF free RPMI medium supplemented with 2 mM glutamine and 10% FCS. Each sample (native or mutant EPO pre-treated with protease as described in part A above) were subjected to a two-fold serial dilution into 96-deep-well plates and EPO dilutions (30 μl) were added to each well containing 70 μl of TF-1 cells with a final concentration ranging from 70000 to 34.2 pg/ml. Each EPO sample dilution was assessed in triplicate. No GM-CSF was added to the last row (“G” row) of the flat-bottomed 96-well plates in order to evaluate basal absorbance of non-proliferative cells. A 2-fold serial dilution (70000 to 34.2 pg/ml) of internal positive controls including both the second international standard for EPO (NIBSC, 88/574) and the first international standard for GM-CSF (NIBSC, 88/646) also were performed and added in triplicate to the 96-well plate and assayed in order to standardize proliferation results.

The plates were incubated for 48 hours at 37° C. in a humidified, 7% CO₂ atmosphere. After 48 hours of growth, 20 μl of Cell titer 96 Aqueous one solution reagent (Promega) was added to each well and incubated 3 hours at 37° C. in an atmosphere of 7% CO₂. To measure the amount of colored soluble formazan produced by cellular reduction of the MTS, the absorbance of the dye was measured using an ELISA plate reader (Spectramax) at 490 nm.

### II. Results

The methods described above to assess protease resistance of candidate LEAD polypeptides followed by assessment of residual activity were applied to thousands of polypeptides. Results are provided below for assessment of protease resistance of some exemplary candidate LEAD and SuperLEAD polypeptides. The data are not meant to be representative of all proteases, but are exemplary data showing the resistance to proteolysis to an exemplary protease cocktail as described in the methods above. Thus, the data are not comprehensive and are not meant to be indicative that other polypeptides do not exhibit protease resistance.

A. Interferon-Alpha

A total of 184 variants of interferon-alpha (IFN-α2b) candidate LEAD polypeptides were generated and tested based on the predetermined property of increased protease resistance.

Prior to the assessment of protease resistance, IFN-α candidate LEADs were assessed for activity in an anti-viral assay using the EMCV replication assay in HeLa cells as described above. Native IFN-α and the selected leads were tested in a series of dilutions ranging from 3000 pg/mL to 1.46 pg/mL. The specific activity (i.e. activity per unit protein mass; units/mg protein) of each IFN-α mutant was compared with that of native IFN-α and expressed as million of international units (MIU) determined from experimental EC₅₀.

The anti-viral specific activity (expressed in MIU/mg) measurements for four exemplary LEAD polypeptides, including IFN-α-2b(E41Q) (lead 11), in comparison with native IFN-α are as follows: 280 MIU/mg for native IFN-α and between 200 and 400 MIU/mg for the selected LEADs. The candidate E41Q IFN-α-2b LEAD had a specific activity of 300 MIU/mg. Thus it was concluded that IFN-α-2b(E41Q) exhibited antiviral activity equivalent to native IFN-α.

Candidate LEADs were tested in vitro for protease resistance by incubating 100 μl of 1500 pg/ml (500 U/ml) of IFN-α-2b with a cocktail of proteases as described above. Following protease treatment, residual activity was assessed by an anti-viral assay. Table 23 shows the results of some of the tested polypeptides. The resistance to proteolysis is indicated as “no change” or “increased” as compared to the residual activity of the respective native polypeptide under the same protease treatment conditions. The results show that many of the polypeptides tested exhibited an increased resistance to protease in vitro as compared to native IFN-α.

TABLE 23
Resistance to Proteolysis of
Candidate Interferon-alpha LEADS

| | Resistance to |
|---|---|
| Mutant | proteolysis |
| F27V | No change |
| R33H | No change |
| E41Q | Increased |
| E41H | No change |
| E58Q | Increased |
| E58H | Increased |
| E78Q | Increased |
| E78H | Increased |
| Y89H | No change |
| E107Q | Increased |
| E107H | Increased |
| P109A | No change |
| L110V | No change |
| M111V | No change |
| E113H | Increased |
| L117V | Increased |
| L117I | Increased |
| K121Q | Increased |
| R125H | Increased |
| R125Q | Increased |
| K133Q | Increased |
| E159H | Increased |
| E159Q | Increased |

One variant polypeptide having the amino acid replacement E41Q was chosen for further kinetic analysis. The decreased susceptibility to proteases was evaluated by exposing the E41Q mutant IFN-α-2b and native IFN-α-2b to the following proteolytic treatments: a) human blood lysate; b) human serum; c) chymotrypsin (10% w/w); d) a protease mixture (1% w/w of α-chymotrypsin, carboxypeptidase, endoproteinase Arg-C, endoproteinase Asp-N, endoproteinase Glu-C, endoproteinase Lys-C and trypsin) as described above in the methods for assaying resistance to proteases, except that incubation with protease mixture was done for variable time from 0-48 hours. The protease reaction was stopped at 0.5 h, 1 h, 4 h, 8 h, 16 h, 24 h and 48 hours of incubation with protease, followed by assessment of residual anti-viral activity. The results were depicted as a percentage of the anti-viral activity of native IFN-α-2b without incubation by protease. The results show that the native IFN-α lost greater than 30% activity within 1 hour of treatment with blood lysate or the protease pool, 50% activity within 1 hour of treatment with serum and 60% activity within 1 hour of treatment with chymotrypsin. The level of activity of native IFN-α-2b was maintained at only 25-30% of its activity at 24 hours under all protease incubation conditions tested. In contrast, the results show that mutant IFN-α maintained approximately 100% anti-viral activity within 1 hour of protease treatment under all conditions tested, and 50-90% of the activity was maintained depending on the protease incubation condition for up to 24 hours compared to native IFN-α.

B. Growth Hormone

A total of 222 variants of human Growth Hormone (hGH) were generated and tested based on the predetermined property of increased protease resistance. Candidate LEADs and SuperLEADs of hGH were tested in vitro for protease resistance by incubating 15 ng of native hGH or variants with a cocktail of proteases as described above, followed by assessment of residual proliferative activity on Nb2-11 cells as described above. Table 24 shows the results of some of the tested polypeptides. The resistance to proteolysis is indicated as “no change” or “increased” as compared to the residual activity of the respective native polypeptide under the same protease treatment conditions. The results show that many of the polypeptides tested exhibited an increased resistance to protease in vitro as compared to native hGH.

TABLE 24
Resistance to Proteolysis of Candidate Growth
Hormone LEADS and SuperLEADs

| | Resistance to | | Resistance to |
|---|---|---|---|
| Mutation | proteolysis | Mutation | proteolysis |
| F1I/P2A | increase | F92V | no changes |
| F1I/P2S | no changes | L93I | no changes |
| F1V/P2A | no changes | L93V | no changes |
| F1V/P2S | no changes | R94H | no changes |
| P5A | no changes | R94Q | no changes |
| P5S | increase | F97I | no changes |
| L6I | no changes | F97V | no changes |
| L6V | no changes | L101I | no changes |
| R8H | no changes | L101V | increase |
| R8Q | no changes | Y103H | no changes |
| L9I | no changes | Y103I | no changes |
| L9V | increase | D107N | no changes |
| F10I | no changes | D107Q | no changes |
| F10V | no changes | Y111H | no changes |
| D11N | increase | Y111I | increase |
| D11Q | no changes | D112N | increase |
| M14I | no changes | D112Q | no changes |
| M14V | increase | L113I | no changes |
| L15I | no changes | L113V | no changes |
| L15V | no changes | L114I | no changes |
| R16H | increase | L114V | no changes |
| R16Q | no changes | K115N | no changes |
| R19H | no changes | K115Q | no changes |
| R19Q | no changes | D116N | no changes |
| L20I | no changes | D116Q | increase |
| L20V | no changes | L117I | no changes |
| L23I | increase | L117V | no changes |
| L23V | increase | E118Q | no changes |
| F25I | no changes | E118H | no changes |
| F25V | no changes | E118N | no changes |
| D26N | increase | E119Q | increase |
| D26Q | no changes | E119H | no changes |
| Y28H | no changes | E119N | no changes |
| Y28I | no changes | L124I | no changes |
| E30Q | no changes | L124V | increase |
| E30H | no changes | M125I | increase |
| E30N | no changes | M125V | increase |
| F31I | no changes | R127H | no changes |
| F31V | no changes | R127Q | no changes |
| E32Q | no changes | L128I | no changes |
| E32H | no changes | L128V | no changes |
| E32N | no changes | E129Q | no changes |
| E33Q | no changes | E129H | no changes |
| E33H | no changes | E129N | no changes |
| E33N | no changes | D130N | no changes |
| Y35H | no changes | D130Q | no changes |
| Y35I | no changes | P133A | increase |
| P37A | no changes | P133S | no changes |
| P37S | no changes | R134H | increase |
| K38N | increase | R134Q | no changes |
| K38Q | no changes | F139I | no changes |
| E39Q | no changes | F139V | no changes |
| E39H | no changes | K140N | increase |
| E39N | no changes | K140Q | no changes |
| K41N | no changes | Y143H | no changes |
| K41Q | increase | Y143I | no changes |
| Y42H | increase | K145N | no changes |
| Y42I | increase | K145Q | no changes |
| F44I | no changes | F146I | no changes |
| F44V | no changes | F146V | no changes |
| L45I | no changes | D147N | increase |
| L45V | no changes | D147Q | increase |
| P48A | no changes | D153N | increase |
| P48S | no changes | D153Q | no changes |
| L52I | no changes | D154N | no changes |
| L52V | no changes | D154Q | no changes |
| F54I | no changes | L156I | increase |
| F54V | no changes | L156V | no changes |
| E56Q | increase | L157I | increase |
| E56H | no changes | L157V | no changes |
| E56N | increase | K158N | increase |
| P59A | no changes | K158Q | no changes |
| P59S | no changes | Y160H | no changes |
| P61A | no changes | Y160I | no changes |
| P61S | no changes | L162I | increase |
| R64H | no changes | L162V | no changes |
| R64Q | no changes | L163I | no changes |
| E65Q | increase | L163V | no changes |
| E65H | no changes | Y164H | no changes |
| E65N | no changes | Y164I | no changes |
| E66Q | increase | F166I | increase |
| E66H | no changes | F166V | no changes |
| E66N | no changes | R167H | increase |
| K70N | no changes | R167Q | increase |
| K70Q | no changes | K168N | increase |
| L73I | no changes | K168Q | increase |
| L73V | increase | D169N | no changes |
| E74Q | no changes | D169Q | increase |
| E74H | no changes | M170I | no changes |
| E74N | increase | M170V | no changes |
| L75I | no changes | D171N | increase |
| L75V | no changes | D171Q | increase |
| L76I | no changes | K172N | no changes |
| L76V | no changes | K172Q | increase |
| R77H | no changes | E174Q | increase |
| R77Q | no changes | E174H | increase |
| L80I | no changes | E174N | increase |
| L80V | no changes | F176I | no changes |
| L81I | no changes | F176V | no changes |
| L81V | increase | L177I | increase |
| L82I | no changes | L177V | increase |
| L82V | no changes | R178H | no changes |
| W86H | no changes | R178Q | increase |
| W86S | no changes | R183H | no changes |
| L87I | no changes | R183Q | no changes |
| L87V | increase | E186Q | no changes |
| E88Q | no changes | E186H | no changes |
| E88H | no changes | E186N | no changes |
| E88N | no changes | F191I | increase |
| P89A | no changes | F191V | no changes |
| P89S | no changes | hGH WT | |
| F92I | no changes | hGH NIBSC | |

C. Interferon Gamma

A total of 148 variants of interferon-gamma (IFN-gamma) were generated and tested based on the predetermined property of increased protease resistance. IFN-gamma candidate LEADs were tested in vitro for protease resistance by incubating 15 ng of native IFN-gamma or variants with a cocktail of proteases as described above, followed by assessment of residual anti-viral activity as described above. Table 25 shows the results of some of the tested polypeptides. The resistance to proteolysis is indicated as “no change” or “increased” as compared to the residual activity of the respective native polypeptide under the same protease treatment conditions. The results show that many of the polypeptides tested exhibited an increased resistance to protease in vitro as compared to native IFN-gamma.

TABLE 25
Resistance to Proteolysis of Candidate Interferon-gamma
LEADs

| | Resistance | | Resistance | | |
|---|---|---|---|---|---|
| | to Pro- | | to Pro- | | Resistance to |
| Mutation | teolysis | Mutation | teolysis | Mutation | Proteolysis |
| D5N | no change | D44N | ND | D94N | ND |
| D5Q | no change | D44Q | ND | D94Q | ND |
| P6A | no change | R45H | no change | F95I | ND |
| P6S | no change | R45Q | no change | F95V | ND |
| Y7H | no change | F57I | no change | E96Q | Increase |
| Y7I | no change | F57V | increase | E96H | Increase |
| K9N | no change | K58N | no change | E96N | Increase |
| K9Q | no change | K58Q | no change | K97N | no change |
| E10Q | no change | L59I | no change | K97Q | no change |
| E10H | no change | L59V | no change | L98I | Increase |
| E10N | no change | F60I | no change | L98V | Increase |
| E12Q | no change | F60V | ND | Y101H | no change |
| E12H | no change | K61N | no change | Y101I | no change |
| E12N | no change | K61Q | no change | D105N | Increase |
| L14I | no change | F63I | increase | D105Q | Increase |
| L14V | no change | F63V | increase | L106I | Increase |
| K15N | no change | K64N | ND | L106V | Increase |
| K15Q | no change | K64Q | increase | E115Q | ND |
| K16N | no change | D65N | no change | E115H | ND |
| K16Q | no change | D65Q | no change | E115N | ND |
| Y17H | no change | D66N | increase | E122Q | ND |
| Y17I | no change | D66Q | increase | E122H | ND |
| F18I | no change | K71N | no change | E122N | ND |
| F18V | no change | K71Q | no change | L123I | ND |
| D24N | ND | E74Q | no change | L123V | ND |
| D24Q | ND | E74H | no change | P125A | Increase |
| D27N | ND | E74N | no change | P125S | Increase |
| D27Q | ND | K77N | no change | K128N | no change |
| L31I | ND | K77Q | no change | K128Q | no change |
| L31V | ND | E78Q | no change | K131N | ND |
| F32I | ND | E78H | no change | K131Q | ND |
| F32V | ND | E78N | no change | R132H | no change |
| L33I | increase | D79N | no change | R132Q | no change |
| L33V | increase | D79Q | ND | K133N | Increase |
| L36I | no change | K83N | no change | K133Q | Increase |
| L36V | no change | K83Q | no change | R134H | Increase |
| K37N | increase | F84I | ND | R134Q | Increase |
| K37Q | increase | F84V | ND | M137I | ND |
| W39H | ND | K89N | increase | M137V | Increase |
| W39S | ND | K89Q | increase | L138I | ND |
| K40N | no change | K90N | increase | L138V | Increase |
| K40Q | no change | K90Q | increase | F139I | Increase |
| E41Q | increase | K91N | ND | F139V | Increase |
| E41H | increase | K91Q | ND | R142H | Increase |
| E41N | increase | R92H | no change | R142Q | Increase |
| E42Q | no change | R92Q | no change | R143H | Increase |
| E42H | no change | D93N | ND | R143Q | Increase |
| E42N | no change | D93Q | ND | | |

ND: Not determined

D. Erythropoietin

A total of 199 variants of erythropoietin (EPO) were generated and tested based on the predetermined property of increased protease resistance. EPO candidate LEADs were tested in vitro for protease resistance by incubating 557.2 ng of native EPO or variants with a cocktail of proteases containing a 1.5% protease mixture (wt/wt) as described above, followed by assessment of residual anti-proliferative activity on TF-1 cells as described above. Table 26 shows the results of some of the tested polypeptides. The resistance to proteolysis is indicated as “no change” or “increased” as compared to the residual activity of the respective native polypeptide under the same protease treatment conditions. The results show that many of the polypeptides tested exhibited an increased resistance to protease in vitro as compared to native EPO.

TABLE 26
Resistance to Proteolysis of Candidate EPO LEADs

| | | Resistance |
|---|---|---|
| Nemo | | to |
| Code # | Mutation | proteases |
| 1 | P2S | ND |
| 2 | P2A | no change |
| 3 | P3S | no change |
| 4 | P3A | increase |
| 5 | R4H | increase |
| 6 | R4Q | increase |
| 7 | C7S | ND |
| 8 | C7V | ND |
| 9 | D8Q | increase |
| 10 | D8H | no change |
| 11 | R10H | increase |
| 12 | R10Q | no change |
| 13 | L12V | no change |
| 14 | L12I | no change |
| 15 | E18Q | increase |
| 16 | E18H | no change |
| 17 | K20Q | increase |
| 18 | K20T | no change |
| 19 | E21Q | increase |
| 20 | E21H | no change |
| 21 | E23Q | no change |
| 22 | E23H | no change |
| 23 | C29S | ND |
| 24 | C29V | ND |
| 25 | E31Q | increase |
| 26 | E31H | no change |
| 27 | L35V | no change |
| 28 | L35I | no change |
| 29 | E37Q | increase |
| 30 | E37H | ND |
| 31 | P42S | ND |
| 32 | P42A | ND |
| 33 | D43Q | ND |
| 34 | D43H | ND |
| 35 | K45Q | no change |
| 36 | K45T | increase |
| 37 | F48I | no change |
| 38 | F48V | increase |
| 39 | Y49H | no change |
| 40 | Y49I | increase |
| 41 | W51S | no change |
| 42 | W51H | ND |
| 43 | K52Q | increase |
| 44 | K52T | no change |
| 45 | R53H | increase |
| 46 | R53Q | ND |
| 47 | M54V | no change |
| 48 | M54I | no change |
| 49 | E55Q | no change |
| 50 | E55H | ND |
| 51 | E62Q | no change |
| 52 | E62H | no change |
| 53 | W64S | increase |
| 54 | W64H | no change |
| 55 | L69V | no change |
| 56 | L69I | increase |
| 57 | E72Q | ND |
| 58 | E72H | ND |
| 59 | L75V | ND |
| 60 | L75I | increase |
| 61 | R76H | no change |
| 62 | R76Q | no change |
| 63 | L80V | no change |
| 64 | L80I | increase |
| 65 | P87S | increase |
| 66 | P87A | increase |
| 67 | W88S | no change |
| 68 | W88H | ND |
| 69 | E89Q | increase |
| 70 | E89H | increase |
| 71 | P90S | increase |
| 72 | P90A | ND |
| 73 | L93V | increase |
| 74 | L93I | increase |
| 75 | D96Q | increase |
| 76 | D96H | increase |
| 77 | K97Q | ND |
| 78 | K97T | ND |
| 79 | L102V | increase |
| 80 | L102I | increase |
| 81 | R110H | increase |
| 82 | R110Q | increase |
| 83 | L112V | ND |
| 84 | L112I | ND |
| 85 | K116Q | increase |
| 86 | K116T | increase |
| 87 | P121S | ND |
| 88 | P121A | increase |
| 89 | P122S | increase |
| 90 | P122A | increase |
| 91 | D123Q | ND |
| 92 | D123H | increase |
| 93 | P129S | increase |
| 94 | P129A | increase |
| 95 | L130V | ND |
| 96 | L130I | increase |
| 97 | R131H | increase |
| 98 | R131Q | increase |
| 99 | D136Q | ND |
| 100 | D136H | ND |
| 101 | R143H | increase |
| 102 | R143Q | increase |
| 103 | Y145H | ND |
| 104 | Y145I | ND |
| 105 | R150H | increase |
| 106 | R150Q | increase |
| 107 | K152Q | ND |
| 108 | K152T | ND |
| 109 | K154Q | no change |
| 110 | K154T | ND |
| 111 | L155V | ND |
| 112 | L155I | ND |
| 113 | E159Q | no change |
| 114 | E159H | ND |
| 115 | R162H | ND |
| 116 | R162Q | ND |
| 117 | C29A | ND |
| 118 | C29I | ND |
| 119 | C29T | ND |
| 120 | C7A | ND |
| 121 | C7I | ND |
| 122 | C7T | ND |
| 123 | D123N | increase |
| 124 | D136N | increase |
| 125 | D43N | ND |
| 126 | D96N | no change |
| 127 | E159N | increase |
| 128 | E18N | ND |
| 129 | E21N | ND |
| 130 | E23N | ND |
| 131 | E31N | ND |
| 132 | E37N | ND |
| 133 | E55N | increase |
| 134 | E62N | ND |
| 135 | E72N | ND |
| 136 | E89N | ND |
| 137 | K116N | increase |
| 138 | K152N | ND |
| 139 | K154N | ND |
| 140 | K20N | ND |
| 141 | K45N | increase |
| 142 | K52N | increase |
| 143 | K97N | ND |
| 144 | D8N | ND |
| 145 | D165Q | increase |
| 146 | D165H | increase |
| 147 | D165N | increase |
| 148 | R166H | increase |
| 149 | R166Q | increase |
| 354 | L5I | ND |
| 355 | L5V | no change |
| 356 | E13Q | ND |
| 357 | E13H | ND |
| 358 | E13N | ND |
| 359 | R14H | ND |
| 360 | R14Q | ND |
| 361 | Y15H | ND |
| 362 | Y15I | ND |
| 363 | L16I | increase |
| 364 | L16V | ND |
| 365 | L17I | ND |
| 366 | L17V | ND |
| 367 | L67I | increase |
| 368 | L67V | ND |
| 369 | L70I | ND |
| 370 | L70V | ND |
| 371 | L81I | ND |
| 372 | L81V | ND |
| 373 | L91I | ND |
| 374 | L91V | increase |
| 375 | R103H | ND |
| 376 | R103Q | ND |
| 377 | L105I | increase |
| 378 | L105V | ND |
| 379 | L108I | ND |
| 380 | L108V | ND |
| 381 | L109I | ND |
| 382 | L109V | no change |
| 383 | E117Q | ND |
| 384 | E117H | ND |
| 385 | E117N | ND |
| 386 | F138I | ND |
| 387 | F138V | ND |
| 388 | R139H | ND |
| 389 | R139Q | ND |
| 390 | K140N | ND |
| 391 | K140Q | ND |
| 392 | L141I | ND |
| 393 | L141V | ND |
| 394 | F142I | ND |
| 395 | F142V | ND |
| 396 | F148I | ND |
| 397 | F148V | ND |
| 398 | L149I | ND |
| 399 | L149V | ND |
| 400 | L153I | ND |
| 401 | L153V | increase |
| 402 | Y156H | ND |
| 403 | Y156I | ND |

ND: Not determined

In a second set of experiments, resistance to proteolysis was measured as described above, except that a higher concentration of proteases containing 3% protease mixture (wt/wt) containing each of the following proteases, α-chymotrypsin, Endoproteinase GluC and trypsin (Sigma), was used to assess resistance to proteolysis. Table 27 shows the results of some of the tested polypeptides. The data are expressed as relative resistance to proteases among the samples tested: (+), (++), or (+++), with (+++) indicating the highest resistance to proteases, and (−) indicating no change compared to native erythropoietin.

TABLE 27
Resistance to Proteolysis of Candidate EPO LEADS

| | | Resistance |
|---|---|---|
| Nemo | | to |
| Code # | Mutation | proteases |
| 1 | P2S | — |
| 2 | P2A | — |
| 3 | P3S | — |
| 4 | P3A | +++ |
| 5 | R4H | + |
| 6 | R4Q | + |
| 7 | C7S | ND |
| 8 | C7V | ND |
| 9 | D8Q | — |
| 10 | D8H | — |
| 11 | R10H | + |
| 12 | R10Q | — |
| 13 | L12V | — |
| 14 | L12I | — |
| 15 | E18Q | + |
| 16 | E18H | — |
| 17 | K20Q | +++ |
| 18 | K20T | — |
| 19 | E21Q | + |
| 20 | E21H | — |
| 21 | E23Q | — |
| 22 | E23H | — |
| 23 | C29S | ND |
| 24 | C29V | ND |
| 25 | E31Q | + |
| 26 | E31H | — |
| 27 | L35V | — |
| 28 | L35I | — |
| 29 | E37Q | + |
| 30 | E37H | — |
| 31 | P42S | ND |
| 32 | P42A | ND |
| 33 | D43Q | — |
| 34 | D43H | — |
| 35 | K45Q | — |
| 36 | K45T | ++ |
| 37 | F48I | ++ |
| 38 | F48V | + |
| 39 | Y49H | — |
| 40 | Y49I | + |
| 41 | W51S | — |
| 42 | W51H | — |
| 43 | K52Q | ++ |
| 44 | K52T | + |
| 45 | R53H | + |
| 46 | R53Q | ND |
| 47 | M54V | — |
| 48 | M54I | — |
| 49 | E55Q | — |
| 50 | E55H | — |
| 51 | E62Q | — |
| 52 | E62H | — |
| 53 | W64S | — |
| 54 | W64H | — |
| 55 | L69V | — |
| 56 | L69I | + |
| 57 | E72Q | ND |
| 58 | E72H | ND |
| 59 | L75V | — |
| 60 | L75I | + |
| 61 | R76H | — |
| 62 | R76Q | — |
| 63 | L80V | — |
| 64 | L80I | ++ |
| 65 | P87S | + |
| 66 | P87A | + |
| 67 | W88S | — |
| 68 | W88H | ND |
| 69 | E89Q | ++ |
| 70 | E89H | ++ |
| 71 | P90S | ++ |
| 72 | P90A | + |
| 73 | L93V | + |
| 74 | L93I | +++ |
| 75 | D96Q | +++ |
| 76 | D96H | + |
| 77 | K97Q | — |
| 78 | K97T | — |
| 79 | L102V | — |
| 80 | L102I | — |
| 81 | R110H | — |
| 82 | R110Q | + |
| 83 | L112V | — |
| 84 | L112I | ND |
| 85 | K116Q | + |
| 86 | K116T | ++ |
| 87 | P121S | — |
| 88 | P121A | + |
| 89 | P122S | + |
| 90 | P122A | + |
| 91 | D123Q | ++ |
| 92 | D123H | + |
| 93 | P129S | + |
| 94 | P129A | + |
| 95 | L130V | ++ |
| 96 | L130I | +++ |
| 97 | R131H | + |
| 98 | R131Q | ++ |
| 99 | D136Q | — |
| 100 | D136H | — |
| 101 | R143H | ++ |
| 102 | R143Q | +++ |
| 103 | Y145H | ND |
| 104 | Y145I | ND |
| 105 | R150H | +++ |
| 106 | R150Q | + |
| 107 | K152Q | — |
| 108 | K152T | — |
| 109 | K154Q | — |
| 110 | K154T | — |
| 111 | L155V | — |
| 112 | L155I | — |
| 113 | E159Q | — |
| 114 | E159H | — |
| 115 | R162H | — |
| 116 | R162Q | — |
| 117 | C29A | ND |
| 118 | C29I | ND |
| 119 | C29T | — |
| 120 | C7A | ND |
| 121 | C7I | ND |
| 122 | C7T | ND |
| 123 | D123N | ++ |
| 124 | D136N | ++ |
| 125 | D43N | + |
| 126 | D96N | + |
| 127 | E159N | +++ |
| 128 | E18N | — |
| 129 | E21N | — |
| 130 | E23N | — |
| 131 | E31N | — |
| 132 | E37N | — |
| 133 | E55N | — |
| 134 | E62N | — |
| 135 | E72N | — |
| 136 | E89N | + |
| 137 | K116N | ++ |
| 138 | K152N | — |
| 139 | K154N | ND |
| 140 | K20N | — |
| 141 | K45N | ++ |
| 142 | K52N | ++ |
| 143 | K97N | — |
| 144 | D8N | — |
| 145 | D165Q | ++ |
| 146 | D165H | ++ |
| 147 | D165N | ++ |
| 148 | R166H | ++ |
| 149 | R166Q | + |
| 354 | L5I | — |
| 355 | L5V | — |
| 356 | E13Q | — |
| 357 | E13H | — |
| 358 | E13N | — |
| 359 | R14H | — |
| 360 | R14Q | — |
| 361 | Y15H | — |
| 362 | Y15I | — |
| 363 | L16I | ++ |
| 364 | L16V | ++ |
| 365 | L17I | ++ |
| 366 | L17V | + |
| 367 | L67I | ND |
| 368 | L67V | ND |
| 369 | L70I | ND |
| 370 | L70V | ND |
| 371 | L81I | — |
| 372 | L81V | — |
| 373 | L91I | — |
| 374 | L91V | — |
| 375 | R103H | — |
| 376 | R103Q | — |
| 377 | L105I | + |
| 378 | L105V | — |
| 379 | L108I | — |
| 380 | L108V | — |
| 381 | L109I | — |
| 382 | L109V | — |
| 383 | E117Q | — |
| 384 | E117H | — |
| 385 | E117N | — |
| 386 | F138I | ND |
| 387 | F138V | — |
| 388 | R139H | +++ |
| 389 | R139Q | +++ |
| 390 | K140N | — |
| 391 | K140Q | — |
| 392 | L141I | — |
| 393 | L141V | — |
| 394 | F142I | ND |
| 395 | F142V | ND |
| 396 | F148I | ND |
| 397 | F148V | ND |
| 398 | L149I | — |
| 399 | L149V | — |
| 400 | L153I | — |
| 401 | L153V | ++ |
| 402 | Y156H | — |
| 403 | Y156I | ND |

ND: Not determined

E. Interferon Beta

A total of 340 variant LEADs of Interferon beta (IFN-β) and 38 SuperLEADs were generated and tested based on the predetermined property of increased protease resistance. IFN-β candidate LEADs and SuperLEADs were tested in vitro for protease resistance using the method as described above, except that the resistance to protease was tested over time. Briefly, 100 μl of IFN-β at 400 and 800 pg/ml were incubated for variable times (0 h, 0.5 h, 2 h, 4 h, 8 h, 12 h, 24 h and 48 h) with a cocktail of proteases as described above, followed by assessment of residual anti-viral activity. The time of incubation with proteases required to give 50% of total activity (anti-viral) compared to the absence of incubation with proteases was determined. Table 28 (LEADs) and Table 29 (SuperLEADs) depicts the results of kinetic analysis of residual activity of some exemplary non-limiting IFN-β LEADs and SuperLEADs following treatment with protease and the time of incubation required to give 50% of total anti-viral activity. Also depicted in Table 28 and Table 29 is the rate of increased proteolysis, which is a ratio of time at 50% activity of the modified IFN-β polypeptide compared to the native IFN-β polypeptide.

TABLE 28
IFN-β LEADS Exhibiting Increased Resistance to Proteolysis

| NEMO | | Proteolysis Resistance | Rate: |
|---|---|---|---|
| Code | Mutant | Time at 50% Total Activity | Mutant/Wild-type |
| 9 | L5Q | 10.00 | 5 |
| 58 | M36I | 16.00 | 8 |
| 72 | L47I | 8.00 | 4 |
| 104 | K105S | 9.00 | 4.5 |
| 110 | K108S | 8.00 | 4 |
| 111 | K108H | 9.00 | 4.5 |
| 169 | L5D | 12.00 | 6 |
| 170 | L5E | 14.00 | 7 |
| 247 | L5N | 10.00 | 5 |
| 250 | L6E | 10.00 | 5 |
| 253 | L6Q | 8.00 | 4 |
| 254 | L6R | 10.00 | 5 |
| 255 | L6S | 12.00 | 6 |
| 257 | L9N | 9.00 | 4.5 |
| 259 | Q10D | 22.00 | 11 |
| 266 | S13D | 10.00 | 5 |
| 286 | C17N | 22.00 | 11 |
| 290 | C17T | 8.00 | 4 |
| 299 | N86Q | 12.00 | 6 |
| 301 | N86T | 6.00 | 3 |
| 321 | Q94D | 4.00 | 2 |
| 326 | Q94S | 8.00 | 4 |
| 336 | H97D | 11.00 | 5.5 |
| 369 | N90C | 24.00 | 12 |
| | WT | 2.0 | 1.0 |

TABLE 29
IFN-β SuperLEADS Exhibiting Increased Resistance to Proteolysis

| | | Proteolysis Resistance | |
|---|---|---|---|
| NEMO | | Time at 50% Total | Rate: |
| Code | Mutant | Activity | Mutant/Wild-type |
| 525 | L5E/Q10D | 16 | 8 |
| 528 | L5E/K108S | 24 | 12 |
| 532 | L6Q/L47I | 24 | 12 |
| 533 | L5D/K108S | 24 | 12 |
| 534 | L5N/L6E | 24 | 12 |
| 538 | L5Q/K108S | 16 | 8 |
| 540 | L5N/Q10D | 24 | 12 |
| 541 | L6Q/M36I | 16 | 8 |
| 542 | L5D/N86Q | 20 | 10 |
| 543 | L5N/K108S | 10 | 5 |
| 544 | L5D/L6Q | 10 | 5 |
| 545 | L6E/Q10D | 24 | 12 |
| 547 | L5Q/N86Q | 16 | 8 |
| 548 | L6E/K108S | 20 | 10 |
| 551 | L5D/L47I | 20 | 10 |
| 553 | L6Q/K108S | 16 | 8 |
| 554 | L5N/L6Q | 24 | 12 |
| 557 | L6E/N86Q | 24 | 12 |
| 558 | L5Q/L6Q | 20 | 10 |
| 559 | L5D/M36I | 24 | 12 |
| 560 | L5N/L47I | 24 | 12 |
| 561 | L6Q/N86Q | 24 | 12 |
| 523 | WT | 2.0 | 1.0 |
| 523 | WT | 2.0 | 1.0 |
| 523 | WT | 2.0 | 1.0 |
| 523 | WT | 2.0 | 1.0 |
| 523 | WT | 2.0 | 1.0 |
| 523 | WT | 2.0 | 1.0 |
| 523 | WT Average | 2.0 | 1.0 |

### Example 2

Cloning and Expression of IFN-α-2b and IFN-α-2b Mutants

Interferon alpha 2b (IFN-α-2b) cDNA were cloned for expression in mammalian cells and for expression in a prokaryotic expression system. The IFN-α-2b cDNA cloned for expression in mammalian cell encoded the precursor IFN-α-2b polypeptide containing the signal sequence, to facilitate optimal expression. The IFN-α-2b cDNA cloned for expression in prokaryotic cells encoded only the mature IFN-α-2b polypeptide. IFN-α-2b mutants were subsequently generated by site-directed mutagenesis in both the mammalian and prokaryotic systems.

### A. Precursor IFN-α-2b and IFN-α-2b Mutants for Mammalian Expression

Nucleic acid encoding the precursor form of IFN-α-2b was cloned into a mammalian expression vector for subsequent expression in mammalian cells. The plasmid encoding the precursor form of IFN-α-2b was used in a series of site-directed mutagenesis reactions to generate the IFN-α-2b candidate LEADs described above in Example 1.

1. Cloning of Precursor IFN-α-2b cDNA

Native IFN-α-2b cDNA encoding the precursor polypeptide was generated by PCR amplification and cloned into the mammalian expression vector, pSSV9-2EcoRI, prior to site-directed mutagenesis.

a. Generation of the Mammalian Expression Vector, pSSV9-2EcoRI

The mammalian expression vector, pSSV9-2EcoRI, was generated from the pSSV9 CMV 0.3 pA vector (see, Du et al. (1996) Gene Ther 3:254-261), by removing the inverted terminal repeat (ITR) functions and introducing a new EcoRI restriction enzyme site. Briefly, the pSSV9 CMV 0.3 pA vector DNA was digested with PvuII to remove the fragment containing the ITR functions, and then re-ligated. A new EcoRI restriction site was introduced by site-directed mutagenesis using the Quickchange Site-Directed mutagenesis kit (Stratagene), according to the manufacturer's instructions, with specifically-designed complementary oligonucleotides which served as primers that incorporated an EcoRI site into the newly synthesized DNA. The complementary oligonucleotide primers were:

EcoRI forward primer:

(SEQ ID NO: 2072)

5′-GCCTGTATGATTTATTGGATGTTGGAATTCCCTGATGCGGTATTTTC
TCCTTACG-3′
EcoRI reverse primer:

(SEQ ID NO: 2073)

5′-CGTAAGGAGAAAATACCGCATCAGGGAATTCCAACATCCAATAAATC
ATACAGGC-3′

The QuikChange method involves linear amplification of template DNA by the PfuUltra high-fidelity DNA polymerase. The EcoRI forward primer and EcoRI reverse primer, both containing the EcoRI site, were extended during cycling using the pSSV9 CMV 0.3 pA vector (which had previously been digested with PvuII and re-ligated) as a template. Extension of the primers resulted in incorporation of the EcoRI into the newly synthesized strands, and resulted in a mutated plasmid with staggered nicks. Following amplification, the nucleic acid was treated with DpnI, which digests the dam-methylated parental strands of the E. coli-derived pSSV9 CMV 0.3 pA vector. This resulted in “selection” of the newly-synthesized mutated plasmids, which were not methylated. The vector DNA containing the new EcoRI site was transformed into XL10-Gold ultracompetent E. coli cells, where bacterial ligase repaired the nicks and allowed normal replication to occur.

The inclusion of the new EcoRI site into the resulting construct was confirmed by sequencing using the following oligonucleotides:

Seq ClaI forward primer:

(SEQ ID NO: 2074)

5′-CTGATTATCAACCGGGGTACATATGATTGACATGC-3′
Seq XmnI reverse primer:

(SEQ ID NO: 2075)

5′-TACGGGATAATACCGCGCCACATAGCAGAAC-3′

The XmnI-ClaI fragment containing the newly introduced EcoRI site was cloned into pSSV9 CMV 0.3 pA to replace the corresponding wild-type fragment and produce the construct pSSV9-2EcoRI.

b. Generation of IFN-α-2b Precursor cDNA

To facilitate optimal expression in mammalian cells, nucleic acid encoding the IFN-α-2b precursor polypeptide was first generated. This was achieved by introducing the nucleotides encoding the 17 amino acid IFN-α-2b signal peptide (first 17 amino acids of SEQ ID NO: 2078) to the 5′ end of the mature IFN-α-2b sequence (SEQ ID NO: 2079), using two PCR amplifications. Native IFN-α-2b cDNA encoding the mature polypeptide was obtained from the pDG6 construct (ATCC). The sequence of the IFN-α-2b cDNA in pDG6 was confirmed through sequencing using the following primers:

| | |
|---|---|
| Seq forward primer: | |
| 5′-CCTGATGAAGGAGGACTC-3′ | (SEQ ID NO: 2076) |
| Seq reverse primer: | |
| 5′-CCAAGCAGCAGATGAGTC-3′ | (SEQ ID NO: 2077) |

Following sequence verification, the mature IFN-α-2b cDNA was amplified by PCR from the pDG6 construct using the following primers:

Seq IFN-α-2b forward primer:

(SEQ ID NO: 2080)

5′-TCAGCTGCAAGTCAAGCTGCTCTGTGGGCTG-3′
Seq IFN-α-2b reverse XbaI primer
(XbaI site underlined):

(SEQ ID NO: 2081)

5′-GCTCTAGATCATTCCTTACTTCTTAAACTTTCTTGCAAGTTTGTT
GAC-3′

The resulting PCR product was then used as a template in a PCR reaction in which the “forward” oligonucleotide primer was used to introduce the signal sequence to the 5′ end of the mature IFN-α-2b cDNA during amplification. The primers used in this reaction were as follows:

IFN-α-2b 80bp forward HindIII primer (HindIII site
is underlined; IFN-α-2b signal peptide sequence
is in bold):

(SEQ ID NO: 2082)

5′-CCCAAGCTTATGGCCTTGACCTTTGCTTTACTGGTGGCCCTCCTGGT
GCTCAGCTGCAAGTCAAGCTGCTCTGTGGGCTG-3′
IFN-α-2b reverse XbaI primer (XbaI site is
underlined):

(SEQ ID NO: 2081)

5′-GCTCTAGATCATTCCTTACTTCTTAAACTTTCTTGCAAGTTTGTT
GAC-3′

The precursor IFN-α-2b cDNA sequence contained in the resulting PCR product was isolated by digestion with HindIII and XbaI and cloned into the pTOPO-TA vector (Invitrogen; SEQ ID NO: 2070) to generate pTOPO-IFN-α-2b. The sequence of the precursor IFN-α-2b was confirmed by automatic DNA sequencing. The pTOPO-IFN-α-2b vector was digested with HindIII and XbaI and the fragment containing the precursor IFN-α-2b cDNA sequence was cloned into the corresponding sites of pSSV9-2EcoRI (described above) to generate the construct pAAV-EcoRI-IFN-α-2b (pNB-AAV-IFN-α-2b).

2. Generation of Precursor IFN-α-2b Mutants by Site-Directed Mutagenesis

As described in Example 1 above, IFN-α-2b candidate LEADs were generated based on the predetermined property of protease resistance using the 2D-scanning technology as described herein and also described in published U.S. Pat. Pub. Nos. US2004/0132977 and US2005/0202438. The mutants were generated by site-directed mutagenesis using mutagenic primers that were designed to generate the appropriate site-specific mutations in the IFNα-2b cDNA. Mutagenesis reactions were performed with the Chameleon mutagenesis kit (Stratagene) using pNB-AAV-IFNα-2b as the template. Each individual mutagenesis reaction was designed to generate one single mutant protein.

Each individual mutagenesis reaction contains one mutagenic primer. For each reaction, 25 pmoles of each (phosphorylated) mutagenic primer were mixed with 0.25 pmoles of template, 25 pmoles of EcoRI-selection primer (this primer changed one nonessential unique EcoRI-restriction site into a new EcoRV restriction site), and 2 μl of 10× mutagenesis buffer (100 mM Tris-acetate pH 7.5; 100 mM MgOAc; 500 mM KOAc pH 7.5) into each well of 96 well-plates. To allow DNA annealing of the mutagenic and selection primers to the DNA template, PCR plates were incubated at 98° C. during 5 min and immediately placed 5 min on ice, before incubating at room temperature during 30 min. Elongation and ligation reactions were allowed by addition of 7 μl of nucleotide mix (2.86 mM each nucleotide; 1.43× mutagenesis buffer) and 3 μl of a freshly prepared enzyme mixture of dilution buffer (20 mM Tris HCl pH7.5; 10 mM KCl; 10 mM β-mercaptoethanol; 1 mM DTT; 0.1 mM EDTA; 50% glycerol), native T7 DNA polymerase (0.025 U/μl), and T4 DNA ligase (1 U/μl) in a ratio of 1:10, respectively. Reactions were incubated at 37° C. for 1 h before inactivation of T4 DNA ligase at 72° C. during 15 min.

In order to eliminate the parental plasmid, 30 μl of a mixture containing 1× enzyme buffer and 10 U of restriction enzyme (EcoRI) was added to the mutagenic reactions followed by incubation at 37° C. for at least 3 hours. Next, 90 μl aliquots of XLmutS competent cells (Stratagene) containing 25 mM β-mercaptoethanol were place in ice-chilled deep-well plates. Then, plates were incubated on ice for 10 min with gentle vortex every 2 min. Transformation of XL-mut competent cells was performed by adding aliquots of the restriction reactions ( 1/10 of reaction volume) and incubating on ice for 30 min. A heat pulse was performed in a 42° C. water bath for 45 s, followed by incubation on ice for 2 minutes. Preheated SOC medium (0.45 ml) was added to each well and plates were incubated at 37° C. for 1 h with shaking. In order to enrich for mutated plasmids, 1 ml of 2×YT broth medium supplemented with 100 μg/ml ampicillin was added to each transformation mixture followed by overnight incubation at 37° C. with shaking. Plasmid DNA isolation was performed by alkaline lysis using Nucleospin Multi-96 Plus Plasmid Kit (Macherey-Nagel) according to the manufacturer's instructions. Selection of mutated plasmids was performed by digesting 500 μg of plasmid preparation with 10 U of selection endonuclease in an overnight incubation at 37° C. A fraction of the digested reactions ( 1/10 of the total volume) was transformed into 40 μl of Epicurian coli XL1-Blue competent cells (Stratagene) supplemented with 25 mM β-mercaptoethanol.

Transformation was performed was as described above. Transformants were selected on LB-ampicillin agar plates incubated overnight at 37° C. Isolated colonies were picked up and grown overnight at 37° C. into deep-well plates. Four clones per reaction were screened by endonuclease digestion of a new restriction site (EcoRV) that was introduced by the selection primer. The cDNA from the selected mutant clones was sequenced to confirm the presence of the mutations.

B. Mature IFN-α-2b and IFN-α-2b(E41Q) for Prokaryotic Expression

Nucleic acid encoding the mature form of IFN-α-2b was cloned and codon-optimized by site-directed mutagenesis. The codon-optimized IFN-α-2b DNA was then used a template to generate the IFN-α-2b(E41Q) variant, which could be expressed in prokaryotic cells.

1. Cloning of Mature IFN-α-2b for Prokaryotic Expression

To express IFN-α-2b in E. coli, cDNA encoding the mature form of IFN-α-2b (SEQ ID NO: 2079) was initially cloned into the pTOPO-TA vector (Invitrogen) before being codon optimized. The codon-optimized mature IFN-α-2b sequence was then cloned into pET11 (Novagen; SEQ ID NO: 2183), and then subcloned into a pET24 vector that had been modified by removal of the f1 region and His-tag.

Briefly, the mature IFN-α-2b cDNA was amplified by PCR from pAAV-EcoRI-IFN-α-2b (described above) using Herculase DNA-polymerase (Stratagene) and the following primers:

Seq IFNA forward primer:

(SEQ ID NO: 2083)

5′-AACATATGTGTGATCTGCCTCAAACCCACAGCCTGGGTAGC-3′
Seq IFNA reverse primer:

(SEQ ID NO: 2084)

5′-AAGGATCCTCATTCCTTACTTCTTAAACTTTCTTGCAAGTTTG
TTG-3′

The resulting PCR product was cloned into pTOPO-TA vector (Invitrogen) to generate pTOPO-IFN-α-2b mature. The sequence was verified through sequencing using the M13 forward primer. The pTOPO-IFN-α-2b mature vector DNA was digested with NdeI and BamHI and the resulting fragment was subcloned into pET11 (Novagen; SEQ ID NO: 2183) to generate pET11-IFN-α-2b. The DNA sequence of the resulting pET11-IFN-α-2b construct was verified through sequencing.

a. Codon Optimization

In order to achieve maximum protein expression levels in E. coli, codon optimization was performed on the mature IFN-α-2b transcript using the QuikChange Site-Directed mutagenesis kit (Stratagene; described above). The sequences encoding the arginines at amino acid positions 12, 13, 22, 23 and 33 of the mature IFN-α-2b sequence set forth in SEQ ID NO: 2079 were mutated such that an optimal “CGT” arginine codon was introduced at each site. The sequence encoding the proline at amino acid position 39 was mutated such that a “CCG” proline codon was introduced. The stop codon also was optimized by mutagenesis, replacing the endogenous sequence with a “TAA” stop codon. The codon optimization was performed in a series of four mutagenic reactions, in which the mutagenized plasmid from the previous reaction was used a template for the next reaction. E. coli clones containing the mutagenized plasmids were screened after each step to select one containing the new mutation(s). The plasmid DNA of the selected clone was then isolated and used a a template in the next reaction. Thus, new mutations were introduced during each reaction until the final mature IFN-α-2b sequence contained 8 optimized codons.

The first site-directed mutagenesis was performed to optimize the codons encoding the arginine residues at amino acid positions 12 and 13 of the mature IFN-α-2b sequence. The pTOPO-IFN-α-2b mature plasmid DNA was used as the template, with the following primers:

ARG12/13-5′:

(SEQ ID NO: 2085)

CACAGCCTGGGTAGCCGTCGTACCTTGATGCTCCTGGC
ARG12/13-3′:

(SEQ ID NO: 2086)

GCCAGGAGCATCAAGGTACGACGGCTACCCAGGCTGTG

Following screening, the clone designated A1 was selected and used as a template for the second site-directed mutagenesis reaction. This reaction was performed to optimize the codons encoding the arginine residues at amino acid positions 22 and 23 of the mature IFN-α-2b sequence, and used the following primers:

ARG22/23-5′:

(SEQ ID NO: 2087)

ATGCTCCTGGCACAGATGCGTCGTATCTCTCTTTTCTCCTGC
ARG22/23-3′:

(SEQ ID NO: 2088)

GCAGGAGAAAAGAGAGATACGACQCATCTGTGCCAGGAGCAT

Following screening, the clone designated A1a2 was selected and used as a template for the third site-directed mutagenesis reaction. This reaction was performed to optimize the codons encoding the arginine residue at amino acid position 33 and the praline residue at amino acid position 39 of the mature IFN-α-2b sequence, and used the following primers:

ARG33/PRO39-5′:

(SEQ ID NO: 2089)

CTCCTGCTTGAAGGACCGTCATGACTTTGGATTTCCGCAGGAGGAG
TTTGGC
ARG33/PRO39-3′:

(SEQ ID NO: 2090)

GCCAAACTCCTCCTGCGGAAATCCAAAGTCATGACGGTCCTTCAAG
CAGGAG

Following screening, the clone designated A1a2b34 clone was selected. The plasmid was named pTOPO-IFN-α-2b-MUT4 and was used in the final site-directed mutagenesis reaction in which the IFN-α stop codon was modified. The following primers were used in this reaction:

(SEQ ID NO: 2091)

| | |
|---|---|
| IFNATAA5′: | GTTTAAGAAGTAAGGAATAAGGATCCTTAAGGGCG |

(SEQ ID NO: 2092)

| | |
|---|---|
| IFNATAA3′: | CGCCCTTAAGGATCCTTATTCCTTACTTCTTAAAC |

The resulting plasmid containing 8 optimized codons was designated pTOPO-IFN-α-2b-MUT4MUTSTOP3.

b. Cloning of the Codon-Optimized IFN-α-2B Sequence into pET24

The codon-optimized IFN-α-2b cDNA (IFN-α-2b-MUT4MUTSTOP3) was cloned into the expression vector, pET24a(+) (Novagen; SEQ ID NO: 2069), for prokaryotic expression of the mature polypeptide. Prior to cloning, however, the His-tag region was removed from the pET24a(+) vector. Following cloning of IFN-α-2b-MUT4MUTSTOP3 into pET24a(+), the f1 origin of replication also was removed.

The His-tag region in pET24a(+) is flanked by BlpI and XhoI restriction sites. The pET24a(+) vector was digested with BlpI and XhoI to removed the His-tag, and the remaining 5.2 kb DNA fragment was treated with Klenow fragment of DNA polymerase I to create blunt ends. Following purification, the 5.2 kb DNA fragment was religated to form pET24aΔHis1. IFN-α-2b-MUT4MUTSTOP3 was introduced into pET24aΔHis1 by first digesting pTOPO-IFN-α-2b-MUT4MUTSTOP3 with NdeI and BamHI and isolating the 500 bp fragment. The pET24aΔHis1 plasmid also was digested with NdeI and BamHI, and the 500 bp fragment containing IFN-α-2b-MUT4MUTSTOP3 was inserted into the plasmid by ligation. The resulting plasmid was designated pET24aΔHis1-IFN-α-2b-MUT4MUTSTOP1.

Two SpeI sites flanking the f1 origin of replication in pET24aΔHis1-IFN-α-2b-MUT4MUTSTOP1 were introduced by QuikChange site-directed mutagenesis to facilitate removal of this region. The first reaction used pET24aΔHis1-IFN-α-2b-MUT4MUTSTOP1 as the template, and the first SpeI site was introduced using the following primers:

(SEQ ID NO: 2093)

| | |
|---|---|
| 1SPEI5′: | GCTGAAAGGAGGAACTAGTTCCGGATTGGCGAATGG |

(SEQ ID NO: 2094)

| | |
|---|---|
| 1SPEI3′: | CCATTCGCCAATCCGGAACTAGTTCCTCCTTTCAGC |

The resulting pET24aΔHis1-IFN-α-2b-MUT4MUTSTOP1Mut1SpeIC11 clone was then used a template in a second reaction. The following primers were used to introduce the second SpeI site:

(SEQ ID NO: 2095)

| | |
|---|---|
| 2SPEI5′: | CAATTTCAGGTGGCACTAGTCGGGGAAATGTGCGC |

(SEQ ID NO: 2096)

| | |
|---|---|
| 2SPEI3′: | GCGCACATTTCCCCGACTAGTGCCACCTGAAATTG |

The resulting pET24aΔHis1-IFN-α-2b-MUT4MUTSTOP1Mut1+2SpeI Cln^{o}3B plasmid was digested with SpeI to remove the f1 replication of origin. The remaining 5.2 kb DNA fragment was purified and religated. The resulting pET24aΔHis1-IFN-α-2b-MUT4MUTSTOP1Mut1+2SpeI Cln^{o}3B plasmid was sequenced completely using the T7 primer at the following 10 primers:

| | |
|---|---|
| IFNAT71: | |
| CTG GGA GGT TGT CAG AGC | (SEQ ID NO: 2097) |
| pET24T72: | |
| ACC ATG AGT GAC GAC TG | (SEQ ID NO: 2098) |
| pET24T73: | |
| CCC GAC ATT ATC GCG AGC | (SEQ ID NO: 2099) |
| pET24T74: | |
| TCA AGA CGA TAG TTA CCG | (SEQ ID NO: 2100) |
| pET24T75: | |
| CTC CTT ACG CAT CTG TGC | (SEQ ID NO: 2101) |
| pET24T76: | |
| TAC TGA TGA TGA ACA TGC | (SEQ ID NO: 2102) |
| pET24T77: | |
| TCA TCG TCG CGC TCC AGC | (SEQ ID NO: 2103) |
| pET24T78: | |
| ATC CGC ACC AAC GCG CAG | (SEQ ID NO: 2104) |
| pET24T79: | |
| TCG TTC TAC CAT CGA CAC | (SEQ ID NO: 2105) |
| pET24T710: | |
| GCA TGC AAG GAG ATG GCG | (SEQ ID NO: 2106) |

Following verification of the sequence, the plasmid was designated pET24NAUT-IFN-α-2b-WT.

The pET24NAUT-IFN-α-2b-WT plasmid was transformed into competent BLR(DE3) E. coli cells (Novagen) and 40% glycerol stock culture of BLR/pET24NAUT-IFN-α-2b-cells was generated and stored in a cryotube at −80° C.

2. Generation of the IFN-α-2b(E41Q) Variant

To generate the IFN-α-2b(E41Q) variant, glutamic acid codon was substituted with a glutamine codon at the nucleic acid sequence that encodes for amino acid position 41 of the sequence set forth in SEQ ID NO: 2067. This was achieved using the QuikChange Site-Directed Mutagenesis kit (Stratagene) with pET24NAUT-IFN-α-2b(E41Q) vector DNA as the template and the following primers:

(SEQ ID NO: 2107)

| | |
|---|---|
| E41QMUT4: | GACTTTGGATTTCCGCAGCAGGAGTTTGGCAACCAG |

(SEQ ID NO: 2108)

| | |
|---|---|
| E41QMUT4C: | CTGGTTGCCAAACTCCTGCTGCGGAAATCCAAAGTC. |

One of the resulting clones, pET24NAUT-IFN-α-11c, was selected and the plasmid was designated pET24NAUT-IFN-α-2b(E41Q) (SEQ ID NO: 2068). The pET24NAUT-IFN-α-2b(E41Q) DNA was then transformed into competent BLR(DE3) E. coli cells (Novagen). The BLR/pET24NAUT-IFN-α-2b(E41Q) cells were grown in YP broth and a 40% glycerol stock was generated and stored at −80° C. The BLR/pET24NAUT-IFN-α-2b(E41Q) cells also were grown in LB broth to generate a 40% glycerol stock that was stored at −80° C.

### Example 3

### Laboratory-Scale Production of IFN-α-2b(E41Q)

For the production of IFN-α-2b(E41Q) for initial toxicology studies, the BLR/pET24NAUT-IFN-α-2b(E41Q) cells were cultured in LB medium (LB Broth Base powder (Lennox L Broth Base), 5 g/L NaCl, 10 g/L Select Peptone 140 (pancreatic digest of casein), 5 g/L Select Yeast Extract autolyzed low sodium (Invitrogen)). For subsequent studies, the BLR/pET24NAUT-IFN-α-2b(E41Q) cells were cultured in YP medium (1% Yeast Extract (Bio Springer), 2% A3 Soybean Peptone (Organotechnie), 2% NaCl (Sigma)). The IFN-α-2b(E41Q) protein was then purified from the culture, solubilized and refolded. Additional purification steps using chromatographical techniques were performed before a final filtration step to obtain purified IFN-α-2b(E41Q) protein.

1. Culture of 1 L Batch and Over-Expression Induction of IFN-α-2b(E41Q)

A cryotube containing the glycerol stock of the BLR/pET24NAUT-IFN-α-2b(E41Q) cells (previously grown in LB broth) was cultured in 300 mL LB supplemented with 30 μg/mL kanamycin (Sigma) for 15-16 h at 37° C. A 1 liter solution of LB/kanamycin was inoculated with a volume of pre-culture sufficient to reach OD₆₀₀ₙₘ=0.07 and grown at 37° C. When the culture reached OD₆₀₀ₙₘ=0.6 (estimated 8.70×10⁶ cells/mL), over-expression of IFN-α-2b(E41Q) was induced by adding 250 μl of 2 M IPTG (Amersham). The induced culture was grown for an additional 3 h at 37° C. The estimated doubling time was 45 min. The final OD₆₀₀ₙₘ was 1.8 to 2.2 with an estimated yield of approximately 30 to 40 mg/L. The culture was centrifuged at 4° C., 5,000×g, for 15 min, and the bacterial pellet was harvested and stored at −20° C.

2. Purification

The IFN-α-2b(E41Q) protein was initially purified from the cell lysate then solubilized and refolded. Anion-exchange chromatography, hydrophobic interaction chromatography and size-exclusion chromatography were performed before a final filtration step to obtain purified IFN-α-2b(E41Q) protein.

A. Cell Removal, Separation and Disruption

The bacterial pellet was thawed at room temperature prior to mechanical lysis. The thawed cells were resuspended in lysis buffer (lysozyme, 10 mM CaCl₂ and 1 U/mL DNase I) and sonicated for 8 min on ice. The lysate was centrifuged at 4° C., 10,000×g, for 30 min. The supernatant was removed and the pellet was stored at −20° C. The yield of this step was 99% and the purity was estimated to be 60%.

Due to the over-expression in E. coli and the hydrophobic nature of the protein, IFN-α-2b(E41Q) protein accumulated in inclusion bodies upon bacterial lysis. The frozen lysate pellet containing the inclusion bodies was thawed at room temperature. Lipid contaminants were removed from the inclusion bodies with two consecutive washing steps (each wash step was repeated twice). In the first washing step, the inclusion bodies were resuspended in 0.5% Triton X100 (Carlo Erba) and homogenized using an UltraTurrax homogenizer at 4,000 rpm for 8 sec. The homogenized sample was centrifuged at 4° C., 8,000×g for 30 min and the supernatant was removed. In the second washing step, the inclusion bodies were resuspended in 5% glycerol (Sigma) and homogenized using an UltraTurrax homogenizer at 4,000 rpm for 8 sec. The homogenized sample was centrifuged at 4° C., 8,000×g for 30 min and the supernatant was removed. The pellet was resuspended in 5% glycerol and stored at −20° C. The yield after the two washing steps was 95% and the purity was estimated to be 70%.

A final washing step was performed by mixing IFN-α-2b(E41Q) protein with an equal volume 6 M guanidinium chloride (Merck). The mixture was vortexed for 30 seconds and centrifuged at 13,000×g for 10 minutes, producing a yield of 95% with an estimated purity of 75%. The yield was estimated by comparing the recovery yield between total protein and IFN-α-2b(E41Q) (OD₂₈₀), while the purity was estimated by a combination of Coomassie Blue and silver nitrate stained SDS-PAGE. Additionally, the integrity of the protein was verified through antiviral specific activity assays as described in Example 1. These methods for determining yield, purity and integrity also were used in the subsequent purification steps described below.

b. IFN-α-2b(E41Q) Solubilization and Refolding

The inclusion bodies were denatured for 30 minutes in denaturation buffer (6 M guanidinium chloride) a 5:1 ratio by volume. The protein diluting in 50 times the volume of renaturation buffer (50 mM Tris pH 8.0, 10 mM EDTA, 0.2 M L-Arg (Sigma) and 5% glycerol) to effect refolding. The excess guanidinium chloride was removed by a first dialysis step in 20 times the volume of renaturation buffer for 15-16 hours, followed by a second dialysis in 80 times the volume 10 mM Tris buffer pH 8.0 for 2.5 hours at 4° C. (repeated twice) and subsequent filtration with a 0.45 μm PES membrane. The yield and purity following these steps were both estimated to be around 85%.

c. Chromatographic Purification Steps

Following the solubilization and refolding steps described above, IFN-α-2b(E41Q) was further purified through chromatography. The first chromatographic step was done using a 20 mL anion exchange column (AEX) Q Sepharose FF (Amersham) at a 120 cm/h flow rate, at 4° C. The column was equilibrated with 5 column volumes (CV) of 50 mM Tris pH 8.5. After the sample was loaded, the column was washed with 4 CV of 50 mM Tris pH 8.5. The first elution was performed with 3 CV of a 50 mM Tris pH 8.5, 200 mM NaCl solution. A second elution was performed with 2 CV of a 50 mM Tris pH 8.5, 1 M NaCl solution. Regeneration was performed with 5 CV of 1 M NaOH followed by a wash step with 3 CV of 50 mM Tris pH 8.5. The purity after the first chromatographic step was estimated to be 90%. The first elution fraction was stored at 4° C. for immediate use (i.e. less than 1 week) or at −20° C. for extended periods (i.e. greater than 1 week).

The second chromatographic step was done using a 5 mL Hydrophobic Interaction Column (HIC) Hi Trap Phenyl Sepharose HP (Amersham) at a 180 cm/h flow rate, at 4° C. The column was equilibrated with 5 CV of 50 mM Tris pH 8.5, 0.8 M (NH₄)₂SO₄. After the sample was loaded, the column was washed with 4 CV of 50 mM Tris pH 8.5, 0.8 M (NH₄)₂SO₄. The first elution was performed with 8 CV of 50 mM Tris pH 8.5, 0.4 M (NH₄)₂SO₄. A second elution was performed with 4 CV of 50 mM Tris pH 8.5. Regeneration was performed with 5 CV of 1M NaOH followed by a wash step with 3 CV of 50 mM Tris pH 8.5, 0.8 M (NH₄)₂SO₄. This chromatographic step removed traces of endotoxins and DNA, giving an estimated purity of 95%. The first elution fraction was stored at 4° C. for immediate use (i.e. less than 1 week) or at −20° C. for extended periods (i.e. greater than 1 week).

In order to control the level of acetylated protein in the final material, an LP-AEX chromatographic improvement step was introduced after the second chromatographic step. This acylation control step was set up with a 1 mL AEX Q HP column (Amersham) at a 1.27 cm/min flow rate, in a 20 mM Tris pH 8.5 at 20° C. A washing step of 0.2 CV was done with the same buffer. A first gradient was operated between 20 mM Tris pH 8.5 and 20 mM Tris pH 7.2 for 10 CV. A second gradient was operated between 20 mM Tris pH 7.2 and 20 mM bis-Tris pH 6.0 for 20 CV, followed by application of 60 CV of 20 mM bis-Tris pH 6.0. Fractionation was operated at a level of 1 CV. IFN-α-2b(E41Q) proteins were pooled until the level of acetylation reached a maximum 10%, as determined by RP-HPLC.

The third chromatographic step was done using a Size Exclusion Chromatography (SEC) Superdex 75 XK 16/70 column (Amersham) at a 24 cm/h flow rate, at 4° C. The column was equilibrated with 280 mL of 50 mM Tris pH 7.5, 150 mM NaCl. Following equilibration, 0.5 mL of sample (˜1 mg/mL) was loaded onto the column and IFN-α-2b(E41Q) proteins were harvested between 76 min and 85 min (elution peak between 0.6 CV and 0.8 CV). This chromatographic step removed traces of small proteins having the same isoelectric point and hydrophobicity properties as those of IFN-α, giving an estimated purity of 97%.

d. Final Purification

In the final purification stage, the liquid bulk was filtered using a 0.22 μm PES membrane, concentrated to between 0.4 mg/mL to 0.6 mg/mL IFN-α-2b(E41Q) using the Centricon Plus-80 system (Millipore), and stored at 4° C. for immediate use (<1 week) or at −20° C. (>1 week). This purified liquid substance was used in the subcutaneous toxicology studies for IFN-α-2b(E41Q) described in Examples 8, 15, 16.B.3, 16.C.1-2, 17.A-B, 18.A-C.

Additional 4, 10, and 12 L batches of IFN-α-2b(E41Q) liquid culture were generated for use in other animal investigations (see Examples 9a, 16.A.3). The methods for expression and purification of IFN-α-2b(E41Q) from these batches were identical to the methods for expression and purification of the 1 L batch described above, with the exception that the SEC chromatographic step was omitted prior to final purification. In addition, three native IFN-α-2b batches of 4, 10 and 11 L were produced and partially purified (without the SEC step) according to the procedures above. The various lab-scale batches of IFN-α-2b(E41Q) and native IFN-α-2b are summarized in Table 30 below.

TABLE 30
Laboratory-scale batches of IFN-α-2b(E41Q) and Native IFN-α-2b

| | | Starting | | | |
|---|---|---|---|---|---|
| | | Culture | Production | Drug | Used in |
| Product | Batch No. | Volume | Method | Form | Examples: |
| IFN-α- | IFN 001- | 4 L | Lab-scale; | Liquid | 8, 15, |
| 2b(E41Q) | 05; IFN | | Complete | | 16.B.3, |
| | 003-05 | | purification | | 16.C.1-2, |
| | | | | | 17.A-B, |
| | | | | | 18.A-C |
| IFN-α- | IFN 005- | 4 L | Lab-scale; | Liquid | 9 |
| 2b(E41Q) | 05 | | Partial | | |
| | | | purification | | |
| IFN-α- | IFN 007- | 10 L | Lab-scale; | Liquid | 9 |
| 2b(E41Q) | 05 | | Partial | | |
| | | | purification | | |
| IFN-α- | IFN 008- | 12 L | Lab-scale; | Liquid | 16.A.3 |
| 2b(E41Q) | 05 | | Partial | | |
| | | | purification | | |
| IFN-α-2b | IFN 004- | 4 L | Lab-scale; | Liquid | 9 |
| | 05 | | Partial | | |
| | | | purification | | |
| IFN-α-2b | IFN 006- | 10 L | Lab-scale; | Liquid | 9 |
| | 05 | | Partial | | |
| | | | purification | | |
| IFN-α-2b | IFN 002- | 11 L | Lab-scale; | Liquid | 8, 9, 11, |
| | 06 | | Partial | | 20.C |
| | | | purification | | |

### Example 4

### Preparation of IFN-α-2b(E41Q) for Pre-Clinical and Clinical Use

IFN-α-2b(E41Q) was prepared from BLR/pET24NAUT-IFN-α-2b(E41Q) cells for pre-clinical and clinical use according to the procedures described below. Using pharmaceutical quality materials, the scaled-up production of IFN-α-2b(E41Q) for pre-clinical and clinical use resulted in a final product suitable for use in humans. The methods for master cell bank (MCB) production, fermentation, biomass harvest, bacterial lysis, inclusion body (IB) treatment, purification of IFN-α-2b(E41Q) and liquid bulk formulation of IFN-α-2b(E41Q) for pre-clinical and clinical use are described below.

1. Master Cell Bank (MCB) Production Process

Six YP agar/kanamycin (30 μg/mL) plates were inoculated with BLR/pET24NAUT-IFN-α-2b(E41Q) YP glycerol stock (see Example 2B). After incubation on plates for 15-30 h at 37° C., one single colony was transferred from the plate into 450 mL YP/Kanamycin (30 μg/mL) medium. The culture was incubated at 37° C. for 8-12 h, until OD₆₀₀ₙₘ=2.0±0.3 was reached. Cells were harvested by adding cryopreservation media to a final concentration of 15% (v/v) and aliquoted in 1 mL samples into 240 labeled vials. The MCB aliquots were cryopreserved using a controlled rate freezer, and the vials were transferred to storage below −70° C. in vapor phase liquid nitrogen.

2. Fermentation

Large quantities of BLR/pET24NAUT-IFN-α-2b(E41Q) cells were grown by fermentation. For pre-culture, a frozen MCB aliquot was thawed and 830 μl was mixed with 40 mL YPN 2× growth medium (1% Yeast Extract, 2% Soybean Peptone, 2% NaCl), and 8 mL aliquots were distributed into 5×500 mL shake flasks. The flasks were then incubated with agitation at 250 rpm at 35° C. for about 17 h.

For batch culture, 100 L YPN 2× growth medium in a B. Braun Biotech International 150 L bioreactor was inoculated with the pre-culture at a starting OD₆₀₀ₙₘ=0.007. The fermentation proceeded at 37° C. approximately 7.30 h, and was performed at constant agitation (500 rpm), 50 L/min air flow, high sensitivity anti-foam, without antibiotics and without the use of pO₂ and pH regulation.

When OD₆₀₀ₙₘ reached a value of 3.2±0.5, the expression of IFN-α-2b(E41Q) was induced by the addition of 100 mL of 0.5 M IPTG (induction agent).

3. Harvest of Biomass

At 3 h post-induction, the culture was harvested and the biomass separated from the medium by tangential flow filtration (TFF) through hollow fibers (Amersham UFP-500-E-55, 2.1 m², NMWC 500,000) using a Watson-Marlow 704 U/R high flow peristaltic pump (equipped with a second pump head RX 701, in/out manometers, and an auxiliary low flow peristaltic pump). Specifically, the bacterial suspension was first concentrated 20-25 times, and then diafiltrated at constant volume against 4 to 5 volumes of 10 mM Tris 1 mM EDTA pH 8.0. The circuit was rinsed with 10 mM Tris 1 mM EDTA pH 8.0 and the rinse solution was pooled with the diafiltrated biomass. The harvest operation was performed at room temperature. The diafiltrated biomass (10±2 L) was transferred in one bag and stored at −20° C. until the lysis step.

4. Bacterial Lysis

A mechanical lysis step was performed to extract the IFN-α-2b(E41Q) protein from the bacterial cells. The bacterial biomass was thawed for 36 to 72 h at 4° C. or at room temperature. The biomass volume was adjusted to 12 L by addition of 10 mM Tris +1 mM EDTA, pH 8.0. In order to disrupt aggregates and reduce the viscosity of the suspension, the biomass was vigorously homogenized by means of an Ultra-Turrax T50 homogenizer for 15 min at 10,000 rpm. Bacterial cells were then mechanically disrupted by three runs through a Panda 2K cell disrupter at 750 bars. The resulting bacterial lysate was centrifuged for 30 min at 10° C. and 15,000×g. The supernatant was removed and the resulting pellets of inclusion bodies were either stored at 4° C. for less than 24 h or frozen at −20° C. until the next purification step.

5. Inclusion Body (IB) Treatment

a. IB Wash

In order to remove lipid contaminants, the inclusion body (IB) pellets were subjected to four wash steps. The IB pellets were resuspended in 0.5% Triton X100 and centrifuged for 25 min at 4° C. and 15,000×g. The pellets were harvested and the supernatant was removed. A second wash with 0.5% Triton X100 was performed, followed by an additional two washes with 5% glycerol.

After the fourth wash, the pellets were resuspended in 400 mL of 5% glycerol and divided into two sets of the same volume (dispensed into two plastic biotainers). Both suspensions were stored at −20° C. until the next purification step. The storage was typically about one month.

b. Denaturation of the IB and Renaturation of IFN-α-2b(E41Q)

In order to release the protein from the inclusion bodies, it was necessary to perform a denaturing step followed by a renaturing step to refold the protein. The following steps were performed successively on each IB suspension biotainer stored at −20° C. The IB suspension was diluted with denaturation buffer (6 M guanidinium chloride, 50 mM Tris 10 mM EDTA, 2 mM DTT, pH 8.0) to reach a volume that was 100 times greater than the weight of IB. The solution of denatured IB was then incubated under agitation for 1 hr at room temperature. The IB solution was added to the renaturation buffer (0.2 M L-Arginine, 50 mM Tris, 10 mM EDTA, 5% glycerol, pH 8.0) at a flow rate of 25 mL/min (dilution 1/50). The solution was incubated under agitation at 4° C. overnight.

c. Concentration and Diafiltration of the Renatured Protein Solution

Concentration and diafiltration were performed at 4° C. by tangential flow filtration (TFF), using a peristaltic pump, 2 manometers and a Pellicon®2 “Maxi” Filter Biomax PES membrane (EFA: 2.5 m², cut off: 10 kDa).

The membrane was first rinsed with WFI (water for injection), then with 10 L of 0.1 M NaOH without recirculation. Membrane sanitization was performed using 10 L of 0.1 M NaOH with a 2 hr duration of contact. The system was then purged of 0.1 M NaOH and rinsed with 20 L of 20 mM NaOH for 5 min. The system was purged of 20 mM NaOH and rinsed with 10 L of renaturation buffer without recirculation. The system was equilibrated with 10 L of renaturation buffer until the permeate reached pH 8.0. Residual particles in the renatured protein solution were eliminated by filtration using a 0.45 μm and 0.2 μm Sartopore 2 filter capsule (EFA=0.2 m², PES membrane, Sartorius). The clarified solution was concentrated 5 to 10 times, depending on the turbidity of the retentate. The solution was then exchanged against 5 volumes of 20 mM Tris pH 8.0. Pressure conditions were set in order to get a trans-membrane pressure of 0.5 bar and a flow rate of the permeate of between 0.3 and 1 L/min.

At the end of the diafiltration, the retentate was harvested and the TFF system was washed with 3-5 L of 20 mM Tris pH 8.0 which was added to the retentate solution. Residual precipitates were eliminated by filtration using a 0.45 μm and 0.2 μm Sartopore 2 filter capsule (EFA=0.45 m², PES membrane, Sartorius). The clarified solution was stored at 4° C. until proceeding to purification steps.

6. Purification

In order to increase the purity of the IFN-α-2b(E41Q) solution, seven purification steps were performed as described below. The chromatography system used for the purification steps of IFN-α-2b(E41Q) was an Akta Pilot from Amersham.

a. Step 1: Anion Exchange Chromatography (AEX)

The first purification step was conducted at 4° C., using a Q Sepharose FF resin, packed with WFI in a BPG100 column at a flow rate of 240 mL/min. Characteristics of the resin were as follows: section: 78.5 cm²; height: 15 cm; volume: 1,965 cm³. A flow rate of 200 mL/min was used for the loading, washing, elution and regeneration steps.

Before use, the column was sanitized with 2 column volumes (CV) of 1 M NaOH for 2 h followed by 2-3 CV of 20 mM NaOH with a flow rate of 200 mL/min. The column was stored in 20 mM NaOH until use (maximum of 28 days at 4° C.).

The column was equilibrated with 2-3 CV of 20 mM Tris 1 M NaCl pH 8.0 followed by 2-3 CV 20 mM Tris pH 8.0. IFN-α-2b(E41Q) solution was loaded onto the column followed by a wash step with 3-4.5 CV of 20 mM Tris, pH 8.0. The protein was first eluted with 2.5-3.5 CV of a solution containing 20 mM Tris and 100 mM NaCl, pH 8.0, followed by a second elution with 2.5-7.5 CV of a solution containing 20 mM Tris, 150 mM NaCl, pH 8.0. The column was washed with 2.5-3.5 CV of 20 mM Tris, 200 mM NaCl, pH 8.0, regenerated with 2.5-3.5 CV of 20 mM Tris +1 M NaCl, pH 8.0, and re-sanitized according to the above-mentioned procedure. The product of wash and elution were stored at 4° C. for 1-5 days.

b. Step 2: Hydrophobic Interaction Chromatography (HIC)

The second purification step was conducted at 4° C., using a Phenyl Sepharose FF resin, packed with WFI in a BPG100 column at a flow rate of 240 mL/min. Characteristics of the resin were as follows: section: 78.5 cm²; height: 20 cm; volume: 1,570 cm³. A flow rate of 200 mL/min was used for the loading, washing, elution, regeneration and sanitization steps.

Before use, the column was sanitized with 2 column volumes (CV) of 1 M NaOH for 2 hrs followed by 2-3 CV of 20 mM NaOH with a flow rate of 200 mL/min. The column was stored in 20 mM NaOH until use (maximum 28 days at 4° C.).

The column was equilibrated with 3-5 CV of a solution containing 20 mM Tris and 0.8 M (NH₄)₂SO₄, pH 8.0. The elution fractions from the previous step (AEX step) were pooled and adjusted to the same conductivity as the equilibration buffer by addition of a solution of 3.4 M (NH₄)₂SO₄. IFN-α-2b(E41Q) solution was loaded onto the column followed by four wash steps: 1) 2-4 CV of 20 mM Tris, 0.8 M (NH₄)₂SO₄, pH 8.0; 2) 3-5 CV 20 mM Tris, 0.6 M (NH₄)₂SO₄, pH 8.0; 3) 3-5 CV of 20 mM Tris, 0.5 M (NH₄)₂SO₄, pH 8.0; and 4) 3-5 CV 20 mM Tris, 0.4 M (NH₄)₂SO₄, pH 8.0. The four wash steps were followed by three elutions: 1) 8-11 CV of 20 mM Tris, 0.3 M (NH₄)₂SO₄, pH 8.0; 2) 4-6 CV of 20 mM Tris, 0.2 M (NH₄)₂SO₄, pH 8.0; and 3) 3-5 CV of 20 mM Tris, 0.1 M (NH₄)₂SO₄, pH 8.0. The three elution steps were followed by three regeneration steps: 1) 3-5 CV of 20 mM Tris, 0.05 M (NH₄)₂SO₄, pH 8.0; 2) 3-5 CV of 20 mM Tris, pH 8.0; and 3) 4-10 CV WFI (water for injection). The column was re-sanitized according to the above-described procedure. The product of elution was stored at 4° C. for 1-5 days until the next purification step (diafiltration).

c. Step 3: Diafiltration

The elution fractions from the above HIC purification step were diafiltrated, using a peristaltic pump, 2 manometers and a TFF P2B010A05 cartridge in PES (Millipore, PMNL: 10 kDa, EFA: 0.5 m²) placed in a Pellicon2 XX42P0060 carter. The entire step was conducted at 4° C.

Before use, the membrane was rinsed with WFI prior to sanitization. The membrane was rinsed again with 5 L of 0.1 M NaOH without recirculation. The sanitization was performed using 10 L of NaOH 0.1 M with for 2 h. The system was purged of 0.1 M NaOH and rinsed with 5 L of 20 mM NaOH for 5 min. The system was then purged of 20 mM NaOH and rinsed with 2 L of 20 mM Tris pH 8.5 without recirculation, after which it was equilibrated with 10 L of 20 mM Tris, pH 8.5, until the permeate reached pH 8.5. The elution fractions from HIC were pooled and diafiltrated against at least 4 volumes of 20 mM Tris, pH 8.5 (=exchange buffer), at a trans-membrane pressure of 0.5 bar.

At the end of the diafiltration, the retentate was harvested and the TFF system was washed with 1-2 L of 20 mM Tris, pH 8.5, which was added to retentate. The entire system was re-sanitized as described above.

d. Step 4: LP-AEX Column (Q Sepharose HP)

The fourth purification step was conducted at room temperature, using a Q Sepharose High Performance (Q HP) resin, packed with WFI in a BPG100 column at a flow rate of 100 mL/min. Characteristics of the resin were as follows: section: 78.5 cm²; height: 12 cm; volume: 943 cm³. A flow rate of 100 mL/min was used for the loading, washing, elution, regeneration and sanitization steps.

Before use, the column was sanitized with 2 column volumes (CV) of 1 M NaOH for 2 h followed by 2-3 CV of 20 mM NaOH with a flow rate of 100 mL/min and subsequent storage of the column in 20 mM NaOH until use (maximum 28 days at 4° C.).

The column was equilibrated with 2-5 CV of 20 mM Tris, 1 M NaCl, pH 8.5, followed by 2-3 CV of 20 mM Tris, pH 8.5. The IFN-α-2b(E41Q) solution was loaded onto the column followed by a wash step with 0.5-1.0 CV of 20 mM Tris, pH 8.5. Each elution step was collected in fractions. Samples from each fraction were pooled in order to get pool samples representative of different recovery yields (e.g., 20%, 25%, 30%, 35%, 40%, etc). Then each pool sample was analyzed to determine the acetylation level (specification: <2%). The first elution was performed with a pH gradient in 10 CV from 20 mM Tris, pH 8.5, to 20 mM Tris, pH 7.2, followed by a second elution with a pH gradient in 20 CV from 20 mM Tris, pH 7.2, to 20 mM bis-Tris, pH 5.9. The column was washed with 30 CV of 20 mM bis-Tris, pH 5.9, regenerated with 3-5 CV of 1 M NaCl, and re-sanitized according to the above-mentioned procedure. The product of wash and elution were stored at 4° C. for 1-5 days. All fractions of the first and second elution were stored at 4° C. until needed.

e. Step 5: Pre-SEC Concentration

A concentration step was performed using tangential flow filtration (TFF). The elution fractions from the above Q Sepharose HP purification step were pooled in order to get a final acetylation level <2%. The resulting pool step was concentrated using a peristaltic pump, 2 manometers and a TFF P2B005A05 cartridge in PES (Millipore, PMNL: 5 kDa, EFA: 0.5 m²) placed in a Pellicon2 XX42P0060 carter. The entire step was conducted at 4° C.

Before use, the membrane was rinsed with WFI and rinsed again with 5 L of 0.1 M NaOH without recirculation. Sanitization was performed using 10 L of 0.1 M NaOH for 2 h. The system was purged of 0.1 M NaOH and rinsed with 5 L of 20 mM NaOH for 5 min. The system was purged of 20 mM NaOH and rinsed with 2 L of 20 mM bis-Tris +150 mM NaCl, pH 5.9, without recirculation.

The system was equilibrated with 10 L of 20 mM bis-Tris +150 mM NaCl, pH 5.9, until the permeate reached pH 5.9. The elution fractions from the Q Sepharose HP purification step were pooled and adjusted to 150 mM NaCl by addition of 2 M NaCl in order to decrease the risk of precipitation of the proteins during the concentration. The resulting pool was concentrated to reach a final concentration of about 1 mg/mL, at a trans-membrane pressure of 0.5 bar.

At the end of the concentration, the retentate was harvested and the TFF system was washed with 0.6 L of 20 mM bis-Tris +150 mM NaCl, pH 5.9, which was added to the retentate. The resulting IFN-α-2b(E41Q) was filtered using a 0.45 μm+0.2 μm cartridge filter (Sartopore 2 filter, 300 cm², Sartorius). The entire system was sanitized as described above.

f. Step 6: Size Exclusion Chromatography (SEC)

The sixth purification step was conducted at 4° C., using a Superdex 75 Sepharose resin, packed with WFI in a BPG140 column. Characteristics of the resin were as follows: section: 153.9 cm²; height: 72 cm; volume: 1,108 cm³. A flow rate of 40 mL/min was used for the loading, washing, elution, regeneration and sanitization steps.

Packing of the resin was performed according to the following procedure. The resin was first packed with a flow rate of 40 mL/min for 1.00-1.30 h. The piston was pulled down and adjusted to 0.5 cm of the gel height. The flow rate was increased to reach a constant pressure of 4.5 bar for 20 min to 1 h. The piston was pulled down onto the gel. Finally, the quality of the packing was tested by measuring the Height Equivalent to the Theoretical Plate (HETP), HETP>1000, and the asymmetry (As), As=0.7-1.7, with 110.8 mL of 1% acetone.

Sanitization of the column was performed by running 0.5 M NaOH through the column for at least 120 min followed by storage in 20 mM NaOH until next use (max. 28 days at 4° C.).

The column was equilibrated with 16-18 L SEC buffer (1.69 g/L NaH₂PO₄.2H₂O, 1.8 g/L Na₂HPO₄, 7.5 g/L NaCl and 0.1 g/L EDTA Na₂) with a flow rate of 40 mL/min. 250 mL of IFN-α-2b(E41Q) solution was loaded onto the column followed by elution with the SEC buffer. The elution peak was collected in fractions for analysis. Each collected fraction was analyzed with SDS-PAGE. Only the fractions showing the higher purity level were kept and pooled together.

After salt elution, the column was sanitized with 0.5 M NaCl according to the above-mentioned procedure. Several runs were necessary to treat the complete bulk of IFN-α-2b(E41Q) (250 mL/run). All fractions were stored at 4° C. until results of purity measurements were obtained.

g. Step 7: Post-SEC Concentration

After purity testing, selected fractions from the above SEC purification step were pooled. The resulting pool was concentrated by tangential flow filtration (TFF), using the same system as in Step 5 (i.e. a peristaltic pump, 2 manometers and a TFF P2B005A05 cartridge in PES (Millipore, PMNL: 5 kDa, EFA: 0.5 m²) placed in a Pellicon2 XX42P0060 carter). The entire process was conducted at 4° C.

Before use, the TFF system was sanitized using 10 L of 0.1 M NaOH for 2 hrs. The system was purged of 0.1 M NaOH and rinsed with 5 L of 20 mM NaOH for 5 min. The system was then purged of 20 mM NaOH and rinsed with 2 L of SEC buffer without recirculation.

The system was equilibrated with 5 L of SEC buffer until the permeate reached pH 6.7. The selected elution fractions from the SEC purification step were pooled. The resulting pool was concentrated to reach a final concentration of 0.4 mg/mL, at a trans-membrane pressure of 0.5 bar.

At the end of the concentration, the retentate was harvested and the TFF system was washed with 0.2-0.4 L of SEC buffer which was added to retentate. The entire system was sanitized as described above. The resulting IFN-α-2b(E41Q) was filtered using a 0.45 μm and 0.2 μm cartridge filter (Sartopore 2 filter, 300 cm², Sartorius) and stored at 4° C. until proceeding to the formulation operations (=Purified Bulk).

7. Liquid Bulk-Formulation

The concentration of IFN-α-2b(E41Q) in the concentrated retentate was determined on the basis of the UV absorbance at 280 nm (Extinction Coefficient=0.985). The concentrated retentate was diluted with SEC buffer to obtain a final concentration of IFN-α-2b(E41Q) of 300±60 μg/mL. Sucrose was added to the IFN-α-2b(E41Q) Purified Bulk to get a sucrose concentration of 14.7 mg/mL. The formulated liquid bulk was sterilized by filtration through 0.2 μm using a sterilizing Sartopore 2 capsule (EFA=150 cm², PES membrane, Sartorius). The conditioned filtered Final Bulk was stored at −20° C. before proceeding to one of two lyophilization schemes outlined in Example 5.

Several batches of IFN-α-2b(E41Q) were produced by the methods described in this Example, including two batches that were lyophilized by the procedure outlined in Example 5.1 below (Method 1) for preparation of an enteric-coated capsule prototype and three batches that were lyophilized by the procedure outlined in Example 5.2 below (Method 2) for preparation of enteric-coated tablets. The various pre-clinical and clinical batches of IFN-α-2b(E41Q) are summarized in Table 31 below.

TABLE 31
Pre-clinical and clinical-scale batches of IFN-α-2b(E41Q)

| | | Starting | | | |
|---|---|---|---|---|---|
| | | Culture | Production | Drug | Used in |
| Product | Batch No. | Volume | Method | Form | Examples: |
| IFN-α- | IFN-001-06 | 100 L | Pre-clinical | Capsule; | 9, 10, 11, |
| 2b(E41Q) | | | (Method 1 | Injectable | 16.A.1-2, 20.B |
| | | | lyophilization) | solution | |
| IFN-α- | IFN 003-06 | 100 L | Pre-clinical | Capsule | n/a |
| 2b(E41Q) | | | (Method 1 | | |
| | | | lyophilization) | | |
| IFN-α- | G055/060118 | 100 L | Clinical | Tablet | 8, 11, 12, |
| 2b(E41Q) | | | (Method 2 | | 16.B.1, 20.A.3 |
| | | | lyophilization) | | |
| IFN-α- | G055/LPC1/060221 | 100 L | Clinical | Tablet | 11, 20.C |
| 2b(E41Q) | | | (Method 2 | | |
| | | | lyophilization) | | |
| IFN-α- | G055/LC1/060330 | 100 L | Clinical | Tablet | n/a |
| 2b(E41Q) | | | (Method 2 | | |
| | | | lyophilization) | | |

### Example 5

### Lyophilized Drug Substance

A lyophilized powder of IFN-α-2b(E41Q) was produced for the manufacture of enteric-coated capsules. Two methods of lyophilization were employed, as described below.

1. Pre-Clinical Batch Lyophilization (Method 1)

IFN-α-2b(E41Q) pre-clinical batch Nos. IFN-001-06 and IFN 003-06 (Table 31) were used to produce a lyophilized powder for the manufacture of enteric-coated capsule. The liquid bulk solution was filtered through a sterile filter (0.2 μm) using a peristaltic pump and silicone tubing followed by a freezing step at −80° C. for 90 min. The filtered solution was distributed in 5 mL sterile glass vials (about 3 mL per vial). During the primary lyophilization stage, the sample was brought to −30° C. for 60 min until the pressure reached 10 Pa (0.1 mbar). The room temperature was adjusted to 0° C. to facilitate evaporation. Approximately 100 mL of liquid bulk solution was lyophilized for 24 to 30 h. Lyophilization of 1 L solutions takes 3 to 5 days. During the secondary lyophilization stage, the temperature was increased by 5° C. every 20 min to reach 25° C. (room temperature) until the process was complete (6 hrs). The vials were closed with robust freeze-dry stoppers, sealed with aluminum capsule seals, and stored at 4° C.

Native IFN-α-2b liquid bulk from laboratory-scale batch Nos. IFN-004-05, IFN-006-05 and IFN-002-06 (Table 30) also were lyophilized according to the method above for use as a control in subsequent studies.

2. Clinical Batch Lyophilization (Method 2)

IFN-α-2b(E41Q) batch Nos. G055/060118, G055/LPC1/060221, G055/LC1/060330 (Table 31) were lyophilized for the manufacture of enteric-coated tablets. In addition to the IFN-α-2b(E41Q) protein, the powder contained the following:

TABLE 32

| | | Concentration |
|---|---|---|
| Excipients | Grades | (mg/mL) |
| NaCl | EP, for ACS analysis, ISO | 7.5 |
| Na phosphate dibasic | EP, for ACS analysis | 1.8 |
| Na phosphate monobasic | EP, for ACS analysis | 1.3 |
| EDTA disodium | EP, for ACS analysis, ISO | 0.1 |
| Sucrose | EP | 14.7 |

Frozen IFN-α-2b(E41Q) liquid bulk solution (stored at ≦−20° C.) was thawed for 26 hrs at room temperature then 14 hrs at 5-8° C., followed by an additional 3 hr incubation at room temperature. The solution was filtered through a Pall KA1DFLP sterile filter (0.2 μm) (Pall) using a peristaltic pump and a silicone tubing and filled directly into autoclaved Lyoguard® trays (WL Gore Associates). Five trays were filled with approximately 600 g solution/tray. In total, the yield of filtered solution was about 3,351 g. Trays were loaded into the freeze-dryer on pre-cooled shelves (−30° C.) and then frozen for 5 hrs.

The primary stage of the drying process was performed over 89 hrs. The frozen product was dried at −30° C. for 1 ht at 10 Pa (0.1 mbar), then at 0° C. for 1 hr at 10 Pa (0.1 mbar), and finally at 0° C. for 87 hrs at 10 Pa (0.1 mbar).

The secondary stage of the drying process was performed at a low vacuum at 5° C. for 20 min at 10 Pa (0.1 mbar), then at 5° C. for 1.5 h with the lowest possible pressure (about ≦0.001 mbar). The temperature was raised by 5° C. increments and held at each level for 60-90 min until the temperature reached 25° C. with the lowest possible pressure (about 0.001 mbar). This process took approximately 9 hrs.

At the end of the freeze-drying cycle, the trays were removed from the freeze-dryer and unloaded. The yield was determined at 88.6 g lyophilizate within the trays. The content of all trays was mixed and the lyophilizate was stored and protected from moisture until the next processing step.

### Example 6

### Manufacture of Tablets, Capsules and Solutions for Injection

IFN-α-2b(E41Q) was formulated as a solution for injection, an enteric-coated capsule and an enteric-coated tablet, for use in pre-clinical studies and/or clinical studies.

1. Solutions for Injection

IFN-α-2b(E41Q) lyophilized powder from the IFN 001-06 batch (described above) was used to formulate solutions for Per os (PO) and intravenous (IV) administrations in pre-clinical pharmacokinetic (PK) studies (Examples 9, 11). The IFN-α-2b(E41Q) lyophilized powder was resuspended in milliQ water for PO administration, or resuspended in PBS for intravenous administration. The respective amounts of IFN-α-2b(E41Q) lyophilized powder and diluent used to achieve a particular concentration, are provided in Table 33.

TABLE 33
Solutions for Injection

| | | Lyophilized | Volume |
|---|---|---|---|
| | Concentration | IFN-α-2b(E41Q) | (milliQ water) |
| Administration | (mg/mL) | (mg) | (mL PBS) |
| PO | 2.00 | 33.33 | 1 |
| | 1.00 | 16.65 | 1 |
| | 0.75 | 12.50 | 1 |
| | 0.50 | 8.32 | 1 |
| | 0.20 | 3.33 | 1 |
| IV | 0.10 | 1.66 | 1 (mL PBS) |

For additional studies in rats (see Examples 9, 16.A.3), solutions for injection also were prepared from the laboratory-scale batches of IFN-α-2b(E41Q): IFN 005-05, IFN 007-05 and IFN 008-05 (Example 3, Table 30).

2. Enteric-Coated Capsules

Enteric-coated capsules used in rat and monkey studies (Examples 9, 11, 16.A.1-2, 20B) were manufactured using the IFN-α-2b(E41Q) lyophilized powder from the IFN 001-06 pre-clinical batch. The capsules were filled with the appropriate amount of lyophilized IFN-α-2b(E41Q) and the weight was completed with sucrose. The capsules were enteric-coated for delivery into the intestine, with the coating serving to maintain capsule integrity at an acidic pH. The coating was made of 3 layers of a solution containing cellulose/acetate/phatalate in acetone (12.5% m/v). Each layer was dried separately. Clear Torpac (size 9) and Opaque Capsugel capsules (size 9) were used for the studies.

IFN-α-2b(E41Q) was formulated in enteric-coated capsules at 4 doses: 0.20 mg, 0.40 mg, 0.60 mg and 0.80 mg. The protein content was determined by OD 280 nm after dilution with PBS. The compositions of the four capsules are described in Table 34.

TABLE 34
Enteric-coated capsules

| | | | IFN-α- |
|---|---|---|---|
| | Unit dose | | 2b(E41Q) |
| Raw materials | (mg) | Capsule type | content (mg) |
| Lyophilized powder | 7.14 | Torpac | 0.20 |
| Mannitol | 21.86 | | |
| Lyophilized powder | 14.81 | Capsugel | 0.40 |
| Mannitol | 6.69 | | |
| Lyophilized powder | 21.75 | Capsugel | 0.60 |
| Lyophilized powder | 29.00 | Torpac | 0.80 |

The enteric-coating of each of the capsules was evaluated by maintaining the capsules in a solution at pH 1.2 for 1 hr at 37° C. This solution simulated the intestinal fluid environment. The solution and capsules were stirred throughout the incubation, after which the capsules were transferred to PBS and maintained at 37° C. All of the enteric-coated capsules tested showed no visual leaks during the 1 hr exposure to the acidic solution. Disintegration of the enteric-coated capsule was observed within 30 min when the capsule was transferred to PBS.

3. Enteric-Coated Tablets

IFN-α-2b(E41Q) lyophilized bulk (clinical batch No. G055/LC1/060330) was formulated as an enteric-coated capsule at three dosages; 40 μg IFN-α-2b(E41Q)/enteric-coated tablet, 200 μg IFN-α-2b(E41Q)/enteric-coated tablet, and 450 μg IFN-α-2b(E41Q)/enteric-coated tablet.

The enteric-coated tablets were manufactured by direct compression on an eccentric press. The IFN-α-2b(E41Q) lyophilized powder was first mixed with lactose monohydrate and triturated. Microcrystalline cellulose, povidone and crospovidone were then mixed into the powder, followed by magnesium stearate. The final powder mixture was compressed on an eccentric press using an oblong 11.0×4.4 mm tool that contained a breaking score on one side. The resulting tablet core weight was 150 mg and its size dimensions were 11.0×4.4 mm. The film used for coating the tablet was prepared by mixing isopropylic alcohol, acetone and purified water. Eudragit and triethylcitrate were then dissolved into this mixture. A separate solution of isopropylic alcohol, talc, titanium dioxide and magnesium stearate was prepared, and then the two solutions were mixed together to obtain the coating solution. The tablet cores were then coated with the coating solution until a final weight of 165.7 mg per tablet was obtained.

The composition of the IFN-α-2b(E41Q) enteric-coated tablet formulated for clinical use is shown in Table 35 below. The purity grades of each of the constituents also are shown (GMP is Good Manufacturing Practice, EP is European Pharmacopeia; USP is United States Pharmacopeia).

TABLE 35
Enteric-coated tablets (batch No. G055/LC1/060330)

| | | Strength 40 | Strength | Strength |
|---|---|---|---|---|
| | | μg/ | 200 μg/ | 450 μg/ |
| | | unit dose | unit dose | unit dose |
| Raw materials | Grades | (mg) | (mg) | (mg) |
| Drug substance: | | | | |
| *Lyophilized IFN-α- | GMP | 10.00 | 30.00 | 60.00 |
| 2b(E41Q) | material | 0.04 | 0.20 | 0.45 |
| Excipients: | | | | |
| *Lactose 1 H20 | EP | 105.80 | 85.80 | 55.80 |
| *Microcrystalline | EP | 27.00 | 27.00 | 27.00 |
| cellulose 0.1 mm | | | | |
| *Povidone (Polyvidone | USP | 1.80 | 1.80 | 1.80 |
| K25) | | | | |
| *Crospovidone | USP | 3.60 | 3.60 | 3.60 |
| *Magnesium stearate | USP | 1.80 | 1.80 | 1.80 |
| Enteric coating: | | | | |
| Eudragit L | USP | 8.40 | 8.40 | 8.40 |
| Thriethylcitrate | USP | 0.84 | 0.84 | 0.84 |
| Talcum micron | EP/USP | 4.20 | 4.20 | 4.20 |
| Titanium dioxide | USP | 1.50 | 1.50 | 1.50 |
| Magnesium stearate | USP | 0.75 | 0.75 | 0.75 |
| Isopropylic alcohol* | USP/EP | 70.00 | 70.00 | 70.00 |
| Acetone* | EP | 70.00 | 70.00 | 70.00 |
| Purified water* | USP | 0.42 | 0.42 | 0.42 |
| Total tablet mass | | 150.0 | 150.0 | 150.0 |

For use in the studies described in Examples 8, 11, 12, 16.B.1, 20.A.3 below, enteric-coated tablets were manufactured using the IFN-α-2b(E41Q) lyophilized bulk from clinical batch No. G055/060118 at a dose of 600 μg/tablet. The composition of the IFN-α-2b(E41Q) enteric-coated tablets used in these Examples is shown in Table 36 below.

TABLE 36
Enteric-coated tablets (batch No. G055/060118)

| | | Strength 600 μg/ |
|---|---|---|
| Raw materials | Grades | Unit dose (mg) |
| Drug substance: | | |
| *Lyophilized IFN-α-2b(E41Q) | | 51.70 |
| | | 0.60 |
| Excipients: | | |
| *Lactose 1 H20 | EP | 64.10 |
| *Microcrystalline cellulose 0.1 mm | EP | 27.00 |
| *Povidone (Polyvidone K25) | USP | 1.80 |
| *Crospovidone | USP | 3.60 |
| *Magnesium stearate | USP | 1.80 |
| Enteric coating: | | |
| *Eudragit S-100 | USP | 8.40 |
| *Triethylcitrate | USP | 0.84 |
| *Talc | EP/USP | 4.20 |
| *Titanium dioxide | USP | 1.50 |
| *Magnesium stearate | USP | 0.75 |
| *Purified Water | USP/EP | 0.42 |
| *Isopropylic alcohol | EP | 70.00 |
| *Acetone | USP | 70.00 |
| Total tablet mass | | 150.0 |

### Example 7

### Quality Control Results

Specifications and quality control (QC) data were collected for the clinical batches of IFN-α-2b(E41Q) at four stages of production: 1) purified liquid bulk (Example 4.6); 2) formulated liquid bulk (Example 4.7); 3) lyophilized powder (Example 5.2), and 4) tablet formulation (Example 6.3). Specifications and quality control data also were collected for the enteric-coated capsule prototype (Example 6.2) manufactured from the pre-clinical batch.

1. Purified Liquid Bulk Specifications and QC

Specifications and quality control data for the IFN-α-2b(E41Q) purified liquid bulk substance are shown in Table 37 below.

TABLE 37
Purified liquid bulk specifications and QC

| Analyses | Tests | Specifications |
|---|---|---|
| Appearance | Visual determination | Colorless, clear liquid |
| liquid bulk | | |
| Protein content | OD_{280 nm}, μBCA and RP-HPLC | For information |
| Identity | Peptide mapping | Conform to IFN-α-2b(E41Q) |
| | | pattern |
| | 15 N-terminal amino acid | Conform to reference amino acid |
| | sequence | sequence |
| | Isoelectric pH (IEF) | For information |
| | | (theoretical value: ~6.78/6.34) |
| | Western Blot | 1 specific band between 15 and 20 |
| | | kDa |
| | Molecular Weight by SDS- | 1 major band between 15 and 20 |
| | PAGE silver stain in reducing | kDa |
| | and non-reducing conditions | |
| Purity | SDS-PAGE under reducing and | ≧98% |
| | non-reducing conditions | |
| | Coomassie Blue staining | |
| | SE-HPLC | ≧98% |
| pH | Potensiometric measure | For information |
| Potency | Antiviral activity | ≧3.00 × 10⁸ IU/mg (A549 lung |
| (Texcell) | | cells)* |
| Related impurities | | |
| Acetylated form | RP-HPLC | No more than 2% of acetylated |
| | | IFN-α-2b(E41Q) |
| Aggregates | SE-HPLC | ≦1% |
| DNA content | Picogreen method | <10 ng/dose equiv¹ |
| HCP content | ELISA method | <10 ng/dose equiv¹ |
| GuaCl content | HPLC | <LOD² |
| Contaminants | | |
| detection | | |
| Bacterial | LAL Test/EP method | <10 EU/dose equiv* |
| endotoxins | | |

*Potency determination for the GMP G1 Clinical batch was performed by CPE on A549 lung cells
¹dose equivalent = 100 μg of protein
²LOD = limit of detection

2. Formulated Liquid Bulk Specifications and QC

Specifications and quality control data for the IFN-α-2b(E41Q) formulated liquid bulk substance (after sucrose addition) are shown in Table 38 below.

TABLE 38
Formulated liquid bulk specifications and QC

| Tests | Methods | Specifications |
|---|---|---|
| Appearance | Clarity, color | Colorless, clear liquid |
| Identity | Detection of IFN-α-2b(E41Q) | At least 1 specific band |
| | (Western Blot) | between 15 and 20 kDa |
| | (reducing conditions) | |
| Sterility test | TSB (20-25° C.) | Absence of growth |
| | FTM (30-35° C.) | Absence of growth |
| Abnormal toxicity | In mice | Absence |
| | In guinea-pigs | Absence |
| Endotoxin content | LAL | <10 IU/dose equiv¹ |
| Protein content | OD_{280 nm} | For information |
| | μBCA | 300 ± 60 μg/mL |
| Sucrose content | Spectrometry | 14.7 mg/mL |
| Potency | Antiviral activity | ≧3.00 × 10⁸ IU/mg (A549 lung |
| (Texcell) | | cells) |
| Electrophoretical | SDS-PAGE | 1 major band between 15 and |
| pattern/EP Standard | AgNO₃ - reducing & non- | 20 kDa |
| IFN-α | reducing conditions | |

¹dose equivalent = 100 μg of protein

3. Lyophilized Bulk Specifications and QC

Specifications and quality control data for the IFN-α-2b(E41Q) lyophilized bulk are shown in Table 39 below.

TABLE 39
Lyophilized bulk specifications and QC

| Analyses | Methods | Specifications |
|---|---|---|
| Appearance | Visual determination | White powder |
| Identity | LC-MS (RP-HPLC | Conform to EP pattern |
| (M-Scan, Switzerland) | and Mass | |
| | spectrometry on the | |
| | intact protein) | |
| | Peptide mapping | Conform to EP pattern |
| Strength | LC-UV | 0.9% (w/w) ± 0.1% |
| (M-Scan, Switzerland) | | |
| Purity | RP-HPLC | >98% |
| (M-Scan, Switzerland) | | |
| Dimers and HMW | RP-HPLC | ≦2% |
| (M-Scan, Switzerland) | | |
| Potency | Antiviral activity | 3.5 × 10⁸-7.5 × 10⁸ IU/mg |
| (NewLab BioQuality, Germany) | (CPE/A549 lung | (TBC) |
| | cells) | |
| Water content | Karl Fischer method | <10% |
| (Wulfing Pharma, Germany) | | |

4. Enteric-Coated Tablet and QC

Specifications and quality control data for the IFN-α-2b(E41Q) enteric-coated tablets are shown in Table 40 below.

TABLE 40
Enteric-coated tablet specifications and QC

| Analyses | Methods | Specifications |
|---|---|---|
| Appearance* | Visual | White tablets |
| | determination | |
| Identity* | LC-MS (RP-HPLC | Conform to EP pattern |
| (M-Scan, Switzerland) | and Mass | |
| | spectrometry on the | |
| | intact protein) | |
| | Peptide mapping | Conform to EP pattern |
| Strength* | LC-UV | 40, 200 and 450 μg ± 10% |
| (M-Scan, Switzerland) | | |
| Purity* | RP-HPLC | >95% |
| (M-Scan, Switzerland) | | |
| Dimers and HMW* | RP-HPLC | ≦5% |
| (M-Scan, Switzerland) | | |
| Potency* | Antiviral activity | 1.1 × 10⁸-2.9 × 10⁸ IU/mg |
| (NewLab BioQuality, Germany) | (CPE/A549 lung | |
| | cells) | |
| Dissolution | USP 29 <711> | >2 h in gastric condition |
| (CBA, Germany)* | | <45 min in neutral |
| | | condition |
| Microbiological examination | EP/USP (USP 29 | |
| (Wulfing Pharma, Germany)* | <61> methods) | |
| | Total aerobic | <10³ CFU/mg |
| | microbial count | |
| | Total combined | <10² CFU/mg |
| | yeasts/molds count | |
| | Absence E. coli | Absence E. coli |
| Content uniformity* | USP 29 < 905> by LC- | |
| (M-Scan, Switzerland) | UV quantification | |
| | (M-Scan) | |
| Water content | Karl Fischer method | <10% |
| (Wulfing Pharma, Germany)** | | |
| Disintegration | EP 5.6 (2.9.1) | Gastric fluid > 2 h |
| (Wulfing Pharma, Germany)** | | Intestinal fluid < 30 min |
| Average mass | Weight measured | 165 mg ± 5% |
| (Wulfing Pharma, Germany)** | | |
| Thickness | Calliper system | 3 mm ± 10% |
| (Wulfing Pharma, Germany)** | | |

*Release tests
**For characterization

5. Enteric-Coated Capsule Specifications and QC

Specifications and quality control data for the IFN-α-2b(E41Q) enteric-coated capsule prototype (from the pre-clinical batch) are shown in Table 41 below.

TABLE 41
Enteric-coated capsule specifications and QC

| Analyses | Tests | Specifications |
|---|---|---|
| Appearance of enteric- | Visual determination | White capsule |
| coated capsules | | |
| Content uniformity | OD_{280 nm} | Desired concentration of |
| | | IFN-α-2b(E41Q) in |
| | | formulated |
| | | powder*/enteric-coated |
| | | capsule |
| Mass determination | Weight measurement | 28.00 mg**/enteric- |
| | | coated capsule or 27.30 |
| | | mg***/enteric-coated |
| | | capsule |

*The enteric-coated capsule formulation was prepared with 0.20 mg, 0.40 mg, 0.60 mg and 0.80 mg of IFN-α-2b(E41Q) per capsule. The protein content was determined by OD_{280 nm} after dilution with PBS
**Torpac capsule = 28.00 mg correspond to 19.00 mg formulated powder + 9.00 mg capsule weight
***Capsugel capsule = 27.30 mg correspond to 21.50 mg formulated powder + 5.80 mg capsule weight

### Example 8

### IFN-α-2b(E41Q) Screening with HCV Replicon System

The anti-viral activity of the IFN-α-2b(E41Q) preparations was assessed using the hepatitis C virus (HCV) replicon system. This is an in vitro cell culture system in which HCV is propagated following transfection of the cells with an HCV replicon (see e.g. Lohmann et al. (1999) Science 285:26-30). HCV virus does not infect cells in culture, so self-amplifying replicons are used to produce HCV proteins and RNAs in the cells. The HCV replicons are RNA constructs that typically contain the genes encoding the non-structural proteins, an IRES (internal ribosome entry site) to facilitate translation, and a selectable marker. Transfection of these replicons into suitable mammalian cells, such as Huh7 and Huh7.5.1 cells, results in autonomous production of HCV RNA and protein. The system can therefore be used to evaluate the antiviral properties various drugs and molecules, including IFN-α-2b(E41Q).

The antiviral activities of IFN-α-2b(E41Q) laboratory-scale batch No. IFN-003-05 and clinical batch No. G055/060118 were evaluated using the HCV replicon system, and compared to the activities native IFN-α (IFN 002-06) and an IFN-α-2b internal control (LAB21, Cambridge CB4 0GA, United Kingdom). Cells were transfected with HCV sub-type 1a and 1b replicons, and were then treated with the IFN-α-2b(E41Q), IFN-α-2b and IFN-α samples at 1, 5, 20, 100 and 500 IU/mL for 3 days. The level of HCV sub-type 1a and 1b replicon replication was determined by reverse transcription real-time PCR, which measured the amount of viral RNA produced in the cells. GAPDH mRNA also was quantified as used as endogenous control to normalize results for differences in the amount of total RNA added to each reaction. The EC50 (50% effective dose) were calculated by regression using the line generated from the mean data value at each concentration. The CC50 (50% cytotoxic concentration) is the concentration of IFN required reduce cell viability by 50%. The Selectivity Index (SI) was determined by measuring the CC50/EC50 ratio.

A dose-dependent inhibition of HCV replicon replication was observed when the IFN-α-2b(E41Q), IFN-α-2b or IFN-α samples were added to the cell culture (Table 42). Similar results were obtained in the both the HCV 1a and 1b sub-type replicon systems. Both batches of IFN-α-2b(E41Q) showed anti-viral activity comparable to the activity shown by standard IFN-α control samples.

TABLE 42
Inhibition of HCV replicon replication

| | | | GAPDH | | | |
|---|---|---|---|---|---|---|
| | HCV | HCV | CC50 | | | Anti- |
| | 1a | 1b | (1a and | SI | SI | viral |
| Sample | EC₅₀ | EC₅₀ | 1b)* | 1a** | 1b** | effect |
| Native | 3.9 | 5.5 | >500.0 | >128.2 | >90.0 | Active |
| IFN-α | | | | | | |
| IFN 002-06 | IU/mL | IU/mL | IU/mL | | | |
| IFN-α- | 2.5 | 6.0 | >500.0 | >200.0 | >83.3 | Active |
| 2b(E41Q) | IU/mL | IU/mL | IU/mL | | | |
| G055/ | | | | | | |
| 060118 | | | | | | |
| IFN-α- | 0.8 | 1.5 | >500.0 | >625.0 | >333.3 | Active |
| 2b(E41Q) | IU/mL | IU/mL | IU/mL | | | |
| IFN-003-05 | | | | | | |
| LAB21 | 1.0 | 2.7 | >500.0 | >500.0 | >185.2 | Active |
| IFN-α- | IU/mL | IU/mL | IU/mL | | | |
| 2b internal | | | | | | |
| control | | | | | | |

### Example 9

### In Vivo Pharmacokinetic (PK) Studies of IFN-α-2b(E41Q) in Normal Rats Compared to Native IFN-α Following Various Routes of Administration

Pharmacokinetic studies were performed on 8-week old Sprague-Dawley male rats (301-325 g) to compare various routes of administration, including intraduodenal (ID) subcutaneous (SC), per os (PO, liquid gavage and enteric-coated capsules), and intravenous (IV) administrations. The following dosages were tested for each administration as follows: IV (10 μg per rat of IFN-α-2b(E41Q) or native IFN-α), ID (2 mg per rat of IFN-α-2b(E41Q) or native IFN-α), SC (50 μg per rat of IFN-α-2b(E41Q) or native IFN-α), PO (2 mg per rat of IFN-α-2b(E41Q) or native IFN-α, 1, 0.75, 0.5 and 0.25 mg per rat of IFN-α-2b(E41Q) for the enteric-coated capsule formulation; 2 mg per rat of IFN-α-2b(E41Q) or native IFN-α and 3.4, 1.9, 1 and 0.5 mg per rat of IFN-α-2b(E41Q) for the gavage liquid formulation).

1. General Procedures

At various time points following each administration, a blood sample (200 μl) was taken from all rats via a jugular vein catheter (inserted 18-24 hours before protein administration) for determination of remaining antiviral activity levels of IFN-α-2b(E41Q) or native IFN-α in plasma. The blood samples were placed in ice-cold collection tubes containing lithium-heparin and gently mixed followed by immediate addition of anti-protease solution (30 μl). Tubes were kept on ice (within 30 minutes of collection) and centrifuged at 2000 g for 10 minutes at 4° C. Plasma was harvested in 3 tubes of at least 40 μl each and kept frozen at −20° C. or −80° C. until use. The antiviral activity assay was performed using 20 μl plasma for IV route, 10 μl plasma for SC route or 30 μl plasma for ID and PO route diluted in 1 ml of culture medium followed by 2-fold serial dilutions. After a 16 h treatment of HeLa cells with diluted plasma samples, cells were infected with the EMC virus. At 48 h post-infection, the number of living cells was determined by viable cell staining (methylene blue) and OD measurement at a wavelength of 660 nm.

EC₅₀ and specific activity (IU/ml of animal plasma) were calculated for each kinetic point using NEMO in-house software with the following equation: [((A/B)×C)×(10001V)]/[D/B], with A being the remaining specific activity detected in plasma of the injected protein, B the specific activity of the IFN-α-2a NIBSC standard control (NIBSC code 95/650) determined in the assay, C the NIBSC quantity (in IU) present in the volume added to cells during the assay, D the specific activity of the injected protein and V the plasma volume tested. The antiviral activity of IFN-α-2b(E41Q) or native IFN-α samples (expressed as number of IU/mg of protein) was determined as the concentration needed for 50% protection of the cells against the EMC virus-induced cytopathic effect. Using PK solution software (SummitPK), PK parameters including Tₘₐₓ, Cₘₐₓ, half-life, and AUC (area under the curve) were determined for each pharmacokinetic profile. AUC refers the commonly used PK parameter which measures the area under the curve in a plot of concentration of drug in plasma against time.

2. Drug Batches

For the pharmacokinetic studies presented in this Example, three different IFN-α-2b(E41Q) batches were used: IFN-005/05, IFN-007/05 and IFN-001/06 (described in Examples 3 and 4). To compare the pharmacokinetic activity of IFN-α-2b(E41Q) to unmodified IFN-α-2b, three different native IFN-α-2b batches also were used: IFN-006/05, IFN-004/05 and IFN-002/06 (described in Example 3). Specifications and routes of administration for each batch are presented in Tables 43(a-g) below.

TABLE 43a
IFN-α-2b(E41Q) Batch # IFN-005/05

| | |
|---|---|
| Correspondence | 227 MIU = 1 mg |
| MIU vs. mg | |
| Presentation | Vials containing 22.7 MIU of IFN-α-2b(E41Q) |
| | solubilized in 24% Tris buffer pH 8.5 (50 mM) and |
| | 76% PBS solution containing 0.1% BSA at a |
| | nominal concentration of 22.7 MIU/ml (100 μg/ml) |
| Number of vials | 1 (IV) and 4 vials (SC) |
| Volume per vial | 1 ml |
| Administrations | Intravenous and subcutaneous |

TABLE 43b
IFN-α-2b(E41Q) Batch # IFN-007/05

| | |
|---|---|
| Correspondence | 250 MIU = 1 mg |
| MIU vs. mg | |
| Presentation | Vials containing 500 MIU of IFN-α-2b (E41Q) |
| | solubilized in 60% Tris buffer pH 8.5 (50 mM) and |
| | 40% PBS solution containing 0.1% BSA at a |
| | nominal concentration of 500 MIU/ml (2 mg/ml) |
| Number of vials | 7 vials |
| Volume per vial | 1 ml |
| Administration | Intraduodenal |

TABLE 43c
IFN-α-2b(E41Q) Batch # IFN-001/06

| | |
|---|---|
| Correspondence | 335 MIU = 1 mg |
| MIU vs. mg | |
| Presentation | Vials containing 1139, 670, 636.5, 335 and 167.5 |
| | MIU of IFN-α-2b(E41Q) re-solubilized in PBS |
| | buffer containing NaCl (7.5 g/l), sodium phosphate |
| | dibasic (1.8 g/l), sodium phosphate mono basic |
| | (1.3 g/l) EDTA disodium (0.1 g/l) and sucrose (14.7 |
| | g/l) pH 6.8-7 at a nominal concentration of 1139 |
| | MIU/ml (3.4 mg), 670 MIU/ml (2 mg/ml); 636.5 |
| | MIU/ml (1.9 mg/ml); 335 MIU/ml (1 mg/ml) and |
| | 167.5 MIU/ml (0.5 mg/ml) |
| Number of vials | 7 vials for each dose |
| Volume per vial | 1 ml |
| Administration | Per-os |

TABLE 43d
IFN-α-2b(E41Q) Batch # IFN-001/06

| | |
|---|---|
| Correspondence | 335 MIU = 1 mg |
| MIU vs. mg | |
| Presentation | Capsules containing 1139 MIU (3.4 mg), 670 MIU |
| | (2 mg), 636.5 MIU (1.9 mg), 335 MIU (1 mg) and |
| | 167.5 MIU (0.5 mg) of IFN-α-2b(E41Q) lyophilized |
| | with NaCl (7.5 g/l), sodium phosphate dibasic (1.8 |
| | g/l), sodium phosphate mono basic (1.3 g/l) EDTA |
| | disodium (0.1 g/l) and sucrose (14.7 g/l) pH 6.8-7 |
| Number of vials | 7 vials for each dose |
| Volume per vial | 1 ml |
| Administration | Capsule |

TABLE 43e
Native IFN-α-2b Batch # IFN-004/05

| | |
|---|---|
| Correspondence | 190 MIU = 1 mg |
| MIU vs. mg | |
| Presentation | Vials containing 50 MIU of native IFN-α-2b |
| | solubilized in 27% Tris buffer pH 8.5 (50 mM) and |
| | 73% PBS solution containing 0.1% BSA at a |
| | nominal concentration of 19 MIU/ml (100 μg/ml) |
| Number of vials | 4 vials |
| Volume per vial | 1 ml |
| Administrations | Subcutaneous and intravenous |

TABLE 43f
Native IFN-α-2b Batch # IFN-006/05

| | |
|---|---|
| Correspondence | 200 MIU = 1 mg |
| MIU vs. mg | |
| Presentation | Vials containing 800 MIU of native IFN-α-2b |
| | solubilized in 52% Tris buffer pH 8.5 (50 mM) and |
| | 48% PBS solution containing 0.1% BSA at a |
| | nominal concentration of 400 MIU/ml (2 mg/ml) |
| Number of vials | 7 vials |
| Volume per vial | 2 ml |
| Administrations | Intraduodenal (ID) and single dose per-os (liquid) |

TABLE 43g
Native IFN-α-2b Batch # IFN-002/06

| | |
|---|---|
| Correspondence | 388 MIU = 1 mg |
| MIU vs. mg | |
| Presentation | Vials containing 776 MIU of native IFN-α-2b |
| | protein solubilized in PBS solution containing 0.1% |
| | BSA at a nominal concentration of 776 MIU/ml (2 |
| | mg/ml) |
| Number of vials | 7 vials |
| Volume per vial | 1 ml |
| Administrations | Subcutaneous and intravenous |

3. ID Administration

For the intraduodenal study, a single dose liquid formulation containing 2 mg of IFN-α-2b(E41Q) or native IFN-α-2b was administered per rat by intraduodenal (ID) route to 8-week old Sprague-Dawley male rats. In addition to the vein catheter described in (1), an intraduodenal catheter was inserted into the animal groups involved in the ID study, 18-24 hours before protein administration. Dosage information and experimental design details are presented in Table 44 below.

TABLE 44
Dosage and experimental detail for ID study

| | Number | | | | |
|---|---|---|---|---|---|
| Group | of | | | Administration | Dose and |
| number | animals | Surgery | Treatment | Route | volume |
| 1 | 6 | Intraduodenal | Native | Intraduodenal | 2 mg |
| | | catheterization; | IFN-α-2b | catheter | (1800MIU/kg-6 mg/kg) |
| | | jugular vein | | | 1 mL |
| | | catheterization | | | |
| 2 | 6 | Intraduodenal | IFN-α- | Intraduodenal | 2 mg |
| | | catheterization; | 2b(E41Q) | catheter | (1800MIU/kg-(6 mg/kg) |
| | | jugular vein | | | 1 mL |
| | | catheterization | | | |

Collection of blood samples (200 μL) was done via a jugular vein catheter at the following time-points: 0, 0.5, 1, 2, 4 and 8 h. The amount of remaining IFN-α-2b(E41Q) in animal plasma was determined by an antiviral activity assay based on the protection from the CPE of EMCV following the infection of HeLa cells, as described in detail above.

The PK parameters calculated from the average of six animal PK profiles after ID administration to rats (2 mg/rat) are presented in Table 45 below.

TABLE 45
PK parameters for ID administration

| | IFN-α-2b(E41Q) | Native IFN-α |
|---|---|---|
| Cₘₐₓ (IU/mL) | 16390 | 1592 |
| Half-life (h) | 3.2 | 0.9 |
| AUC_{(0-∞)} (IU-h/mL) | 89987 | 2991 |
| AUC₍₀₋ₜ₎ (IU-h/mL) | 81623 | 2486 |
| Tₘₐₓ (h) | 2 | 0.5 |

Following intraduodenal administration, a large increase in the antiviral activity was detected in IFN-α-2b(E41Q) rat plasma samples compared to native IFN-α-2b plasma samples. As shown in Table 45 above, IFN-α-2b(E41Q) Cₘₐₓ and AUC_{(0-∞)} parameters were respectively about 10.3-fold and 30-fold increased compared to those observed for native IFN-α, due to the increased absorption of IFN-α-2b(E41Q) from the duodenal compartment to the systemic circulation. In addition, an increase of about 3.6-fold in IFN-α-2b(E41Q) half-life in the systemic blood compartment compared to that of native IFN-α was observed, suggesting a sustained delivery of IFN-α-2b(E41Q) from the intestinal compartment to the systemic circulation most likely due to a slow absorption coupled with decreased susceptibility to intestinal protease degradation.

4. SC Administration

For the subcutaneous study, a single dose liquid formulation containing 50 μg of IFN-α-2b(E41Q) or native IFN-α-2b was administered per rat by subcutaneous (SC) route to 8-week old Sprague-Dawley male rats. Dosage information and experimental design details are presented in Table 46 below.

TABLE 46
Dosage and experimental detail for SC study

| | Number | | | | |
|---|---|---|---|---|---|
| Group | of | | | Administration | Dose and |
| number | animals | Surgery | Treatment | Route | volume |
| 3 | 6 | Jugular vein | Native IFN-α- | SC | 50 μg |
| | | catheterization | 2b | | (45MIU/kg-150 μg/kg) |
| | | | | | 0.5 mL |
| 4 | 6 | Jugular vein | IFN-α- | SC | 50 μg |
| | | catheterization | 2b(E41Q) | | (45MIU/kg-150 μg/kg) |
| | | | | | 0.5 mL |

Collection of blood samples (200 μL) was done via a jugular vein catheter at the following time-points: 0, 0.5, 1, 2, 4 and 8 h. PK parameters including Cₘₐₓ, Tₘₐₓ, half-life and AUC were determined for each PK profile and presented in Table 47 below.

TABLE 47
PK parameters for SC administration

| | IFN-α-2b(E41Q) | Native IFN-α |
|---|---|---|
| Cₘₐₓ (IU/ml) | 20431 | 17671 |
| Half-life (hr) | n/d | 0.9 |
| AUC_{(0-∞) expo} (IU-hr/ml) | n/d | 27834 |
| AUC₍₀₋ₜ₎ (IU-hr/ml) | 116475 | 26723 |
| Tₘₐₓ (hr) | 0.5 | 0.5 |

n/d corresponds to non determinable PK parameters due to no antiviral activity loss monitored at 24 h in rat plasma - pharmacokinetic curves show a “plateau” at 24 h

Administration of native IFN-α and IFN-α-2b(E41Q) protein by subcutaneous route led to a fast absorption of protein from the presystemic tissue compartment to the systemic blood circulation with a maximal concentration at 0.5 hours post-injection about 17671 IU/mL and 20431 IU/mL, respectively. Native IFN-α-2b also was rapidly eliminated from blood with a half-life about 0.9 h whereas PK profiles obtained for IFN-α-2b(E41Q) showed a plateau at 8 h post-injection. The increase in half-life observed for IFN-α-2b(E41Q) compared to native IFN-α was correlated with an increase in AUC₍₀₋ₜ₎ parameter about 4.3-fold.

5. PO Administration of Liquid Formulation

To determine the oral bioavailability of liquid formulations of IFN-α-2b(E41Q) after per os (PO) administration (i.e. PO liquid gavage), a single dose PO administration study was performed followed by a multi-dose study.

a. Single Dose PO Administration

For the initial PO administration study, a single dose liquid formulation containing 2 mg of IFN-α-2b(E41Q) or native IFN-α-2b was administered per rat by (PO) route to 8-week old Sprague-Dawley male rats. Dosage information and experimental design details are presented in Table 48 below.

TABLE 48
Dosage and experimental detail for PO (liquid, single dose) study

| | Number | | | | |
|---|---|---|---|---|---|
| Group | of | | | Administration | Dose and |
| number | animals | Surgery | Treatment | Route | volume |
| 1 | 6 | Jugular vein | Native IFN- | PO | 2 mg |
| | | catheterization | α-2b | Gavage (liquid) | (1800MIU/kg-6 mg/kg) |
| | | | | | 1 mL |
| 2 | 6 | Jugular vein | IFN-α- | PO | 2 mg |
| | | catheterization | 2b(E41Q) | Gavage (liquid) | (1800MIU/kg-6 mg/kg) |
| | | | | | 1 mL |

Collection of blood samples (200 μL) was done via a jugular vein catheter at the following time-points: 0, 0.5, 1, 2, 4 and 8 h. PK parameters including Cₘₐₓ, Tₘₐₓ, half-life and AUC were determined for each PK profile and presented in Table 49 below.

TABLE 49
PK parameters for PO (liquid, single dose) administration

| | IFN-α-2b(E41Q) | Native IFN-α-2b |
|---|---|---|
| Cₘₐₓ (IU/ml) | 21326 | — |
| Half-life (hr) | 3.7 | — |
| AUC_{(0-∞)} (IU-hr/ml) | 128174 | — |
| AUC₍₀₋ₜ₎ (IU-hr/ml) | 99909 | — |
| Tₘₐₓ (hr) | 0.5 | — |

A significant antiviral activity was detected in rat plasma following PO administration of IFN-α-2b(E41Q) liquid formulation with Cₘₐₓ values about 21326 IU/ml and AUC_{(0-∞) expo} parameter value about 128174 IU-hr/ml at a dose of 2 mg/rat. However, no antiviral activity detection was observed in the plasma of rats administered with the liquid formulation of native IFN-α-2b at the same dose. IFN-α-2b(E41Q) half-life was about 3.7 h in systemic blood circulation which was similar to the data obtained from intraduodenal injection.

b. Multi-Dose PO Administration

Following the initial single dose PO administration study, a decreasing multi-dose PK study was performed to determine the oral bioavailability of liquid formulations of IFN-α-2b(E41Q) after PO administration. A liquid formulation containing native IFN-α at a dose of 2 mg/rat or IFN-α-2b(E41Q) at doses of 3.4, 2, 1.9, 1 or 0.5 mg/rat were administered PO to rats. Dosage information and experimental design details are presented in Table 50 below.

TABLE 50
Dosage and experimental detail for PO (liquid, multi-dose) study

| | Number | | | | |
|---|---|---|---|---|---|
| Group | of | | | Administration | Dose and |
| number | animals | Surgery | Treatment | Route | volume |
| 1 | 5 | Jugular vein | Native IFN- | PO | 2 mg |
| | | catheterization | α-2b | Gavage (liquid) | (1800MIU/kg-6 mg/kg) |
| | | | | | 1 mL |
| 2 | 5 | Jugular vein | IFN-α- | PO | 3.4 mg |
| | | catheterization | 2b(E41Q) | Gavage (liquid) | (3000MIU/kg-10.2 mg/kg) |
| | | | | | 1 mL |
| 3 | 5 | Jugular vein | IFN-α- | PO | 2 mg |
| | | catheterization | 2b(E41Q) | Gavage (liquid) | (1800MIU/kg-6 mg/kg) |
| | | | | | 1 mL |
| 4 | 5 | Jugular vein | IFN-α- | PO | 1.9 mg |
| | | catheterization | 2b(E41Q) | Gavage (liquid) | (1710MIU/kg-5.7 mg/kg) |
| | | | | | 1 mL |
| 5 | 5 | Jugular vein | IFN-α- | PO | 1 mg |
| | | catheterization | 2b(E41Q) | Gavage (liquid) | (900MIU/kg-3 mg/kg) |
| | | | | | 1 mL |
| 6 | 5 | Jugular vein | IFN-α- | PO | 0.5 mg |
| | | catheterization | 2b(E41Q) | Gavage (liquid) | (450MIU/kg-1.5 mg/kg |
| | | | | | 1 ml |

Collection of blood samples (200 μL) was done via a jugular vein catheter at the following time-points: 0, 0.5, 1, 2, 4 and 6 h. PK parameters including Cₘₐₓ, Tₘₐₓ, half-life and AUC were determined for each PK profile and presented in Table 51 below.

TABLE 51
PK parameters for PO (liquid, multi-dose) administration

| | | | | | | Native |
|---|---|---|---|---|---|---|
| | IFN-α-2b(E41Q) | | | | | IFN-α |
| Dose (mg) | 3.4 | 2 | 1.9 | 1 | 0.5 | 2 |
| Cₘₐₓ (IU/mL) | 49074 | 29938 | 33211 | 11676 | 15531 | — |
| Half-life (h) | 4.3 | 3.6 | 3.4 | 2.8 | 2.1 | — |
| AUC_{(0-∞)} (IU-h/mL) | 220140 | 137531 | 154234 | 38212 | 31666 | — |
| AUC₍₀₋ₜ₎ (IU-h/mL) | 142022 | 89227 | 115048 | 38212 | 31666 | — |
| Tₘₐₓ (h) | 1 | 0.5 | 0.5 | 0.5 | 0.5 | — |

As shown in Table 51 above, a significant and dose-dependent antiviral activity was detected in rat plasma following PO administration of IFN-α-2b(E41Q) liquid formulation with Cₘₐₓ values about 49074, 29938, 33211, 11676 and 15531 IU/ml at doses of 3.4, 2, 1.9, 1 and 0.5 mg/rat, respectively. However, no antiviral activity detection was observed in plasma of rats administered with liquid formulation of native IFN-α at a dose of 2 mg/rat. These observations also were correlated with a dose-dependent increase of AUC_{(0-∞)expo} parameter with values about 31666, 38212, 154234, 137531 and 220140 IU-hr/mL obtained for doses of 0.5, 1, 1.9, 2 and 3.4 mg of IFN-α-2b(E41Q) liquid formulation, respectively. These data were in accordance with the data obtained from PO administration of liquid formulation of both IFN-α-2b(E41Q) and native IFN-α at a single dose (2 mg/rat) (described above).

In addition, a dose-related half-life was obtained for IFN-α-2b(E41Q) in the systemic blood compartment with values about 4.3, 3.6, 3.4, 2.8 and 2.1 h at doses of 3.4, 2, 1.9, 1 and 0.5 mg/rat, respectively, supporting the existence of a sustained delivery of IFN-α-2b(E41Q) from the intestinal compartment to the systemic circulation due to a slow absorption coupled with a resistance to intestinal protease degradation.

6. PO Administration of Enteric-Coated Capsule

A decreasing multi-dose PK study was performed to determine the oral bioavailability of enteric-coated capsule formulations of IFN-α-2b(E41Q) after per os (PO) administration. An enteric-coated capsule formulation containing native IFN-α at a dose of 2 mg/rat or IFN-α-2b(E41Q) at doses of 2, 1, 0.75, 0.5, 0.25 mg/rat were administered PO to rats using a gavage tube. Dosage information and experimental design details are presented in Table 52 below.

TABLE 52
Dosage and experimental detail for PO (enteric-coated capsule) study

| | Number | | | | |
|---|---|---|---|---|---|
| Group | of | | | Administration | Dose and |
| number | animals | Surgery | Treatment | Route | volume |
| 7 | 5 | Jugular vein | Native IFN- | PO | 2 mg |
| | | catheterization | α-2b | Enteric-coated | (1800MIU/kg-6 mg/kg) |
| | | | | Capsule | 1 mL |
| 8 | 5 | Jugular vein | IFN-α- | PO | 2 mg |
| | | catheterization | 2b(E41Q) | Enteric-coated | (1800MIU/kg-6 mg/kg) |
| | | | | Capsule | 1 mL |
| 9 | 5 | Jugular vein | IFN-α- | PO | 1 mg |
| | | catheterization | 2b(E41Q) | Enteric-coated | (900MIU/kg-3 mg/kg) |
| | | | | Capsule | 1 mL |
| 10 | 5 | Jugular vein | IFN-α- | PO | 0.75 mg |
| | | catheterization | 2b(E41Q) | Enteric-coated | (675MIU/kg-2.25 mg/kg) |
| | | | | Capsule | 1 mL |
| 11 | 5 | Jugular vein | IFN-α- | PO | 0.5 mg |
| | | catheterization | 2b(E41Q) | Enteric-coated | (450MIU/kg-1.5 mg/kg) |
| | | | | Capsule | 1 mL |
| 12 | 5 | Jugular vein | IFN-α- | PO | 0.25 mg |
| | | catheterization | 2b(E41Q) | Enteric-coated | (225MIU/kg-0.75 mg/kg) |
| | | | | Capsule | 1 mL |

Collection of blood samples (200 μL) was done via a jugular vein catheter at the following time-points: 0, 1, 1.5, 2, 4 and 6 h. PK parameters including Cₘₐₓ, Tₘₐₓ, half-life and AUC were determined for each PK profile and presented in Table 53 below.

TABLE 53
PK parameters for PO (enteric-coated capsule) administration

| | | | | | | Native |
|---|---|---|---|---|---|---|
| | IFN-α-2b(E41Q) | | | | | IFN-α |
| Dose (mg) | 2 | 1 | 0.75 | 0.5 | 0.25 | 2 |
| Cₘₐₓ (IU/mL) | 40202 | 34690 | 27491 | 25977 | 18034 | — |
| Half-life (h) | 4.7 | 3 | 4.3 | 1.24 | 0.63 | — |
| AUC_{(0-∞)} (IU-h/mL) | 303209 | 181015 | 190419 | 86139 | 39537 | — |
| AUC₍₀₋ₜ₎ (IU-h/mL) | 165892 | 151368 | 103686 | 61335 | 23795 | — |
| Tₘₐₓ (h) | 1 | 1.5 | 1 | 1 | 1 | — |

Similar to the liquid formulation, a significant and dose-dependent antiviral activity was detected in rat plasma following PO administration of the IFN-α-2b(E41Q) enteric-coated capsule formulation with Cₘₐₓ values about 40202, 34690, 27491, 25977, and 18034 IU/mL at doses of 2, 1, 0.75, 0.5 and 0.25 mg/rat, respectively. However, no antiviral activity detection was observed in plasma of rats administered with capsule formulation of native the IFN-α at a dose of 2 mg/rat. These observations also correlated with a dose-dependent increase of the AUC_{(0-∞) expo} parameter with values about 39537, 86139, 190419, 181015 and 303209 IU-hr/mL obtained for doses of 0.25, 0.5, 0.75, 1 and 2 mg/rat of the IFN-α-2b(E41Q) capsule formulation, respectively. These data were in accordance with previous data obtained following PO administration of IFN-α-2b(E41Q) liquid formulation showing an increase in absorption of IFN-α-2b(E41Q) from the intestinal compartment to the systemic circulation.

In addition, a dose-related half-life was obtained for IFN-α-2b(E41Q) in the systemic blood compartment with values about 4.7, 3, 1.24 and 0.63 h at doses of 2, 1, 0.5 and 0.25 mg/rat except for dose 0.75 mg/rat showing a half-life value about 4.3 hours. The dose-related relationship and half-life values observed following PO administration of IFN-α-2b(E41Q) capsule formulation were similar to data obtained with ID administered liquid formulations (compare to Table 45).

7. Bioavailability Analysis of PO Administration Versus Intravenous (IV) Administration

For each dose of IFN-α-2b(E41Q) administered PO (enteric-coated capsule or liquid formulations) to rats, the percentage of IFN-α-2b(E41Q) bioavailability compared to IV administration was determined. PK results for PO administration of liquid formulations or enteric coated capsule formulations are described above in (3) and (4). For IV administrations, rats were injected intravenously at a dosage of 10 μg per rat of IFN-α-2b(E41Q) or native IFN-α. Dosage information and experimental design details for IV administration are presented in Table 54 below.

TABLE 54
Dosage and experimental detail for IV study

| | Number | | | | |
|---|---|---|---|---|---|
| Group | of | | | Administration | Dose and |
| number | animals | Surgery | Treatment | Route | volume |
| 1 | 5 | Jugular vein | Native IFN- | IV | 10 μg |
| | | catheterization | α-2b | | (9MIU/kg-30 μg/kg) |
| | | | | | 100 μl |
| 2 | 3 | Jugular vein | IFN-α- | IV | 10 μg |
| | | catheterization | 2b(E41Q) | | (9MIU/kg-30 μg/kg) |
| | | | | | 100 μl |

Collection of blood samples (200 μL) was done via a jugular vein catheter at the following time-points: 0, 0.08, 0.50, 1, 2 and 4 h. PK parameters including Cₘₐₓ and AUC were determined for each PK profile and compared to the PO administrations of IFN-α-2b(E41Q) or native IFN-α.

The percentage of IFN-α-2b(E41Q) bioavailability (BAV) following PO administration compared to IV administration was calculated relative to Cₘₐₓ and AUC using the following equations:

BAV(%) related to Cₘₐₓ=((Cₘₐₓ/dose)_{IFN-α-2b(E41Q)}/(Cₘₐₓ/dose)_{IFN-α-2b(E41Q)-IV)}×100

BAV(%) related to AUC=((AUC/dose)_{IFN-α-2b(E41Q)}/(AUC/dose)_{IFN-α-2b(E41Q)-IV)}×100

BAV(%) related to AUC and to native IFN-α=((AUC/dose)_{IFN-α-2b(E41Q)}/(AUC/dose)_{native-IV)}×100

Bioavailability analysis (in %) performed on IFN-α-2b(E41Q) enteric-coated capsule formulation after PO administration to rats is presented in Table 55 below.

TABLE 55

| | Per-os administration (capsule formulation) | | | | | | IV administration | |
|---|---|---|---|---|---|---|---|---|
| | IFN-α-2b(E41Q) | | | | | native IFNα | IFN-α-2b(E41Q) | Native IFNα |
| Dose (mg) | 2 | 1 | 0.75 | 0.5 | 0.25 | 2 | 0.01 | 0.01 |
| AUC_{(0-∞) expo} (UI-hr/ml) | 303209 | 181015 | 190419 | 86139 | 39537 | — | 119465 | 45220 |
| Cₘₐₓ (UI/ml) | 40202 | 34690 | 27491 | 25977 | 18034 | — | 66372 | 93956 |
| | | | | | | — | | |
| Cmax/dose | 20101 | 34690 | 36655 | 51954 | 72136 | — | 6637200 | 9395600 |
| BA | | | | | | | | |
| ((Cmax/dose)_{Bel}/(Cmax/dose)_{Bel-IV}) × 100 | 0.3 | 0.5 | 0.6 | 0.8 | 1.1 | — | | |
| | | | | | | — | | |
| AUC/dose | 151605 | 181015 | 253892 | 172278 | 158148 | — | 11946500 | 4522000 |
| BA | | | | | | | | |
| (AUC/dose)_{Bel}/(AUC/dose)_{Bel-IV}) × 100 | 1.3 | 1.5 | 2.1 | 1.4 | 1.3 | — | | |
| BA relative | | | | | | | | |
| (AUC/dose)_{Bel}/(AUC/dose)_{natif-IV}) × 100 | 3.4 | 4.0 | 5.6 | 3.8 | 3.5 | — | | |

Bioavailability analysis (in %) performed on IFN-α-2b(E41Q) liquid formulation after PO administration to rats is presented in Table 56 below.

TABLE 56

| | Per-os administration (liquid formulation) | | | | | | IV administration | |
|---|---|---|---|---|---|---|---|---|
| | | | | | | native | | Native |
| | IFN-α-2b(E41Q) | | | | | IFNα | IFN-α-2b(E41Q) | IFNα |
| Dose (mg) | 3.4 | 2 | 1.9 | 1 | 0.5 | 2 | 0.01 | 0.01 |
| AUC_{(0-∞) expo} (UI-hr/ml) | 220140 | 137531 | 154234 | 38212 | 31666 | — | 119465 | 45220 |
| Cₘₐₓ (UI/ml) | 49074 | 29938 | 33211 | 11676 | 15531 | — | 66372 | 93956 |
| | | | | | | — | | |
| Cmax/dose | 14434 | 14969 | 17479 | 11676 | 31062 | — | 6637200 | 9395600 |
| BA | | | | | | | | |
| ((Cmax/dose)_{Bel}/(Cmax/dose)_{Bel-IV}) × | 0.2 | 0.2 | 0.3 | 0.2 | 0.5 | — | | |
| 100 | | | | | | — | | |
| AUC/dose | 64747 | 68766 | 81176 | 38212 | 63332 | — | 11946500 | 4522000 |
| BA | | | | | | | | |
| (AUC/dose)_{Bel}/(AUC/dose)_{Bel-IV}) × 100 | 0.5 | 0.6 | 0.7 | 0.3 | 0.5 | — | | |
| BA relative | | | | | | | | |
| (AUC/dose)_{Bel}/(AUC/dose)_{natif-IV}) × 100 | 1.4 | 1.5 | 1.8 | 0.8 | 1.4 | — | | |

8. Summary

For both liquid and enteric-coated capsule formulations, a significant and dose-dependent antiviral activity was detected in rat plasma following PO administration of IFN-α-2b(E41Q) whereas no signal was observed in plasma of native IFN-α protein administered rats. Similar results were obtained with ID administration of IFN-α-2b(E41Q) liquid formulation showing a large increase in Cₘₐₓ and AUC_{(0-∞) expo} parameters about 10.3-fold and 30-fold, respectively, for IFN-α-2b(E41Q) compared to native IFN-α. In addition, a dose-dependent increase in half-life was observed for IFN-α-2b(E41Q) following PO administration (with both capsule and liquid formulations) compared to IV route. Similarly, an increased half-life was observed for IFN-α-2b(E41Q) following ID versus IV administration compared to native protein which strongly supports the existence of a sustained delivery of IFN-α-2b(E41Q) from the intestinal compartment to the systemic circulation most likely related to a slow absorption coupled with resistance to intestinal protease degradation.

All these data demonstrate the capability of IFN-α-2b(E41Q) to be delivered into the systemic circulation following oral administration compared to native IFN-α.

### Example 10

### Pharmacokinetics and Immunogenicity of IFN-α-2b(E41Q) Following Oral Administration in Rats

As part of the toxicity study described in Example 16 below, pharmacokinetic profiles were determined for oral administrations of IFN-α-2b(E41Q). The purpose of this study was to assess the pharmacokinetics, immunogenicity and influence of anti-IFN-α-2b(E41Q) antibodies on the PK profile of IFN-α-2b(E41Q) when administered daily by PO route to rats via enteric-coated capsules for 2 weeks. Daily oral administrations of enteric-coated capsule formulations of IFN-α-2b(E41Q) with doses of 0.2, 0.4 and 0.6 mg per animal were performed in rats (specific pathogen free Sprague-Dawley rats) over 14 days. The Sprague-Dawley rat was chosen because of its utility as a predictor of toxic effects of drugs in humans and its recognition by regulatory authorities as a suitable species for toxicity studies. For each dose, six males (250-350 g) and six females (150-250 g, nulliparous and non-gravid) were orally administered 0.2, 0.4 or 0.6 mg doses of IFN-α-2b(E41Q) every day. The control group (6 males and 6 females) received the Placebo (enteric-coated capsules with the same formulation as IFN-α-2b(E41Q) enteric-coated capsules without IFN-α-2b(E41Q)).

1. General Procedures

On administration days 1, 7 and 14 and for each dose immediately before administration and at 1, 2, 4, 8 and 24 h after administration, blood samples were drawn from the retro-orbital sinus of 3 males and 3 females, sampled per time-point and per group, as illustrated in Table 57. A full pharmacokinetic profile of one IFN-α-2b(E41Q) dose was collected using 12 rats (6 males and 6 females), sampled as follows: one group of 3 males and 3 females drawn at pre-dose, 2 and 8 h and another group of 3 males and 3 females at 1, 4 and 24 h.

TABLE 57

| | | Time | | | | | |
|---|---|---|---|---|---|---|---|
| | Rat ID | Predose | 1 H | 2 H | 4 H | 8 H | 24 H |
| IFN-α- | M - 200611872 | X | | X | | X | |
| 2b(E41Q) | M - 200611807 | X | | X | | X | |
| 0.2 mg/rat | M - 200611808 | X | | X | | X | |
| | M - 200611809 | | X | | X | | X |
| | M - 200611810 | | X | | X | | X |
| | M - 200611811 | | X | | X | | X |
| | F - 200611854 | X | | X | | X | |
| | F - 200611855 | X | | X | | X | |
| | F - 200611856 | X | | X | | X | |
| | F - 200611857 | | X | | X | | X |
| | F - 200611858 | | X | | X | | X |
| | F - 200611859 | | X | | X | | X |
| IFN-α- | M - 200611812 | X | | X | | X | |
| 2b(E41Q) | M - 200611813 | X | | X | | X | |
| 0.4 mg/rat | M - 200611814 | X | | X | | X | |
| | M - 200611815 | | X | | X | | X |
| | M - 200611816 | | X | | X | | X |
| | M - 200611817 | | X | | X | | X |
| | F - 200611860 | X | | X | | X | |
| | F - 200611861 | X | | X | | X | |
| | F - 200611862 | X | | X | | X | |
| | F - 200611863 | | X | | X | | X |
| | F - 200611864 | | X | | X | | X |
| | F - 200611865 | | X | | X | | X |
| IFN-α- | M - 200611818 | X | | X | | X | |
| 2b(E41Q) | M - 200611819 | X | | X | | X | |
| 0.6 mg/rat | M - 200611820 | X | | X | | X | |
| | M - 200611821 | | X | | X | | X |
| | M - 200611822 | | X | | X | | X |
| | M - 200611823 | | X | | X | | X |
| | F - 200611866 | X | | X | | X | |
| | F - 200611867 | X | | X | | X | |
| | F - 200611868 | X | | X | | X | |
| | F - 200611869 | | X | | X | | X |
| | F - 200611870 | | X | | X | | X |
| | F - 200611871 | | X | | X | | X |

Blood samples from the retro-orbital bleeds were stored at room temperature for a few hours. Serum was prepared by centrifugation for 15 minutes at 3000 rpm (1620 g), at 4° C.±2° C. Serum was divided into 3 polypropylene tubes (70-80 μl each) and immediately frozen and stored frozen (<−70° C.) until use. The blood samples were taken from all the animals for i) the determination of plasma levels of IFN-α-2b(E41Q) (PK studies, see methods in Example 9) and ii) the measurement of both total and neutralizing anti-IFN-α antibody levels (antibody determination assays) in rat serum. For each tested dose, both total and neutralizing anti-IFN-α-2b(E41Q) antibodies were determined using the human anti-IFN-α antibody ELISA (for measuring total antibodies) according to the manufacturer's instructions (A4-203—MDS Pharma Services, Zürich, Switzerland) and Kawade's assay (for measuring neutralizing antibodies; Kawade et al., J. Interferon Research, 1987, Grossberg et al., J. Interferon Cytokines Research; 2001; Grossberg et al., Biotherapy; 1997; Kawade Y., J. Interferon Research, 1980; 1:61-70; Kawade et al., J. Immunological Methods, 2003).

2. Drug Batches

For the pharmacokinetic and immunogenicity studies presented in this Example, one batch of IFN-α-2b(E41Q) was used: IFN-001-06. As a control, enteric-coated placebo capsules were used. Specifications and routes of administration for the experimental and placebo batches are presented in Tables 58(a and b) below.

TABLE 58a
IFN-α-2b(E41Q) Batch # IFN-001-06

| | |
|---|---|
| Correspondence | 335 MIU = 1 mg |
| MIU vs. mg | |
| Presentation | Enteric-coated capsules (size 9) containing 0.2, 0.4 |
| | and 0.6 mg of IFN-α-2b(E41Q) lyophilized in NaCl |
| | 7.5 mg/mL; sodium phosphate dibasic 1.8 mg/mL; |
| | sodium phosphate monobasic 1.3 mg/ml; EDTA |
| | disodium 0.1 mg/mL and sucrose 14.7 mg/ml pH |
| | 6.8-7. Capsule filling is completed if necessary with |
| | mannitol powder |
| Administrations | Per-os |

TABLE 58b
Enteric-coated placebo capsules

| | |
|---|---|
| Correspondence MIU vs. mg | None |
| Presentation | Enteric-coated capsules (size 9) containing |
| | mannitol powder up to 28-30 mg |
| Administrations | Per-os |

3. Neutralizing Anti-IFN-α-2b(E41Q) Antibodies

Quantitative determination of neutralizing antibodies was performed according to Kawade's assay. Specifically, 10 LU (Laboratory Units) of IFN-α-2b(E41Q) were treated with 2-fold serial dilutions of rat serum samples (ranging from 14- to 28672-fold). After incubation for 1 h at 37° C., mixtures were placed 16 h on HeLa cells and then, cells were infected with EMC-Virus. At 48 h post-infection, the number of living cells was determined by viable cells staining (Methylene blue) and OD measurement.

Neutralizing antibody levels were determined as 10-fold reducing unit per mL of rat serum (TRU/mL) corresponding to the dilution of rat serum required to neutralize the activity of 10 LU of corresponding IFN-α-2b(E41Q) by calculating the neutralizing EC₅₀ value (nEC₅₀).

Results representing neutralizing antibody levels measured for each IFN-α-2b(E41Q) enteric-coated capsule dose (0.2, 0.4 and 0.6 mg) at day 15 are shown in Tables 59(a-c), respectively. A neutralizing antibody titer >20 TRU/ml (Ten-fold Reducing Unit) was considered as the threshold for positivity.

TABLE 59a
0.2 mg/rat

| Dose 0.2 mg/rats | TRU/ml |
|---|---|
| F854 | 0.0 |
| F855 | 0.0 |
| F856 | 0.0 |
| F857 | 0.0 |
| F858 | 0.1 |
| F859 | 0.0 |
| M807 | 81.3 |
| M808 | 0.0 |
| M809 | 0.0 |
| M810 | 0.0 |
| M811 | 0.0 |
| M872 | 0.0 |

TABLE 59b
0.4 mg/rat

| Dose 0.4 mg/rats | TRU/ml |
|---|---|
| F860 | 0.0 |
| F861 | 0.0 |
| F862 | 0.0 |
| F863 | 0.0 |
| F864 | 118.1 |
| F865 | 0.0 |
| M812 | 53.1 |
| M813 | 0.0 |
| M814 | 0.0 |
| M815 | 0.0 |
| M816 | 0.0 |
| M817 | 0.0 |

TABLE 59c
0.6 mg/rat

| Dose 0.6 mg/rats | TRU/ml |
|---|---|
| F866 | 0.0 |
| F867 | 0.0 |
| F868 | 0.0 |
| F869 | 0.0 |
| F870 | 0.0 |
| F871 | 0.0 |
| M818 | 0.0 |
| M819 | 0.0 |
| M820 | 0.0 |
| M821 | 0.0 |
| M822 | 0.0 |
| M823 | 0.0 |

No detectable levels of neutralizing antibodies were measured in rat serum at day 1 for doses of 0.2, 0.4 and 0.6 mg and for placebo in all animals tested.

As shown in Tables 59(a-c), at day 15, no detectable neutralizing antibody (Nab) levels were measured in 11 rats orally administered with 0.2 mg of IFN-α-2b(E41Q) enteric-coated capsule, only one male (M807) exhibited positive titer of 81.3 TRU/ml (Table 59a). Similarly, with a dose of 0.4 mg/rat (Table 59b), 10 rats exhibited no detectable levels of NAb whereas female F864 and male M812 showed detectable amounts of NAb of 118.1 TRU/ml and 53.1 TRU/ml, respectively. No titer of NAb was detected in all rats administered orally with a dose of 0.6 mg/rat (Table 59c).

4. Total Anti-IFN-α-2b(E41Q) Antibodies

Total anti-IFN-α antibody concentrations contained in rat serum samples were determined using the human anti-IFN-α antibody ELISA. The samples were diluted 9 times according to the manufacturer's protocol (IFN-alpha ELISA Kit Manual) and results were expressed in IU/mL after extrapolation from standard curve of OD value measured with spectrophotometer at 450 nm (SOP-AU-212-IFNα2b at IPM GmbH, Hamburg: determination of total anti-IFN alpha samples by MDS Pharma Services ELISA).

Results representing total antibodies levels measured for each IFN-α-2b(E41Q) enteric-coated capsule dose (0.2, 0.4 and 0.6 mg) are shown in Tables 60(a-c), respectively. The limit of detection of anti-IFN-α was determined to be 0.24 IU/mL. The calculated overall coefficient of variation in inter assays was 7.4% with a nominal concentration of 0.35 IU/mL and 2.5% with 4.2 IU/mL (determination made by the manufacturer of ELISA kits).

TABLE 60a
0.2 mg/rat

| Dose 0.2 mg/rats | IU/ml |
|---|---|
| F854 | 0.0 |
| F855 | 0.0 |
| F856 | 0.0 |
| F857 | 0.0 |
| F858 | 8.3 |
| F859 | 0.0 |
| M807 | 0.3 |
| M808 | 0.0 |
| M809 | 0.0 |
| M810 | 0.0 |
| M811 | 0.0 |
| M872 | 0.0 |

TABLE 60b
0.4 mg/rat

| Dose 0.4 mg/rats | IU/ml |
|---|---|
| F860 | 0.0 |
| F861 | 0.0 |
| F862 | 0.0 |
| F863 | 0.0 |
| F864 | 78.5 |
| F865 | 0.0 |
| M812 | 0.0 |
| M813 | 0.0 |
| M814 | 0.0 |
| M815 | 0.0 |
| M816 | 0.0 |
| M817 | 0.0 |

TABLE 60c
0.6 mg/rat

| Dose 0.6 mg/rats | IU/ml |
|---|---|
| F866 | 0.0 |
| F867 | 0.0 |
| F868 | 0.0 |
| F869 | 0.0 |
| F870 | 0.0 |
| F871 | 0.0 |
| M818 | 0.0 |
| M819 | 0.0 |
| M820 | 0.0 |
| M821 | 0.0 |
| M822 | 0.0 |
| M823 | 0.0 |

No detectable concentrations of total antibodies were measured in rat serum at day 1 for doses of 0.2, 0.4 and 0.6 mg and for placebo in all animals tested.

As shown in Tables 60(a-c), at day 15, no total anti-IFN-α-2b(E41Q) antibodies were detected in 10 rats orally administered with 0.2 mg of IFN-α-2b(E41Q) enteric-coated capsule. Only two animals, female F858 and male M807, exhibited low concentrations of total antibodies about 8.3 and 0.3 IU/ml, respectively (Table 60a). Similarly, with a dose of 0.4 mg/rat (Table 60b), 11 rats exhibited no detectable concentration levels of total antibodies except for female F864 with a detectable amount of total antibodies about 78.5 IU/ml. Of note, animal M812 showing positive NAb titer with a dose of 0.4 mg (53.1 TRU/ml) exhibited no detectable levels of total antibodies. An absence of total antibodies was observed in all rats orally administered with a dose of 0.6 mg/animal (Table 60c).

5. Remaining Antiviral Activity in Rat Serum Following Daily PO Administration of IFN-α-2b(E41Q) Enteric-Coated Capsule Formulation

PK parameters of IFN-α-2b(E41Q) enteric-coated capsule formulation after PO administration to rats at day 1 with doses of 0.2, 0.4 and 0.6 mg per animal are provided in Table 61 below. Means of both 3 male and 3 female rat PK parameters for each dose of IFN-α-2b(E41Q) are shown.

TABLE 61
Day 1

| | Cₘₐₓ | Tₘₐₓ | AUC_{(0-∞)} | AUC₍₀₋ₜ₎ |
|---|---|---|---|---|
| Enteric-coated capsules | (IU/mL) | (h) | (h-IU/mL) | (h-IU/mL) |
| IFN-α-2b(E41Q) - 0.2 mg | 4909 | 1 | 20945 | 20945 |
| IFN-α-2b(E41Q) - 0.4 mg | 53248 | 1 | 576002 | 501338 |
| IFN-α-2b(E41Q) - 0.6 mg | 180173 | 1 | 1731959 | 1730200 |

PK parameters of IFN-α-2b(E41Q) enteric-coated capsule formulation after PO administration to rats at day 7 with doses of 0.2, 0.4 and 0.6 mg per animal are provided in Table 62 below. Means of both 3 male and 3 female rat PK parameters for each dose of IFN-α-2b(E41Q) are shown.

TABLE 62
Day 7

| | Cₘₐₓ | Tₘₐₓ | AUC_{(0-∞)} | AUC₍₀₋ₜ₎ |
|---|---|---|---|---|
| Enteric-coated capsules | (IU/mL) | (h) | (h-IU/mL) | (h-IU/mL) |
| IFN-α-2b(E41Q) - 0.2 mg | 4379 | 1 | 16386 | 16386 |
| IFN-α-2b(E41Q) - 0.4 mg | 50144 | 1 | 413264 | 413264 |
| IFN-α-2b(E41Q) - 0.6 mg | 195917 | 1 | 1698113 | 1698113 |

PK parameters of IFN-α-2b(E41Q) enteric-coated capsule formulation after PO administration to rats at day 14 with doses of 0.2, 0.4 and 0.6 mg per animal are provided in Table 63 below. Means of both 3 male and 3 female rat PK parameters for each dose of IFN-α-2b(E41Q) are shown.

TABLE 63
Day 14

| | Cₘₐₓ | Tₘₐₓ | AUC_{(0-∞)} | AUC₍₀₋ₜ₎ |
|---|---|---|---|---|
| Enteric-coated capsules | (IU/mL) | (h) | (h-IU/mL) | (h-IU/mL) |
| IFN-α-2b(E41Q) - 0.2 mg | 4434 | 1 | 19673 | 19673 |
| IFN-α-2b(E41Q) - 0.4 mg | 38829 | 1 | 289805 | 289805 |
| IFN-α-2b(E41Q) - 0.6 mg | 112320 | 1 | 1175233 | 1175233 |

No impairment of PK profiles was observed at day 1 and day 7 with doses of 0.2, 0.4 and 0.6 mg. At day 14, AUC₍₀₋ₜ₎ and Cₘₐₓ mean values measured from all PK profiles were similar compared to day 1 for each administered IFN-α-2b(E41Q) dose tested except for animals treated with a dose of 0.6 mg presenting reduction in Cₘₐₓ and AUC₍₀₋ₜ₎ values (4909 IU/mL versus 4434 IU/mL for Cₘₐₓ at days 1 and 14 respectively with a dose of 0.2 mg; 180173 IU/mL versus 112320 IU/mL for Cₘₐₓ at day 1 and 14 respectively with a dose of 0.6 mg; 20945 IU-hr/mL versus 19673 IU/mL for AUC₍₀₋ₜ₎ at day 1 and 14 respectively with a dose of 0.2 mg and 1730200 IU-hr/mL versus 1175233 IU/mL for AUC₍₀₋ₜ₎ at days 1 and 14 respectively with a dose of 0.6 mg). However, no neutralizing antibody or total antibody was detected in rats treated with a dose of 0.6 mg at day 15 suggesting that the decrease in PK parameters observed between day 1 and day 14 was not due to the presence of antibodies but more likely due to animal heterogeneity and inter individual variability.

With a dose of 0.4 mg, a moderate but significant reduction of Cₘₐₓ and AUC₍₀₋ₜ₎ values also was observed between day 1 and day 14 (53248 IU/mL versus 38829 IU/mL for Cₘₐₓ at day 1 and day 14, respectively; 501338 IU-hr/mL versus 289805 IU-hr/mL for AUC₍₀₋ₜ₎ at day 1 and day 14, respectively).

These findings correlate well with the presence of positive levels of total and neutralizing antibodies in rat serum with a dose of 0.4 mg at day 15 (two out of the twelve animals).

6. Summary

In summary, no neutralizing and total antibody levels were detected with doses of 0.2 and 0.6 mg per animal in rat serum after daily PO administration of IFN-α-2b(E41Q) enteric-coated capsules over 2 weeks. This finding was supported by the absence of strong impairment of the IFN-α-2b(E41Q) pharmacokinetic profile observed at day 14 or at day 7 with doses of 0.2, 0.4 and 0.6 mg per animal after daily administration by oral route of IFN-α-2b(E41Q) enteric-coated capsules over 2 weeks. With a dose of 0.4 mg, only two out of the twelve animals showed positive neutralizing antibody titers (with only one animal presenting significant total antibody levels). A moderate reduction of Cₘₐₓ and AUC₍₀₋ₜ₎ values was observed between day 1 and day 14 with this dose.

### Example 11

### In Vivo Pharmacokinetic Study of IFN-α-2b(E41Q) in Cynomolgus Monkeys (Macaca fascicularis) Following IV and Oral Routes of Administration

An in vivo pharmacokinetic study was performed in Cynomolgus monkeys to assess the PK profiles of IFN-α-2b(E41Q) following IV and PO routes of administration. This data was then compared to previous PK profiles obtained for subcutaneous administration of native IFN-α and the commercial native IFN-α product Intron A® interferon. The results obtained from intravenous (IV) administration of IFN-α-2b(E41Q) and native IFN-α at a dose of 50 μg/kg were used to calculate the percentage of both IFN-α-2b(E41Q) and native IFN-α bioavailability following PO administration.

1. General Procedures

Pharmacokinetic studies were performed on plasma samples of monkeys administered with IFN-α-2b(E41Q) and native IFN-α. The PK profiles were based on the amount of remaining IFN-α-2b(E41Q) in animal plasma was determined by an antiviral activity assay (as described in Example 9).

2. Drug Batches

For the pharmacokinetic studies presented in this Example, three different IFN-α-2b(E41Q) batches were used: G055/LPC1/060221, IFN-001/06 and G055/060118T (described in Example 4). To compare the pharmacokinetic activity of IFN-α-2b(E41Q) to unmodified IFN-α-2b, a native IFN-α-2b batches also was used: IFN-002/06 (described in Example 3). Specifications and routes of administration for each batch are presented in Table 64(a-e) below.

TABLE 64a
IFN-α-2b(E41Q) Batch # G055/LPC1/060221

| | |
|---|---|
| Correspondence | 181 MIU = 1 mg |
| MIU vs. mg | |
| Presentation | Vials containing 18.1 MIU of IFN-α-2b(E41Q) |
| | solubilized in NaCl 7.5 mg/ml; sodium phosphate |
| | dibasic 1.8 mg/ml; sodium phosphate monobasic |
| | 1.3 mg/ml; EDTA disodium 0.1 mg/ml; Tween-80 |
| | 0.1 mg/ml and m-cresol 1.5 mg/ml at a nominal |
| | concentration of 18.1 MIU/ml (100 μg/ml) |
| Number of vials | 10 vials |
| Volume per vial | 1 ml |
| Administration | Intravenous |

TABLE 64b
Native IFN-α Batch # IFN-002/06

| | |
|---|---|
| Correspondence | 388 MIU = 1 mg |
| MIU vs. mg | |
| Presentation | Vials containing 38.8 MIU of native IFN-α |
| | solubilized in NaCl 7.5 mg/ml; sodium phosphate |
| | dibasic 1.8 mg/ml; sodium phosphate monobasic |
| | 1.3 mg/ml; EDTA disodium 0.1 mg/ml; Tween-80 |
| | 0.1 mg/ml and m-cresol 1.5 mg/ml at a nominal |
| | concentration of 38.8 MIU/ml (100 μg/ml) |
| Number of vials | 5 vials |
| Volume per vial | 1 ml |
| Administration | Intravenous |

TABLE 64c
Native IFN-α Batch # IFN-002/06

| | |
|---|---|
| Correspondence | 388 MIU = 1 mg |
| MIU vs. mg | |
| Presentation | Enteric-coated capsules (size 4) containing 2 × 0.3 mg |
| | of native IFN-α lyophilized in NaCl 7.5 mg/ml; |
| | sodium phosphate dibasic 1.8 mg/ml; sodium |
| | phosphate monobasic 1.3 mg/ml; EDTA disodium |
| | 0.1 mg/ml and sucrose 14.7 mg/ml pH 6.8-7. |
| Number of | 4 enteric-coated capsules (size 4) with inside 2 size |
| capsules | 9 enteric-coated capsules |
| Administration | Per-os |

TABLE 64d
IFN-α-2b(E41Q) Batch # IFN-001/06

| | |
|---|---|
| Correspondence | 335 MIU = 1 mg |
| MIU vs. mg | |
| Presentation | Enteric-coated capsules (size 4) containing 2 × |
| | 0.3 mg of IFN-α-2b(E41Q) lyophilized in NaCl |
| | 7.5 mg/ml; sodium phosphate dibasic 1.8 mg/ml; |
| | sodium phosphate monobasic 1.3 mg/ml; EDTA |
| | disodium 0.1 mg/ml and sucrose 14.7 mg/ml pH 6.8-7. |
| Number of | 4 enteric-coated capsules (size 4) with inside 2 size |
| capsules | 9 enteric-coated capsules |
| Administrations | Per-os |

TABLE 64e
IFN-α-2b(E41Q) Batch # G055/060118T

| | |
|---|---|
| Correspondence | 335 MIU = 1 mg |
| MIU vs. mg | |
| Presentation | Enteric-coated tablets containing 0.603 mg of |
| | lyophilized IFN-α-2b(E41Q) and lactose |
| | monohydrate 64.1 mg; microcrystalline cellulose 27 |
| | mg; polyvidone kollidon 25 1.8 mg; crospovidone |
| | polyplasdone XL 3.6 mg; magnesium stearate 1.8 |
| | mg. Enteric-coated tablets are coated with gastric- |
| | resistant coating Eudragit S100, Triethylcitrate, |
| | Talcum micron, Titanium Dioxide and Magnesium |
| | stearate |
| Number of tablets | 40 tablets (total mass tablet 150 mg) |
| Administrations | Per-os |

3. PK Profiles for IV and PO Administration

The objective of this study was to investigate the PK profiles of IFN-α-2b(E41Q) following IV injection or oral administration (as enteric-coated tablets or enteric-coated capsules) to Cynomolgus monkeys. Several doses (0.8, 0.55, 0.45 and 0.2 mg/kg) of an IFN-α-2b(E41Q) enteric-coated tablet formulation, a single dose (0.9 mg/kg) of an IFN-α-2b(E41Q) enteric-coated capsule formulation, and a single dose (0.9 mg/kg) of a native IFN-α enteric-coated capsule formulation were tested by PO administration in Cynomolgus monkeys (Macaca fascicularis) in order to compare the PK and the systemic exposure profiles of the two proteins following oral administration.

As shown in Table 65 below, a total of six purposely bred Cynomolgus monkeys (Macaca fascicularis), three males and three females weighing between 2.8 and 5.4 kg were divided into three groups (one male and one female per group). Each group was used in three separate dosing experiments. Each dosing was separated by a wash-out period of 7-days to allow clearance of the protein from the blood of the animals between dosings. Each dose administered by the IV route was expressed in μg/kg and adjusted to individual body weight on the basis of body weight measured on the day of administration. Each dose administered by the oral route was expressed in mg/kg calculated on the basis of body weight measured on D-6. For the first dosing experiment, the three groups of naïve animals (referred to in Table 65 as Groups 1, 2 and 2 Duplicate), were injected IV with either 50 μg/kg of IFN-α-2b(E41Q) or native IFN-α (day 1) and monitored at time intervals over a period of 16 hours.

For the second dosing experiment, 7 days later, the three groups (the same animals from Groups 1, 2 and 2. Duplicates are referred to in Table 65 as Groups 3, 4 and 5, respectively, for the second dosing experiment), were given a dose of either 0.8 mg/kg of the enteric-coated tablet formulation of IFN-α-2b(E41Q), 0.9 mg/kg of the enteric-coated capsule formulation of IFN-α-2b(E41Q) or native IFN-α by PO route (day 9) and monitored at time intervals over a period of 24 hours.

For the third dosing experiment, 7 days later, the three groups (the same animals from Groups 3, 4 and 5, are referred to in Table 65 as Groups 6, 7 and 8, respectively, for the third dosing experiment), were given a dose of either 0.55, 0.45 or 0.2 mg/kg of the enteric-coated tablet formulation of IFN-α-2b(E41Q) by PO route (day 17) and monitored at time intervals over a period of 24 hours.

TABLE 65
Dosage and experimental detail for IV and PO study

| | | Group/ | | | | | |
|---|---|---|---|---|---|---|---|
| Group | Number | Treatment | | | | | Formulation |
| Number | of animals | Day | Route | Test item | Dose-level | | & Dosage |
| 1 | 1 male | Group 1/D1 | IV | Native IFN-α | 50 | μg/kg | Liquid 0.5 mL/kg |
| | 1 female | | | | | | Liquid 0.5 mL/kg |
| 2 | 1 male | Group 2/D1 | IV | IFN-α-2b(E41Q) | 50 | μg/kg | Liquid 0.5 mL/kg |
| | 1 female | | | | | | Liquid 0.5 mL/kg |
| 2 Duplicat | 1 male | Group 3/D1 | IV | IFN-α-2b(E41Q) | 50 | μg/kg | Liquid 0.5 mL/kg |
| | 1 female | | | | | | Liquid 0.5 mL/kg |
| 3 | 1 male | Group 1/D9 | PO | native IFN-α | 0.9 | mg/kg | 4 enteric-coated |
| | 1 female | | | | 0.9 | mg/kg | capsules/animal |
| | | | | | | | 3 enteric-coated |
| | | | | | | | capsules/animal |
| 4 | 1 male | Group 2/D9 | PO | IFN-α-2b(E41Q) | 0.9 | mg/kg | 3 enteric-coated |
| | 1 female | | | | 0.9 | mg/kg | capsules/animal |
| | | | | | | | 3 enteric-coated |
| | | | | | | | capsules/animal |
| 5 | 1 male | Group 3/D9 | PO | IFN-α-2b(E41Q) | 0.8 | mg/kg | 4 enteric-coated |
| | 1 female | | | | 0.8 | mg/kg | tablets/animal |
| | | | | | | | 4 enteric-coated |
| | | | | | | | tablets/animal |
| 6 | 1 male | Group 1/D17 | PO | IFN-α-2b(E41Q) | 0.55 | mg/kg | 5 enteric-coated |
| | 1 female | | | | 0.55 | mg/kg | tablets/animal |
| | | | | | | | 4 enteric-coated |
| | | | | | | | tablets/animal |
| 7 | 1 male | Group 2/D17 | PO | IFN-α-2b(E41Q) | 0.45 | mg/kg | 3 enteric-coated |
| | 1 female | | | | 0.45 | mg/kg | tablets/animal |
| | | | | | | | 3 enteric-coated |
| | | | | | | | tablets/animal |
| 8 | 1 male | Group 3/D17 | PO | IFN-α-2b(E41Q) | 0.2 | mg/kg | 1 enteric-coated tablet/animal |
| | 1 female | | | | 0.2 | mg/kg | 1 enteric-coated tablet/animal |

IV = intravenous;
PO = oral route;
D = day
Six animals in three groups were used repeatedly

At each time-point (day −1, 0, 0.033, 0.083, 0.17, 0.5, 1, 2, 4, 6, 8 and 16 h post-injection for IV administration and day −1, 0, 0.5, 1, 1.5, 2, 4, 6, 8, 12, 16 and 24 h post-administration for oral administration), a blood sample (1 mL) was taken from the saphenous or cephalic veins of all monkeys for the determination of the remaining antiviral activity levels of IFN-α-2b(E41Q) or native IFN-α in plasma. Blood samples were placed into pre-chilled tubes without anticoagulant and mixed with 70 μl of antiprotease solution. The blood samples were placed on wet ice or 4° C. followed by centrifugation at 4° C. for 10 minutes at 3000 rpm. Serum was divided into 3 propylene tubes (2 tubes with 100 μl and one tube with the rest) and immediately frozen and stored at −20° C.±2° C. until use.

Each animal was checked for mortality and clinical signs at least twice a day during the treatment period. The body weight of each animal was recorded at least twice during the pre-treatment period. PK parameters of liquid formulation of either IFN-α-2b(E41Q) and native IFN-α following IV administration to monkeys (50 μg/kg) are presented in Table 66 below (first dosing experiment).

TABLE 66
PK parameters for IV administration

| | Cᵢ | AUC_{(0-∞)} | Half-life |
|---|---|---|---|
| IV | (IU/mL) | (h-IU/mL) | (h) |
| IFN-α-2b(E41Q)- 50 μg/kg | 421031 | 322229 | 2 |
| Native IFN-α - 50 μg/kg | 375109 | 235200 | 0.5 |

The initial concentration (Ci) value obtained for IFN-α-2b(E41Q) was similar compared to native IFN-α (421031 IU/mL compared to 375109 IU/mL). An increase in AUC_{(0-∞)} of about 1.4-fold was observed for monkeys injected with IFN-α-2b(E41Q) compared to native IFN-α (322229 IU-h/mL for IFN-α-2b(E41Q) versus 235200 IU-h/mL for native IFN-α).

PK parameters of IFN-α-2b(E41Q) and native IFN-α after PO administration of the enteric-coated capsule formulation (0.9 mg/kg) to monkeys are presented in Table 67 below (data from second dosing experiment).

TABLE 67
PK parameters for PO (enteric-coated capsule) administration

| | Cₘₐₓ | Tₘₐₓ | AUC_{(0-∞)} | Half-life |
|---|---|---|---|---|
| Enteric-coated capsules | (IU/mL) | (h) | (h-IU/mL) | (h) |
| Native IFN-α - 0.9 mg/kg | — | — | — | — |
| IFN-α-2b(E41Q) - 0.9 mg/kg | 11795 | 4 | 55803 | 2.7 |

Similar to data obtained with rats after the oral administration of IFN-α-2b(E41Q) enteric-coated capsules (0.9 mg/kg), a significant antiviral activity was detected in monkey plasma with Cₘₐₓ values about 11795 IU/mL and AUC_{(0-∞)} parameter value about 55803 IU-hr/mL, whereas no activity was detected in animals treated with native IFN-α at the same dose (0.9 mg/kg).

PK parameters of IFN-α-2b(E41Q) enteric-coated tablet formulation after PO administration of several doses (0.8, 0.55, 0.45 and 0.2 mg/kg) to monkeys are presented in Table 68 below (data compiled from second and third dosing experiments).

TABLE 68
PK parameters for PO (enteric-coated tablet) administration

| | Cₘₐₓ | Tₘₐₓ | AUC_{(0-∞)} | Half-life |
|---|---|---|---|---|
| Enteric-coated tablets | (IU/mL) | (h) | (h-IU/mL) | (h) |
| Tablet - 0.8 mg/kg | 17165 | 1.5 | 84505 | 3.4 |
| Tablet - 0.55 mg/kg | 20300 | 1 | 100332 | 3.5 |
| Tablet - 0.45 mg/kg | 18686 | 1 | 54752 | 3.4 |
| Tablet - 0.2 mg/kg | 4190 | 1.5 | 10928 | 1.6 |

Significant antiviral activity was detected in monkey plasma after the administration of enteric-coated tablet formulation of IFN-α-2b(E41Q) for all doses. Values of Cₘₐₓ and AUC_{(0-∞)} obtained for IFN-α-2b(E41Q) administration at a dose of 0.8 mg/kg by enteric-coated tablets were higher compared to parameters obtained at a dose of 0.9 mg/kg by enteric-coated capsules (17165 IU/mL compared to 11795 IU/mL, respectively for Cₘₐₓ and 84505 IU-hr/mL compared to 55802 IU-hr/mL, respectively for AUC_{(0-∞)}). For the enteric-coated tablet formulation, both Cₘₐₓ and AUC_{(0-∞)} appeared to be dose-dependent and to saturate at a dose between 0.55 and 0.8 mg/kg. In addition, a dose-related half-life was obtained for IFN-α-2b(E41Q) in the systemic blood compartment with values about 4.3, 3.8, 3.4 and 1.6 h at doses of 0.8, 0.55, 0.45 and 0.2 mg/kg supporting the existence of a sustained delivery of IFN-α-2b(E41Q) from the intestinal compartment to the systemic circulation due to a slow absorption coupled with decreased susceptibility to intestinal protease degradation.

4. Comparison of PK Profiles for PO Administered IFN-α-2b(E41Q) and SC Administered Native IFN-α and Intron A®

PK parameter data previously obtained for SC administration of Intron A® interferon or native IFN-α was compared to the PK data for PO administration of IFN-α-2b(E41Q) (enteric-coated tablets) and is presented in Table 69 below.

TABLE 69
Comparison of PK parameters for PO and SC administration

| | Cₘₐₓ | Tₘₐₓ | AUC(_{0-∞}) |
|---|---|---|---|
| | (IU/mL) | (h) | (h-IU/mL) |
| Tablet - 0.8 mg/kg | 17165 | 1.5 | 84505 |
| Tablet - 0.55 mg/kg | 20300 | 1 | 100332 |
| Tablet - 0.45 mg/kg | 18686 | 1 | 54752 |
| Native IFN-α - SC - 0.03 mg/kg | 23785 | 2 | 86618 |
| Intron A ® - SC - 0.03 mg/kg | 24809 | 1 | 102454 |

This comparison showed a similar AUC_{(0-∞)} after the SC administration of native IFN-α (0.03 mg/kg) and the oral administration of IFN-α-2b(E41Q) (0.55 mg/kg).

This result indicated that the amount of IFN-α-2b(E41Q) needed after oral administration to achieve a similar AUC as measured for the SC administration of native IFN-α was only 15-fold higher in monkeys.

5. Bioavailability Analysis of PO Administration Versus IV Administration

The percentage bioavailability of either IFN-α-2b(E41Q) and native IFN-α following PO administration was calculated by comparison to the PK data for IV administration of IFN-α-2b(E41Q) and native IFN-α presented above in (1). For each dose and formulation of IFN-α-2b(E41Q) administered PO (enteric-coated capsule or tablet formulation) to monkeys, the percentage of IFN-α-2b(E41Q) bioavailability compared to IV administration was calculated relative to Cₘₐₓ and AUC_{(0-∞)}. Results are presented in Tables 70 and 71 below for enteric-coated capsules and enteric-coated tablets, respectively.

TABLE 70
Capsule formulation

| | PO administration | | IV administration | |
|---|---|---|---|---|
| | IFN-α- | native | IFN-α- | Native |
| capsule formulation | 2b(E41Q) | IFNα | 2b(E41Q) | IFNα |
| Dose (mg/kg) | 0.9 | 0.9 | 0.05 | 0.05 |
| AUC_{(0-∞)} (UI-hr/ml) | 55802 | — | 322229 | 235200 |
| Cₘₐₓ (UI/ml) | 11795 | — | 421031 | 375109 |
| | | — | | |
| Cmax/dose | 13106 | — | 8420620 | 7502180 |
| BA | 0.2 | — | | |
| ((Cmax/dose)_{Bel}/ | | — | | |
| (Cmax/dose)_{Bel-IV}) × | | | | |
| 100 | | | | |
| AUC/dose | 62002 | — | 6444580 | 4704000 |
| BA | 1.0 | — | | |
| (AUC/dose)_{Bel}/ | | | | |
| (AUC/dose)_{Bel-IV}) × | | | | |
| 100 | | | | |
| BA relative | 1.3 | — | | |
| (AUC/dose)_{Bel}/ | | | | |
| (AUC/dose)_{natif-IV}) × | | | | |
| 100 | | | | |

TABLE 71
Tablet formulation

| | PO administration | | | | | IV administration | |
|---|---|---|---|---|---|---|---|
| Tablet formulation | IFN-α-2b(E41Q) | | | | native IFNα | IFN-α-2b(E41Q) | Native IFNα |
| Dose (mg) | 0.8 | 0.55 | 0.45 | 0.2 | — | 0.05 | 0.05 |
| AUC_{(0-∞)} (UI-hr/ml) | 84505 | 100332 | 54752 | 10928 | — | 322229 | 235200 |
| Cₘₐₓ (UI/ml) | 17165 | 20300 | 18686 | 4190 | — | 421031 | 375109 |
| | | | | | — | | |
| Cmax/dose | 21456 | 36909 | 41524 | 20950 | — | 8420620 | 7502180 |
| BA | 0.3 | 0.4 | 0.5 | 0.2 | — | | |
| ((Cmax/dose)_{Bel}/(Cmax/dose)_{Bel-IV}) × 100 | | | | | | | |
| | | | | | — | | |
| AUC/dose | 105631 | 182422 | 121671 | 54640 | — | 6444580 | 4704000 |
| BA | 1.6 | 2.8 | 1.9 | 0.8 | — | | |
| (AUC/dose)_{Bel}/(AUC/dose)_{Bel-IV}) × 100 | | | | | | | |
| BA relative | 2.2 | 3.9 | 2.6 | 1.2 | — | | |
| (AUC/dose)_{Bel}/(AUC/dose)_{natif-IV}) × 100 | | | | | | | |

The percentage of bioavailability calculated for IFN-α-2b(E41Q) enteric-coated tablet formulation at a dose of 0.8 mg/kg is higher compared to IFN-α-2b(E41Q) enteric-coated capsule formulation at a dose of 0.9 mg/kg after PO administration to monkeys (1.6% versus 1.0%, respectively).

Calculation of bioavailability of IFN-α-2b(E41Q) enteric-coated tablet formulation after PO administration at several doses (0.8, 0.55, 0.45 and 0.2 mg/kg) showed that doses of 0.55 mg/kg and to a lesser extent of 0.45 mg/kg are optimum strengths (the bioavailability percentage is about 2.6% and 1.9% at doses of 0.55 mg/kg and 0.45 mg/kg, respectively compared to 1.6% and 0.8% at doses of 0.8 mg/kg and 0.2 mg/kg, respectively).

6. Summary

IFN-α-2b(E41Q) can be efficiently delivered to monkey blood circulation by the oral route, as a lyophilized protein (enteric-coated capsules or enteric-coated tablets).

A significant antiviral activity was observed in the systemic blood circulation of monkeys after PO administration of both enteric-coated tablet and enteric-coated capsule formulations of IFN-α-2b(E41Q) whereas no antiviral activity was observed with native IFN-α given PO. The results in monkeys were in accord with previous data obtained in rats after gavage and PO administrations of liquid and enteric-coated capsule formulations of IFN-α-2b(E41Q), respectively. In addition, the PK profile observed after oral delivery mimicked the biphasic profile previously observed in rats indicative of sustained delivery over time, from an “intestinal compartment” into the blood. This also was supported by the dose-related half-life observed for IFN-α-2b(E41Q) in the systemic blood compartment.

The percentage of bioavailability calculated for the IFN-α-2b(E41Q) enteric-coated tablet formulation at the highest dose was increased about 60% compared to the one seen for the enteric-coated capsule formulation at a similar dose. Enteric-coated tablet formulation administration of IFN-α-2b(E41Q) at several doses was dose-dependent between 0.2 mg/kg to 0.55 mg/kg and saturated at doses between 0.55 mg/kg and 0.8 mg/kg. The bioavailability percentage was about 2.6% (relative to IV IFN-α-2b(E41Q) AUC_{(0-∞)}) and 3.2% (relative to native IFN-α AUC_{(0-∞)}) with an optimum strength of about 0.55 mg/kg of IFN-α-2b(E41Q).

### Example 12

### Pharmacokinetics and Immunogenicity of IFN-α-2b(E41Q) Following Oral Administration in Cynomolgus Monkeys

The purpose of this study was to assess the pharmacokinetics, immunogenicity and influence of anti-IFN-α-2b(E41Q) antibodies on PK profile of IFN-α-2b(E41Q) when administered daily by PO route to Cynomolgus (Macaca fascicularis) monkeys via enteric-coated tablets for 2 weeks. The Macaca fascicularis monkey was chosen because of its acceptance as a predictor of pharmacological effects for this class of drugs in humans.

1. General Procedures

Repeated PO administrations of IFN-α-2b(E41Q) enteric-coated tablet formulations with doses of 0.6, 1.2 and 2.4 mg per animal were performed daily over 2 weeks in eighteen Cynomolgus monkeys (Macaca fascicularis), nine males and nine females, bred in captivity. The females were nulliparous and nonpregnant. At the start of the treatment, the animals were 2 to 3 years old, and their weight was between 2 and 3 kg. Blood samples were taken from all the animals for the determination of plasma levels of IFN-α-2b(E41Q) (pharmacokinetic studies) and for the measurement of both total and neutralizing anti-IFN-α antibody levels (antibody determination assays) in monkey serum.

The following treatment groups were established according to Table 72 below:

TABLE 72
Treatment groups for PO (enteric-coated tablet) administration

| | | Dose level | | | |
|---|---|---|---|---|---|
| | | | Number of | | |
| | | | IFN-α-2b(E41Q) | | |
| | | | enteric-coated | | |
| | | | tablet (B) and | | |
| | | mg | enteric-coated | No. animals | |
| | | protein/ | placebo | | Fe- |
| Group | Treatment | animal | tablet (P) | Males | males |
| 1 | Control | — | 4 P | 3 | 3 |
| 2 | IFN-α-2b(E41Q) | 0.6 | 1B + 3P | 3 | 3 |
| 3 | IFN-α-2b(E41Q) | 1.2 | 2B + 2 P | 3 | 3 |
| 4 | IFN-α-2b(E41Q) | 2.4 | 4B | 3 | 3 |

Repeated PO administration of IFN-α-2b(E41Q) enteric-coated tablet formulation with doses of 0.6 mg, 1.2 mg and 2.4 mg per animal were performed daily over 2 weeks. Since the weight of male and female monkeys included in this study is about 3 and 2 kg, respectively, the dose of 0.6 mg/animal corresponded to 0.3 mg/kg and 0.2 mg/kg for females and males respectively; the dose of 1.2 mg/animal corresponded to 0.6 mg/kg and 0.4 mg/kg for females and males respectively; and the dose of 2.4 mg/animal corresponded to 1.2 mg/kg and 0.8 mg/kg for females and males respectively. For each dose level, three males and three females were used. The control group received enteric-coated placebo tablets only.

Blood samples (2 mL) were taken from all the animals at pre-dose and 24 h after administration at days 1, 6, and 13. The samples were stored in ice baths/cryoracks followed by centrifugation at 1620 g for 15 minutes at 4° C. in order to obtain serum. Samples were transferred to two fresh polypropylene vials (approx. 300 μl) and stored at −70° C. until use. The plasma samples were used for i) the determination of plasma levels of IFN-α-2b(E41Q) and ii) the measurement of both total and neutralizing anti-IFN-α-2b(E41Q) antibodies in monkey serum. For each tested dose, both total and neutralizing anti-IFN-α-2b(E41Q) antibodies were determined using the human anti-IFN-α antibody ELISA (for measuring total antibodies) according to the manufacturer's instructions (A4-203—MDS Pharma Services, Zurich, Switzerland) and Kawade's assay (for measuring neutralizing antibodies; Kawade et al., J. Interferon Research, 1987, Grossberg et al., J. Interferon Cytokines Research; 2001; Grossberg et al., Biotherapy; 1997; Kawade Y., J. Interferon Research, 1980; 1:61-70; Kawade et al., J. Immunological Methods, 2003).

2. Drug Batches

For the pharmacokinetic and immunogenicity studies presented in this Example, one IFN-α-2b(E41Q) batch was used: G055/060118T (described in Example 4). As a control, enteric-coated placebo tablets were used. Specifications and routes of administration for the experimental and placebo batches are presented in Tables 73(a and b) below.

TABLE 73a
IFN-α-2b(E41Q) Batch # G055/060118T

| | |
|---|---|
| Correspondence | 335 MIU = 1 mg |
| MIU vs. mg | |
| Presentation | Enteric-coated tablets containing 0.603 mg of |
| | lyophilized IFN-α-2b(E41Q) and lactose |
| | monohydrate 64.1 mg; microcrystalline cellulose 27 |
| | mg; polyvidone kollidon 25 1.8 mg; crospovidone |
| | polyplasdone XL 3.6 mg; magnesium stearate 1.8 |
| | mg. Enteric-coated tablets are coated with gastric- |
| | resistant coating Eudragit S100; triethyl citrate, |
| | Talcum micron, Titanium Dioxide and Magnesium |
| | stearate. |
| Number of tablets | 774 tablets (total mass tablet 150 mg) |
| Administrations | Per-os |

TABLE 73b
Placebo Batch # P-2154/060711T

| | |
|---|---|
| Correspondence | None |
| MIU vs. mg | |
| Presentation | Enteric-coated tablets containing lactose |
| | monohydrate 64.1 mg; microcrystalline cellulose 27 |
| | mg; polyvidone kollidon 25 1.8 mg; crospovidone |
| | polyplasdone XL 3.6 mg; magnesium stearate 1.8 |
| | mg. Enteric-coated tablets are coated with gastric- |
| | resistant coating Eudragit S100; triethyl citrate, |
| | Talcum micron, Titanium Dioxide and magnesium |
| | stearate. |
| Number of tablets | 202 tablets (total mass tablet 150 mg) |
| Administrations | Per-os |

3. Neutralizing and Total Anti-IFN-α-2b(E41Q) Antibodies

Using the human anti-IFN-α antibody ELISA and Kawade's assay for measuring total and neutralizing antibodies, respectively (both assays are described in Example 10), no anti-IFN-α-2b(E41Q) antibodies (total or neutralizing antibodies) were detected in the pre-dose serum or in the samples taken at days 2, 7 and 14 after daily administration of IFN-α-2b(E41Q) enteric-coated tablets. In each case, the antibody concentrations (for total antibodies) or Kawade titers (for neutralizing antibodies) measured in all monkey serum were below the background levels measured in the pre-dose samples.

A previous serum sample taken at day D26 from male 12 monkey administered subcutaneously twice a week with IFN-α-2b(E41Q) at dose of 1.26 MIU/kg was used as positive control in total antibody titer determination assay (ELISA from MDS Pharma Services). The value obtained for this sample was 12.6±1.7 IU/ml. The corresponding value obtained with Bender Medsystems Immunoassay Kit was 4299 ng/ml. The positive titer determined for male 12 monkey validated the data showing no total antibody detected in monkey serum from the two-week oral (enteric-coated tablets) toxicity study.

4. PK Profile for Day 1 Administration

Plasma samples were taken at intervals for 24 h following the day 1 dose with enteric-coated placebo tablets and IFN-α-2b(E41Q) treated animals for the determination of IFN-α-2b(E41Q) PK. The amount of remaining IFN-α-2b(E41Q) in plasma of monkeys given enteric-coated placebo tablets or 0.6, 1.2 and 2.4 mg IFN-α-2b(E41Q) enteric-coated tablets per animal, was determined by the antiviral activity assay as described in Example 1 above.

PK parameters of IFN-α-2b(E41Q) enteric-coated tablet formulation after PO administration to monkeys at day 1 with doses of 0.6, 1.2 and 2.4 mg per animal are provided in Table 74 below. Means of both male and female monkey PK parameters for each dose of IFN-α-2b(E41Q) are shown.

TABLE 74
PK parameters for PO (enteric-coated tablet) administration Day 1

| | Cₘₐₓ | Tₘₐₓ | AUC_{(0-∞)} | AUC₍₀₋ₜ₎ |
|---|---|---|---|---|
| Enteric-coated tablets | (IU/mL) | (h) | (h-IU/mL) | (h-IU/mL) |
| IFN-α-2b(E41Q) - 0.6 mg | 1473 | 1 | 7767 | 7767 |
| IFN-α-2b(E41Q) - 1.2 mg | 1876 | 1 | 12948 | 12662 |
| IFN-α-2b(E41Q) - 2.4 mg | 5496 | 1 | 32923 | 32918 |

Similar to previous data obtained from the pharmacokinetic study (Example 11) performed in Cynomolgus monkeys after PO administration of either enteric-coated capsule or enteric-coated tablet formulations of IFN-α-2b(E41Q), a significant antiviral activity was detected in all monkey plasma (both male and female) samples for each dose.

A good dose-dependent correlation was observed between the 0.6 mg and 2.4 mg tested doses of IFN-α-2b(E41Q) administered to monkeys and Cₘₐₓ measured from the respective PK profiles. For monkeys injected with 0.6 mg, the Cₘₐₓ mean value was 1473 IU/mL compared to 5496 IU/mL for the 2.4 mg dose. Similarly, the AUC₍₀₋ₜ₎ mean value for monkeys administered with 0.6 mg was about 7767 IU-hr/mL compared to 32918 IU-hr/mL for the 2.4 mg dose. A dose-dependent relationship also was observed with the intermediate dose (1.2 mg per animal) for the AUC₍₀₋ₜ₎ parameter (12662 IU-hr/mL versus 7767 IU-hr/mL with a dose of 0.6 mg) and a lesser correlation was observed for Cₘₐₓ (1876 IU/mL versus 1473, for a dose of 0.6 mg per animal).

5. PK Profile for Day 14 Administration

Plasma samples were taken at intervals for 24 h following the day 14 dose with enteric-coated placebo tablets and IFN-α-2b(E41Q) treated animals for the determination of the IFN-α-2b(E41Q) PK profile. The amount of remaining IFN-α-2b(E41Q) in plasma of monkeys given enteric-coated placebo tablets or 0.6, 1.2 and 2.4 mg IFN-α-2b(E41Q) enteric-coated tablets per animal, was determined by the antiviral activity assay as described in Example 1.

PK parameters of IFN-α-2b(E41Q) enteric-coated tablet formulation after PO administration to monkeys at day 14 with doses of 0.6, 1.2 and 2.4 mg per animal are provided in Table 75 below. Means of both male and female monkey PK parameters for each dose of IFN-α-2b(E41Q) are shown.

TABLE 75
PK parameters for PO (enteric-coated tablet) administration Day 14

| | Cₘₐₓ | Tₘₐₓ | AUC_{(0-∞)} | AUC₍₀₋ₜ₎ |
|---|---|---|---|---|
| Enteric-coated tablets | (U/mL) | (h) | (h-IU/mL) | (h-IU/mL) |
| IFN-α-2b(E41Q) - 0.6 mg | 1977 | 1 | 11021 | 10975 |
| IFN-α-2b(E41Q) - 1.2 mg | 3488 | 1 | 27021 | 26981 |
| IFN-α-2b(E41Q) - 2.4 mg | 5360 | 2 | 40834 | 40768 |

Similar to the PK profiles obtained with IFN-α-2b(E41Q) enteric-coated tablet administration at day 1, a good correlation was observed between all tested doses of IFN-α-2b(E41Q) (0.6, 1.2 and 2.4 mg) administered to monkeys and the Cₘₐₓ measured from the respective PK profiles. For monkeys injected with 0.6 mg, the Cₘₐₓ mean value was 1977 IU/mL compared to 3488 IU/mL with a dose of 1.2 mg and 5360 IU/mL with a dose of 2.4 mg. Similarly, the AUC₍₀₋ₜ₎ mean value for monkeys administered with 0.6 mg is about 10975 IU-hr/mL compared to 26981 IU-hr/mL with a dose of 1.2 mg and 40768 IU-hr/mL with a dose of 2.4 mg.

In addition, no impairment of PK profiles obtained for each dose of IFN-α-2b(E41Q) enteric-coated tablets (0.6, 1.2 and 2.4 mg per animal) was observed at day 14 compared to day 1. This observation was supported by the AUC₍₀₋ₜ₎ and Cₘₐₓ mean values measured from all PK profiles which were very closed for all IFN-α-2b(E41Q) doses tested from day 1 to day 14 (1473 IU/mL versus 1977 IU/mL for the Cₘₐₓ at day 1 and 14 respectively with a dose of 0.6 mg; 5496 IU/mL versus 5360 IU/mL for the Cₘₐₓ at day 1 and 14 respectively with a dose of 2.4 mg; 7767 IU-hr/mL versus 10975 IU/mL for the AUC₍₀₋ₜ₎ at day 1 and 14 respectively with a dose of 0.6 mg and 32918 IU-hr/mL versus 40768 IU/mL for the AUC₍₀₋ₜ₎ at days 1 and 14, respectively with a dose of 2.4 mg).

Furthermore, all these PK observations strongly supported the lack of total and neutralizing antibodies evidenced in monkey serum at day 14 after daily PO administration of the IFN-α-2b(E41Q) enteric-coated tablet formulation at doses of 0.6, 1.2 and 2.4 mg per animal over 2 weeks.

6. Summary

No neutralizing or total antibody levels to IFN-α-2b(E41Q) were detected with doses of 0.6, 1.2 and 2.4 mg per animal in monkey serum after daily PO administration of IFN-α-2b(E41Q) enteric-coated tablets over 2 weeks. Similar to previous data obtained from the pharmacokinetic study (Example 11) performed in Cynomolgus monkeys after PO administration of either enteric-coated capsule or enteric-coated tablet formulations of IFN-α-2b(E41Q), a significant antiviral activity was detected in all monkey plasma (both male and female) samples for each tested dose. No impairment of the pharmacokinetic profile of IFN-α-2b(E41Q) was observed at day 14 with doses of 0.6, 1.2 and 2.4 mg after daily administration by the oral route of IFN-α-2b(E41Q) enteric-coated tablets over 2 weeks, supporting the lack of detection of antibodies in monkey serum at day 14.

### Example 13

### Pharmacokinetic Study Subcutaneous (SC) Administration of IFN-α-2b(E41Q) in Mice

The PK profile of subcutaneously administered IFN-α-2b(E41Q) in mice compared to native IFN-α and Pegasys® interferon, a commercial IFN-α-2a protein, which is modified by pegylation, was determined. A single dose pharmacokinetic profile of SC administered IFN-α-2b(E41Q) (0.4 MIU/mouse; 1.3 μg/mouse or 65 μg/kg) was compared in vivo to native IFN-α (0.4 MIU/mouse dose) and Pegasys® interferon (18 μg/mouse, 720 μg/kg or 36 μg/mouse, 1440 μg/kg). 12 male mice were used for each subcutaneous administration. All mice received injections on the same day. Blood (200 μl) was collected at 0.5, 1, 2, 4, 6, 8, 10, 12, 16, 24, 36 and 48 hours after injection. PK profiles were determined as described in Example 9. PK parameters for IFN-α-2b(E41Q), native IFN-α, and Pegasys® interferon are presented in Table 76 below.

TABLE 76
PK parameters for SC administration

| | | | | Half-life (t_{1/2}) |
|---|---|---|---|---|
| | Cₘₐₓ | Tₘₐₓ | AUC_{(0-∞)} | Elimination |
| Molecule | (U/mL) | (h) | (h-IU/mL) | time (h) |
| IFN-α-2b(E41Q) | 60.2 | 1 | 3487 | 56.2 |
| Native IFN-α | 88.7 | 1 | 536 | 1.6 |
| Pegasys ® (18 μg/mouse) | 38.0 | 12 | 1312 | 39.0 |
| Pegasys ® (36 μg/mouse) | 65.7 | 12 | 2870 | 35.1 |

The PK profile in mice receiving SC IFN-α-2b(E41Q) compared to native IFN-α showed a higher AUC_{(0-∞)} and t_{1/2}. For AUC_{(0-∞)}, a 6.5-fold increase was observed, 3,487 for IFN-α-2b(E41Q) versus 536 for native IFN-α. For half-life, a 35-fold increase was observed, 56 h for IFN-α-2b(E41Q) versus 2 h for native IFN-α. The PK parameters obtained from SC administration of IFN-α-2b(E41Q) compared to Pegasys® interferon showed a slightly higher AUC_{(0-∞)} (a 1.45-fold increase of 3,487 for IFN-α-2b(E41Q) versus 2,870 for Pegasys® interferon and a longer half-life t_{1/2} (a 1.3-fold increase of 56 h for IFN-α-2b(E41Q) versus 39 h for Pegasys® interferon.

### Example 14

### Pharmacokinetic and Pharmacodynamic Toxicity Study of Multi-Dose SC Administration of IFN-α-2b(E41Q) in Non-Human Primates

The PK profile and pharmacodynamic effects of IFN-α-2b(E41Q) following subcutaneous administration was determined for separate administrations of a multi-dose regimen.

1. Comparison of IFN-α-2b(E41Q), Native IFN-α, and Intron A®

The PK profile of subcutaneously administered IFN-α-2b(E41Q) in Cynomolgus monkeys (Macaca fascicularis) compared to native IFN-α and the commercial recombinant IFN-α-2b protein Intron A® interferon was determined. The study was designed to investigate the pharmacokinetic, pharmacodynamic, preliminary clinical safety/pathology and immunogenicity, on dose-dependent effects in monkeys. For each group, IFN-α-2b(E41Q), native IFN-α or Intron A® interferon, 1 male and 1 female monkey was used. The following dosing regimen for subcutaneous administration of IFN-α-2b(E41Q), native IFN-α and Intron A® interferon, shown in Table 77 below were employed.

TABLE 77
Dosing regimen for SC administration (IFN-α-
2b(E41Q); native IFN-α; Intron A ®)

| Day | IFN-α-2b(E41Q) | | Native IFN-α | | Intron A ® | |
|---|---|---|---|---|---|---|
| 1 | 0.03 | mg/kg | 0.03 | mg/kg | buffer | |
| | (9 | MIU/kg) | (9 | MIU/kg) | | |
| 4 | 0.30 | mg/kg | 0.30 | mg/kg | buffer | |
| | (90 | MIU/kg) | (90 | MIU/kg) | | |
| 8 | 1.00 | mg/kg | 1.00 | mg/kg | 0.03 | mg/kg |
| | (300 | MIU/kg) | (300 | MIU/kg) | (9 | MIU/kg) |
| 12 | 3.00 | mg/kg | 3.00 | mg/kg | 0.30 | mg/kg |
| | (900 | MIU/kg) | (900 | MIU/kg) | (90 | MIU/kg) |

Washout Period

| | | | | | | |
|---|---|---|---|---|---|---|
| 66 | 0.03 | mg/kg | 0.03 | mg/kg | 0.03 | mg/kg |
| | (9 | MIU/kg) | (9 | MIU/kg) | (9 | MIU/kg) |
| 72 | 0.03 | mg/kg | 0.03 | mg/kg | 0.03 | mg/kg |
| | (9 | MIU/kg) | (9 | MIU/kg) | (9 | MIU/kg) |
| 78 | 0.30 | mg/kg | 0.30 | mg/kg | 0.30 | mg/kg |
| | (90 | MIU/kg) | (90 | MIU/kg) | (90 | MIU/kg) |

The following assessments were performed: (i) determination of serum concentration from in vitro antiviral assay (IFN-α-2b(E41Q) and native IFN-α at days 1, 4 over 72 h; at days 66, 72, 78 over 120 h; Intron A® interferon at days 8, 12 over 72 h; at days 66, 72, 78 over 120 h); (ii) RT-PCR assay for mRNA expression of IFN-α inducible genes which are markers of antiviral activity, such as 2′5′ OAS, neopterin, and MxA, (IFN-α-2b(E41Q) and native IFN-α at days 1, 4 over 72 h; at days 66, 72, 78 over 24 h (neopterin over 120 h); Intron A® at days 8, 12 over 72 h; at days 66, 72, 78 over 24 h (neopterin over 120 h)); (iii) urinary excretion: pre-dose, days 3, 10; (iv) measurement of total and neutralizing antibodies (pre-dose and at days 16, 21 and 28); (v) assays for various safety and lab parameters such as clinical signs, mortality, body weight, and post-mortem exam. PK parameters calculated from the in vitro antiviral assay results are presented in Table 78 below.

TABLE 78
PK parameters for SC administration

| | Cₘₐₓ | Tₘₐₓ | AUC₍₀₋₇₂₎ | AUC_{(0-∞)} | Half-life |
|---|---|---|---|---|---|
| Molecule | (μg/mL) | (h) | (h-IU/mL) | (h-IU/mL) | (t_{1/2}) (h) |
| IFN-α-2b(E41Q) | 25382 | 1-4 | 1023638 | 1579965 | 44.9 |
| Native IFN-α | 23785 | 2 | 86618 | 86916 | 2.02 |
| Intron A ® | 24809 | 1 | 102454 | 102692 | 2.28 |

From these studies, it was observed that the AUC_{(0-∞)} for SC administration of IFN-α-2b(E41Q) was 15 times larger than Intron A® interferon and 18 times larger than native IFN-α. The half-life t_{1/2} for SC administration of IFN-α-2b(E41Q) was 22 times longer than native IFN-α, and 20 times longer than Intron A®. IFN-α-2b(E41Q) induced markers of antiviral activity similar to Intron A® and native IFN-α. Urinary excretion was not remarkable and no relevant signs of clinical pathology or toxicity were evidenced, though all compounds induced antibody production.

2. Comparison of IFN-α-2b(E41Q) and Pegasys® Interferon

In a separate experiment, the PK profile of subcutaneously administered IFN-α-2b(E41Q) in Cynomolgus monkeys (Macaca fascicularis) was compared to the pegylated IFN-α-2a product Pegasys® interferon. For each group, IFN-α-2b(E41Q) or Pegasys®, 1 male and 1 female monkey was used. The following dosing regimen for subcutaneous administration of IFN-α-2b(E41Q) or Pegasys® interferon shown in Table 79 below was employed.

TABLE 79
Dosing regimen for SC
administration (IFN-α-2b(E41Q); Pegasys ®)

| | IFN-α- | Pegasys ® |
|---|---|---|
| Day | 2b(E41Q) | interferon |
| 1 | 0.03 mg/kg | buffer |
| | (9 MIU/kg) | |
| 7 | 0.03 mg/kg | 0.30 mg/kg |
| | (9 MIU/kg) | |
| 13 | 0.30 mg/kg | 0.30 mg/kg |
| | (90 MIU/kg) | |

The following assessments were performed: (i) determination of serum concentration from in vitro antiviral assay (IFN-α-2b(E41Q) and Pegasys® interferon at days 1, 7, 13 over 120 h); (ii) RT-PCR assay for mRNA expression of IFN-α inducible genes which are markers of antiviral activity, such as 2′5′ OAS, neopterin, and MxA (IFN-α-2b(E41Q) and Pegasys® interferon at days 1, 7, 13 over 24 h (neopterin over 120 h)); (iii) urinary excretion (pre-dose, days 3, 10); (iv) measurement of total and neutralizing antibodies (pre-dose, days 21, 28, 35); (v) assays for various safety and lab parameters such as clinical signs, mortality, body weight, and post-mortem exam. PK parameters calculated from the in vitro antiviral assay results are presented in Table 80 below.

TABLE 80
PK parameters for SC administration

| | Cₘₐₓ | Tₘₐₓ | AUC₍₀₋₁₂₀₎ | AUC_{(0-∞)} | Half-life |
|---|---|---|---|---|---|
| Molecule | (μg/mL) | (h) | (h-IU/mL) | (h-IU/mL) | (t_{1/2}) (h) |
| IFN-α-2b(E41Q) | 83700 | 2-4 | 4304238 | 6382257 | 44.7 |
| Pegasys ® | 47351 | 48 | 2256184 | 2279809 | 33.9 |

From these studies, it was observed that IFN-α-2b(E41Q) had an AUC_{(0-∞)} 3 times larger than Pegasys® interferon, and a similar half-life t_{1/2}. IFN-α-2b(E41Q) (0.03 mg/kg) and Pegasys® interferon (0.30 mg/kg) displayed similar plasma profiles for markers of antiviral activity. Urinary excretion was not remarkable and no relevant signs of clinical pathology or toxicity were evidenced, though both compounds induced antibody production.

### Example 15

### Toxicity Study of SC Administered IFN-α-2b(E41Q) in Cynomolgus Monkeys

The toxicokinetic profile of IFN-α-2b(E41Q) was evaluated as part of a 13-week, repeated dose toxicity study in Cynomolgus monkeys treated subcutaneously twice a week at doses of 0.14 MIU/kg (0.47 μg/kg), 0.42 MIU/kg (1.40 μg/kg) and 1.26 MIU/kg (4.20 μg/kg). For each group 3 male and 3 female monkey were used. Sample of peripheral blood for determination of IFN-α-2b(E41Q) levels were obtained from all monkeys at pre-dose, 0.5, 1, 2, 4, 8, and 24 after dosing on day 1. Peripheral blood samples for cumulative exposure also were obtained at these same time points on days 26 and 89. The level of IFN-α-2b(E41Q) in the blood was determined using an in vitro antiviral assay (see Example 9). PK parameters for IFN-α-2b(E41Q) calculated from the in vitro antiviral assay results for days 1, 26 and 89 are presented in Tables 81(a-c), respectively.

TABLE 81a
PK parameters for SC administration (day 1)

| | 0.14 MIU/kg* | | 0.42 MIU/kg | | 1.26 MIU/kg | |
|---|---|---|---|---|---|---|
| Day 1 | Females | Males | Females | Males | Females | Males |
| Cₘₐₓ (IU/ml) | 3164 | 3699 | 5918 | 6718 | 30549 | 30272 |
| AUC₍₀₋ₜ₎ (IU- | 55412 | 63067 | 132774 | 149281 | 571157 | 658053 |
| hr/ml) | | | | | | |

TABLE 81b
PK parameters for SC administration (day 26)

| | |
|---|---|
| 0.42 MIU/kg | 1.26 MIU/kg |

| Day 26 | Females | Males | Females | Males |
|---|---|---|---|---|
| Cₘₐₓ (IU/ml) | 1564 | 1121 | 3428 | 3911 |
| AUC₍₀₋ₜ₎ (IU-hr/ml) | 29267 | 24572 | 76290 | 78016 |

TABLE 81c
PK parameters for SC administration (day 89)

| | |
|---|---|
| 0.42 MIU/kg | 1.26 MIU/kg |

| Day 89 | Female | Male | Females | Males |
|---|---|---|---|---|
| Cₘₐₓ (IU/ml) | 23256 | 13546 | 14328 | 22781 |
| AUC₍₀₋ₜ₎ (IU-hr/ml) | 312930 | 226514 | 310767 | 448896 |

*No PK parameter values were calculated for the 0.14 MIU/kg dose at days 26 and 89 since IFN-α-2b(E41Q) serum levels were below the detection range limits of the antiviral assay.

As a result of this study, it was observed that SC injection of IFN-α-2b(E41Q) at doses of 0.14, 0.42 and 1.26 MIU/kg was associated with dose-related increases in Cₘₐₓ and AUC after the first administration. After 4 and 13 weeks of treatment, the detection of IFN serum levels was impeded by the development of neutralizing antibody titers in some animals.

### Example 16

### Toxicology Studies

### A. Rats

1. Acute Toxicity Study in the Rat by the Oral Route

The acute toxicity of IFN-α-2b(E41Q) was evaluated in rats following a single oral route administration by gavage cannula of 2.4 mg/rat (9 mg/kg) of protein by administration of three enteric-coated capsules (0.8 mg each; size 9 enteric-coated capsules). Six female rats (7-9 week SPF Sprague-Dawley rats, each weighing 255.2 g to 274.3 g), with free access to food and water, were used for the experiment. Following administration with IFN-α-2b(E41Q) for 14 days, mortality, clinical signs, functional and neurobehavioral tests, body temperature and body weight were monitored. At the end of the observation period, all animals were sacrificed and post-mortem examinations were performed.

No animals dosed with enteric-coated capsules of IFN-α-2b(E41Q) administered at 2.4 mg/rat (9 mg/kg) died during the observation period. No effect on body temperature was seen in females and, for the majority of animals, no effect on body weight gain was observed. Four out of six females showed decrease in grip strength and five out of six females showed decrease in body tone and abdominal tone 24 h post-dose. No macroscopic findings were recorded at necropsy.

In summary, IFN-α-2b(E41Q) administered once by the oral route at a dose of 2.4 mg/rat (9 mg/kg) in the Sprague-Dawley females did not induce mortality but induced a decrease in muscle tone 24 h post-dose. The median lethal dose (LD₅₀) of IFN-α-2b(E41Q) after oral administration to female rats observed over a period of 14 days was greater than 9 mg/kg of body weight.

2. 2-Week Oral Toxicity Study in Rats by the Oral Route

The study involved assessment of four main groups of rats and four satellite groups of rats following administration with various doses of IFN-α-2b(E41Q) by oral route. The main groups were used to assess toxicity based on mortality and clinical signs. The satellite groups were used for toxicokinetic assessment (see results in Example 10) and antibody analysis (see results in Example 20.B).

A total of forty-eight (SPF) Sprague-Dawley rats at 8-weeks old (twenty-four males and twenty-four females, divided equally among the groups (six animals/sex/group); weighing 250-350 g) were used in the study. The four treatment groups (for each of the main and satellite groups, for a total of eight groups) are set forth in Table 82 below:

TABLE 82
Toxicology Treatment Groups

| | | | | Satellite | |
|---|---|---|---|---|---|
| | | Main (No. of | | (No. of | |
| | | mice/group) | | mice/group) | |
| Group (IFN-α-2b(E41Q) dose) | | Male | Female | Male | female |
| 1. | vehicle alone (one size 9 | 6 | 6 | 6 | 6 |
| | enteric-coated placebo capsule) | | | | |
| 2. | 0.2 mg/rat (1 mg/kg for | 6 | 6 | 6 | 6 |
| | females and 0.6 mg/kg for | | | | |
| | males) | | | | |
| 3. | 0.4 mg/rat (2 mg/kg for | 6 | 6 | 6 | 6 |
| | females and 1.2 mg/kg for | | | | |
| | males) | | | | |
| 4. | 0.6 mg/rat (3 mg/kg for | 6 | 6 | 6 | 6 |
| | females and 1.8 mg/kg for | | | | |
| | males) | | | | |

Animals received oral administration of IFN-α-2b(E41Q) enteric-coated capsules (one size 9 enteric-coated capsule) daily for 2 weeks at the dose indicated above for the respective group, for a total of fourteen administrations. Animals from the main groups were observed twice a day for mortality and clinical signs. They were weighed at days −1, 3, 7, 10 and 14 and before sacrifice. Body temperature was recorded before administrations at days 1, 7, 14 and also 1 h after treatment. Opthalmoscopic examinations and blood analyses were performed at pre-dose and at days 7 and 14 of the administration period. Hematology, clinical biochemistry and urinalysis examinations were conducted prior to the first dose and at days 8 and 15. At the end of the treatment period, a full necropsy and a histopathologic examination were conducted on all animals of the main groups.

The rats from the satellite groups were separately assessed for toxicokinetic and immunogenicity studies. Blood samples for the determination of the plasma levels of IFN-α-2b(E41Q) (toxicokinetics) were taken at days corresponding to administrations days 1, 7 and 14 at pre-dose, 1, 2, 4, 8 and 24 h. Methods and results are reported separately in Example 10. Blood samples for immunogenicity studies (assessment of total and neutralizing antibodies) were taken at pre-dose and at day 15 after administration. Methods and results are reported in Example 20.B.

a. Toxicity Results in Main Group

i. Unscheduled Deaths

No mortality or unscheduled deaths were observed in the rats upon treatment with any dose of IFN-α-2b(E41Q) tested or vehicle alone. One male dosed with 0.6 mg/rat IFN-α-2b(E41Q) presented with a difficulty in respiration and was euthanized for ethical reasons and necropsied at day 10. The animal had lesions in the esophagus and around the trachea likely as a result of the dosing.

ii. Clinical Signs

A decrease in body tone was observed in two males and two females dosed at 0.2 mg/rat with IFN-α-2b(E41Q), one male and two females dosed at 0.4 mg/rat with IFN-α-2b(E41Q) and three males dosed at 0.6 mg/rat with IFN-α-2b(E41Q). In addition, one male and one female dosed at 0.2 mg/rat with IFN-α-2b(E41Q), one female dosed at 0.4 mg/rat with IFN-α-2b(E41Q) and three males dosed at 0.6 mg/rat with IFN-α-2b(E41Q) showed a decrease in abdominal tone. Similar observations were made with the vehicle alone placebo for one male (in body tone) and some females (in abdominal tone and body tone).

Finally, a decrease in limb tone in one male of each group dosed with IFN-α-2b(E41Q) and in grip strength for two females dosed at 0.2 mg/rat with IFN-α-2b(E41Q) and for one female dosed at 0.4 mg/rat also with IFN-α-2b(E41Q) was observed.

At necropsy, one mass observed near the trachea was observed in one male dosed with the placebo (vehicle only) and near to the esophagus in one female dosed with IFN-α-2b(E41Q) at 0.4 mg/rat. The male dosed with IFN-α-2b(E41Q) at 0.6 mg/rat and euthanized at D10 showed adhesion between trachea and esophagus and between thymus and heart.

iii. Body Weight

No particular effect was observed in males and females treated with both the vehicle and IFN-α-2b(E41Q). A statistically significant decrease in absolute and relative weight of pituitary gland was observed in males with IFN-α-2b(E41Q) at 0.6 mg/rat (−20% and −18% respectively). In females dosed with IFN-α-2b(E41Q) at 0.2 mg/rat, statistically significant increase in relative weight of liver was observed (+11%).

iv. Body Temperature

No particular effect was observed in males and females treated with both the vehicle and IFN-α-2b(E41Q) at all doses tested.

v. Opthalmoscopic Examinations

No particular abnormality was noticed in males and females treated with both the vehicle and IFN-α-2b(E41Q).

vi. Blood Analysis

No particular change related to the treatment was observed in males and females dosed with IFN-α-2b(E41Q) at any dose tested. A statistically significant increase in eosinophils (+144%) was observed at day 8 in males dosed with IFN-α-2b(E41Q) at 0.4 mg/rat.

vii. Urinary Parameters

All the parameters were within the range of values usually found in this species for all animals treated with both IFN-α-2b(E41Q) and the vehicle.

vii. Histopathology

There were no treatment-related lesions in rats treated with IFN-α-2b(E41Q) at 0.6 mg/rat. Histopathology changes were observed in all treatment groups and included a) edematous inflammation, mural chronic inflammation, hemorrhage in the esophagus, b) edematous inflammation of trachea and c) abscess containing ingesta. These changes were not related to the dose of IFN-α-2b(E41Q) but rather the trauma associated with the mechanical dosing. The greatest esophagus changes were present in the female control group.

b. Conclusions

No mortalities and no relevant abnormal findings were observed under these experimental conditions with IFN-α-2b(E41Q) at any of the administered doses. The NOAEL (No Observed Adverse Effect Level) of IFN-α-2b(E41Q) was 0.6 mg/rat when administered by the oral route in male and female rats.

3. Single and Repeated Dose Toxicity in Rats by the Intraduodenal (ID) Route

The toxicity of IFN-α-2b(E41Q) was evaluated in rats after both single and repeated oral administrations. For each group (Groups 1-4), six male rats at 8-weeks (SPF Sprague-Dawley rats, weighing 215.2 g to 263.0 g), with free access to food and water, were used for the experiment. The rats were catheterized into the duodenal lumen 18-24 h before administration of IFN-α-2b(E41Q) or vehicle. The animals were treated under anesthesia according to the procedures below.

Groups 1 and 2 were assessed for toxicity after a single administration via intraduodenal catheter. Groups 1 received 6 mg/kg of IFN-α-2b(E41Q) in 1 mL (2 mg/mL) and Group 2 received 1 mL of vehicle only as a control. A daily clinical examination and weighing were performed in order to detect any signs of toxicity until day 7. At day 7, the animals were euthanized and the intestine prepared for histopathologic analysis.

Groups 3 and 4 were assessed for toxicity after repeated administrations daily for seven days via intraduodenal catheter. Groups 3 received 6 mg/kg of IFN-α-2b(E41Q) in 1 mL (2 mg/mL) each day for seven days and Group 4 received 1 mL of vehicle only as a control daily for seven days. A daily clinical examination and weighing were performed in order to detect any signs of toxicity until day 10. At day 10, the animals were euthanized and the intestine prepared for histopathologic analysis.

No animals died during the observation period. Slightly ruffled fur was observed in one animal. The body weights of the remaining animals were within the range commonly recorded for this strain and age. No abnormal clinical signs were observed.

Analysis of the animals showed evidence of localized effects likely associated with the route of administration and presumably due to mechanical, traumatic disruption (catheterization) and resultant inflammation. These effects were separated from more widespread effects on the mucosa, which more likely is due to test article toxicity. For example, upon histologic examination, all the specimens (Groups 1, 2, 3 and 4) except one rat (rat no. 1, group 4) had focal disruption/inflammation of the duodenal wall, usually accompanied by mild local peritonitis. These were not interpreted as evidence of product toxicity but rather an indication of mechanical trauma resulting from the gavage process.

Most specimens (Groups 1, 2, 3 and 4) had minimal fusion of villi, often fused at the tips. In addition, most specimens had a minimal increase in leukocytes (lymphocytes and plasma cells) in the lamina propria of the villi. Fused villi also can occasionally be observed in normal animals. Increased leukocytes in the lamina propria must also be interpreted cautiously, as some leukocytes are normally present.

Thus, due to the frequent transmural injury observed in all groups, including granulomatous inflammation and mild peritonitis in the duodenum, the fusion of the villi and leukocytic infiltration, administration of IFN-α-2b(E41Q) by intraduodenal administration at the tested doses and schedules was not considered to be toxic.

### B. Cynomolgus Monkeys

1. 2-Week Oral Toxicity Study in Cynomolgus Monkeys (Macaca fascicularis) by the Oral Route

The study involved assessment of four groups of monkeys following oral administration daily with various doses of IFN-α-2b(E41Q). A total of twenty-four Cynomolgus monkeys (Macaca fascicularis) at 24-36 months of age weighing 1.8-2.8 kg (twelve males and twelve females, divided equally among the groups (three animals/sex/group)) were used in the study. The four treatment groups are set forth in Table 83 below:

TABLE 83
Toxicology Treatment Groups

| | | No. of | |
|---|---|---|---|
| | | mice/group | |
| Group (IFN-α-2b(E41Q) dose) | | Male | Female |
| 1. | vehicle alone (four enteric-coated placebo tablets) | 3 | 3 |
| 2. | 0.6 mg/monkey (0.26 mg/kg; one 600 mg IFN-α- | 3 | 3 |
| | 2b(E41Q) enteric-coated tablet + three | | |
| | enteric-coated placebo tablets) | | |
| 3. | 1.2 mg/monkey (0.52 mg/kg; two 600 mg IFN-α- | 3 | 3 |
| | 2b(E41Q) enteric-coated tablets + two | | |
| | enteric-coated placebo tablets) | | |
| 4. | 2.4 mg/monkey (1.04 mg/kg; four 0.6 mg IFN-α- | 3 | 3 |
| | 2b(E41Q) enteric-coated tablets) | | |

The animals were dosed daily for two consecutive weeks by oral administration of 4 enteric-coated tablets as indicated above for each group. Animals were observed twice a day for mortality and clinical signs. They were weighed once a week during the acclimatization period and on treatment days 1, 8, and 14 and before sacrifice. Body temperature was recorded before administrations 1, 7, 13 and also at 0.5, 1, 2, 4 h after treatment. Opthalmoscopic examinations and blood analyses were performed at pre-dose and at week 2 of the administration period. Hematology, clinical biochemistry and urinalysis examinations were conducted prior to the first dose and at week 2. At the end of the treatment period, a full necropsy and a histopathologic examination were conducted on all animals.

No mortalities, no relevant clinical signs, no ocular alteration, no variations in body weight or temperature and no microscopic lesions were noted during and at the end of the entire study period for all animals. In addition, blood analysis and urinary analyses did not show any relevant differences among the groups. There were no significant histopathologic alterations that distinguished controls from treated animals.

In addition to toxicity assessment, blood samples were taken prior to the first dose and after administration at day 1, day 6 and day 13 for immunogenicity studies. Methods and results are reported separately in Example 20.A.3. Blood samples for the determination of the plasma levels of IFN-α-2b(E41Q) (toxicokinetics) were taken at days corresponding to administration days 1 and day 14. Methods and results are reported separately in Example 12.

2. Comparison of Toxicity of IFN-α-2b(E41Q) to Commercial IFN-Alpha—Intron A® Interferon and Pegasys® Interferon Following Subcutaneous Administration

Toxicity analysis was performed on Cynomolgus monkeys (Macaca fascicularis) receiving various doses of IFN-α-2b(E41Q), Intron A® interferon, Pegasys® interferon and native IFN-α-2b following administration by subcutaneous route as described in Example 14. In the studies in Example 14, numerous safety parameters were evaluated, including general safety (e.g., mortality, body weight, food consumption, rectal temperature); hematology (e.g., erythrocyte, hemoglobin, mean cell volume, packed cell volume, mean cell hemoglobin concentration, mean cell hemoglobin, thrombocytes, leucocytes, differential white cell count, prothrombin time, fibrinogen, activated partial thromboplastin time); blood chemistry (e.g., sodium, potassium, chloride, calcium, inorganic phosphorus, glucose, urea, creatinine, total bilirubin, cholesterol, triglycerides, total proteins, albumin, albumin/globulin ratio, alkaline phosphatase, aspartate aminotransferase, alanine aminotransferase, creatine kinase, lactate dehydrogenase, g-glutamyl transferase); and clinical pathology (e.g., organ weight and status at necropsy). There were no treatment-related deaths or premature sacrifices during the study. There were no treatment-related clinical signs and no changes in body weight in any group during the study. Food consumption also was unaffected during the treatment period.

The results of the analysis showed only a slight increase in body temperature (range: −0.3 to +2.1° C.) within 2 h after each treatment, which did not appear to be dose-related and were of similar amplitude across the different treatment groups. Hematological and blood parameters were not affected by the treatment in any group during the study. There were no treatment-related modifications of any urinalysis parameter in any group. Also, there were no treatment-related effects noted in organ weights, nor abnormal findings related to treatment in the macroscopic post-mortem examination.

A low to moderate urinary levels of IFN-α were generally detected during part 1 of the study (see Example 14 for study details), except for one female administered with native IFN-α that showed a marked level of IFN-α in the urine. During Part 2 of the study (see Example 14 for study details), the only animals showing relevant levels of IFN-α in the urine were observed in the two animals given Pegasys® interferon.

3. 13-Week Dose Toxicity Study in Cynomolgus Monkeys (Macaca fascicularis) by the Subcutaneous (SC) Route

The study involved assessment of four groups of monkeys following subcutaneous injections twice a week (at days 1 and 5 of each week) for thirteen weeks with various doses of IFN-α-2b(E41Q) (0.5 mL/kg body weight) in physiological saline. A total of twenty-four Cynomolgus monkeys (Macaca fascicularis) at 42-60 months of age weighing 2.1-3.6 kg (twelve males and twelve females, divided equally among the groups (three animals/sex/group)) were used in the study. Injections were given alternately on four previously shaved areas on the back, two on each side of the mediodorsal line, for a total of twenty-six administrations. The four treatment groups are set forth in Table 84 below:

TABLE 84
Treatment Groups

No. of
mice/group

| Group (IFN-α-2b(E41Q) dose) | Male | Female |
|---|---|---|
| 1. vehicle alone | 3 | 3 |
| 2. 0.14 MIU/kg (0.47 μg/kg) | 3 | 3 |
| 3. 0.42 MIU/kg (1.40 μg/kg) | 3 | 3 |
| 4. 1.26 MIU/kg (4.20 μg/kg) | 3 | 3 |

Animals were observed daily for mortality and clinical signs. They were weighed twice a week and once before sacrifice. Body temperature was recorded before and after administrations 1, 3, 7, 12 and 26. Opthalmoscopic examinations and blood analyses were performed at pre-dose and at weeks 6 and 13 of the administration period. Urinalysis examinations were conducted at weeks 6 and 13. At the end of the treatment period, a full necropsy and a histopathologic examination were conducted on all animals.

a. Results

i. Toxicity Analysis

No unscheduled deaths or mortalities resulted from the treatments. There were no differences in the clinical signs or mean body-weight gain between treatment groups. There were no relevant differences in the mean body temperature values between groups. No ocular alterations were observed. Also, the results of the studies showed no differences in urinary parameters between groups, and no relevant differences in histopathology. There were no macroscopic alterations or variation in organ weight.

ii. Toxicokinetics

Blood samples also were taken for the determination of the plasma levels of IFN-α-2b(E41Q) (toxicokinetics) at days corresponding to administrations 1 (day 1), 8 (day 26) and 26 (day 89). The results showed no differences in hematological and haemostatic parameters between groups. A very slight increase in serum globulin levels was observed in females administered with 0.42 MIU/kg or 1.26 MIU/kg IFN-α-2b(E41Q), with no variations in the physical conditions nor in the behavior of the animals. A dose of 1.26 MIU/kg can be considered as the no observed effect level (NOEL).

Toxicokinetics: Twice a week SC injection of IFN-α-2b(E41Q) at doses of 0.14, 0.42 and 1.26 MIU/Kg in a repeat-dose toxicity study was associated with linear, dose-related increases in Cₘₐₓ and AUC after the first administration. After 13 weeks of treatment, the detection of IFN-α-2b(E41Q) serum levels was impeded by the development of neutralizing antibodies in some animals.

iii. Immunogenicity

Blood samples also were taken for immunogenicity studies (assessment of total and neutralizing antibodies) at pre-dose (day 1) and after administrations 4 (day 12), 8 (day 26), 12 (day 40) and 26 (day 89). The results show that across all dose groups, neutralizing antibodies appeared in 6-18 animals by 40 days and in 14-18 animals by 89 days. The appearance was later and at lower levels in the low dose group, and was earlier and highest in the mid-dose group.

Both total and binding anti-IFN-α antibodies increased with duration of treatment and dose administered. The results show that up to 26 days, nearly no antibodies were evidenced in any dose group, but by day 40, antibodies had appeared in 3-5 animals in each dose group (n=6). The intermediate dose (0.42 MIU/kg) appeared to elicit more antibodies than the highest dose (1.26 MIU/kg). Thus, antibody interference must be considered in toxicology studies where the treatment duration is longer than one month.

In conclusion, IFN-α-2b(E41Q) was well tolerated by monkeys when injected SC at repeated doses up to 1.26 MIU/kg for 13 weeks twice a week.

### C. Mice

1. Acute Toxicity in Mice by the Intravenous (IV) Route

The acute toxicity of IFN-α-2b(E41Q) was evaluated in mice after a single IV injection of 50 MIU/kg (5 mL/kg) of IFN-α-2b(E41Q) in 0.9% sodium chloride by administration into the caudal vein. Six female mice (HanRcc: NMRI-SPF) age 7-8 weeks and weighing 26.1-30.1 g, with free access to food and water, were used for the experiment. Mortality, clinical signs and body weight were monitored for 14 days. At the end of the observation period, all animals were sacrificed by intraperitoneal (IP) injection of Vetanarcol (dose ≧2.0 mL/kg) and post-mortem examinations were performed.

No animals died during the observation period and no abnormal clinical signs or local reactions were observed. A slight increase in body weight (4.6%) was observed in one animal between day 8 and day 15, whereas no significant increase in body weight was observed in another animal throughout the entire observation period. The body weight of the remaining animals was within the range commonly recorded for this strain and age. No macroscopic findings were recorded at necropsy.

The results show that IFN-α-2b(E41Q) was well tolerated in mice at the tested dose of 50 MIU/kg. The median lethal dose (LD₅₀) of IFN-α-2b(E41Q) after IV administration to female mice observed over a period of 14 days was greater than 50 MIU/kg of body weight.

2. Acute Toxicity in Mice by the Subcutaneous (SC) Route

The acute toxicity of IFN-α-2b(E41Q) was evaluated in mice after a single SC injection of 50 MIU/kg (5 mL/kg body weight) of IFN-α-2b(E41Q) in 0.9% sodium chloride into the neck region. Six female mice (HanRcc: NMRI-SPF) age 7-8 weeks and weighing 26.1-30.1 g, with free access to food and water, were used for the experiment. Mortality, clinical signs and body weight were monitored for 14 days. At the end of the observation period, all animals were sacrificed by intraperitoneal (IP) injection of Vetanarcol (dose ≧2.0 mL/kg) and post-mortem examinations were performed.

No animals died during the observation period. Slightly ruffled fur was observed in one animal. A slight decrease in body weight (2.4 and 1.8%) was observed in two animals during the course of the study. The body weight of the remaining animals was within the range commonly recorded for this strain and age. No macroscopic findings were recorded at necropsy.

In conclusion, IFN-α-2b(E41Q) was well tolerated at the tested dose of 50 MIU/kg. The median lethal dose of IFN-α-2b(E41Q) after SC administration to female mice observed over a period of 14 days (LD₅₀) was greater than 50 MIU/kg of body weight.

### Example 17

### Genotoxicity (Mutagenicity) Studies

A. Salmonella typhimurium Reverse Mutation Assay

The purpose of this assay was to evaluate genotoxicity by measuring the ability of IFN-α-2b(E41Q) to induce reverse mutations at selected loci. The bacterial strains used in the assay were 5 strains of Salmonella typhimurium (TA1535, TA1537, TA98, TA100 and TA102), which have a unique mutation that has turned off histidine biosynthesis. As a result of this mutation, the bacteria require exogenous histidine to survive and will starve to death if grown without histidine. The assay relies on the ability of bacteria to undergo a reverse mutation turning the essential gene back on thereby permitting the cell to grow in the absence of histidine. Each bacterial strain was created by a specific mutation. For example, the strains used were constructed to differentiate between base pair (TA 1535, TA10 and TA102) and frameshift (TA1537 and TA98) mutations. Thus, the experiment was performed to assess the potential of IFN-α-2b(E41Q) to induce gene mutation in these 5 strains of Salmonella typhimurium (TA1535, TA1537, TA98, TA100 and TA102).

The assay was performed in two independent experiments both with and without S9 mix, which is a cofactor-supplemented post-mitochondrial mix prepared from the livers of rodents and which functions as a metabolic activation system in this assay. In each of the experiments, various concentrations of IFN-α-2b(E41Q) (batch number # IFN 003-05) were tested in triplicate at concentrations ranging from 0.0390625; 0.078125; 0.15625; 0.3125; 0.625; 1.25; 2.5 and 5 μg/plate. Controls also were tested in triplicate.

Bacteria were plated on agar plates in the presence or absence of the indicated doses of IFN-α-2b(E41Q), with or without the S9 mix. The results showed normal background growth of bacteria on all plates containing concentrations of IFN-α-2b(E41Q) up to 5 μg/plate, with and without S9 mix, in all strains used. No toxic effects, evident as a reduction in the number of revertants, occurred in the test groups with and without metabolic activation.

No substantial increase in revertant colony numbers of any of the five tester strains was observed after treatment with IFN-α-2b(E41Q) at any dose level, neither in the presence nor absence of metabolic activation (S9 mix). There also was no tendency of higher mutation rates with increasing concentrations in the range below the generally acknowledged border of biological relevance.

During the described mutagenicity test and under the experimental conditions reported, the test item did not induce gene mutations by base pair changes or frameshifts in the genome of the strains used. Therefore, IFN-α-2b(E41Q) is considered to be non-mutagenic in this Salmonella typhimurium reverse mutation assay.

### B. Mouse Micronucleus Assay

The mutagenic potential of IFN-α-2b(E41Q) was determined by its ability to induce micronuclei in polychromatic erythrocytes (PCE) in the bone marrow of mice. Seventy-two NMRI mice at 9-11 weeks old (thirty-six males, thirty-six females; weight: mean values 33.3 g for males and 34.7 for females), were divided in six treatment groups (six males and six females per group). The mice received a single oral administrations of IFN-α-2b(E41Q) (in 0.9% NaCl; 10 mL/kg body weight) at the various doses, or controls as follows:
Group 1: Vehicle (negative control, 0.9% NaCl)
Group 2: IFN-α-2b(E41Q) 1.25 MIU/kg body weight (bone marrow collection after 24 h)
Group 3: IFN-α-2b(E41Q) 2.5 MIU/kg body weight (bone marrow collection after 24 h)
Group 4: IFN-α-2b(E41Q) 5.0 MIU/kg body weight (bone marrow collection after 24 h)
Group 5: IFN-α-2b(E41Q) 5.0 MIU/kg body weight (bone marrow collection after 48 h)
Group 6: Cyclophosphamide (positive control) 40 mg/kg body weight (bone marrow collection after 24 h).

Bone marrow cells were collected at the indicated times for micronuclei analysis. At least 2000 polychromatic erythrocytes (PCEs) per animal were scored for micronuclei. In order to determine a cytotoxic effect due to the treatment, the ratio between polychromatic and total erythrocytes was determined in the same sample and reported as the number of PCEs per 2000 erythrocytes. After treatment with IFN-α-2b(E41Q) at the doses and times indicated above, the number of PCEs was not substantially decreased as compared to the mean value of PCEs of the vehicle control, thus indicating that IFN-α-2b(E41Q) did not exert any cytotoxic effects in the bone marrow.

In comparison to the corresponding vehicle controls, there was no biologically relevant or statistically significant enhancement in the frequency of the detected micronuclei at any preparation interval after administration of IFN-α-2b(E41Q) at any dose level used. Whereas oral administration of the positive control to mice (40 mg/kg body weight cyclophosphamide) resulted in a substantial increase of induced micronucleus frequency, IFN-α-2b(E41Q) did not induce micronuclei in the experimental condition described for any dose tested, and can therefore be considered not mutagenic.

### Example 18

### Safety Pharmacology

### A. Modified Irwin Screen Test in Mice

An observational screening test (modified Irwin's test) was performed to determine if administration of IFN-α-2b(E41Q) induced any behavioral changes in treated animals. Eighteen male HanRcc: NMRI mice at 5 weeks of age and weighing 30-35 g (six animals/group) were assessed for behavioral changes immediately after and at 0.5, 1, 2 and 4 h after a single subcutaneous (SC) injection in the neck region of IFN-α-2b(E41Q) (5 mL/Kg body weight) at dosages of 1) 0.05 MIU/kg body weight (0.167 μg/kg), 2) 0.15 MIU/kg body weight (0.50 μg/kg) and 3) 0.45 MIU/kg body weight (1.50 μg/kg). A control group of six animals was treated with vehicle only (physiologic saline). Body weights were recorded before dosing.

The mice were observed for responses in their home cage as well as for responses to a new environment (arena) and handling. Alertness, abnormal body carriage (hunched posture), abnormal gait (walking lower than its limbs), exploratory activity, pain response, aggression, vocalization increases were recorded before dosing, immediately after dosing, and respectively at 0.5, 1, 2, and 4 h after dosing. Where similar behaviors were observed, and with similar frequency, before and after IFN-α-2b(E41Q) dosing or in the vehicle control group such behaviors were considered not to be related to the administration of IFN-α-2b(E41Q).

After treatment with IFN-α-2b(E41Q), the maximum increased pain response and alertness was observed immediately and 0.5 h after dosing in the 0.45 MIU/kg group, in two out of six animals. Additionally, an abnormal body carriage, demonstrated as a hunched posture, was recorded immediately after dosing and lasted for up to 2 h after dosing. None of these findings reached statistical significance and were considered not to be related to IFN-α-2b(E41Q).

A single SC injection of IFN-α-2b(E41Q) at doses up to 0.15 MIU/kg body weight (0.50 μg/kg) was without significant effects on the general behavior using a modified Irwin screen test in male NMRI mice. After treatment with 0.45 MIU/kg body weight (1.50 μg/kg) of IFN-α-2b(E41Q), there was some evidence of increased alertness, abnormal body carriage and an increased pain response but these behaviors were not significant and were not with sufficient frequency to be statistically significant.

B. Effect on HERG-1 Tail Currents Recorded from Stably Transfected HEK 293 Cells

The purpose of this study was to investigate the potential for IFN-α-2b(E41Q) to block the HERG-1 potassium channels, which is associated with ventricular arrhythimias. HERG-1 tail currents were recorded in transfected HEK 293 cells stably expressing HERG-1 potassium channels. The effects of IFN-α-2b(E41Q) at concentration levels of 100, 1000 and 10000 IU/mL were investigated using the whole-cell patch clamp technique at room temperature. Each concentration was tested in at least four isolated cells from different culture dishes in order to avoid contamination. At least four separate cells were treated with vehicle alone (137 mmol NaCl/1, 4 mmol/l KCl, 1.8 mmol/l CaCl₂, 1 mmol/l MgCl, 10 mmol/l HEPES (N-2-hydroxyethylpiperazine-N′-2-ethanesulfonic acid), 10 mmol/l D-Glucose) as a negative control. Two additional cells were treated with 100 nM of the selective I_{Kr} blocker E-4031, which is a known blocker of HERG-1 channels, in order to assure accuracy of the method.

This study showed that at the concentrations of 100, 1000 and 10000 IU/mL, IFN-α-2b(E41Q) had no effect on HERG-1 tail currents. Thus, IFN-α-2b(E41Q) does not interact with the HERG-1 channel at the concentration tested and under the conditions described, and should have no effect on ventricular repolarization.

### C. Telemetry Study in Cynomolgus Monkeys

Five male Cynomolgus monkeys (Macaca fasciculata) at 5-7 years of age (sexually mature), weighing 4-6.1 kg, were anesthetized with ketamine-HCl at 9 mg/kg and diazepam at 0.5 mg/kg via intramuscular (IM) injection. Surgical level anesthesia was induced and maintained by continuous ketamine intravenous (IV) drip (approx. 50-70 mg/kg/h as 1% solution) into the left vena cephalica during the entire cardiovascular recording procedure. Following anesthesia administration, the animals were allowed to rest for 15 minutes to stabilize circulatory functions. They were then treated first with the vehicle (0.9% NaCl solution) and then with increasing dose levels of IFN-α-2b(E41Q) (2 mL/kg body weight) [0.14 MIU/kg (0.47 μg/kg), 0.42 MIU/kg (1.40 μg/kg), and 1.26 MIU/kg (4.20 μg/kg)], administered by subcutaneous (SC) bolus injections into the dorsal region at intervals of 90 minutes (60 minute observation period and 30 minute stabilization period). Before the first administration and at further suitable times after the administration, noradrenaline (2 μg/kg) and isoproterenol (2 μg/kg) were administered intravenously, in order to confirm that the cardiovascular reactions to noradrenaline and isoproterenol were influenced by administration of IFN-α-2b(E41Q) into the animals.

The animals were assessed for the following parameters: peripheral arterial blood pressure (systolic, diastolic and mean blood pressures), pulmonary arterial blood pressure, heart rate, electrocardiography, cardiac output and cardiac stroke volume, left ventricular pressure, central venous pressure, respiration (respiration rate and minute volume) and blood gas analysis.

The data showed no evidence of any effect on the systolic, diastolic and mean arterial blood pressure after SC administration of IFN-α-2b(E41Q) at doses of 0.14 MIU/kg and 0.42 MIU/kg. Under the test conditions, a marginal, though not significant (p≦0.01), decrease of the systolic, diastolic and the mean arterial blood pressure was noted 30 and 60 minutes after the SC administration of IFN-α-2b(E41Q) at a dose of 1.26 MIU/kg. Since hypotension is a well-known effect of interferons, this reduction is considered as being related to IFN-α-2b(E41Q). No influence was noted on any of the following cardiovascular parameters: systolic and diastolic pulmonary arterial blood pressure, capillary blood pressure (wedge pressure), heart rate, QRS-interval, QT-interval, QTc-interval, P-interval, cardiac output, stroke volume, systolic left ventricular pressure, dp/dTmax, central venous pressure as well as respiratory rate and volume, blood pH value and blood gases. No evidence was noted for a prolongation of the QT-interval. The cardiovascular reactions to noradrenaline and isoproterenol were not influenced.

IFN-α-2b(E41Q) did not exert any statistically significant influence on any of the cardiovascular and respiratory parameters which were taken into consideration. A statistically non-significant decrease in arterial blood pressure (systolic, diastolic and mean) was attributed to IFN-α-2b(E41Q), since hypotension is a well known effect of interferon.

### Example 19

### Immunogenicity Prediction

Both in the initial selection of candidate LEAD polypeptides and in the development program itself, the immunogenic potential of IFN-α-2b(E41Q) has been examined. This has included an in silico analysis of B- and T-cell epitopes for IFN-α-2b(E41Q) compared to those for native IFN-α as well as a determination of the presence of neutralizing and total antibodies to IFN-α as described in Example 20 below.

The prediction of B-cell and T-cell epitope changes caused by the single amino acid replacement E41Q in IFN-α-2b was performed in silico using two algorithms: B cell epitope algorithm (BCEPRED) and T cell epitope algorithm (SYFPEITHI), respectively.

### A. B-Cell Epitope Prediction

The BCEPRED algorithm (Saha S. et al., in G. Nicosia, V. Cutello, P. J. Bentley and J. Timis (Eds.) ICARIS 2004, LNCS 3239, 197-204, Springer, 2004) is used to predict B-cell epitope regions in an antigen sequence. This prediction relies on physico-chemical properties (Ronni et al., J. Immunol., 1993; 150:1715-1726). The BCEPRED algorithm can predict continuous B-cell epitopes with 58.7% accuracy using Flexibility, Hydrophilicity, Polarity, and Surface properties combined at a threshold of 2.38.

This algorithm plots the residue properties along the protein backbone. The peak of the amino acid residue segment above the threshold value (default is 2.38) is considered as a predicted B-cell epitope. No difference in B-cell epitope predictions was observed between IFN-α-2b(E41Q) and native IFN-α.

### B. T-Cell Epitope Prediction

The SYFPEITHI algorithm was used to predict the ligation strength to a defined HLA type for a sequence of amino acids (Rammensee et al. (1999) Immunogenetics, 50:213-9; www.syfpeithi.de). The algorithm is based on the book “MHC Ligands and Peptide Motifs” by H. G. Rammensee, J. Bachmann and S. Stevanovic. The probability of being processed and presented is given in order to predict T-cell epitopes.

Using the algorithm, no evidence for new T-cell epitope was observed with IFN-α-2b(E41Q) compared to native IFN-α. In addition, four regions, beginning at amino acid positions 34, 38, 40 and 41 on native IFN-α-2b, and which were predicted to be T-cell epitopes, showed a processing probability score higher on native IFN-α-2b than the corresponding region on IFN-α-2b(E41Q). The predicted T-cell epitope in those regions and the predicted scores for native IFN-α-2b and IFN-α-2b(E41Q) mutation are shown in Table 85 below. The amino acid residue corresponding to the E41Q mutation is indicated in bold.

TABLE 85

Predicted T-cell epitopes for IFN-α-2b(E41Q)

| | | | | | |
|---|---|---|---|---|---|
| | | | | IFN-α- | |
| | | SEQ | Native | 2b | % of re- |
| Posi- | T-cell | ID | IFN-α | (E41Q) | duction of |
| tion | epitope | NO: | score | score | immunogenicity |

| | | | | | |
|---|---|---|---|---|---|
| 34-43 | HDFGFPQQEF | 2158 | 12 | 10 | 17 |
| 38-47 | FPQQEFGNQF | 2159 | 11 | 10 | 17 |
| 40-49 | QQEFGNQFQK | 2160 | 11 | 1 | 99 |
| 41-50 | QEFGNQFQKA | 2161 | 15 | 5 | 66 |

All B-cell and T-cell prediction data supported the fact that IFN-α-2b(E41Q) did not have a larger immunogenic potential than native IFN-α.

### Example 20

### Analysis of Immunogenicity in Pharmacokinetic and Pharmacodynamic Animal Model Studies

Animals from the pharmacokinetic and toxicity studies described in Examples 10, 12, 14, 15, and 16 also were assessed for the presence of total and neutralizing antibodies as an assessment of the immunogenicity of IFN-α-2b(E41Q). The results below show 1) determination of neutralizing and binding antibodies for IFN-α-2b(E41Q), native IFN-α, Intron A® interferon and Pegasys® interferon in a PK/PD/clinical safety ascending dose study in monkeys; 2) determination of neutralizing and binding antibodies in a 13-week repeat dose toxicology study in monkeys; 3) determination of neutralizing and binding antibodies in a 2-week repeat dose toxicity study in monkeys; 4) determination of neutralizing and binding antibodies in a 2-week repeat dose toxicity study in rats; 5) determination of a generation of new epitopes in transgenic mice; and 6) cross-reactivity studies between IFN-α-2b(E41Q) antibodies and native IFN-α.

### A. Monkeys

1. Immunogenicity Assessment in Toxicity Study of IFN-α-2b(E41Q), Commercial IFN-Alpha—Intron A® and Pegasys® Following Subcutaneous Administration

In the toxicity study described in Example 14 and Example 16.B.2 above, groups of 1 male and 1 female monkey were given escalating single doses of IFN-α-2b(E41Q), native IFN-α, Intron A® interferon and Pegasys® interferon. Blood samples were taken for preliminary examination on specified days after administration for assessment of antibody formation.

a. Measurement of Neutralizing Antibodies

The quantification of neutralizing antibodies was performed according to Kawade's assay (Kawade et al., J. Interferon Research, 1987; Grossberg et al., J. Interferon Cytokines Research; 2001; Grossberg et al., Biotherapy; 1997; Kawade Y., J. Interferon Research, 1980; 1:61-70; Kawade et al., J. Immunological Methods, 2003). Briefly, 10 LU (Laboratory Units) of native IFN-α or IFN-α-2b(E41Q) were added to 12 serial (2-fold) dilutions of monkey blood samples that had been collected on specified days after administration of protein. After incubation for 4 h, the mixtures were placed on EMCV infected HeLa cells to measure the remaining antiviral activity as described in Example 1. Neutralizing antibody levels were determined as 10-fold reducing unit per mL of monkey serum (TRU/mL) corresponding to the dilution of monkey serum necessary to obtain 50% of total activity of IFN-α-2b(E41Q) and native IFN-α not treated with serum.

The results are depicted in Tables 86 and 87 below. Low levels of IFN-α-neutralizing antibodies were observed after the end of the dosing period in all treated groups except for animals given Intron A® interferon. During Step 2 (after the washing period), IFN-α-neutralizing antibody levels were detected from the beginning of the treatment at similar or higher levels than those noted during Step 1. No IFN-α neutralizing antibodies were detected in animals given Intron A® interferon as well as in naïve animals given Pegasys® interferon and IFN-α-2b(E41Q).

Levels of neutralizing anti-IFN-α antibodies of IFN-α-2b(E41Q) (TRU/mL) obtained in Part 1 (Steps 1 and 2) compared to native IFN-α and Intron A® interferon are shown in Table 86 below.

TABLE 86
Neutralizing antibodies (part 1)

| | Native IFN-α | | Intron A ® | | IFN-α-2b(E41Q) | |
|---|---|---|---|---|---|---|
| | (TRU/mL) | | (TRU/mL) | | (TRU/mL) | |
| | M | F | M | F | M | F |
| Day − 3 | ND | ND | ND | ND | ND | ND |

Step 1: injection at days 1, 4, 8 and 12 with doses of 0.03, 0.3, 1.0 and 3.0
mg/kg for native IFN-α and IFN-α-2b(E41Q); 0.0, 0.0, 0.03 and
0.3 mg/kg for Intron A ® interferon

| | | | | | | |
|---|---|---|---|---|---|---|
| Day 16 | ND | ND | ND | ND | ND | ND |
| Day 21 | ND | ND | ND | ND | ND | ND |
| Day 28 | 125 | 111 | 14 | ND | 27 | ND |
| Day 51 | 50 | 43 | ND | ND | 67 | 29 |

Step 2: injection at days 66, 72 and 78 with doses of 0.03, 0.03 and 0.3
mg/kg; non-naïve animals

| | | | | | | |
|---|---|---|---|---|---|---|
| Day 86 | 83 | 40 | ND | ND | 37 | 125 |
| Day 93 | 83 | 50 | ND | ND | 29 | 26 |
| Day | 1667 | 111 | ND | ND | 100 | 26 |
| 100 | | | | | | |

TRU = Ten-fold Reducing Units;
ND = Not Detectable

Levels of neutralizing anti-IFN-α antibodies of IFN-α-2b(E41Q) (TRU/mL) obtained in Part 2 compared to Pegasys® interferon are shown in Table 87 below.

TABLE 87
Neutralizing antibodies (part 2)

| | |
|---|---|
| | IFN-α- |
| Pegasys ® | 2b(E41Q) |
| (TRU/mL) | (TRU/mL) |

| | M | F | M | F |
|---|---|---|---|---|
| Day - 8 | ND | ND | ND | ND |

Injections at days 1, 7 and 13 with doses of 0.03, 0.03 and 0.3
mg/kg for IFN-α-2b(E41Q) and 0.0, 0.3 and 0.3 mg/kg for
Pegasys ® interferon

| | | | | |
|---|---|---|---|---|
| Day 21 | ND | ND | ND | ND |
| Day 28 | ND | ND | ND | ND |
| Day 35 | ND | ND | ND | ND |

TRU = Ten-fold Reducing Units;
ND = Not Detectable

b. Quantitation of Total Anti-IFN-α Antibodies

Quantitative determination of total anti-IFN-α antibodies was determined in monkeys administered with various IFN-α proteins as described in Example 14. The antibody levels in were assayed using human anti-IFN-α Bender Medsystems Immunoassay Kit (BMS217-Tebu, France). Briefly, monkey plasma was collected at specified times after administration as indicated in Example 14, and the plasma samples were diluted 5 times according to the manufacturer's protocol. The optical density (OD) at 450 nm in the samples was measured using a spectrophotometer. The Results are expressed in ng/mL. The results are depicted in Tables 88 and 89 below. No antibodies against IFN-α were observed before the first administration under any of the conditions or dosing regimes tested. During Part 1, Step 1, an elevated level of total antibodies against IFN-α was noted in all treated animals at the end of the treatment period, except in animals given Intron A® interferon. The lack of total antibodies detected with Intron A® interferon likely was because the highest dose for this compound was 10-fold lower compared to the other groups.

During Step 2 antibodies were detected at similar or higher levels than those noted during Step 1. Low to moderate total antibody levels were noted in animals given Intron A® interferon, whereas in Part 2 high levels were detected in naïve animals given Pegasys® interferon and IFN-α-2b(E41Q) but at a lesser degree than in non naïve animals from Step 2 as shown in Tables 88-89 below.

Titers of total anti-IFN-α antibodies of IFN-α-2b(E41Q) (ng/mL) obtained in Part 1 (Steps 1 and 2) compared to native IFN-α and Intron A® interferon are shown in Table 88 below:

TABLE 88
Total antibodies (part 1)
Determination of total anti-IFN-α antibody concentration
(Bender Medsystem Immunoassay)

| | Native IFN-α | | Intron A ® | | IFN-α-2b(E41Q) | |
|---|---|---|---|---|---|---|
| | (ng/mL) | | (ng/mL) | | (ng/mL) | |
| | M | F | M | F | M | F |
| Day − 3 | 0.0 | 0.0 | 0.0 | 0.0 | 8.9 | 0.0 |

Step 1: injections performed at days 1, 4, 8 and 12 with doses of 0.03, 0.3,
1.0 and 3.0 mg/kg for native IFN-α and IFN-α-2b(E41Q); 0.0, 0.0, 0.03
and 0.3 mg/kg for Intron A ® interferon

| | | | | | | |
|---|---|---|---|---|---|---|
| Day 16 | 3334 | 2314 | 0.0 | 0.0 | 284 | 2221 |
| Day 21 | 2300 | 1886 | 5.3 | 136 | 1113 | 1879 |
| Day 28 | 1787 | 1915 | 0.0 | 48 | 1696 | 1364 |
| Day 51 | 2101 | 722 | 14 | 98 | 1525 | 1587 |

Step 2: injections at days 1, 7 and 13 with doses of 0.03, 0.03 and
0.3 mg/kg; non-naïve animals

| | | | | | | |
|---|---|---|---|---|---|---|
| Day 21 | 3270 | 2726 | 16 | 539 | 1646 | 1448 |
| Day 28 | 2875 | 3470 | 23 | 495 | 2171 | 1670 |
| Day 35 | 3324 | 2688 | 16 | 336 | 2338 | 1818 |

Levels of total anti-IFN-α antibodies of IFN-α-2b(E41Q) (ng/mL) obtained in Part 2 compared to Pegasys® interferon interferon are shown in Table 89 below:

TABLE 89
Total antibodies (part 2)

| | |
|---|---|
| | IFN-α- |
| Pegasys ® | 2b(E41Q) |
| (ng/mL) | (ng/mL) |

| | M | F | M | F |
|---|---|---|---|---|
| Day - 8 | 0.0 | 0.0 | 0.0 | 0.0 |

Injections at days 1, 7 and 13 with doses of 0.03, 0.03
and 0.3 mg/kg for IFN-α-2b(E41Q) and 0.0, 0.3 and
0.3 mg/kg for Pegasys ® interferon

| | | | | |
|---|---|---|---|---|
| Day 21 | 1268 | 308 | 531 | 751 |
| Day 28 | 1881 | 2049 | 301 | 632 |
| Day 35 | 1872 | 2073 | 277 | 780 |

No relevant differences of total and neutralizing antibody levels were noted between IFN-α-2b(E41Q), native IFN-α and Pegasys® interferon. Comparisons were made with Intron A® interfere since the highest dose for Intron A® interferon was 10-fold lower.

2. 13-Week Repeated Dose Toxicity Study in Cynomolgus Monkeys (Macaca fascicularis) by the SC Route—Total and Neutralizing Antibodies

During the 13-week toxicology study in monkeys (described in Example 15), IFN-α-2b(E41Q) was administered by subcutaneous (SC) injection for 13 weeks twice a week. Blood samples for immunogenicity studies (assessment of total and neutralizing antibodies) were taken at pre-dose (day 1) and after administrations 4 (day 12), 8 (day 26), 12 (day 40) and 25 (day 89). Both total and neutralizing anti IFN-α antibodies in monkey serum were measured at study days 1, 12, 26, 40 and 89 days.

a. Measurement of Neutralizing Antibody

Quantitative determination of neutralizing antibodies was performed according to the well known Kawade's assay as described in Example 20.A.1.a above, except that incubation was for 1 hour before the mixtures were placed on EMCV infected HeLa cells to measure the remaining antiviral activity as described in Example 1. The neutralizing antibody levels (TRU/mL) were determined as described above.

The results are depicted in Tables 90(a-c) below. At days 1 and 12, no neutralizing antibodies were detected at any of the doses tested. At day 26, neutralizing antibodies were detected in only two animals, one in the intermediate dose group (0.42 MIU/kg) and one in the high dose group (1.26 MIU/kg), with marginally positive values of 27 TRU/mL and 23 TRU/mL, respectively. At day 40, no neutralizing antibodies were found in animals in the lowest dose group. In the intermediate dose group (0.42 MIU/kg), antibody titers of 240465 TRU/mL were found in four animals. In the high dose group (1.26 MIU/kg), antibody titers of 85386 TRU/mL were found in two animals. At day 89, five animals in the low dose group (0.14 mg/kg) had attained clearly positive levels of neutralizing antibodies with titers of 77348 TRU/mL. In the intermediate dose group (0.42 MIU/kg), four animals reached titers of 5012198 TRU/mL, and in the high dose group, three animals reached titers of 203929 TRU/mL.

TABLE 90a
Dose of 0.14 MIU/kg

TRU/mL

| Day | F16 | F17 | F18 | M4 | M5 | M6 |
|---|---|---|---|---|---|---|
| 1 | Nd | nd | nd | nd | nd | nd |
| 12 | Nd | nd | nd | nd | nd | nd |
| 26 | Nd | nd | nd | nd | nd | nd |
| 40 | Nd | nd | nd | nd | 4 | nd |
| 89 | 150 | 227 | nd | 87 | 348 | 77 |

TABLE 90b
Dose of 0.42 MIU/kg

TRU/mL

| Day | F19 | F20 | F21 | M7 | M8 | M9 |
|---|---|---|---|---|---|---|
| 1 | Nd | nd | nd | nd | nd | nd |
| 12 | Nd | nd | nd | nd | nd | nd |
| 26 | 2 | 3 | nd | nd | 27 | 19 |
| 40 | 240 | 437 | nd | 17 | 465 | 366 |
| 89 | 2052 | 2198 | 18 | 501 | 1779 | 1951 |

TABLE 90c
Dose of 1.26 MIU/kg

TRU/mL

| Day | F22 | F23 | F24 | M10 | M11 | M12 |
|---|---|---|---|---|---|---|
| 1 | nd | nd | nd | nd | nd | nd |
| 12 | nd | nd | nd | nd | nd | nd |
| 26 | nd | nd | 1 | nd | nd | 23 |
| 40 | 14 | nd | 386 | nd | nd | 85 |
| 89 | 203 | 3 | 929 | nd | 73 | 427 |

nd = not detectable
threshold of positivity = neutralizing antibody titer >20 TRU/mL
TRU/mL = ten fold reducing units/mL (values from quantitative neutralization assay)

b. Quantitation of Total Anti-IFN-α Antibodies

Quantitative determination of total anti-IFN-α antibodies was determined using human anti-IFN-α Bender Medsystems Immunoassay Kit (BMS217-Tebu, France) (SOP-TST-003/06 rev1) as described in Example 20.A.1.b above. The lower limit of detection of anti-IFN-α antibodies was determined to be 1.38 ng/mL. The calculated overall coefficient of inter-assay variation is 4.1% (determined by the manufacturer).

The results are depicted in Tables 91(a-d) below. Total anti-IFN-α antibodies determined for each dose of IFN-α-2b(E41Q) (0.14, 0.42 and 1.26 MIU/kg) and dose control are shown in the Tables. Male and female monkeys are indicated by the animal number preceded by M or F, respectively.

At days 1, 12 and 26 at the 0.14 MIU/kg dose, no detectable levels of total antibodies, (corresponding to values below the lower limit of the standard curve of the assay, i.e. values similar to dose control that ranged between 109 and 759 ng/mL) were observed, except for animal M5, which showed an antibody concentration of 1744 ng/mL at day 26. At the intermediate and higher dose, antibody was detectable in some animals: animal M9 had a total antibody level of 11943 ng/mL at day 26 at the 0.42 MIU/kg dose and animals F22 and M12 had antibody levels of 2423 and 4299 ng/mL, respectively, at the 1.26 MIU/kg dose at day 26.

At day 40, all animals who received the 0.14 MIU/kg dose had total antibodies less than 2800 ng/mL (no levels were detected in animals F18 and F17 and M4). For the 0.42 MIU/kg dose, four monkeys presented concentration levels between 10000 and 25000 ng/mL (no levels were detected in F21 and less than 2700 ng/mL in M7).

For the 1.26 MIU/kg dose, three monkeys showed antibody concentrations less than 1,000 ng/mL, F22 presented a titer about 2909 ng/mL and only one monkey (F24) showed a concentration of 28000 ng/mL.

At day 89, less than 21000 ng/mL of total antibodies were detected in monkey serum at 0.14 MIU/kg and 1.26 MIU/kg doses, except for F24 which showed 60497 ng/mL at the 1.26 MIU/kg dose. At the 0.42 MIU/kg dose, four out of the six monkeys showed total antibody concentrations between 40000 and 70000 ng/mL and one monkey (F21) presented detectable total antibodies of less than 1000 ng/mL.

Thus, Similar to the results for neutralizing anti-IFN-α antibodies, total anti-IFN-α antibodies increased with doses and duration of treatments and were more pronounced in the intermediate dose group than in the high dose group and considerable animal to animal variation was apparent.

TABLE 91a
Dose of 0.14 MIU/kg

| | Total anti-IFN-α antibodies (ng/mL) | | | | | | | | | | | |
|---|---|---|---|---|---|---|---|---|---|---|---|---|
| Day | F16 | SD | F17 | SD | F18 | SD | M4 | SD | M5 | SD | M6 | SD |
| 1 | 258 | 59 | 324 | 156 | 47 | nd | 276 | 98 | 723 | 177 | 762 | 132 |
| 12 | 199 | nd | 320 | nd | 30 | nd | 546 | nd | 705 | 297 | 410 | 228 |
| 26 | 340 | nd | 415 | nd | 610 | nd | nd | nd | 1744 | 257 | 739 | 368 |
| 40 | 2787 | 169 | 23 | nd | nd | nd | nd | nd | 1931 | 285 | 1579 | 122 |
| 89 | 11487 | 296 | 11746 | 25 | nd | nd | 7431 | 29 | 20494 | 1475 | 5838 | 103 |

estimated value X < 610 ng/ml (inferior limit of the standard curve)
nd = not detected
SD = standard deviation

TABLE 91b
Dose of 0.42 MIU/kg

| | Total anti-IFN-α antibodies (ng/mL) | | | | | | | | | | | |
|---|---|---|---|---|---|---|---|---|---|---|---|---|
| Day | F16 | SD | F17 | SD | F18 | SD | M4 | SD | M5 | SD | M6 | SD |
| 1 | nd | nd | nd | nd | 31 | nd | 42 | 25 | 584 | nd | 346 | 247 |
| 12 | 466 | nd | nd | nd | 495 | nd | 253 | nd | 315 | 59 | 177 | nd |
| 26 | 1814 | 5 | 3409 | 158 | nd | nd | 1513 | 210 | 5548 | 23 | 11943 | 1042 |
| 40 | 24321 | 152 | 16026 | 921 | 343 | 315 | 2648 | 1162 | 21349 | 724 | 13948 | 1111 |
| 89 | 68256 | 3724 | 53059 | 2546 | 939 | 177 | 18685 | 180 | 64022 | 5288 | 41508 | 1559 |

estimated value X < 584 ng/mL (inferior limit of the standard curve)
nd = not detected
SD = standard deviation

TABLE 91c
Dose of 1.26 MIU/kg

| | Total anti-IFN-α antibodies (ng/mL) | | | | | | | | | | | |
|---|---|---|---|---|---|---|---|---|---|---|---|---|
| Day | F22 | SD | F23 | SD | F24 | SD | M10 | SD | M11 | SD | M12 | SD |
| 1 | nd | nd | 206 | nd | nd | nd | 252 | 179 | 283 | 144 | 60 | nd |
| 12 | 87 | nd | 8 | 5 | nd | nd | 119 | nd | 102 | nd | 129 | 91 |
| 26 | 2423 | 26 | nd | nd | 896 | 36 | nd | nd | 60 | nd | 4299 | 60 |
| 40 | 2909 | 179 | 387 | 1 | 27976 | 1131 | 108 | 52 | 890 | 140 | 8945 | 192 |
| 89 | 11987 | 1188 | 372 | 3 | 60497 | 1075 | 351 | nd | 2202 | 147 | 16761 | 279 |

estimated value X < 351 ng/mL (inferior limit of the standard curve)
nd = not detected
SD = standard deviation

TABLE 91d
Dose control

| | Total anti-IFN-α antibodies (ng/mL) | | | | | | | | | | | |
|---|---|---|---|---|---|---|---|---|---|---|---|---|
| Day | F13 | SD | F14 | SD | F15 | SD | M1 | SD | M2 | SD | M3 | SD |
| 1 | nd | nd | 224 | 54 | 708 | nd | 237 | nd | 298 | 130 | 759 | nd |
| 12 | 283 | nd | 571 | nd | 171 | nd | nd | nd | 851 | 87 | 315 | 95 |
| 26 | 260 | nd | 328 | 279 | nd | nd | nd | nd | 655 | nd | 702 | 701 |
| 40 | 199 | nd | 191 | nd | nd | nd | 109 | nd | 440 | 88 | 423 | nd |
| 89 | 125 | nd | nd | nd | 520 | nd | nd | nd | 344 | nd | 642 | nd |

estimated value X < 759 ng/mL (inferior limit of the standard curve)
nd = not detected
SD = standard deviation

No neutralizing antibody levels were detected at doses of 0.14, 0.42 and 1.26 MIU/kg in injected monkeys until day 26 of the study. Similarly, low amounts of total antibodies were detected at day 26 in blood samples of two animals (with no negative effect on pharmacokinetic profile of IFN-α-2b(E41Q)). At day 40 of the study, a moderate increase in the amount of both total and neutralizing antibodies was observed in four animals.

At day 89 of the study, a high heterogeneity in individual antibody responses to repeated SC administration of IFN-α-2b(E41Q) was observed in monkeys given the 0.42 MIU/kg dose and to a lesser extent with the 1.26 MIU/kg dose for both total and neutralizing antibodies. An impairment of pharmacokinetic profile was observed in several monkeys showing high levels of neutralizing antibodies (two males and two females for the 0.42 MIU/kg dose and one female at the 1.26 MIU/kg dose).

To conclude, no induction of immunogenicity leading to an impairment of the pharmacokinetic profile of IFN-α-2b(E41Q) was observed up to day 26 of the study with doses of 0.14, 0.42 and 1.26 MIU/kg twice a week.

c. Comparative Immunogenicity Study on Antibody-Positive Samples

Blood samples showing positive values for total and neutralizing antibodies from the studies above were assessed for determinations of binding antibodies (BAb) and neutralizing antibody (NAb) titers using RIA and CPE assays, respectively (performed by BioMonitor ApS, Copenhagen, DK). An additional cross-reactivity assay also was performed for both binding and neutralizing antibodies directed against IFN-α-2b(E41Q) and native IFN-α.

i. RIA Assay

After serial dilution of blood samples in buffer (1:10, 1:100, 1:1000 and 1:10000), the RIA titration assay was performed by incubating 50 μL of diluted serum overnight with 50 μL of ¹²⁵I-labeled IFN-α-2b(E41Q) (4000-4500 cpm) at 4° C. The binding antibodies were detected after elution of the sample from a Protein G column. Counts (cpm) bound to antibodies were expressed as percentages of total eluted cpm. The results were compared to the results from the total anti-IFN-α antibody concentrations in monkey plasma samples using the human anti-IFN-α Bender Medsystems Immunoassay Kit (BMS, 217-Tebu, France) as set forth in part ii above. The samples were diluted 5-fold according to the manufacturer's protocol. Results were expressed in ng/mL after extrapolation from standard OD curve measured with a spectrophotometer at 450 nm.

Total antibody data obtained from RIA and ELISA assays correlated well for all monkey blood samples. The statistical analysis demonstrated that the two data sets were nearly super-imposable (p<0.0001). Samples with low amounts of total antibodies determined by ELISA presented a low percentage of binding antibodies following RIA assays. Similarly, samples showing a high amount of total antibodies in the ELISA assay presented a high percentage of binding antibodies. The differences in absolute values obtained for each samples with the two assays resulted from the different methodologies used.

ii. CPE Assay

A cytopathic effect (CPE) assay was performed to measure the ability of blood samples from above to protect the viability of MC-5 cells (a subclone of A549 lung cells) exposed to an otherwise toxic level of encephalomyocarditis virus (CPE-EMCV). Serial 2-fold dilutions of IFN-α-2b(E41Q) were tested in order to determine the highest dilution resulting in a reduction of IFN activity from 10 to 1 LU/mL (1 LU (Laboratory Unit)/mL is the amount of IFN inducing 50% protection against challenge virus). Serum samples obtained from treated animals above were incubated with IFN-α-2b(E41Q) at a final concentration of 10 LU/mL for 1 h at 37° C. After this incubation period, EMC virus was added, and the cell plates were incubated overnight at 37° C. in a 5% CO₂ incubator, after which MTS was added and further incubated for 2 h at 37° C. in a 5% CO₂ incubator. The results were given as Kawade titer.

The results of the CPE assay were compared to the results obtained from the quantitative determination of neutralizing antibodies specific to IFN-α-2b(E41Q) as described above using the Kawade's assay. Monkey blood samples with high neutralizing antibody titers in Kawade's assay using HeLa cells also presented a high amount of neutralizing antibody levels in the assay performed using A549 lung cells. Similarly, samples with low or intermediate levels of NAb detected using HeLa cells also were well correlated with results obtained from the A549 lung cell assay. The differences in absolute values obtained for each samples with assay using HeLa cells and A549 lung cells were probably due to a higher sensitivity of HeLa cells in Kawade's assay.

iii. Cross-Reactivity Assays

To define more precisely the immunogenic properties of IFN-α-2b(E41Q) compared to native IFN-α, cross-reactivity assays were performed on serum samples from treated monkeys for binding and neutralizing antibodies. Neutralizing and binding antibodies specific to IFN-α-2b(E41Q) and native IFN-α were titrated using Kawade and RIA assays, respectively.

The samples titrated for neutralizing antibodies exhibited equal amounts of IFN-α-2b(E41Q) and native IFN-α-specific antibodies, except for 3 blood samples which showed a clear increase in neutralizing antibodies specific to native IFN-α compared to IFN-α-2b(E41Q). Similarly, no significant differences were observed in the percentage of binding antibodies specific to IFN-α-2b(E41Q) compared to native IFN-α for all positive monkey blood samples tested. Thus, the results show that neutralizing and binding antibodies specific to IFN-α-2b(E41Q) and native IFN-α cross-reacted with excellent correspondence (p value <0.0001 in both cases).

d. Summary

These results provided demonstrate that the IFN-α-2b(E41Q) molecular mutation induced no new immunogenic epitope compared to native IFN-α. This observation was in accordance with data obtained from the in silico immunogenicity prediction showing a decrease in T-cell epitope score resulting from the IFN-α-2b(E41Q) mutation. In the studies undertaken, the immunologic behavior of IFN-α-2b(E41Q) and of native IFN-α were nearly super-imposable. Therefore, IFN-α-2b(E41Q) should not present new, increased antigenicity.

3. Total and Neutralizing Antibodies-2-Week Oral (Enteric-Coated Tablets) Toxicity Study in Cynomolgus Monkeys (Macaca fascicularis) by the Oral Route

Monkeys were treated as described in Example 16.B.1. Blood samples were taken from all the animals at pre-dose and 24 h after administrations at days 1, 6, and 13 for each tested dose and both total and neutralizing anti-IFN-α-2b(E41Q) antibodies were determined using the human anti-IFN-α antibodies by ELISA and using Kawade's assay, respectively, as described above.

The results showed no detectable anti-IFN-α-2b(E41Q) antibodies (total and neutralizing) at pre-dose and at days 2, 7 and 14 after daily administration of oral IFN-α-2b(E41Q) enteric-coated tablets. In each case, the antibody concentrations (for total antibodies) or Kawade titers (for neutralizing antibodies) measured in all monkey serum were below the background limits determined with pre-dose samples. Also, no neutralizing or total antibody levels were detected with IFN-α-2b(E41Q) doses of 0.6, 1.2 and 2.4 mg per animal in monkey serum after daily PO administration of IFN-α-2b(E41Q) enteric-coated tablets over 2 weeks. Thus, repeated oral administration of IFN-α-2b(E41Q) (enteric-coated tablet formulation) to Cynomolgus monkeys during 2 weeks induced no total or neutralizing antibodies at doses of 0.6, 1.2 and 2.4 mg per animal.

### B. Rats—Total and Neutralizing Antibodies: 2-Week Oral Toxicity Study in Rats by Oral Administration

Rats were treated as described in Example 16.A.2. Blood samples were taken from all the animals from the satellite group for the measurement of both total and neutralizing anti-IFN-α antibody levels.

1. Neutralizing Antibodies

Neutralizing antibody was determined using the Kawade's assay using HeLa cells as described above. A neutralizing antibody titer >20 TRU/mL (Ten-fold Reducing Unit) was considered as the threshold for positivity.

The results showed no detectable levels of neutralizing antibodies (NAb) in rat serum at day 1 for animals treated with doses of 0.2, 0.4 and 0.6 mg or for enteric-coated placebo tablets in any of the animals tested. At day 15, no detectable NAb levels were measured in eleven rats orally administered with 0.2 mg of IFN-α-2b(E41Q) enteric-coated capsule, and only one male exhibited a positive titer of 81.3 TRU/mL. Similarly, at day 15, no detectable NAb levels were measured in ten rats orally administered with the dose of 0.4 mg/rat, whereas one female and one male showed detectable amounts of NAb of 118.1 TRU/mL and 53.1 TRU/mL, respectively. No titers of NAb were detected in any rats administered orally with the 0.6 mg/animal dose.

2. Total Antibodies

Total antibody was measured using ELISA as described above. No detectable concentrations of total antibodies were measured in rat serum at day 1 from animals treated with doses of 0.2, 0.4 and 0.6 mg, or enteric-coated placebo tablets in all animals tested. At day 15, no total anti-IFN-α-2b(E41Q) antibody was detected in ten rats orally administered with 0.2 mg of IFN-α-2b(E41Q) enteric-coated capsule. At day 15, only two animals, one female and one male, exhibited low concentrations of total antibody of about 8.3 and 0.3 IU/mL, respectively. Similarly, eleven rats administered with a dose of 0.4 mg/rat exhibited no detectable levels of total antibodies, except for one female with a detectable amount of total antibodies about 78.5 IU/mL. Of note, one animal showing positive NAb titer with a dose of 0.4 mg (53.1 TRU/mL), but exhibited no detectable levels of total antibodies. Finally, an absence of total antibody was observed in all rats administered orally with IFN-α-2b(E41Q) dose of 0.6 mg/animal.

3. Summary

No neutralizing and total antibody levels were detected with doses of 0.2 and 0.6 mg per animal in rat serum after daily PO administration of IFN-α-2b(E41Q) enteric-coated capsules over 2 weeks. Based on the pharmacokinetic results from the same animals presented in Example 16.A.2, this finding supports the absence of strong impairment of the IFN-α-2b(E41Q) pharmacokinetic profile observed at day 14 or at day 7 with doses of 0.2, 0.4 and 0.6 mg per animal after daily administration by oral route of IFN-α-2b(E41Q) enteric-coated capsules over 2 weeks. With a dose of 0.4 mg, two animals out of a total of twelve animals showed positive NAb titers (only one animal presenting significant total antibody levels). A moderate reduction of Cₘₐₓ and AUC₍₀₋ₜ₎ values was observed between day 1 and day 14 with this dose.

C. Mice—Immunogenicity of IFN-α-2b(E41Q) in Immune Tolerant IFN-α Transgenic Mice

The immunogenicity of IFN-α-2b was assessed in transgenic mice (University of Utrecht, Prof Schellekens). Immune tolerant transgenic mice have been used to evaluate the introduction of new epitopes in insulin and tissue plasminogen activator. These mice also have been extensively used to study the immunogenicity of IFN-α preparations. To evaluate the possible presence of a new epitope in IFN-α-2b(E41Q) which could induce immunogenicity in humans, the immunogenicity of IFN-α and IFN-α-2b(E41Q) were compared in wild-type and IFN-α-2 immune tolerant transgenic mice.

Groups of five wild-type FVB/N or IFN-α-2 transgenic mice were injected intraperitoneally (IP) with 10 μg of IFN-α-2b(E41Q) or IFN-α-2a 5 days a week for 3 weeks. Blood samples were taken weekly (at day 0 before the first interferon injection and at days 7, 14, 21 and 28) and tested for the presence of antibodies to IFN-α in a bridging ELISA assay from MDS Pharma Services (Zurich, Switzerland). This assay made use of a recombinant IFN-α to catch circulating anti-IFN-α antibodies in serum samples from wild-type and transgenic mice treated with native and mutant IFN-α, respectively. Bound antibodies were detected by the addition of labeled (biotinylated) antigen and streptavidin-coupled detection enzyme.

The results showed that both IFN-α-2b(E41Q) and native IFN-α induced high levels of antibodies in wild-type mice. The immune tolerant mice showed no immune response to IFN-α or IFN-α-2b(E41Q). These results indicated that the single amino acid mutation of IFN-α-2b(E41Q) did not introduce a new epitope since no difference in antibody levels was observed for both immune tolerant and wild-type mice injected with IFN-α-2b(E41Q) versus native IFN-α.

### Example 21

Cloning and Generation of hGH(Y42I)

1. Cloning of Native hGH for Expression in Mammalian Cells

A nucleic acid molecule encoding hGH protein (see, SEQ ID NO: 1260) was cloned into a mammalian expression vector, prior to the generation of the selected mutations. A collection of pre-designed, targeted mutants was then generated such that each individual mutant was created and processed individually, physically separated from each other and in addressable arrays.

The hGH-cDNA was obtained by synthesis in vitro from human pituitary gland mRNA (Clontech) using SMART kit (Clontech). First, hGH-encoding cDNA was cloned by PCR amplification using the following primers:

forward GH1

(SEQ ID NO: 2109)

5′-ATGGCTACAGGCTCCCGGACGTCCCTGCCTCC-3′
and
reverse GH1 REV

(SEQ ID NO: 2110)

5′-CTAGAAGCCACAAGCTGCCCCTCCACAGAGCGGCACT-3′.

The PCR amplified product was cloned into an E. coli vector (pTOPO-TA (SEQ ID NO: 2070) to produce the plasmid designated pTOPO-hGH4 (SEQ ID NO: 2120).

The sequence of the hGH-encoding cDNA was confirmed by sequencing. The sequenced cDNA was then PCR amplified using the following primers:

hGHFORHIND (HindII site underlined)

(SEQ ID NO: 2111)

5′-AAAAAGCTTATGGCTACAGGCTCCCGGACG-3′
and
hGHREV (XbaI site underlined)

(SEQ ID NO: 2112)

5′-GCCCTTCTAGAAGCCACAGCTGCCCTCCACAG-3′.

hGHFORHIND and hGHREV generate HindIII and XbaI restriction sites, respectively, on either end of the clone. After restriction digestion with HindIII and XbaI, the PCR fragment containing the hGH-encoding cDNA was subcloned into the corresponding sites in pUC-CMVhGHpA to produce pNAUT-hGH (SEQ ID NO: 2119).

2. Design of GH Variants by 2D-Scanning

2D-scanning technology, described herein and also described in U.S. application Ser. Nos. 10/658,355 and 11/267,871, was used to design and obtain hGH mutants with improved resistance to proteolysis. Is-HITs were identified based upon (1) protein property to be evolved (i.e., resistance to proteolysis); (2) amino acid sequence; and (3) properties of individual amino acids.

Positions selected (is-HITs) on pituitary hGH (SEQ ID NO:1) were (numbering corresponds to amino acid positions in the mature protein): F1, P2, P5, L6, R8, L9, F10, D11, L15, R16, R19, L20, L23, F25, D26, Y28, E30, F31, E32, E33, Y35, P37, K38, E39, K41, Y42, F44, L45, P48, L52, F54, E56, P59, P61, R64, E65, E66, K70, L73, E74, L75, L76, R77, L80, L81, L82, W86, L87, E88, P89, F92, L93, R94, F97, L10, Y103, D107, Y111, D112, L113, L114, K115, D116, L117, E118, E119, L124, M125, R127, L128, E129, D130, P133, R134, F139, K140, Y143, K145, F146, D147, D153, D154, L156, K158, Y160, L162, L163, Y164, F166, R167, K168, D169, M170, D171, K172, E174, F176, L177, R178, R183, E186, and F191. The residue substitutions performed are listed in Table 92.

TABLE 92
Amino acid replacements

| Amino acid at is-HIT | Replacing amino acids |
|---|---|
| R | H, Q |
| E | H, Q, N |
| K | Q, N |
| D | N, Q |
| M | I, V |
| P | A, S |
| Y | I, H |
| F | I, V |
| W | H, S |
| L | I, V |

Amino acids at is-HITs (left column of Table 92) were replaced by selected replacing amino acids (right column of Table 92) to produce GH variants with increased resistance to proteolysis. A total of 222 variants of hGH were generated (see Example 1.II.B and Table 24).

3. Resistance to Proteolysis

hGH variants were treated with proteases in order to identify resistant molecules. The relative resistance of the mutant proteins compared to the native protein against enzymatic cleavage was determined by exposure to a mixture of proteases (as described in Example 1.I.A). Treated samples were then used to determine residual activity such as proliferative activity in a cell proliferation assay (as described in Example 1.I.C.1; see Table 24 for results). The results show that many of the polypeptides tested exhibited an increased resistance to protease in vitro as compared to native hGH.

4. Cloning of Native hGH for Prokaryotic Expression

To express hGH in E. coli, the hGH encoding cDNA fragment was amplified by PCR from pNAUT-hGH (SEQ ID NO: 2119), using the primers hGHFORPET (SEQ ID NO: 2113) and hGHREVPET (SEQ ID NO: 2114), using Herculases (Invitrogen) DNA-polymerase.

The forward primer hGHFORPET was designed to introduce an NdeI restriction site and to change Proline 2 and Proline 5 codons from human codons to higher usage codons for E. coli (CCA to CCG and CCC to CCG).

hGHFORPET (NdeI site underlined):

(SEQ ID NO: 2113)

5′-GGGAATTCCATATGTTCCCGACCATTCCGTTATCCAGG-3′

The reverse primer hGHREVPET was designed to introduce a BamHI restriction site and to change the STOP codon from TAG to TAA in order to optimize the hGH protein production yield in E. coli.

hGHREVPET (BamHI site underlined):

(SEQ ID NO: 2114)

5′-CGGGATCCCTTAAGAAGCCACAGCTGCCCTCCACAGAGC-3′

The PCR fragment was subcloned into pET-24 (Invitrogen; SEQ ID NO: 2069). In order to further optimize the hGH yield in E. coli, additional changes were made in the hGH sequence. Cycles of mutagenesis were used to change the codons that code for arginine at positions Arg 8 and Arg 16 in the mature hGH polypeptide. The arginine codons were changed to codons to higher usage codons in E. coli. The following mutagenesis primers were used:

Primers for Arg8 codon mutagenesis,

hGHARG8FOR:

(SEQ ID NO: 2115)

5′-CCGACCATTCCGTTACCCGTCTTTTTGACAA-3′
and
hGHARG8REV:

(SEQ ID NO: 2116)

5′-GAGCATAGCGTTGTCAAAAAGACGGGATAACG-3′.

Primers for Arg16 codon mutagenesis,

hGHARG16FOR

(SEQ ID NO: 2117)

5′-GACAACGCTATGCTCCGTGCCCATCGTCTGCACCAG-3′
and
hGHARG16REV

(SEQ ID NO: 2118)

5′-CTGGTGCAGACGATGGGCACGGAGCATAGCGTTGTC-3′.

Following rounds of mutagenesis, the resulting construct was verified by sequencing. The final expression plasmid was designated pET-24Naut-hGH (SEQ ID NO: 2121). Generation of the exemplary mutation, Y42I, in pET-24Naut-hGH was performed using site-directed mutagenesis techniques analogous to those described in EXAMPLE 2d. The design of the mutagenic primer was made to effect mutation of codon at nucleotide positions 4719 to 4721 of SEQ ID NO: 2121 from TAT to ATC.

The pET-24Naut-hGH(Y42I) plasmid has the following sequence of amino acids:

| | |
|---|---|
| 1 | CTAGTCGGGG AAATGTGCGC GGAACCCCTA TTTGTTTATT |
| | TTTCTAAATA |
| 51 | CATTCAAATA TGTATCCGCT CATGAATTAA TTCTTAGAAA |
| | AACTCATCGA |
| 101 | GCATCAAATG AAACTGCAAT TTATTCATAT CAGGATTATC |
| | AATACCATAT |
| 151 | TTTTGAAAAA GCCGTTTCTG TAATGAAGGA GAAAACTCAC |
| | CGAGGCAGTT |
| 201 | CCATAGGATG GCAAGATCCT GGTATCGGTC TGCGATTCCG |
| | ACTCGTCCAA |
| 251 | CATCAATACA ACCTATTAAT TTCCCCTCGT CAAAAATAAG |
| | GTTATCAAGT |
| 301 | GAGAAATCAC CATGAGTGAC GACTGAATCC GGTGAGAATG |
| | GCAAAAGTTT |
| 351 | ATGCATTTCT TTCCAGACTT GTTCAACAGG CCAGCCATTA |
| | CGCTCGTCAT |
| 401 | CAAAATCACT CGCATCAACC AAACCGTTAT TCATTCGTGA |
| | TTGCGCCTGA |
| 451 | GCGAGACGAA ATACGCGATC GCTGTTAAAA GGACAATTAC |
| | AAACAGGAAT |
| 501 | CGAATGCAAC CGGCGCAGGA ACACTGCCAG CGCATCAACA |
| | ATATTTTCAC |
| 551 | CTGAATCAGG ATATTCTTCT AATACCTGGA ATGCTGTTTT |
| | CCCGGGGATC |
| 601 | GCAGTGGTGA GTAACCATGC ATCATCAGGA GTACGGATAA |
| | AATGCTTGAT |
| 651 | GCTCGGAAGA GGCATAAATT CCGTCAGCCA GTTTAGTCTG |
| | ACCATCTCAT |
| 701 | CTGTAACATC ATTGGCAACG CTACCTTTGC CATGTTTCAG |
| | AAACAACTCT |
| 751 | GGCGCATCGG GCTTCCCATA CAATCGATAG ATTGTCGCAC |
| | CTGATTGCCC |
| 801 | GACATTATCG GGAGCCCATT TATACCCATA TAAATCAGCA |
| | TCCATGTTGG |
| 851 | AATTTAATCG CGGCCTAGAG CAAGACGTTT CCCGTTGAAT |
| | ATGGCTCATA |
| 901 | ACACCCCTTG TATTACTGTT TATGTAAGCA GACAGTTTTA |
| | TTGTTCATGA |
| 951 | CCAAAATCCC TTAACGTGAG TTTTCGTTCC ACTGAGCGTC |
| | AGACCCCGTA |
| 1001 | GAAAAGATCA AAGGATCTTC TTGAGATCCT TTTTTTCTGC |
| | GCGTAATCTG |
| 1051 | CTGCTTGCAA ACAAAAAAAC CACCGCTACC AGCGGTGGTT |
| | TGTTTGCCGG |
| 1101 | ATCAAGAGCT ACCAACTCTT TTTCCGAAGG TAACTGGCTT |
| | CAGCAGAGCG |
| 1151 | CAGATACCAA ATACTGTCCT TCTAGTGTAG CCGTAGTTAG |
| | GCCACCACTT |
| 1201 | CAAGAACTCT GTAGCACCGC CTACATACCT CGCTCTGCTA |
| | ATCCTGTTAC |
| 1251 | CAGTGGCTGC TGCCAGTGGC GATAAGTCGT GTCTTACCGG |
| | GTTGGACTCA |
| 1301 | AGACGATAGT TACCGGATAA GGCGCAGCGG TCGGGCTGAA |
| | CGGGGGGTTC |
| 1351 | GTGCACACAG CCCAGCTTGG AGCGAACGAC CTACACCGAA |
| | CTGAGATACC |
| 1401 | TACAGCGTGA GCTATGAGAA AGCGCCACGC TTCCCGAAGG |
| | GAGAAAGGCG |
| 1451 | GACAGGTATC CGGTAAGCGG CAGGGTCGGA ACAGGAGAGC |
| | GCACGAGGGA |
| 1501 | GCTTCCAGGG GGAAACGCCT GGTATCTTTA TAGTCCTGTC |
| | GGGTTTCGCC |
| 1551 | ACCTCTGACT TGAGCGTCGA TTTTTGTGAT GCTCGTCAGG |
| | GGGGCGGAGC |
| 1601 | CTATGGAAAA ACGCCAGCAA CGCGGCCTTT TTACGGTTCC |
| | TGGCCTTTTG |
| 1651 | CTGGCCTTTT GCTCACATGT TCTTTCCTGC GTTATCCCCT |
| | GATTCTGTGG |
| 1701 | ATAACCGTAT TACCGCCTTT GAGTGAGCTG ATACCGCTCG |
| | CCGCAGCCGA |
| 1751 | ACGACCGAGC GCAGCGAGTC AGTGAGCGAG GAAGCGGAAG |
| | AGCGCCTGAT |
| 1801 | GCGGTATTTT CTCCTTACGC ATCTGTGCGG TATTTCACAC |
| | CGCAATGGTG |
| 1851 | CACTCTCAGT ACAATCTGCT CTGATGCCGC ATAGTTAAGC |
| | CAGTATACAC |
| 1901 | TCCGCTATCG CTACGTGACT GGGTCATGGC TGCGCCCCGA |
| | CACCCGCCAA |
| 1951 | CACCCGCTGA CGCGCCCTGA CGGGCTTGTC TGCTCCCGGC |
| | ATCCGCTTAC |
| 2001 | AGACAAGCTG TGACCGTCTC CGGGAGCTGC ATGTGTCAGA |
| | GGTTTTCACC |
| 2051 | GTCATCACCG AAACGCGCGA GGCAGCTGCG GTAAAGCTCA |
| | TCAGCGTGGT |
| 2101 | CGTGAAGCGA TTCACAGATG TCTGCCTGTT CATCCGCGTC |
| | CAGCTCGTTG |
| 2151 | AGTTTCTCCA GAAGCGTTAA TGTCTGGCTT CTGATAAAGC |
| | GGGCCATGTT |
| 2201 | AAGGGCGGTT TTTTCCTGTT TGGTCACTGA TGCCTCCGTG |
| | TAAGGGGGAT |
| 2251 | TTCTGTTCAT GGGGGTAATG ATACCGATGA AACGAGAGAG |
| | GATGCTCACG |
| 2301 | ATACGGGTTA CTGATGATGA ACATGCCCGG TTACTGGAAC |
| | GTTGTGAGGG |
| 2351 | TAAACAACTG GCGGTATGGA TGCGGCGGGA CCAGAGAAAA |
| | ATCACTCAGG |
| 2401 | GTCAATGCCA GCGCTTCGTT AATACAGATG TAGGTGTTCC |
| | ACAGGGTAGC |
| 2451 | CAGCAGCATC CTGCGATGCA GATCCGGAAC ATAATGGTGC |
| | AGGGCGCTGA |
| 2501 | CTTCCGCGTT TCCAGACTTT ACGAAACACG GAAACCGAAG |
| | ACCATTCATG |
| 2551 | TTGTTGCTCA GGTCGCAGAC GTTTTGCAGC AGCAGTCGCT |
| | TCACGTTCGC |
| 2601 | TCGCGTATCG GTGATTCATT CTGCTAACCA GTAAGGCAAC |
| | CCCGCCAGCC |
| 2651 | TAGCCGGGTC CTCAACGACA GGAGCACGAT CATGCGCACC |
| | CGTGGGGCCG |
| 2701 | CCATGCCGGC GATAATGGCC TGCTTCTCGC CGAAACGTTT |
| | GGTGGCGGGA |
| 2751 | CCAGTGACGA AGGCTTGAGC GAGGGCGTGC AAGATTCCGA |
| | ATACCGCAAG |
| 2801 | CGACAGGCCG ATCATCGTCG CGCTCCAGCG AAAGCGGTCC |
| | TCGCCGAAAA |
| 2851 | TGACCCAGAG CGCTGCCGGC ACCTGTCCTA CGAGTTGCAT |
| | GATAAAGAAG |
| 2901 | ACAGTCATAA GTGCGGCGAC GATAGTCATG CCCCGCGCCC |
| | ACCGGAAGGA |
| 2951 | GCTGACTGGG TTGAAGGCTC TCAAGGGCAT CGGTCGAGAT |
| | CCCGGTGCCT |
| 3001 | AATGAGTGAG CTAACTTACA TTAATTGCGT TGCGCTCACT |
| | GCCCGCTTTC |
| 3051 | CAGTCGGGAA ACCTGTCGTG CCAGCTGCAT TAATGAATCG |
| | GCCAACGCGC |
| 3101 | GGGGAGAGGC GGTTTGCGTA TTGGGCGCCA GGGTGGTTTT |
| | TCTTTTCACC |
| 3151 | AGTGAGACGG GCAACAGCTG ATTGCCCTTC ACCGCCTGGC |
| | CCTGAGAGAG |
| 3201 | TTGCAGCAAG CGGTCCACGC TGGTTTGCCC CAGCAGGCGA |
| | AAATCCTGTT |
| 3251 | TGATGGTGGT TAACGGCGGG ATATAACATG AGCTGTCTTC |
| | GGTATCGTCG |
| 3301 | TATCCCACTA CCGAGATATC CGCACCAACG CGCAGCCCGG |
| | ACTCGGTAAT |
| 3351 | GGCGCGCATT GCGCCCAGCG CCATCTGATC GTTGGCAACC |
| | AGCATCGCAG |
| 3401 | TGGGAACGAT GCCCTCATTC AGCATTTGCA TGGTTTGTTG |
| | AAAACCGGAC |
| 3451 | ATGGCACTCC AGTCGCCTTC CCGTTCCGCT ATCGGCTGAA |
| | TTTGATTGCG |
| 3501 | AGTGAGATAT TTATGCCAGC CAGCCAGACG CAGACGCGCC |
| | GAGACAGAAC |
| 3551 | TTAATGGGCC CGCTAACAGC GCGATTTGCT GGTGACCCAA |
| | TGCGACCAGA |
| 3601 | TGCTCCACGC CCAGTCGCGT ACCGTCTTCA TGGGAGAAAA |
| | TAATACTGTT |
| 3651 | GATGGGTGTC TGGTCAGAGA CATCAAGAAA TAACGCCGGA |
| | ACATTAGTGC |
| 3701 | AGGCAGCTTC CACAGCAATG GCATCCTGGT CATCCAGCGG |
| | ATAGTTAATG |
| 3751 | ATCAGCCCAC TGACGCGTTG CGCGAGAAGA TTGTGCACCG |
| | CCGCTTTACA |
| 3801 | GGCTTCGACG CCGCTTCGTT CTACCATCGA CACCACCACG |
| | CTGGCACCCA |
| 3851 | GTTGATCGGC GCGAGATTTA ATCGCCGCGA CAATTTGCGA |
| | CGGCGCGTGC |
| 3901 | AGGGCCAGAC TGGAGGTGGC AACGCCAATC AGCAACGACT |
| | GTTTGCCCGC |
| 3951 | CAGTTGTTGT GCCACGCGGT TGGGAATGTA ATTCAGCTCC |
| | GCCATCGCCG |
| 4001 | CTTCCACTTT TTCCCGCGTT TTCGCAGAAA CGTGGCTGGC |
| | CTGGTTCACC |
| 4051 | ACGCGGGAAA CGGTCTGATA AGAGACACCG GCATACTCTG |
| | CGACATCGTA |
| 4101 | TAACGTTACT GGTTTCACAT TCACCACCCT GAATTGACTC |
| | TCTTCCGGGC |
| 4151 | GCTATCATGC CATACCGCGA AAGGTTTTGC GCCATTCGAT |
| | GGTGTCCGGG |
| 4201 | ATCTCGACGC TCTCCCTTAT GCGACTCCTG CATTAGGAAG |
| | CAGCCCAGTA |
| 4251 | GTAGGTTGAG GCCGTTGAGC ACCGCCGCCG CAAGGAATGG |
| | TGCATGCAAG |
| 4301 | GAGATGGCGC CCAACAGTCC CCCGGCCACG GGGCCTGCCA |
| | CCATACCCAC |
| 4351 | GCCGAAACAA GCGCTCATGA GCCCGAAGTG GCGAGCCCGA |
| | TCTTCCCCAT |
| 4401 | CGGTGATGTC GGCGATATAG GCGCCAGCAA CCGCACCTGT |
| | GGCGCCGGTG |
| 4451 | ATGCCGGCCA CGATGCGTCC GGCGTAGAGG ATCGAGATCT |
| | CGATCCCGCG |
| 4501 | AAATTAATAC GACTCACTAT AGGGGAATTG TGAGCGGATA |
| | ACAATTCCCC |
| 4551 | TCTAGAAATA ATTTTGTTTA ACTTTAAGAA GGAGATATAC |
| | ATATGTTCCC |
| 4601 | GACCATTCCG TTATCCCGTC TTTTTGACAA CGCTATGCTC |
| | CGTGCCCATC |
| 4651 | GTCTGCACCA GCTGGCCTTT GACACCTACC AGGAGTTTGA |
| | AGAAGCCTAT |
| 4701 | ATCCCAAAGG AACAGAAGAT CTCATTCCTG CAGAACCCCC |
| | AGACCTCCCT |
| 4751 | CTGTTTCTCA GAGTCTATTC CGACACCCTC CAACAGGGAG |
| | GAAACACAAC |
| 4801 | AGAAATCCAA CCTAGAGCTG CTCCGCATCT CCCTGCTGCT |
| | CATCCAGTCG |
| 4851 | TGGCTGGAGC CCGTGCAGTT CCTCAGGAGT GTCTTCGCCA |
| | ACAGCCTGGT |
| 4901 | GTACGGCGCC TCTGACAGCA ACGTCTATGA CCTCCTAAAG |
| | GACCTAGAGG |
| 4951 | AAGGCATCCA AACGCTGATG GGGAGGCTGG AAGATGGCAG |
| | CCCCCGGACT |
| 5001 | GGGCAGATCT TCAAGCAGAC CTACAGCAAG TTCGACACAA |
| | ACTCACACAA |
| 5051 | CGATGACGCA CTACTCAAGA ACTACGGGCT GCTCTACTGC |
| | TTCAGGAAGG |
| 5101 | ACATGGACAA GGTCGAGACA TTCCTGCGCA TCGTGCAGTG |
| | CCGCTCTGTG |
| 5151 | GAGGGCAGCT GTGGCTTCTA AGGATCCGAA TTCGAGCTCC |
| | GTCGACAAGC |
| 5201 | TTGCGGCCGC ACTCGATGAG CAATAACTAG CATAACCCCT |
| | TGGGGCCTCT |
| 5251 | AAACGGGTCT TGAGGGGTTT TTTGCTGAAA GGAGGAA |

Another exemplary mutation, Y42H, also was similarly generated by mutagenesis; the design of the mutagenic primer was made to effect mutation of codon at nucleotide positions 4719 to 4721 of SEQ ID NO: 2121 from TAT to CAC.

The pET-24Naut-hGH(Y42I) plasmid has the following sequence of amino acids:

| | |
|---|---|
| 1 | CTAGTCGGGG AAATGTGCGC GGAACCCCTA TTTGTTTATT |
| | TTTCTAAATA |
| 51 | CATTCAAATA TGTATCCGCT CATGAATTAA TTCTTAGAAA |
| | AACTCATCGA |
| 101 | GCATCAAATG AAACTGCAAT TTATTCATAT CAGGATTATC |
| | AATACCATAT |
| 151 | TTTTGAAAAA GCCGTTTCTG TAATGAAGGA GAAAACTCAC |
| | CGAGGCAGTT |
| 201 | CCATAGGATG GCAAGATCCT GGTATCGGTC TGCGATTCCG |
| | ACTCGTCCAA |
| 251 | CATCAATACA ACCTATTAAT TTCCCCTCGT CAAAAATAAG |
| | GTTATCAAGT |
| 301 | GAGAAATCAC CATGAGTGAC GACTGAATCC GGTGAGAATG |
| | GCAAAAGTTT |
| 351 | ATGCATTTCT TTCCAGACTT GTTCAACAGG CCAGCCATTA |
| | CGCTCGTCAT |
| 401 | CAAAATCACT CGCATCAACC AAACCGTTAT TCATTCGTGA |
| | TTGCGCCTGA |
| 451 | GCGAGACGAA ATACGCGATC GCTGTTAAAA GGACAATTAC |
| | AAACAGGAAT |
| 501 | CGAATGCAAC CGGCGCAGGA ACACTGCCAG CGCATCAACA |
| | ATATTTTCAC |
| 551 | CTGAATCAGG ATATTCTTCT AATACCTGGA ATGCTGTTTT |
| | CCCGGGGATC |
| 601 | GCAGTGGTGA GTAACCATGC ATCATCAGGA GTACGGATAA |
| | AATGCTTGAT |
| 651 | GCTCGGAAGA GGCATAAATT CCGTCAGCCA GTTTAGTCTG |
| | ACCATCTCAT |
| 701 | CTGTAACATC ATTGGCAACG CTACCTTTGC CATGTTTCAG |
| | AAACAACTCT |
| 751 | GGCGCATCGG GCTTCCCATA CAATCGATAG ATTGTCGCAC |
| | CTGATTGCCC |
| 801 | GACATTATCG GGAGCCCATT TATACCCATA TAAATCAGCA |
| | TCCATGTTGG |
| 851 | AATTTAATCG CGGCCTAGAG CAAGACGTTT CCCGTTGAAT |
| | ATGGCTCATA |
| 901 | ACACCCCTTG TATTACTGTT TATGTAAGCA GACAGTTTTA |
| | TTGTTCATGA |
| 951 | CCAAAATCCC TTAACGTGAG TTTTCGTTCC ACTGAGCGTC |
| | AGACCCCGTA |
| 1001 | GAAAAGATCA AAGGATCTTC TTGAGATCCT TTTTTTCTGC |
| | GCGTAATCTG |
| 1051 | CTGCTTGCAA ACAAAAAAAC CACCGCTACC AGCGGTGGTT |
| | TGTTTGCCGG |
| 1101 | ATCAAGAGCT ACCAACTCTT TTTCCGAAGG TAACTGGCTT |
| | CAGCAGAGCG |
| 1151 | CAGATACCAA ATACTGTCCT TCTAGTGTAG CCGTAGTTAG |
| | GCCACCACTT |
| 1201 | CAAGAACTCT GTAGCACCGC CTACATACCT CGCTCTGCTA |
| | ATCCTGTTAC |
| 1251 | CAGTGGCTGC TGCCAGTGGC GATAAGTCGT GTCTTACCGG |
| | GTTGGACTCA |
| 1301 | AGACGATAGT TACCGGATAA GGCGCAGCGG TCGGGCTGAA |
| | CGGGGGGTTC |
| 1351 | GTGCACACAG CCCAGCTTGG AGCGAACGAC CTACACCGAA |
| | CTGAGATACC |
| 1401 | TACAGCGTGA GCTATGAGAA AGCGCCACGC TTCCCGAAGG |
| | GAGAAAGGCG |
| 1451 | GACAGGTATC CGGTAAGCGG CAGGGTCGGA ACAGGAGAGC |
| | GCACGAGGGA |
| 1501 | GCTTCCAGGG GGAAACGCCT GGTATCTTTA TAGTCCTGTC |
| | GGGTTTCGCC |
| 1551 | ACCTCTGACT TGAGCGTCGA TTTTTGTGAT GCTCGTCAGG |
| | GGGGCGGAGC |
| 1601 | CTATGGAAAA ACGCCAGCAA CGCGGCCTTT TTACGGTTCC |
| | TGGCCTTTTG |
| 1651 | CTGGCCTTTT GCTCACATGT TCTTTCCTGC GTTATCCCCT |
| | GATTCTGTGG |
| 1701 | ATAACCGTAT TACCGCCTTT GAGTGAGCTG ATACCGCTCG |
| | CCGCAGCCGA |
| 1751 | ACGACCGAGC GCAGCGAGTC AGTGAGCGAG GAAGCGGAAG |
| | AGCGCCTGAT |
| 1801 | GCGGTATTTT CTCCTTACGC ATCTGTGCGG TATTTCACAC |
| | CGCAATGGTG |
| 1851 | CACTCTCAGT ACAATCTGCT CTGATGCCGC ATAGTTAAGC |
| | CAGTATACAC |
| 1901 | TCCGCTATCG CTACGTGACT GGGTCATGGC TGCGCCCCGA |
| | CACCCGCCAA |
| 1951 | CACCCGCTGA CGCGCCCTGA CGGGCTTGTC TGCTCCCGGC |
| | ATCCGCTTAC |
| 2001 | AGACAAGCTG TGACCGTCTC CGGGAGCTGC ATGTGTCAGA |
| | GGTTTTCACC |
| 2051 | GTCATCACCG AAACGCGCGA GGCAGCTGCG GTAAAGCTCA |
| | TCAGCGTGGT |
| 2101 | CGTGAAGCGA TTCACAGATG TCTGCCTGTT CATCCGCGTC |
| | CAGCTCGTTG |
| 2151 | AGTTTCTCCA GAAGCGTTAA TGTCTGGCTT CTGATAAAGC |
| | GGGCCATGTT |
| 2201 | AAGGGCGGTT TTTTCCTGTT TGGTCACTGA TGCCTCCGTG |
| | TAAGGGGGAT |
| 2251 | TTCTGTTCAT GGGGGTAATG ATACCGATGA AACGAGAGAG |
| | GATGCTCACG |
| 2301 | ATACGGGTTA CTGATGATGA ACATGCCCGG TTACTGGAAC |
| | GTTGTGAGGG |
| 2351 | TAAACAACTG GCGGTATGGA TGCGGCGGGA CCAGAGAAAA |
| | ATCACTCAGG |
| 2401 | GTCAATGCCA GCGCTTCGTT AATACAGATG TAGGTGTTCC |
| | ACAGGGTAGC |
| 2451 | CAGCAGCATC CTGCGATGCA GATCCGGAAC ATAATGGTGC |
| | AGGGCGCTGA |
| 2501 | CTTCCGCGTT TCCAGACTTT ACGAAACACG GAAACCGAAG |
| | ACCATTCATG |
| 2551 | TTGTTGCTCA GGTCGCAGAC GTTTTGCAGC AGCAGTCGCT |
| | TCACGTTCGC |
| 2601 | TCGCGTATCG GTGATTCATT CTGCTAACCA GTAAGGCAAC |
| | CCCGCCAGCC |
| 2651 | TAGCCGGGTC CTCAACGACA GGAGCACGAT CATGCGCACC |
| | CGTGGGGCCG |
| 2701 | CCATGCCGGC GATAATGGCC TGCTTCTCGC CGAAACGTTT |
| | GGTGGCGGGA |
| 2751 | CCAGTGACGA AGGCTTGAGC GAGGGCGTGC AAGATTCCGA |
| | ATACCGCAAG |
| 2801 | CGACAGGCCG ATCATCGTCG CGCTCCAGCG AAAGCGGTCC |
| | TCGCCGAAAA |
| 2851 | TGACCCAGAG CGCTGCCGGC ACCTGTCCTA CGAGTTGCAT |
| | GATAAAGAAG |
| 2901 | ACAGTCATAA GTGCGGCGAC GATAGTCATG CCCCGCGCCC |
| | ACCGGAAGGA |
| 2951 | GCTGACTGGG TTGAAGGCTC TCAAGGGCAT CGGTCGAGAT |
| | CCCGGTGCCT |
| 3001 | AATGAGTGAG CTAACTTACA TTAATTGCGT TGCGCTCACT |
| | GCCCGCTTTC |
| 3051 | CAGTCGGGAA ACCTGTCGTG CCAGCTGCAT TAATGAATCG |
| | GCCAACGCGC |
| 3101 | GGGGAGAGGC GGTTTGCGTA TTGGGCGCCA GGGTGGTTTT |
| | TCTTTTCACC |
| 3151 | AGTGAGACGG GCAACAGCTG ATTGCCCTTC ACCGCCTGGC |
| | CCTGAGAGAG |
| 3201 | TTGCAGCAAG CGGTCCACGC TGGTTTGCCC CAGCAGGCGA |
| | AAATCCTGTT |
| 3251 | TGATGGTGGT TAACGGCGGG ATATAACATG AGCTGTCTTC |
| | GGTATCGTCG |
| 3301 | TATCCCACTA CCGAGATATC CGCACCAACG CGCAGCCCGG |
| | ACTCGGTAAT |
| 3351 | GGCGCGCATT GCGCCCAGCG CCATCTGATC GTTGGCAACC |
| | AGCATCGCAG |
| 3401 | TGGGAACGAT GCCCTCATTC AGCATTTGCA TGGTTTGTTG |
| | AAAACCGGAC |
| 3451 | ATGGCACTCC AGTCGCCTTC CCGTTCCGCT ATCGGCTGAA |
| | TTTGATTGCG |
| 3501 | AGTGAGATAT TTATGCCAGC CAGCCAGACG CAGACGCGCC |
| | GAGACAGAAC |
| 3551 | TTAATGGGCC CGCTAACAGC GCGATTTGCT GGTGACCCAA |
| | TGCGACCAGA |
| 3601 | TGCTCCACGC CCAGTCGCGT ACCGTCTTCA TGGGAGAAAA |
| | TAATACTGTT |
| 3651 | GATGGGTGTC TGGTCAGAGA CATCAAGAAA TAACGCCGGA |
| | ACATTAGTGC |
| 3701 | AGGCAGCTTC CACAGCAATG GCATCCTGGT CATCCAGCGG |
| | ATAGTTAATG |
| 3751 | ATCAGCCCAC TGACGCGTTG CGCGAGAAGA TTGTGCACCG |
| | CCGCTTTACA |
| 3801 | GGCTTCGACG CCGCTTCGTT CTACCATCGA CACCACCACG |
| | CTGGCACCCA |
| 3851 | GTTGATCGGC GCGAGATTTA ATCGCCGCGA CAATTTGCGA |
| | CGGCGCGTGC |
| 3901 | AGGGCCAGAC TGGAGGTGGC AACGCCAATC AGCAACGACT |
| | GTTTGCCCGC |
| 3951 | CAGTTGTTGT GCCACGCGGT TGGGAATGTA ATTCAGCTCC |
| | GCCATCGCCG |
| 4001 | CTTCCACTTT TTCCCGCGTT TTCGCAGAAA CGTGGCTGGC |
| | CTGGTTCACC |
| 4051 | ACGCGGGAAA CGGTCTGATA AGAGACACCG GCATACTCTG |
| | CGACATCGTA |
| 4101 | TAACGTTACT GGTTTCACAT TCACCACCCT GAATTGACTC |
| | TCTTCCGGGC |
| 4151 | GCTATCATGC CATACCGCGA AAGGTTTTGC GCCATTCGAT |
| | GGTGTCCGGG |
| 4201 | ATCTCGACGC TCTCCCTTAT GCGACTCCTG CATTAGGAAG |
| | CAGCCCAGTA |
| 4251 | GTAGGTTGAG GCCGTTGAGC ACCGCCGCCG CAAGGAATGG |
| | TGCATGCAAG |
| 4301 | GAGATGGCGC CCAACAGTCC CCCGGCCACG GGGCCTGCCA |
| | CCATACCCAC |
| 4351 | GCCGAAACAA GCGCTCATGA GCCCGAAGTG GCGAGCCCGA |
| | TCTTCCCCAT |
| 4401 | CGGTGATGTC GGCGATATAG GCGCCAGCAA CCGCACCTGT |
| | GGCGCCGGTG |
| 4451 | ATGCCGGCCA CGATGCGTCC GGCGTAGAGG ATCGAGATCT |
| | CGATCCCGCG |
| 4501 | AAATTAATAC GACTCACTAT AGGGGAATTG TGAGCGGATA |
| | ACAATTCCCC |
| 4551 | TCTAGAAATA ATTTTGTTTA ACTTTAAGAA GGAGATATAC |
| | ATATGTTCCC |
| 4601 | GACCATTCCG TTATCCCGTC TTTTTGACAA CGCTATGCTC |
| | CGTGCCCATC |
| 4651 | GTCTGCACCA GCTGGCCTTT GACACCTACC AGGAGTTTGA |
| | AGAAGCCTAT |
| 4701 | ATCCCAAAGG AACAGAAGCA CTCATTCCTG CAGAACCCCC |
| | AGACCTCCCT |
| 4751 | CTGTTTCTCA GAGTCTATTC CGACACCCTC CAACAGGGAG |
| | GAAACACAAC |
| 4801 | AGAAATCCAA CCTAGAGCTG CTCCGCATCT CCCTGCTGCT |
| | CATCCAGTCG |
| 4851 | TGGCTGGAGC CCGTGCAGTT CCTCAGGAGT GTCTTCGCCA |
| | ACAGCCTGGT |
| 4901 | GTACGGCGCC TCTGACAGCA ACGTCTATGA CCTCCTAAAG |
| | GACCTAGAGG |
| 4951 | AAGGCATCCA AACGCTGATG GGGAGGCTGG AAGATGGCAG |
| | CCCCCGGACT |
| 5001 | GGGCAGATCT TCAAGCAGAC CTACAGCAAG TTCGACACAA |
| | ACTCACACAA |
| 5051 | CGATGACGCA CTACTCAAGA ACTACGGGCT GCTCTACTGC |
| | TTCAGGAAGG |
| 5101 | ACATGGACAA GGTCGAGACA TTCCTGCGCA TCGTGCAGTG |
| | CCGCTCTGTG |
| 5151 | GAGGGCAGCT GTGGCTTCTA AGGATCCGAA TTCGAGCTCC |
| | GTCGACAAGC |
| 5201 | TTGCGGCCGC ACTCGATGAG CAATAACTAG CATAACCCCT |
| | TGGGGCCTCT |
| 5251 | AAACGGGTCT TGAGGGGTTT TTTGCTGAAA GGAGGAA |

### Example 22

### Transport Kinetics of hGH(Y42I)

The cellular permeability of hGH(Y42I) was determined using a Caco-2 monolayer assay. Caco-2 is a human colorectal carcinoma cell line that forms a monolayer of differentiated epithelial cells joined by intercellular tight junctions. Because it forms a selective barrier for transcellular transport, the Caco-2 monolayer is commonly used as a model for human drug intestinal permeability. Typically, Caco-2 cells are seeded on a microporous membrane which is suspended in a well. With the monolayer in place, the well is divided into upper (apical) compartment and a lower (basolateral) compartment. To test absorption, the drug sample is applied to the apical compartment and withdrawn from the basolateral compartment at various time points. The apparent permeability coefficient (Pₐₚₚ) can then be calculated from the following equation:

$${P}_{app} ( cm \text{/} s ) = \frac{{V}_{A} \times {C}_{inf}}{dt \times {C}_{T = 0} \times A} \qquad$$

where V_{A} is the volume of the acceptor well, C_{inf} is the concentration of compound (i.e. drug) in the basolateral compartment after transport, A is the surface area (cm²) of the cell monolayer, and C_{T=0} is the concentration in the apical compartment before transport.

a. Permeability Experiments

In this Example, Caco-2 cells were maintained in Dulbecco's modified Eagle medium supplemented with 20% fetal bovine serum, glutamin and gentamicin. Cells were used between 22-28 passages (cells were typically passaged weekly). Cells were seeded at a density of 2×10⁵ cells per well on polycarbonate membrane inserts in 24-well Transwell™ plates (Costar) and used at days 21-23 post-seeding. The integrity of the cell monolayer was validated using TEER (transepithelial electrical resistance) testing wherein the movement of ions across the paracellular pathway is measured. Measurement of TEER across cells grown on permeable membranes provides an indirect assessment of tight junction establishment and stability.

Permeability experiments for hGH(Y42I) compared to native hGH were performed in the apical-basolateral direction (i.e. the concentration of hGH(Y42I) in the basolateral compartment was much lower that that of the apical compartment at the start of the assay). The concentration of hGH(Y42I) in the apical compartment at the start of the assay (C_{T=0}) was 75 μg/mL and the concentration in the basolateral compartment was ˜0. A pre-clinical batch of hGH(Y42I) prepared in accordance with condition #4 as described in Example 23 was used in the assay. The assay was performed in Ringer's buffer, pH 7.8, at 37° C. with shaking. Samples were withdrawn from the basolateral compartment at t=1 hour, 0.2 hours, 3 hours, 4 hours, 5 hours and 6 hours. The concentration of hGH(Y42I) and native hGH at each time point were determined using an hGH ELISA (Mediagnost®). Apparent permeability coefficients (Pₐₚₚ) were calculated for hGH(Y42I) and native hGH at each time point using the equation described above. Approximate Pₐₚₚ values are presented in Table 93:

TABLE 93

| | Pₐₚₚ hGH(Y42I) | Pₐₚₚ native hGH |
|---|---|---|
| Time (hour) | (cm/sec × 10⁸) | (cm/sec × 10⁸) |
| 1 | 0 | 0 |
| 2 | 0.05 | 0.02 |
| 3 | 0.18 | 0.06 |
| 4 | 0.20 | 0.19 |
| 5 | 0.30 | 0.29 |
| 6 | 0.35 | 0.30 |

In a separate experiment, Pₐₚₚ values were determined for various starting concentrations of hGH(Y42I) or native hGH in the apical compartment (C_{T=0}) after 6 hours total transport time. Approximate Pₐₚₚ values are presented in Table 94 below.

TABLE 94

| C_{T=0} | Pₐₚₚ hGH(Y42I) | Pₐₚₚ native hGH |
|---|---|---|
| (μg/ml) | (cm/sec × 10⁸) | (cm/sec × 10⁸) |
| 5 | 0.30 | 0.25 |
| 10 | 0.20 | 0.25 |
| 30 | 0.25 | 0.30 |
| 50 | 0.40 | 0.60 |
| 100 | 1.10 | 1.10 |
| 140 | — | 0.90 |
| 150 | 0.90 | — |
| 200 | 0.90 | — |
| 230 | — | 1.10 |
| 260 | 0.90 | — |
| 270 | — | 0.80 |

As shown in Tables 93 and 94 above, hGH(Y42I) and native hGH show comparable transport kinetics through monolayers of Caco-2 cells.

b. Immunofluorescence Experiments

In order to visualize transport of hGH(Y42I) across Caco-2 cells, immunoflourescence experiments were performed. For these experiments, Caco-2 cells were seeded at a density of 2×10⁵ cells per well on polyester (PET) membrane inserts in 24-well Transwell™ plates (Costar) and used at days 21-23 post-seeding. The permeability assay was conducted as described above except that the starting concentration of hGH(Y42I) in the apical compartment was 300 μg/mL.

After six hours of membrane transport, the cells were fixed in 4% phosphate buffer with sucrose containing 4% formaldehyde for 15 minutes at room temperature. Cells were washed with PBS and stored at 4° C. until staining.

Prior to staining, cells were permeabilized for antibody penetration according to the following method: 1) cells were incubated for 10 minutes at room temperature in TBS containing 1% BSA and 0.1% Triton X-100; 2) cells were washed three times in 1×TBS; and 3) cells were incubated in 1×TBS containing 1% BSA for 2 hours at room temperature.

Cells were stained for: hGH(Y42I), actin (apical membrane marker), transferrin receptor (basal membrane marker) and occludin (tight junction marker). Staining of hGH(Y42I) and cellular markers was performed for 1 hour at room temperature using goat anti-hGH (T20) antibody (Santa Cruz; 10365) at a dilution of 1:50 (for staining of hGH(Y42I)); phalloidin Alexa-568 molecular probe (A12380) at a dilution of 1:200 (for actin staining); mouse anti-transferrin receptor (Zymed; 136800) at a dilution of 1:500 (for transferrin receptor staining); or mouse anti-occludin FITC antibody (Zymed; 33-1511) at a dilution of 1:250 (for occluding staining). Cell monolayers were cut in 50 μl of Vectashield® Mounting Medium with Dapi solution (Vector Laboratories) and cell staining was observed using confocal microscopy. The results showed no difference in fluorescence between native hGH and hGH(Y42I). This supports the results described above that hGH(Y42I) and native hGH show comparable transport kinetics through monolayers of Caco-2 cells.

### Example 23

### hGH(Y42I) Drug Product Development Studies for Pre-Clinical and Clinical Use

Using methods of production analogous to those described for IFN-α-2b(E41Q) herein, a liquid bulk solution containing hGH(Y42I) protein was generated for use in pre-clinical and clinical studies. Several exemplary buffer conditions for lyophilization were tested and the data from these tests are presented below. The lyophilized powder generated from the selected condition was used to manufacture hGH(Y42I) enteric-coated caplets and tablets. Exemplary drug formulations are presented below.

1. Selection of Lyophilization Conditions

Prior to lyophilization of the liquid bulk for manufacture of enteric-coated caplets and tablets, it was necessary to test several buffer conditions of the liquid bulk in order to generate an optimal powder substance for the capsule and tablet formulations. Six exemplary buffer conditions are presented in Table 95 below. Samples of hGH(Y42I) protein obtained purified liquid bulk preparation of E. coli-expressed hGH(Y42I) protein were formulated in the six different buffers. Concentrations of buffer components presented in Table 95 represent the final buffer concentrations prior to lyophilization.

TABLE 95
Lyophilization buffer conditions

| Condition | Buffer Formulation |
|---|---|
| Condition 1 | 20 g/L Mannitol + 1 g/L NaCl + 7.5 mM Na₂HPO₄/ |
| | NaH₂PO₄, pH 7.5 |
| Condition 2 | 2 g/L Mannitol + 15 mM Na₂HPO₄/NaH₂PO₄, pH 7.5 |
| Condition 3 | 7.5 g/L Sucrose + 2 mM H₃PO₄/NaOH, pH 6.5 |
| Condition 4 | 6 g/L Sucrose + 7.5 g/L NaCl + 20 mM Na₂HPO₄/ |
| | NaH₂PO₄, pH 7.5 |
| Condition 5 | 6 g/L Sucrose + 20 mM Na₂HPO₄/NaH₂PO₄, pH 7.5 |
| Condition 6 | 6 g/L Sucrose + 8.5 g/L arginine + 20 mM Na₂HPO₄/ |
| | NaH₂PO₄, pH 7.5 |

The activity, percent yield and percent dimerization of hGH(Y42I) were analyzed for each liquid sample prior to lyophilization. The activity was determined using the hGH cell proliferation assay described in Example 1. SE-HPLC was used for the monomer/dimer determination. All conditions were deemed acceptable for lyophilization according the parameters tested. The results are presented in Table 96:

TABLE 96
Activity, yield, and dimer formation in tested buffers

| | Activity | | |
|---|---|---|---|
| Sample | (EC₅₀, pg/mL) | Yield (%) | Dimer (%) |
| Initial Sample | 83.54 | n/a | 5.92 |
| Condition 1 | 63.26 | 86.74 | 10.23 |
| Condition 2 | 78.82 | 87.11 | 4.40 |
| Condition 3 | 63.32 | 95.03 | 5.49 |
| Condition 4 | 72.84 | 91.55 | 7.46 |
| Condition 5 | 70.45 | 94.65 | 8.83 |
| Condition 6 | 69.69 | 88.98 | 10.23 |
| NIBSC | 101.59 | n/a | n/a |

The samples were lyophilized using procedures analogous to those described for IFN-α-2b(E41Q). Following lyophilization, the appearance of the powder was analyzed for each condition. The results of the analysis are presented Table 97 below.

TABLE 97
Lyophilized powder quality

| Sample | Appearance of Powder |
|---|---|
| Condition 1 | Voluminous; Very lumpy; Marginal adherence to wall of |
| | vial |
| Condition 2 | Fine; Lumpy; Adherence to wall and spatula |
| Condition 3 | More voluminous than condition 4; Very lumpy; No |
| | adherence to wall or spatula |
| Condition 4 | Very fine; Not lumpy; Marginal adherence to wall of vial |
| Condition 5 | More voluminous than condition 4; Marginal lumpiness; |
| | Marginal adherence to wall and spatula |
| Condition 6 | Marginally voluminous; Not lumpy; Very adherent to wall |
| | and spatula |

Based on the physical behavior (i.e., stickiness) of the powder, conditions 2 and 6 were eliminated and conditions 1, 3, 4 and 5 were chosen for further analysis. Following the physical analysis, samples of the lyophilized powder were resuspended and analyzed for activity (by hGH cell proliferation assay), yield (determined by OD₂₈₀ and bicinchoninic acid (BCA) assays) and percent dimerization (by SE-HPLC). The results of these analyses are presented in Table 98 below.

TABLE 98
Activity, yield and dimer data for lyophilized samples

| | | Yield | | | |
|---|---|---|---|---|---|
| | Activity | | | BCA/OD₂₈₀ | |
| | (EC₅₀, | | BCA | (% | |
| Sample | pg/mL) | OD₂₈₀ | (mg/mL) | variance) | Dimer (%) |
| Condition 1 | 188.72 | 0.346 | 0.527 | 34 | 9.97 |
| Condition 2 | 140.97 | 0.296 | n/a* | n/a* | 7.19 |
| Condition 3 | 137.93 | 0.277 | 0.479 | 42 | 6.94 |
| Condition 4 | 121.67 | 0.325 | 0.550 | 41 | 6.72 |
| Condition 5 | 135.96 | 0.282 | 0.510 | 45 | 10.05 |
| Condition 6 | 151.37 | 0.230 | n/a* | n/a* | 10.88 |
| NIBSC | 144.7 | n/a | n/a | n/a | n/a |

*Since condition 2 and condition 6 were eliminated from further consideration, BCA assays were not performed on these samples

2. Addition of Chromatography Purification Step

In a second experiment, a column purification step was added to the lyophilization process prior to the lyophilization step in order to determine the degree of purity that can be achieved with each buffer condition. Samples of hGH(Y42I) protein were prepared as described above in 23.1 using the specified buffer for conditions 1, 3, 4 and 5. The liquid bulk (prior to lyophilization) was purified by size exclusion chromatography (SEC) using Sephacryl S100 columns. Prior to addition of the samples, the columns were equilibrated with the appropriate buffer (without the sugar) for each condition. The protein samples were then loaded, washed and eluted. The elution fractions for each sample were analyzed for yield and purity (determined by peak resolution in mass spectrophotometry (mass spec) analysis). Samples for conditions 1, 4, and 5 exhibited good resolution of the hGH peak when analyzed with mass spec. The sample for condition 3 was found to have poor peak resolution. The results of the analysis are presented in Table 99 below.

TABLE 99
SEC yields and resolution quality

| Sample | Yield (%) | Resolution Quality |
|---|---|---|
| Condition 1 | 82.6 | Good |
| Condition 3 | 78.2 | Poor |
| Condition 4 | 77 | Good |
| Condition 5 | 77.8 | Good |

Following SEC purification, the liquid bulk samples from conditions 1, 3, 4 and 5 were lyophilized as described above. The amount of hGH(Y42I) protein per mg of powder was determined from OD₂₈₀ and BCA measurements. In addition, the maximum amount of powder that could be loaded into a size 9 capsule was measured. Based on these amounts (mg of protein per mg of powder and mg of powder per capsule) the maximum amount of hGH(Y42I) protein that could be contained in a capsule was calculated for each of the four selected conditions. This data is presented in Table 100 below. A summary of the data collected during lyophilization optimization also is presented in Table 100, including powder appearance, hGH(Y42I) activity after lyophilization, % dimerization of hGH(Y42I) before and after lyophilization, and resolution quality of the mass spec peaks after SEC purification.

TABLE 100
Summary of SEC purified lyophilized samples

| | | | | OD₂₈₀ | | | |
|---|---|---|---|---|---|---|---|
| | | Activity | | BCA | Mg | | |
| | Powder | (EC₅₀, | | (mg hGH/ | powder/ | Mg hGH/ | SEC |
| Sample | Appearance | pg/mL) | Dimer (%) | mg powder) | Capsule | Capsule | Purification |
| Condition | Good | 188.72 | Before 10.23 | OD: 0.027 | 22.3 | 0.594 | Good |
| 1 | | | After 9.97 | BCA: 0.041 | | | resolution |
| Condition | Sticky | 140.97 | Before 4.4 | n/a | n/a | n/a | n/a |
| 2 | | | After 7.19 | | | | |
| Condition | Good | 137.93 | Before 5.49 | OD: 0.057 | 26.2 | 1.508 | Good |
| 3 | | | After 6.94 | BCA: 0.099 | | | resolution |
| Condition | Good | 121.67 | Before 7.46 | OD: 0.033 | 36.6 | 1.227 | Good |
| 4 | | | After 6.72 | BCA: 0.057 | | | resolution |
| Condition | Good | 135.96 | Before 8.83 | OD: 0.057 | 24.6 | 1.398 | Good |
| 5 | | | After 10.05 | BCA: 0.103 | | | resolution |
| Condition | Sticky | 151.37 | Before 10.23 | n/a | n/a | n/a | n/a |
| 6 | | | After 10.88 | | | | |
| NIBSC | n/a | 144.76 | n/a | n/a | n/a | n/a | n/a |

As a result of these studies, condition 4 was selected for further drug product formulation of hGH(Y42I) based on its good peak resolution and low dimer percentage.

### Example 24

### Normal Versus Low Moisture Formulation of hGH(Y42I) Tablets

For the tablet formulations of hGH(Y42I), the effect of moisture content on the stability of hGH(Y42I) was examined. Two exemplary hGH(Y42I) tablet formulations, referred to herein as normal and low moisture, were manufactured and tested. The formulations were produced by separate manufacturers. The specifications for these two formulations are presented in Table 101 below.

TABLE 101
Low versus Normal moisture tablet formulations

| | Moisture | Normal | Low Moisture |
|---|---|---|---|
| | Content (%) | Formulation | Formulation |
| Formulation | | | |
| hGH(Y42I) Powder | — | 8.7 | 8.7 |
| Lactose Monohydrate | 4.5-5.5 | 68.5 | — |
| DCL 11 | | | |
| Pharmatose DCL 22 | — | — | 67.9 |
| Avicel PH102 | <5.0 | 18 | — |
| Avicel PH112 | <1.5 | — | 16.8 |
| Povidone K25 | 10.0-15.0 | 1.2 | — |
| Crosspovidone XL | <5.0 | 2.4 | — |
| Crosscarmellose Sodium | <10 | — | 3.0 |
| Pearitol 200 SD | <1.0 | — | 2.0 |
| Glycine | — | — | 0.4 |
| Stearic Acid | — | — | 1.2 |
| Magnesium Stearate | — | 1.2 | — |
| Total | | 100.0 | 100.0 |
| Process Steps | | | |
| 1 | | Dry Blending | Dry Blending |
| 2 | | Compression | Compression |
| 3 | | — | Cooling and |
| | | | Drying |
| 4 | | Enteric Coating | Sub-coating + |
| | | | Enteric Coating |
| Tooling used | | 11.00 mm × | 11.00 mm × |
| | | 4.40 | 4.40 |
| | | mm capsule | mm capsule |
| | | shape tooling | shape tooling |
| PH of Excipient Blend | | 6.676 | 6.415 |
| (10% solution) | | | |
| Tablet Characteristics | | | |
| Tablet Weight (mg) | | 150 ± 3 | 150 ± 3 |
| Tablet Thickness (mm) | | 3.11-3.17 | 3.16-3.25 |
| Tablet Hardness (KP) | | 6.7 | 6.9 |
| Average Compression | | 140 bar | 120 bar |
| Force | | | |
| Disintegration Time as | | | |
| per USP (701) for | | | |
| Delayed | | | |
| Release Tablets | | | |
| (min.: sec.) | | | |
| Enteric-coated Tablets | | 10:47 | 12:09 |
| Coating Parameters | | | |
| Inlet Air Temperature | | 34-36 | 34-36 |
| ° C. | | | |
| Product Temperature | | 26-28 | 26-28 |
| ° C. | | | |
| Weight Gain (%) | | | |
| Sub-coating with Opadry | | — | 2.7 |
| Clear | | | |
| Enteric Coating with | | 13.0 | 13.4 |
| Acryl EZE | | | |

Stability analysis was performed on normal (normal WC) and low moisture (low WC) formulation tablets in a climactic chamber (30° C./60% relative humidity (RH) and 45° C./75% RH). Monomer/dimer determination (SE-HPLC) and activity (hGH cell proliferation assay) data was collected at each time point. The results of the tests are presented in Table 102 below. Based on this study, the low moisture formulation was chosen for the manufacture of the hGH(Y42I) tablets.

TABLE 102
Stability analysis for low versus normal moisture content

| | | | | | Proliferation | |
|---|---|---|---|---|---|---|
| | | | | | Activity | |
| Formu- | | Length | | Dimer | (EC₅₀, pg/mL) | |
| lation | Time | (Week) | Condition | % | Tablet | NIBSC |
| low WC | T0 | 0 | RT | 3.2 | 89.7 | 128.8 |
| normal | T0 | 0 | RT | 4.18 | 95.8 | 128.8 |
| WC | | | | | | |
| low | T1 | 4 | 30° C. 60% RH | 5 | 238 | 126.8 |
| WC | | | 45° C. 75% RH | 6.72 | 290 | 126.8 |
| normal | T1 | 4 | 30° C. 60% RH | 6.42 | 280.3 | 126.8 |
| WC | | | 45° C. 75% RH | 8.6 | 269.8 | 126.8 |
| low WC | T2 | 6 | 30° C. 60% RH | 7.79 | 97.1 | 89.4 |
| | | | 45° C. 75% RH | 9.51 | 208.3 | 89.4 |
| normal | T2 | 6 | 30° C. 60% RH | 19.12 | 485.5 | 89.4 |
| WC | | | 45° C. 75% RH | 17.26 | 351 | 89.4 |

### Example 25

### Exemplary Capsule and Tablet Formulations of hGH(Y42I)

1. Enteric-Coated Capsules

Enteric-coated capsules for use in pre-clinical rat studies were filled with the appropriate amount of lyophilized hGH(Y42I). Filler weight was completed with sucrose. The capsules were enteric-coated (to maintain their integrity at acidic pH) to deliver the product into the intestine (soluble at neutral pH). Clear Torpac (size 9, batch # 1684) and Opaque Capsugel capsules (size 9, batch # 1402) were used for PK rat studies.

The enteric coating was comprised of 3 layers of a solution containing cellulose/acetate/phatalate in acetone (12.5% m/v). Each layer was dried separately. The quality of the enteric coating was evaluated by exposure to pH 1.2 for 1 h at 37° C., with stirring followed by a full dissolution in PBS at 37° C. This procedure was then applied to the different dose strength enteric-coated capsules used in pre-clinical studies. All tested enteric-coated capsules showed no visual leaks during the 1 h exposure. The transfer into PBS elicited disintegration of the enteric-coated capsule within 30 min.

2. Enteric-Coated Tablets

Exemplary formulations for enteric coated hGH(Y42I) tablets are presented in Table 103 below.

TABLE 103
hGH(Y42I) Enteric-coated Tablet Formulation

| | 3.0 mg | | 12.0 mg | | 24.0 mg | |
|---|---|---|---|---|---|---|
| Ingredients | strength | | strength | | strength | |
| hGH(Y42I) Lyophilized Powder | 21.75 | mg | 87.0 | mg | 174.0 | mg |
| Pearlitol 200 SD | 15.0 | mg | 60.0 | mg | 120.0 | mg |
| Glycine | 3.0 | mg | 12.0 | mg | 24.0 | mg |
| Pharmatose DCL 22 | 172.125 | | 112.5 | mg | 33.0 | mg |
| Avicel 112 | 172.125 | | 112.5 | mg | 33.0 | mg |
| Croscarmellose Sodium | 12.0 | mg | 12.0 | mg | 12.0 | mg |
| Stearic Acid | 4.0 | mg | 4.0 | mg | 4.0 | mg |
| Total Tablet Weight | 400.0 | mg | 400.0 | mg | 400.0 | mg |

### Example 26

### Pharmacokinetic (PK) Profiles of Exemplary hGH Protease-Resistant Mutants Intraduodenal Versus Subcutaneous Administration

The pharmacokinetic (PK) profile following a single intraduodenal (ID) administration or subcutaneous injection of either native hGH protein or one of several hGH protease-resistant variants was compared. The hGH variants used in this study are referred to by the following variant numbers (the corresponding hGH mutation is shown in parentheses): variant 15 (D11N), variant 18 (M14V), variant 27 (L231), variant 28 (L23V), variant 57 (Y42H), variant 58 (Y42I), variant 100 (L81V), variant 143 (E119Q) and variant 148 (M125I). The study involved 120 8-week old Sprague-Dawley male rats (20 groups of 6 animals) and was conducted in two parts: 1) ID administration and 2) subcutaneous injection.

1. Part I: Intraduodenal Dosing

For the ID administration, 2 mg per animal of a liquid formulation (1 mL/animal volume) of native hGH or variant hGH (15, 18, 27, 28, 58, 57, 100, 143 or 148) was administered to 8-week old pathogen free Sprague-Dawley rats via an intraduodenal catheter. Blood samples were taken from all the animals for determination of plasma levels of native hGH protein and hGH variants 15, 18, 27, 28, 58, 57, 100, 143 and 148 (pharmacokinetic studies) in rat serum.

The treatment groups (10 groups of 6 male rats including a control group) established for the study are presented in Table 104:

TABLE 104
Dosage and experimental detail for ID study

| Group | | | Dose and | |
|---|---|---|---|---|
| number | Number of animals | Treatment | volume | |
| 1 | 6 males (1 to 6) | Native hGH | 2 | mg |
| | | | 1 | ml |
| 2 | 6 males (7 to 12) | hGH variant 15 | 2 | mg |
| | | | 1 | ml |
| 3 | 6 males (13 to 18) | hGH variant 18 | 2 | mg |
| | | | 1 | ml |
| 4 | 6 males (19 to 24) | hGH variant 27 | 2 | mg |
| | | | 1 | ml |
| 5 | 6 males (25 to 30) | hGH variant 28 | 2 | mg |
| | | | 1 | ml |
| 6 | 6 males (31 to 36) | hGH variant 58 | 2 | mg |
| | | | 1 | ml |
| 7 | 6 males (37 to 42) | hGH variant 57 | 2 | mg |
| | | | 1 | ml |
| 8 | 6 males (43 to 48) | hGH variant 100 | 2 | mg |
| | | | 1 | ml |
| 9 | 6 males (49 to 54) | hGH variant 143 | 2 | mg |
| | | | 1 | ml |
| 10 | 6 males (55 to 60) | hGH variant 148 | 2 | mg |
| | | | 1 | ml |

Blood samples were drawn on administration day 1 for each group immediately before administration and at 0.5, 1, 2, 4 and 8 hours after administration. At each time-point, a blood sample (200 μl) was taken via a jugular vein catheter and placed in an ice-cold collection tube containing lithium-heparin and was gently mixed. 30 μl of anti-protease solution was immediately added. Tubes were kept in ice (within 30 minutes of collection) and centrifuged at 2000×g for 10 minutes at 4° C. Plasma was harvested in 3 tubes of at least 40 μl each and kept frozen at −20° C. or −80° C. until assayed for presence of hGH.

The amount of remaining hGH in animal plasma at each time point was determined using an anti-hGH ELISA kit (Roche Diagnostics GmbH, reference n^{o}11585 878 001), according to the manufacturer's protocol. Human hGH ELISA Kit results were expressed in pg/ml after extrapolation from the standard curve of OD values measured with a spectrophotometer at 405 nm. Using PK solution software (SummitPK, Montrose, Colo., USA), PK parameters including Tₘₐₓ, Cₘₐₓ, half-life and AUC were determined for each pharmacokinetic profile. PK parameters calculated from the average of animal PK profiles obtained following ID liquid formulation administration of hGH variants and native hGH protein are presented in Table 105.

TABLE 105
PK parameters for ID administration

| Intraduodenal | Cₘₐₓ | AUC₍₀₋ₜ₎ | Tₘₐₓ |
|---|---|---|---|
| (2 mg/animals) | (ng/ml) | (h-ng/ml) | (h) |
| Variant 15 | 431.4 | 1548.7 | 0.5 |
| Variant 18 | 367.6 | 1281.6 | 0.5 |
| Variant 27 | 313.1 | 720.4 | 0.5 |
| Variant 28 | 363.3 | 1278.3 | 0.5 |
| Variant 57 | 361.3 | 1363.3 | 1 |
| Variant 58 | 223.0 | 983.8 | 0.5 |
| Variant 100 | 289.9 | 1067.0 | 0.5 |
| Variant 143 | 274.7 | 575.0 | 1 |
| Variant 148 | 266.8 | 1079.0 | 2 |
| Native hGH | 22.3 | 16.2 | 0.5 |

All protease-resistant hGH variants tested exhibited significantly improved pharmacokinetic profiles compared to native hGH per intraduodenal administration. For example, the variants exhibited Cₘₐₓ values that were 12 to 20 times that of native hGH and AUC values that range from 35 to 96 times that of native hGH. Similar to results observe for other protease-resistant polypeptides, such as IFN-α-2b(E41Q) described in the Examples above, protease resistant variants of hGH exhibit PK profiles that suggest a sustained delivery of the protease-resistant polypeptide from the intestinal compartment to the systemic circulation most likely due to a slow absorption coupled with decreased susceptibility to intestinal protease degradation.

2. Part II: Subcutaneous Dosing

For the subcutaneous administration, 50 μg per animal of native hGH or variant hGH (15, 18, 27, 28, 58, 57, 100, 143 or 148) in a volume of 0.5 mL per animal was administered via the subcutaneous route into 8-week old pathogen free Sprague-Dawley rats. Blood samples were taken from all the animals for determination of plasma levels of native hGH protein and hGH variants 15, 18, 27, 28, 58, 57, 100, 143 and 148 (pharmacokinetic studies) in rat serum.

The treatment groups (10 groups of 6 male rats including a control group) established for the study are presented in Table 106:

TABLE 106
Dosage and experimental detail for SC study

| Group | | | Dose and | |
|---|---|---|---|---|
| number | Number of animals | Treatment | volume | |
| 11 | 6 males (61 to 66) | Native hGH | 50 | μg |
| | | | 0.5 | ml |
| 12 | 6 males (67 to 72) | hGH variant 15 | 50 | μg |
| | | | 0.5 | ml |
| 13 | 6 males (73 to 78) | hGH variant 18 | 50 | μg |
| | | | 0.5 | ml |
| 14 | 6 males (79 to 84) | hGH variant 27 | 50 | μg |
| | | | 0.5 | ml |
| 15 | 6 males (85 to 90) | hGH variant 28 | 50 | μg |
| | | | 0.5 | ml |
| 16 | 6 males (91 to 96) | hGH variant 58 | 50 | μg |
| | | | 0.5 | ml |
| 17 | 6 males (97 to 102) | hGH variant 57 | 50 | μg |
| | | | 0.5 | ml |
| 18 | 6 males (103 to 108) | hGH variant 100 | 50 | μg |
| | | | 0.5 | ml |
| 19 | 6 males (109 to 114) | hGH variant 143 | 50 | μg |
| | | | 0.5 | ml |
| 20 | 6 males (115 to 120) | hGH variant 148 | 50 | μg |
| | | | 0.5 | ml |

Blood samples were drawn on administration day 1 for each group immediately before administration and at 0.5, 1, 2, 4 and 8 hours after administration. At each time-point, a blood sample (200 μl) was taken via a jugular vein catheter and placed in an ice-cold collection tube containing lithium-heparin and was gently mixed. 30 μl of anti-protease solution was immediately added. Tubes were kept in ice (within 30 minutes of collection) and centrifuged at 2000 g for 10 minutes at 4° C. Plasma was harvested in 3 tubes of at least 40 μl each and kept frozen at −20° C. or −80° C. until assayed for the presence of hGH.

The amount of remaining hGH in animal plasma at each time point was determined using an anti-hGH ELISA kit (Roche Diagnostics GmbH, reference n^{o}11585 878 001), according to the manufacturer's protocol. Human hGH ELISA Kit results were expressed in pg/ml after extrapolation from the standard curve of OD values measured with a spectrophotometer at 405 nm. Using PK solution software (SummitPK, Montrose, Colo., USA), PK parameters including Tₘₐₓ, Cₘₐₓ, half-life and AUC were determined for each pharmacokinetic profile. PK parameters calculated from the average of animal PK profiles obtained following subcutaneous administration of hGH variants and native hGH protein are presented in Table 107.

TABLE 107
PK parameters for SC administration

| Subcutaneous | Cₘₐₓ | AUC₍₀₋ₜ₎ | Tₘₐₓ |
|---|---|---|---|
| (50 μg/animals) | (ng/ml) | (h-ng/ml) | (h) |
| Variant 15 | 355.6 | 2204.6 | 2 |
| Variant 18 | 353.5 | 2494.3 | 1 |
| Variant 27 | 370.9 | 1917.3 | 1 |
| Variant 28 | 417.6 | 2335.8 | 1 |
| Variant 57 | 433.6 | 2609.3 | 1 |
| Variant 58 | 277.6 | 1845.2 | 1 |
| Variant 100 | 329.5 | 1785.8 | 1 |
| Variant 143 | 321.6 | 2066.9 | 1 |
| Variant 148 | 322.0 | 2144.4 | 2 |
| Native hGH | 362.4 | 205.1 | 0.5 |

Similar to the results observed for the ID administration, all protease-resistant hGH variants tested exhibited significantly improved pharmacokinetic profiles compared to native hGH per subcutaneous administration. For example, the variants exhibited AUC values that range from 8 to 12 times that of native hGH.

### Example 27

### In Vivo Pharmacokinetic (PK) Study of hGH(Y42I) and hGH(Y42H) in Normal Rats Compared to Native hGH Following Per Os (PO) and IV Administrations

Pharmacokinetic studies were performed on 8-week old Sprague-Dawley male rats to compare per os (PO, liquid gavage and capsule formulations) and intravenous (IV) routes of administration. The objective of this study was to investigate the PK profile and calculate the bioavailability of hGH variant 57 (Y42H) and hGH variant 58 (Y42I). The study involved 120 Sprague-Dawley male rats (24 groups of 5 animals) and was conducted in two parts: 1) per os for liquid and capsule formulations and 2) intravenous injection.

1. hGH Formulations

Preparations of native hGH and hGH variants used are set forth in Tables 108(a-k).

TABLE 108a
hGH WT Batch # hGH-004/06
(Group 1)

| | |
|---|---|
| Date of manufacture | June 2006 |
| Expiry date | December 2006 |
| Correspondence MIU vs mg | 171 MIU = 1 mg |
| Presentation | 65 mL of a WT hGH solution in: |
| | 7.5 mg/mL NaCl, |
| | 6 mg/mL sucrose, |
| | 2.38 mg/mL sodium phosphate dibasic, |
| | 0.44 mg/mL sodium phosphate monobasic |
| Administration | Per os |

TABLE 108b
hGH WT Batch # hGH-004/06
(Group 13: 2 capsules)

| | |
|---|---|
| Date of manufacture | June 2006 |
| Expiry date | December 2006 |
| Correspondence MIU vs mg | 171 MIU = 1 mg |
| Presentation | Capsules containing 0.6 mg of lyophilized |
| | WT hGH prepared in (prior to |
| | lyophilization): |
| | 7.5 mg/mL NaCl, |
| | 6 mg/mL sucrose, |
| | 2.38 mg/mL sodium phosphate dibasic, |
| | 0.44 mg/mL sodium phosphate monobasic |
| Administration | Per os |

TABLE 108c
hGH WT Batch # hGH-004/06
(Group 10, 22)

| | |
|---|---|
| Date of manufacture | June 2006 |
| Expiry date | December 2006 |
| Correspondence MIU vs mg | 171 MIU = 1 mg |
| Presentation | Sterile solution at 100 μg/mL |
| | Preparation: |
| | 13.13 mL of WT hGH solution in |
| | 7.5 mg/mL NaCl, |
| | 6 mg/mL sucrose, |
| | 2.38 mg/mL sodium phosphate dibasic, |
| | 0.44 mg/mL sodium phosphate monobasic |
| | added to 51.87 mL of PBS |
| Administration | Intravenous route |

TABLE 108d
hGH(Y42I) Batch # hGH-003/06
(0.3 mg/mL PO gavage (Group 8) and 0.15 mg/mL PO gavage (Group 9))

| | |
|---|---|
| Date of manufacture | June 2006 |
| Expiry date | December 2006 |
| Correspondence MIU vs mg | 138 MIU = 1 mg |
| Presentation | Preparation (0.3 mg/mL): |
| | 35.9 mL of hGH Lead 58 solution in |
| | 7.5 mg/mL NaCl, |
| | 6 mg/mL sucrose, |
| | 2.38 mg/mL sodium phosphate dibasic, |
| | 0.44 mg/mL sodium phosphate monobasic |
| | added to 29.1 mL of PBS |
| | Preparation (0.15 mg/mL): |
| | 18 mL of hGH Lead 58 solution in |
| | 7.5 mg/mL NaCl, |
| | 6 mg/mL sucrose, |
| | 2.38 mg/mL sodium phosphate dibasic, |
| | 0.44 mg/mL sodium phosphate monobasic |
| | added to 47 mL of PBS |
| Administration | Per os |

TABLE 108e
hGH(Y42I) Batch # hGH-003/06
(0.6 mg/capsule (Groups 18, 19), 0.3 mg/capsule (Group 20),
0.15 mg/capsule (Group 21))

| | |
|---|---|
| Date of manufacture | June 2006 |
| Expiry date | December 2006 |
| Correspondence MIU vs mg | 138 MIU = 1 mg |
| Presentation | Capsules containing 0.6, 0.3 and 0.15 mg |
| | of hGH Lead 58 solution in |
| | 7.5 mg/mL NaCl, |
| | 6 mg/mL sucrose, |
| | 2.38 mg/mL sodium phosphate dibasic, |
| | 0.44 mg/mL sodium phosphate monobasic |
| Administration | Per os |

TABLE 108f
hGH(Y42I) Batch # hGH-003/06
(1.2 mg/mL PO gavage (Group 6) and 0.6 mg/mL PO gavage (Group 7))

| | |
|---|---|
| Date of manufacture | June 2006 |
| Expiry date | December 2006 |
| Correspondence MIU vs mg | 138 MIU = 1 mg |
| Presentation | Preparation (1.2 mg/mL): |
| | 47 mL of hGH Lead 58 solution in |
| | 7.5 mg/mL NaCl, |
| | 6 mg/mL sucrose, |
| | 2.38 mg/mL sodium phosphate dibasic, |
| | 0.44 mg/mL sodium phosphate monobasic |
| | added to 18 mL of PBS |
| | Preparation (0.6 mg/mL): |
| | 23.5 mL of hGH Lead 58 solution in |
| | 7.5 mg/mL NaCl, |
| | 6 mg/mL sucrose, |
| | 2.38 mg/mL sodium phosphate dibasic, |
| | 0.44 mg/mL sodium phosphate monobasic |
| | added to 41.5 mL of PBS |
| Administration | Per os |

TABLE 108g
hGH(Y42I) Batch # hGH-003/06
(Group 12, 24)

| | |
|---|---|
| Date of manufacture | June 2006 |
| Expiry date | December 2006 |
| Correspondence MIU vs mg | 138 MIU = 1 mg |
| Presentation | Sterile solution at 100 μg/mL |
| | Preparation: |
| | 13.57 mL of hGH Lead 58 solution in |
| | 7.5 mg/mL NaCl, |
| | 6 mg/mL sucrose, |
| | 2.38 mg/mL sodium phosphate dibasic, |
| | 0.44 mg/mL sodium phosphate monobasic |
| | added to 51.43 mL of PBS |
| Administration | Intravenous route |

TABLE 108h
hGH(Y42H) Batch # hGH57-002/06
(0.3 mg/mL PO gavage (Group 4) and 0.15 mg/mL PO gavage (Group 5))

| | |
|---|---|
| Date of manufacture | May 2006 |
| Expiry date | November 2006 |
| Correspondence MIU vs mg | 141 MIU = 1 mg |
| Presentation | Preparation (0.3 mg/mL): 41.6 mL of hGH |
| | Lead 58 solution in |
| | 7.5 mg/mL NaCl, |
| | 6 mg/mL sucrose, |
| | 2.38 mg/mL sodium phosphate dibasic, |
| | 0.44 mg/mL sodium phosphate monobasic |
| | added to 23.4 mL of PBS |
| | Preparation (0.15 mg/mL): 20.8 mL of |
| | hGH Lead 58 solution in |
| | 7.5 mg/mL NaCl, |
| | 6 mg/mL sucrose, |
| | 2.38 mg/mL sodium phosphate dibasic, |
| | 0.44 mg/mL sodium phosphate monobasic |
| | added to 44.2 mL of PBS |
| Administration | Per os |

TABLE 108i
hGH(Y42H) Batch # hGH57-002/06
(0.6 mg/capsule (Group 14, 15), 0.3 mg/capsule (Group 16),
0.15 mg/capsule
(Group 17))

| | |
|---|---|
| Date of manufacture | May 2006 |
| Expiry date | November 2006 |
| Correspondence MIU vs mg | 141 MIU = 1 mg |
| Presentation | Capsules containing 0.6, 0.3 and 0.15 mg |
| | of hGH Lead 58 solution in |
| | 7.5 mg/mL NaCl, |
| | 6 mg/mL sucrose, |
| | 2.38 mg/mL sodium phosphate dibasic, |
| | 0.44 mg/mL sodium phosphate monobasic |
| Administration | Per os |

TABLE 108j
hGH(Y42H) Batch # hGH57-002/06
(1.2 mg/mL PO gavage (Group 2) and 0.6 mg/mL PO gavage (Group3))

| | |
|---|---|
| Date of manufacture | May 2006 |
| Expiry date | November 2006 |
| Correspondence | 141 MIU = 1 mg |
| MIU vs mg | |
| Presentation | Preparation (1.2 mg/mL): 45.9 mL of hGH Lead 58 |
| | solution in |
| | 7.5 mg/mL NaCl, |
| | 6 mg/mL sucrose, |
| | 2.38 mg/mL sodium phosphate dibasic, |
| | 0.44 mg/mL sodium phosphate monobasic |
| | added to 19.1 mL of PBS |
| | Preparation (0.6 mg/mL): 23 mL of hGH Lead 58 |
| | solution in |
| | 7.5 mg/mL NaCl, |
| | 6 mg/mL sucrose, |
| | 2.38 mg/mL sodium phosphate dibasic, |
| | 0.44 mg/mL sodium phosphate monobasic added |
| | to 42 mL of PBS |
| Administration | Per os |

TABLE 108k
hGH(Y42H) Batch # hGH57-002/06
(Group 12, 24)

| | |
|---|---|
| Date of manufacture | May 2006 |
| Expiry date | November 2006 |
| Correspondence | 141 MIU = 1 mg |
| MIU vs mg | |
| Presentation | Sterile solution at 100 μg/mL |
| | Preparation: 15.82 mL of hGH Lead 58 solution in |
| | 7.5 mg/mL NaCl, 6 mg/mL sucrose, 2.38 mg/mL |
| | sodium phosphate dibasic, 0.44 mg/mL sodium |
| | phosphate monobasic added to 49.18 mL of PBS |
| Administration | Intravenous route |

2. Part I: Per Os (PO) Dosing

For PO administration, 8-week old pathogen free Sprague-Dawley rats (301-325 g) were administered a liquid formulation by liquid gavage or capsule composition. The animals were administered either 1.2 mg of native hGH or 1.2, 0.6, 0.3 and 0.15 mg of the either hGH(Y42H) or hGH(Y42I). The treatment groups are presented in Table 109: groups 1-9 were used for PO administration of the liquid formulation and groups 13-21 for PO administration of the capsule formulation. The numbers in parenthesis represent the animal reference numbers in the study.

TABLE 109
Dosage and experimental detail for oral administration study

| Group | Main study | | |
|---|---|---|---|
| number | Number of animals | Treatment | Dose and volume |
| 1 | 5 (1 to 5) | Native hGH | 1.2 mg in 1 mL |
| 2 | 5 (6 to 10) | hGH(Y42H) | 1.2 mg in 1 mL |
| 3 | 5 (11 to 15) | hGH(Y42H) | 0.6 mg in 1 mL |
| 4 | 5 (16 to 20) | hGH(Y42H) | 0.3 mg in 1 mL |
| 5 | 5 (21 to 25) | hGH(Y42H) | 0.15 mg in 1 mL |
| 6 | 5 (26 to 30) | hGH(Y42I) | 1.2 mg in 1 mL |
| 7 | 5 (31 to 35) | hGH(Y42I) | 0.6 mg in 1 mL |
| 8 | 5 (36 to 40) | hGH(Y42I) | 0.3 mg in 1 mL |
| 9 | 5 (41 to 45) | hGH(Y42I) | 0.15 mg in 1 mL |
| 13 | 5 (61 to 65) | Native hGH | 1.2 mg in 1 mL |
| 14 | 5 (66 to 70) | hGH(Y42H) | 1.2 mg in 1 mL |
| 15 | 5 (71 to 75) | hGH(Y42H) | 0.6 mg in 1 mL |
| 16 | 5 (76 to 80) | hGH(Y42H) | 0.3 mg in 1 mL |
| 17 | 5 (81 to 85) | hGH(Y42H) | 0.15 mg in 1 mL |
| 18 | 5 (86 to 90) | hGH(Y42I) | 1.2 mg in 1 mL |
| 19 | 5 (91 to 95) | hGH(Y42I) | 0.6 mg in 1 mL |
| 20 | 5 (96 to 100) | hGH(Y42I) | 0.3 mg in 1 mL |
| 21 | 5 (101 to 105) | hGH(Y42I) | 0.15 mg in 1 mL |

Blood samples were taken from all of the animals for the determination of plasma levels of the native hGH protein, hGH(Y42H) or hGH(Y42I) in rat serum. Blood samples were drawn on administration day 1 for each group immediately before administration and at 0.5, 1, 2, 4 and 6 hours after administration. 5 males were sampled per time-point and per group. At each time-point, a blood sample (200 μL) was taken and placed in ice cold collection tube containing lithium-heparin and mixed gently. Immediately, 30 μL of an anti-protease solution was added. Tubes were kept on ice and centrifuged at 2000×g for 10 minutes at 4° C. within 30 minutes of collection. The plasma was harvested in three tubes of at least 40 μl each, which was kept frozen at −20° C. or −80° C.

The amount of remaining hGH in animal plasma was determined using an anti-hGH ELISA kit (Mediagnost GmbH, reference E022), according to the manufacturer's protocol. Human GH ELISA Kit results were expressed in pg/ml after extrapolation from the standard curve of OD values measured with a spectrophotometer at 450 nm. Using PK solution software (SummitPK, Montrose, Colo., USA), PK parameters including Tₘₐₓ, Cₘₐₓ, half-life and AUC were determined for each pharmacokinetic profile.

Data obtained after PO administration of liquid formulation for hGH(Y42I) and hGH(Y42H) (at doses of 1.2, 0.6, 0.3 and 0.15 mg per rat) are represented in Tables 110a and 110b, respectively. These data were compared with data obtained for native hGH protein (at a single dose of 1.2 mg per rat). The PK profiles of hGH(Y42I) and native hGH proteins detected in systemic blood circulation by ELISA assay represent the average of male rats for each protein. PK parameters calculated from the average of animal PK profiles obtained following liquid formulation administration of hGH(Y42I), hGH(Y42H) and native hGH protein are presented in Tables 110a and 110b.

TABLE 110a
PK parameters for oral liquid administration

| Liquid formulation | hGH(Y42I) | | | | Native hGH |
|---|---|---|---|---|---|
| Dose (mg) | 1.2 | 0.6 | 0.3 | 0.15 | 1.2 |
| Cₘₐₓ (ng/ml) | 655.9 | 556.9 | 276.9 | 248.2 | — |
| Half-life (h) | 3.0 | 3.3 | 2.3 | 2.9 | — |
| AUC_{expo(0-∞)} (ng- | 2381.1 | 2617.1 | 2055.2 | 2411.1 | — |
| h/ml) | | | | | |
| AUC₍₀₋ₜ₎ (ng-h/ml) | 1625.5 | 1709.4 | 1203.7 | 950.2 | — |
| Tₘₐₓ (h) | 1 | 0.5 | 0.5* | 2 | — |

*Tmax value was considered to be 0.5 h instead of 4 h (PK solution software calculation) taking into account the PK profile heterogeneities between animals.

TABLE 110b
PK parameters for oral liquid administration

| Liquid formulation | hGH(Y42H) | | | | Native hGH |
|---|---|---|---|---|---|
| Dose (mg) | 1.2 | 0.6 | 0.3 | 0.15 | 1.2 |
| Cₘₐₓ (ng/ml) | 1030.1 | 958.6 | 461.7 | 378.0 | — |
| Half-life (h) | 3.1 | 2.5 | 3.0 | 1.9 | — |
| AUC_{expo(0-∞)} ng- | 4392.3 | 3446.1 | 2130.0 | 1280.7 | — |
| h/ml) | | | | | |
| AUC₍₀₋ₜ₎ (ng-h/ml) | 3374.1 | 2657.1 | 1445.7 | 1003.7 | — |
| Tₘₐₓ (h) | 1 | 1 | 0.5 | 0.5 | — |

Data obtained after PO administration of capsule formulation for hGH(Y42I) and hGH(Y42H) (at doses of 1.2, 0.6, 0.3 and 0.15 mg per rat) are represented in Tables 111a and 111b, respectively. These data were compared with data obtained for native hGH protein (at a single dose of 1.2 mg per rat). The PK profiles of hGH(Y42I) and native hGH proteins detected in systemic blood circulation by ELISA assay represent the average of male rats for each protein. PK parameters calculated from the average of animal PK profiles obtained following capsule formulation administration of hGH(Y42I), hGH(Y42H) and native hGH protein are presented in Tables 111a and 111b.

TABLE 111a
PK parameters for oral capsule administration

| Capsule formulation | hGH(Y42I) | | | | Native hGH |
|---|---|---|---|---|---|
| Dose (mg) | 1.2 | 0.6 | 0.3 | 0.15 | 1.2 |
| Cₘₐₓ (ng/ml) | 1360.0 | 916.8 | 732.6 | 829.0 | — |
| Half-life (h) | 4.1 | 2.8 | 2.4 | 0.7 | — |
| AUC_{expo(0-∞)} (ng- | 4970.5 | 3685.0 | 2204.5 | 930.6 | — |
| h/ml) | | | | | |
| AUC₍₀₋ₜ₎ (ng-h/ml) | 2794.2 | 2587.9 | 1755.3 | 804.2 | — |
| Tₘₐₓ (h) | 0.5 | 0.5 | 0.5 | 0.5 | — |

TABLE 111b
PK parameters for oral capsule administration

| Capsule formulation | hGH(Y42H) | | | | Native hGH |
|---|---|---|---|---|---|
| Dose (mg) | 1.2 | 0.6 | 0.3 | 0.15 | 1.2 |
| Cₘₐₓ (ng/ml) | 454.8 | 1082.3 | nd | 586.8 | — |
| Half-life (h) | 3.5 | 2.5 | nd | 1.2 | — |
| AUC_{expo(0-∞)} | 1996.5 | 2908.1 | nd | 1253.1 | — |
| (ng-h/ml) | | | | | |
| AUC₍₀₋ₜ₎ (ng-h/ml) | 1406.3 | 2217.3 | nd | 1031.5 | — |
| Tₘₐₓ (h) | 0.5 | 1 | nd | 1 | — |

nd: no PK parameters have been calculated for PK profile obtained with dose 0.3 mg for variant 57 due to lack of ELISA results obtained for all the rat blood sample at time 2, 4 and 6 h

For both liquid and capsule formulations, a significant and dose-dependent remaining hGH concentration was detected in rat plasma following PO administration of hGH(Y42I) and hGH(Y42H) whereas no signal was observed in plasma of native hGH protein administered rats. In addition, a dose-dependent increase in half-life of hGH(Y42I) and hGH(Y42H) following PO administration with both capsule and liquid formulations compared to intravenous route (see below) which strongly supports the existence of a sustained delivery of hGH(Y42I) and hGH(Y42H) from the intestinal compartment to the systemic circulation probably related to a slow absorption coupled with resistance to intestinal protease degradation.

3. Part II: Intravenous Dosing

For the IV administration, 8-week old pathogen free Sprague-Dawley rats (301-325 g) were injected i.v. with 0.1 mL of a 100 μg/ml hGH solution (either native hGH, hGH(Y42H), or hGH(Y42I)) per animal (i.e., a 10 μg dose). The treatment groups are presented in Table 112 for the IV dosing. The numbers in parenthesis represent the animal reference numbers in the study.

TABLE 112
Dosage and experimental detail for IV study

| | Main study | | |
|---|---|---|---|
| Group number | Number of animals | Treatment | Dose and volume |
| 10 | 5 (46 to 50) | Native hGH | 10 μg in 0.1 mL |
| 11 | 5 (51 to 55) | hGH(Y42H) | 10 μg in 0.1 mL |
| 12 | 5 (56 to 60) | hGH(Y42I) | 10 μg in 0.1 mL |
| 22 | 5 (106 to 110) | Native hGH | 10 μg in 0.1 mL |
| 23 | 5 (111 to 115) | hGH(Y42H) | 10 μg in 0.1 mL |
| 24 | 5 (116 to 120) | hGH(Y42I) | 10 μg in 0.1 mL |

Blood samples were taken from all of the animals for the determination of plasma levels of the native hGH protein, hGH(Y42H) or hGH(Y42I) in rat serum. Blood samples were drawn on administration day 1 for each group immediately before administration and at 0.083, 0.5, 1, 2, and 4 hours after administration. 5 males were sampled per time-point and per group. At each time-point, a blood sample (200 μL) was taken and placed in ice cold collection tube containing lithium-heparin and mixed gently. Immediately, 30 μL of an anti-protease solution was added. Tubes were kept on ice and centrifuged at 2000×g for 10 minutes at 4° C. within 30 minutes of collection. The plasma was harvested in three tubes of at least 40 μl each, which was kept frozen at −20° C. or −80° C.

The amount of remaining hGH in animal plasma was determined using an anti-hGH ELISA kit (Mediagnost GmbH, reference E022), according to the manufacturer's protocol. Human GH ELISA Kit results were expressed in pg/ml after extrapolation from the standard curve of OD values measured with a spectrophotometer at 450 nm. Using PK solution software (SummitPK, Montrose, Colo., USA), PK parameters including Tₘₐₓ, Cₘₐₓ, half-life and AUC were determined for each pharmacokinetic profile.

Data obtained after IV administration of hGH(Y42H), hGH(Y42I), and native hGH proteins (10 μg/animal dose) are presented Table 113. The PK profiles of hGH proteins detected in systemic blood circulation by ELISA assay represent the average of male rats for each protein. PK parameters calculated from the average of animal PK profiles are presented in Table 113.

TABLE 113
PK parameters for IV administration

| | Cᵢ | AUC_{expo (0-∞)} | Half-life |
|---|---|---|---|
| IV | (ng/ml) | (h-ng/ml) | (h) |
| hGH(Y42I) - 10 μg | 1357.4 | 1158.5 | 0.6 |
| hGH(Y42H) - 10 μg | 1153.3 | 1166.3 | 0.7 |
| Native hGH - 10 μg | 2088.3 | 331.0 | 0.11 |

The initial concentration (Cᵢ) value obtained for hGH(Y42I) was similar to hGH(Y42H) (1357.4 ng/ml for hGH(Y42I) versus 1153.3 ng/ml for hGH(Y42H)). However, native hGH protein exhibited higher Cᵢ values compared to both hGH(Y42I) and hGH(Y42H) (2088.3 ng/ml for native hGH compared to 1357.4 ng/ml for hGH(Y42I)). An increase of AUC_{expo(0-∞)} about 3.5-fold was observed for rats injected with either hGH(Y42I) or hGH(Y42H) compared to native hGH protein. Similarly, both hGH(Y42I) or hGH(Y42H) exhibited an increased half-life, 5.5-fold and 6.4-fold, respectively, compared to native hGH protein.

4. Bioavailability Analysis of PO Versus IV Pharmacokinetic Data

For each dose of hGH(Y42I) and hGH(Y42H) administered PO in both capsule and liquid formulations to rats, the percentage of hGH(Y42I) bioavailability compared to IV administration was calculated using the following equations:

### Bioavailability (in %) Related to Cₘₐₓ

((Cₘₐₓ/dose)_{hGH}/(Cₘₐₓ/dose)_{hGH-IV})×100

### Bioavailability (in %) Related to AUC

((AUC/dose)_{hGH}/(AUC/dose)_{hGH-IV})×100

### Bioavailability (in %) Related to AUC and Relative to Native IFN-Alpha

((AUC/dose)_{hGHmut}/(AUC/dose)_{native-IV})×100

For each dose of hGH(Y42I) and hGH(Y42H) liquid formulations administered PO to rats, the percentage of hGH(Y42I) and hGH(Y42H) bioavailability compared to IV administration was calculated related to Cₘₐₓ and AUC. Results are presented in Table 114a for hGH(Y42H) (labeled hgh-57 in Table) and Table 114b for hGH(Y42I) (labeled hgh-58 in Table).

TABLE 114a
Bioavailability data for hGH(Y42H) liquid formulations

| | | | | | Native |
|---|---|---|---|---|---|
| | hgh-57 per-os | | | hgh-57 IV | hgh IV |
| Dose (mg) | 1.2 | 0.6 | 0.3 | 0.01 | 0.01 |
| AUC_{(0-∞)} expo | 4392.3 | 3446.6 | 2130.0 | 1166.3 | 331.0 |
| (ng-hr/ml) | | | | | |
| Cₘₐₓ (ng/ml) | 1030.1 | 958.6 | 461.7 | 1153.3 | 2088.3 |
| Cmax/dose | 858 | 1598 | 1539 | 115330 | 208830 |
| ((Cmax/dose)₅₇/ | 0.7 | 1.4 | 1.3 | | |
| (Cmax/dose)_{57-IV}) × | | | | | |
| 100 | | | | | |
| AUC/dose | 3660 | 5744 | 7100 | 116630 | 33100 |
| (AUC/dose)₅₇/ | 3.1 | 4.9 | 6.1 | | |
| (AUC/dose)_{57-IV}) × | | | | | |
| 100 | | | | | |
| (AUC/dose)₅₇/ | 11.1 | 17.4 | 21.5 | | |
| (AUC/dose)_{natif-IV}) × | | | | | |
| 100 | | | | | |

TABLE 114b
Bioavailability data for hGH(Y42I) liquid formulation

| | | | | | Native |
|---|---|---|---|---|---|
| | hgh-58 per-os | | | hgh-58 IV | hgh IV |
| Dose (mg) | 1.2 | 0.6 | 0.3 | 0.01 | 0.01 |
| AUC_{(0-∞)} expo | 2381.1 | 2617.1 | 2055.2 | 1158.5 | 331.0 |
| (ng-hr/ml) | | | | | |
| Cₘₐₓ (ng/ml) | 655.9 | 556.9 | 276.9 | 1357.4 | 2088.3 |
| Cmax/dose | 547 | 928 | 923 | 135740 | 208830 |
| ((Cmax/dose)₅₈/ | 0.4 | 0.7 | 0.7 | | |
| (Cmax/dose)_{58-IV}) × | | | | | |
| 100 | | | | | |
| AUC/dose | 1984 | 4362 | 6851 | 115850 | 33100 |
| (AUC/dose)₅₈/ | 1.7 | 3.8 | 5.9 | | |
| (AUC/dose)_{58-IV}) × | | | | | |
| 100 | | | | | |
| (AUC/dose)₅₈/ | 6.0 | 13.2 | 20.7 | | |
| (AUC/dose)_{natif-IV}) × | | | | | |
| 100 | | | | | |

For each dose of hGH(Y42I) and hGH(Y42H) capsule formulations administered PO to rats, the percentage of hGH(Y42I) and hGH(Y42H) bioavailability compared to IV administration was calculated related to Cₘₐₓ and AUC. Results are presented in

Table 115a for hGH(Y42H) (labeled hgh-57 in Table) and Table 115b for hGH(Y42I) (labeled hgh-58 in Table).

TABLE 115a
Bioavailability data for hGH(Y42H) capsule formulation

| | | | | Native |
|---|---|---|---|---|
| | hgh-57 capsules | | hgh-57 IV | hgh IV |
| Dose (mg) | 1.2 | 0.6 | 0.01 | 0.01 |
| AUC_{(0-∞)} (ng-hr/ml) | 1996.5 | 2908.1 | 1166.3 | 331.0 |
| Cₘₐₓ (ng/ml) | 454.8 | 1082.3 | 1153.3 | 2088.3 |
| Cmax/dose | 379 | 1804 | 115330 | 208830 |
| ((Cmax/dose)₅₇/ | 0.3 | 1.6 | | |
| (Cmax/dose)_{57-IV}) × 100 | | | | |
| AUC/dose | 1664 | 4847 | 116630 | 33100 |
| (AUC/dose)₅₇/ | 1.4 | 4.2 | | |
| (AUC/dose)_{57-IV}) × 100 | | | | |
| (AUC/dose)₅₇/ | 5.0 | 14.6 | | |
| (AUC/dose)_{natif-IV}) × 100 | | | | |

TABLE 115b
Bioavailability data for hGH(Y42I) capsule formulations

| | | | | | Native |
|---|---|---|---|---|---|
| | hgh-58 capsules | | | hgh-58 IV | hgh IV |
| Dose (mg) | 1.2 | 0.6 | 0.3 | 0.01 | 0.01 |
| AUC_{(0-∞)} (ng-hr/ml) | 4970.5 | 3685.0 | 2204.5 | 1158.5 | 331.0 |
| Cₘₐₓ (ng/ml) | 1360 | 916.8 | 732.6 | 1357.4 | 2088.3 |
| Cmax/dose | 1133 | 1528 | 2442 | 135740 | 208830 |
| ((Cmax/dose)₅₈/ | 0.8 | 1.1 | 1.8 | | |
| (Cmax/dose)_{58-IV}) × | | | | | |
| 100 | | | | | |
| AUC/dose | 4142 | 6142 | 7348 | 115850 | 33100 |
| (AUC/dose)₅₈/ | 3.6 | 5.3 | 6.3 | | |
| (AUC/dose)_{58-IV}) × | | | | | |
| 100 | | | | | |
| (AUC/dose)₅₈/ | 12.5 | 18.6 | 22.2 | | |
| (AUC/dose)_{natif-IV}) × | | | | | |
| 100 | | | | | |

### Example 28

### In Vivo Pharmacokinetic (PK) Study of hGH(Y42I) and hGH(Y42H) in Normal Rats Compared to Native hGH Following SC and IV Administrations

Pharmacokinetic studies were performed on 8-week old Sprague-Dawley male rats to compare per subcutaneous (SC) and intravenous (IV) routes of administration. The objective of this study was to investigate the PK profile for hGH variant 57 (Y42H) and hGH variant 58 (Y42I). The study involved 72 Sprague-Dawley male rats (6 groups of 12 animals) and was conducted in two parts: 1) subcutaneous and 2) intravenous injection.

1. hGH Formulations Used in the Study

Preparations of native hGH and hGH variants used in the study are presented in Tables 116(a-f).

TABLE 116a
hGH WT Batch # hGH-004/06
(Group 1)

| | |
|---|---|
| Date of manufacture | June 2006 |
| Expiry date | December 2006 |
| Correspondence MIU | 171 MIU = 1 mg |
| vs mg | |
| Presentation | Sterile solution at 100 μg/mL. |
| | Preparation (0.1 mg/mL): 9.1 mL of a WT hGH |
| | solution in: |
| | 7.5 mg/mL NaCl, |
| | 6 mg/mL sucrose, |
| | 2.38 mg/mL sodium phosphate dibasic, |
| | 0.44 mg/mL sodium phosphate monobasic |
| | added to 0.9 mL of PBS |
| Administration | Subcutaneous route |

TABLE 116b
hGH WT Batch # hGH-004/06
(Group 4)

| | |
|---|---|
| Date of manufacture | June 2006 |
| Expiry date | December 2006 |
| Correspondence MIU vs mg | 171 MIU = 1 mg |
| Presentation | Sterile solution at 50 μg/mL |
| | Preparation (0.05 mg/mL): |
| | 4.5 mL of a WT hGH solution in |
| | 7.5 mg/mL NaCl, |
| | 6 mg/mL sucrose, |
| | 2.38 mg/mL sodium phosphate dibasic, |
| | 0.44 mg/mL sodium phosphate monobasic |
| | added to 5.5 mL of PBS |
| Administration | Intravenous route |

TABLE 116c
hGH(Y42I) Batch # hGH-003/06
(Group 3)

| | |
|---|---|
| Date of manufacture | June 2006 |
| Expiry date | December 2006 |
| Correspondence MIU vs mg | 138 MIU = 1 mg |
| Presentation | Sterile solution at 100 μg/mL |
| | Preparation (0.1 mg/mL): |
| | 7.8 mL of hGH Lead 58 solution in |
| | 7.5 mg/mL NaCl, |
| | 6 mg/mL sucrose, |
| | 2.38 mg/mL sodium phosphate dibasic, |
| | 0.44 mg/mL sodium phosphate monobasic |
| | added to 2.2 mL of PBS |
| Administration | Subcutaneous route |

TABLE 116d
hGH(Y42I) Batch # hGH-003/06
(Group 6)

| | |
|---|---|
| Date of manufacture | June 2006 |
| Expiry date | December 2006 |
| Correspondence MIU vs mg | 138 MIU = 1 mg |
| Presentation | Sterile solution at 50 μg/mL |
| | Preparation (0.05 mg/mL): |
| | 3.9 mL of hGH Lead 58 solution in |
| | 7.5 mg/mL NaCl, |
| | 6 mg/mL sucrose, |
| | 2.38 mg/mL sodium phosphate dibasic, |
| | 0.44 mg/mL sodium phosphate monobasic |
| | added to 6.1 mL of PBS |
| Administration | Intravenous route |

TABLE 116e
hGH(Y42H) Batch # hGH57-002/06
(Group 2)

| | |
|---|---|
| Date of manufacture | May 2006 |
| Expiry date | November 2006 |
| Correspondence MIU vs mg | 141 MIU = 1 mg |
| Presentation | Sterile solution at 100 μg/mL |
| | Preparation (0.1 mg/mL): |
| | 7.8 mL of hGH Lead 57 solution in |
| | 7.5 mg/mL NaCl, |
| | 6 mg/mL sucrose, |
| | 2.38 mg/mL sodium phosphate dibasic, |
| | 0.44 mg/mL sodium phosphate monobasic |
| | added to 2.2 mL of PBS |
| Administration | Subcutaneous route |

TABLE 116f
hGH(Y42H) Batch # hGH57-002/06
(Group 5)

| | |
|---|---|
| Date of manufacture | May 2006 |
| Expiry date | November 2006 |
| Correspondence MIU vs mg | 141 MIU = 1 mg |
| Presentation | Sterile solution at 50 μg/mL |
| | Preparation (0.05 mg/mL): |
| | 3.9 mL of hGH Lead 57 solution in |
| | 7.5 mg/mL NaCl, |
| | 6 mg/mL sucrose, |
| | 2.38 mg/mL sodium phosphate dibasic, |
| | 0.44 mg/mL sodium phosphate monobasic |
| | added to 6.1 mL of PBS |
| Administration | Intravenous route |

2. Part I: Subcutaneous Dosing

For SC administration, 8-week old pathogen free Sprague-Dawley rats (301-325 g) were administered a liquid formulation via subcutaneous injection. The animals were administered with 0.5 mL of 100 μg/mL solution of either native hGH, hGH(Y42H) or hGH(Y42I). Three groups of 12 animals were used. The treatment groups are presented in Table 117. The numbers in parenthesis represent the animal reference numbers in the study.

TABLE 117
Dosage and experimental detail for SC study

| Group | Main study | | Dose and |
|---|---|---|---|
| number | Number of animals | Treatment | volume |
| 1 | 12 males (1 to 12) | Native hGH | 100 μg/ml |
| | | | 0.5 ml |
| 2 | 12 males (13 to 24) | hGH(Y42H) | 100 μg/ml |
| | | | 0.5 ml |
| 3 | 12 males (25 to 36) | hGH(Y42I) | 100 μg/ml |
| | | | 0.5 ml |

Blood samples were taken from the animals for determination of plasma levels of native hGH, hGH(Y42H) or hGH(Y42I) in rat serum. Blood samples were drawn on administration day 1 for each group immediately before administration and at 0.25, 0.5, 1, 2, 4, 8, 12, 16, 24 and 32 hours after administration. 6 males were sampled per time-point and per group. For the full pharmacokinetic profile for each protein (hGH, hGH(Y42H) or hGH(Y42I)) dose, the group of 12 rats was sampled as follows: one group of 6 males drawn at pre-dose, 0.5, 1, 2, 16 and 48 hours and another group of 6 males at 0.25, 4, 8, 12, 24 and 32 hours. At each time-point, a blood sample (200 μL) was taken and placed in ice cold collection tube containing lithium-heparin and mixed gently. Immediately, 30 μL of an anti-protease solution was added. Tubes were kept on ice and centrifuged at 2000×g for 10 minutes at 4° C. within 30 minutes of collection. The plasma was harvested in three tubes of at least 40 μl each, which was kept frozen at −20° C. or −80° C.

The amount of remaining hGH in animal plasma was determined using an anti-hGH ELISA kit (Mediagnost GmbH, reference E022), according to the manufacturer's protocol. Human GH ELISA Kit results were expressed in pg/ml after extrapolation from the standard curve of OD values measured with a spectrophotometer at 450 nm. Using PK solution software (SummitPK, Montrose, Colo., USA), PK parameters including Tₘₐₓ, Cₘₐₓ, half-life and AUC were determined for each pharmacokinetic profile.

Data obtained after SC administration of hGH(Y42I) and native hGH proteins (at a dose of 100 μg/ml) are presented in Table 118. The PK profiles of hGH(Y42I) and native hGH proteins detected in systemic blood circulation by ELISA assay represent the average of the male rats for each protein. PK parameters calculated from the average of animal PK profiles obtained following SC administration of hGH(Y42I) and native hGH proteins is presented in Table 118. Determination of hGH remaining concentration in rat sera following SC administration with hGH(Y42H) was not performed.

TABLE 118
PK parameters for SC administration

| | Cₘₐₓ | AUC_{(0-∞)} | AUC₍₀₋ₜ₎ | Half-life | Tmax |
|---|---|---|---|---|---|
| SC | (ng/ml) | (h-ng/ml) | (h-ng/ml) | (h) | (h) |
| hGH(Y42I) - | 132.8 | 1706.6 | 1704.4 | 14.5 | 1* |
| 00 μg/m | | | | | |
| Native hGH - | 134.9 | 249.2 | 249.2 | 0.7 | 0.5 |
| 100 μg/ml | | | | | |

*Tₘₐₓ value was considered to be 1 h instead of 2 h (PK solution software calculation) taking into account the PK profile heterogeneities between animals

The Cₘₐₓ value obtained for hGH(Y42I) was similar to native hGH protein (132.8 ng/ml for hGH(Y42I) compared to 134.9 ng/ml for native hGH). An increase of AUC₍₀₋ₜ₎ about 6.8-fold was observed for rats injected with hGH(Y42I) compared to native hGH protein (1706.6 ng-h/ml for hGH(Y42I) versus 249.2 ng-h/ml for native hGH protein). Similarly, half-life for hGH(Y42I) was increased 20.7-fold compared to native protein (14.5 h for hGH(Y42I) versus 0.7 h for native hGH).

3. Part II: Intravenous Dosing

For SC administration, 8-week old pathogen free Sprague-Dawley rats (301-325 g) were administered a liquid formulation via intravenous injection. The animals were administered with 0.3 mL of 50 μg/mL solution of either native hGH, hGH(Y42H) or hGH(Y42I). Three groups of 12 animals were used. The treatment groups are presented in Table 119. The numbers in parenthesis represent the animal reference numbers in the study.

TABLE 119
Dosage and experimental detail for IV study

| | Main study | | Dose and |
|---|---|---|---|
| Group number | Number of animals | Treatment | volume |
| 4 | 12 males (37 to 48) | Native hGH | 50 μg/ml |
| | | | 0.3 ml |
| 5 | 12 males (49 to 60) | hGH variant 57 | 50 μg/ml |
| | | | 0.3 ml |
| 6 | 12 males (61 to 72) | hGH variant 58 | 50 μg/ml |
| | | | 0.3 ml |

Blood samples were taken from the animals for determination of plasma levels of native hGH, hGH(Y42H) or hGH(Y42I) in rat serum. Blood samples were taken from the animals for determination of plasma levels of native hGH, hGH(Y42H) or hGH(Y42I) in rat serum. Blood samples were drawn on administration day 1 for each group immediately before administration and at 0.05, 0.083, 0.25, 0.5, 1, 1.5, 2, 4, 6, 8 and 12 hours after administration. 6 males were sampled per time-point and per group. For the full pharmacokinetic profile for each protein (hGH, hGH(Y42H) or hGH(Y42I)) dose, the group of 12 rats was sampled as follows: one group of 6 males drawn at pre-dose, 0.083, 0.5, 1.5, 6 and 8 hours and another group of 6 males at 0.05, 0.25, 1, 2, 4 and 12 hours.

At each time-point, a blood sample (200 μL) was taken and placed in ice cold collection tube containing lithium-heparin and mixed gently. Immediately, 30 μL of an anti-protease solution was added. Tubes were kept on ice and centrifuged at 2000×g for 10 minutes at 4° C. within 30 minutes of collection. The plasma was harvested in three tubes of at least 40 μl each, which was kept frozen at −20° C. or −80° C.

The amount of remaining hGH in animal plasma was determined using an anti-hGH ELISA kit (Mediagnost GmbH, reference E022), according to the manufacturer's protocol. Human GH ELISA Kit results were expressed in pg/ml after extrapolation from the standard curve of OD values measured with a spectrophotometer at 450 nm. Using PK solution software (SummitPK, Montrose, Colo., USA), PK parameters including Tₘₐₓ, Cₘₐₓ, half-life and AUC were determined for each pharmacokinetic profile.

Data obtained after IV administration of hGH(Y42I) and native hGH proteins (at a dose of 50 μg/ml) is presented in Table 120. The PK profiles of hGH(Y42I) and native hGH proteins detected in systemic blood circulation by ELISA assay represent the average of male rats for each protein. PK parameters calculated from the average of animal PK profiles obtained following IV administration of hGH(Y42I) and native hGH proteins is presented in Table 120. Determination of hGH remaining concentration in rat sera following IV administration with hGH(Y42H) was not performed.

TABLE 120
PK parameters for IV administration

| | Cᵢ | AUC_{(0-∞)} | Half-life |
|---|---|---|---|
| IV | (ng/ml) | (h-ng/ml) | (h) |
| hGH(Y42I) - 50 μg/ml | 303.4 | 209.8 | 0.73 |
| Native hGH - 50 μg/m | 338.0 | 56.1 | 0.25 |

The initial concentration (Ci) value obtained for hGH(Y42I) was similar to native hGH protein (303.4 ng/ml for hGH(Y42I) compared to 338.0 ng/ml for native hGH). An increase of AUC_{(0-∞)} about 3.7-fold was observed for rats injected with hGH(Y42I) compared to native hGH protein (209.8 ng-h/ml for hGH(Y42I) versus 56.1 ng-h/ml for native hGH protein). Similarly, half-life for hGH(Y42I) was increased about 3-fold compared to native protein (0.73 h for hGH(Y42I) versus 0.25 h for native hGH).

### Example 29

### In Vivo Study of hGH(Y42I) Activity in Hypophysectomized Rats Compared to Native hGH Following Oral and SC Administrations

Growth hormone deficiency in humans is associated with increased adiposity and decreased bone mineral density. Hypophysectomized rats are used as a model of pituitary hormone deficiency to study the effects of growth hormone deficiency on bone (Appiagyei-Dankah et al. (2003) Am. J. Physiol. Endocrinol Metab., 284:566-573). Administration of growth hormone in the hypophysectomized rat model has been demonstrated to have stimulatory effects on bone growth and turnover and can prevent hypophysectomized induced skeletal alterations in rats (Cheng et al. (1997) Anat. Rec., 249: 163-172).

The effects of dosage formulations of growth hormone protease resistant polypeptides administered by subcutaneous or oral route on reversal of effects of growth hormone deficiency in the hypophysectomized animal model were assessed. Hypophysetomized Sprauge Dawley rats were administered with liquid formulations (described in Example 29) of native or hGH(Y42I) by oral (liquid gavage) or subcutaneous route. The treatment groups were as follows: 1) 1200 μg/rat native hGH (oral); 2) 300 μg/rat hGH(Y42I) (oral); 3) 600 μg/rat hGH(Y42I) (oral); 4) 16 μg/rat native hGH (subcutaneous); and 5) vehicle only control. Changes in body weight were monitored daily for at 1, 2, 3, 4, 5, 5, 7, and 8 days after administration of the polypeptide.

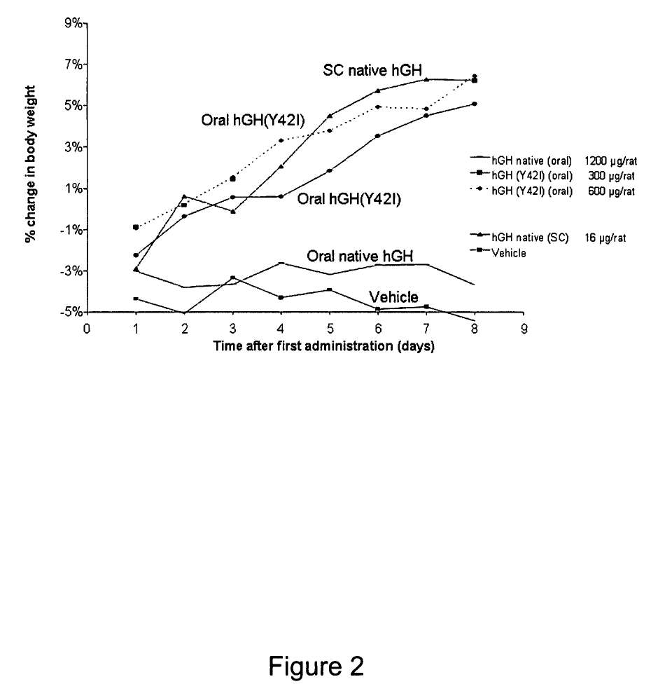

The results are shown in FIG. 2. Results from this experiment demonstrate that treatment of hypophysectomized rats treated subcutaneously with native growth hormone at 16 μg/rat resulted in an increased body weight, thereby evidencing activity of growth hormone in this model. In contrast, administration of native growth hormone orally, even at doses at high as 1200 μg/rat, showed no increase in body weight. Administration of hGH(Y42I) orally at doses of 300 μg/rat and 600 μg/rat resulted in an increased change in body weight at levels that were similar to that seen in rats administered with native hGH subcutaneously. This result establishes that a dosage formulation of a protease resistant hGH of 15-40 times that of the subcutaneous dose of a native hGH is sufficient to yield a similar activity and effect in a model of growth hormone deficiency.

Since modifications will be apparent to those of skill in this art, it is intended that this invention be limited only by the scope of the appended claims.

1. A dosage formulation, comprising an amount of a therapeutic polypeptide sufficient, upon oral administration to a subject, to reach therapeutically effective levels in the bloodstream in the subject, wherein:
the therapeutic protein is modified in its primary sequence of amino acids, whereby it is more protease resistant than in the absence of the modifications, and the protein can reach therapeutically effective levels in the bloodstream upon oral administration; and
the amount is about or is 10-100 times the subcutaneous dose for the same indication and for the same therapeutic polypeptide that is not modified to be protease resistant.

2. The dosage formulation of claim 1, wherein the therapeutic polypeptide contains one or two amino acid sequence substitutions that render it more protease resistant than in the absence of the modifications.

3. The dosage formulation of claim 1, wherein the amount is 10-50 times the subcutaneous dose for the same indication and for the same therapeutic polypeptide.

4. The dosage formulation of claim 1, wherein the amount is 15-40 times the subcutaneous dose for the same indication and for the same therapeutic polypeptide.

5. The dosage formulation of claim 1, wherein:
the dosage formulation comprises a plurality of dosage units; and
each dosage unit comprises a fractional amount of the total amount of the therapeutic polypeptide in the dosage formulation.

6. The dosage formulation of claim 5, wherein the dosage unit is a tablet or capsule.

7. The dosage formulation of claim 6, wherein the dosage unit is enterically coated.

8. The dosage formulation of claim 1, wherein:
the dosage formulation comprises one or more dosage units containing the therapeutic polypeptide; and
each dosage unit is a tablet or capsule.

9. The dosage formulation of claim 8, wherein the dosage unit is a tablet.

10. The dosage formulation of claim 9, wherein the dosage unit is enterically coated.

11. The dosage formulation of claim 1, wherein the therapeutic polypeptide is a cytokine or a blood coagulation factor.

12. The dosage formulation of claim 1, wherein the therapeutic polypeptide is selected from among interleukin-10, an interferon-β, an interferon alpha, interferon gamma, granulocyte colony stimulating, leukemia inhibitory factor, a growth hormone, ciliary neurotrophic factor, leptin, insulin, oncostatin M, relaxin, interleukin-6, interleukin-12, erythropoietin, granulocyte-macrophage colony stimulating factor, interleukin-2, interleukin-3, interleukin-4, interleukin-5, interleukin-13, Flt3 ligand, a monoclonal antibody or fragment thereof and stem cell factor.

13. The dosage formulation of claim 1, wherein the modifications to increase protease resistance are the only modifications in the sequence of amino acids compared to a wild-type polypeptide or polypeptide not modified to exhibit increased protease resistance.

14. The dosage formulation of claim 1, wherein the polypeptide includes further modifications that alter properties other than protease resistance in the sequence of amino acids compared to a wild-type polypeptide or polypeptide not modified to exhibit increased protease resistance.

15. The dosage formulation of claim 1, wherein the polypeptide includes modifications that add new glycosylation sites to the polypeptide, whereby the polypeptide is hyperglycosylated.

16. The dosage formulation of claim 1, wherein the protease-resistant polypeptide is selected from among protease-resistant variants of coagulation factors, cytokines, growth factors, hormones, hydrolases, immunoglobulins, inhibitor polypeptides, nuclear polypeptides and proteases.

17. The dosage formulation of claim 16, wherein the unmodified polypeptide is selected from among interleukin-10 (IL-10; SEQ ID NO: 809), interferon beta (IFN-β; SEQ ID NO: 147), interferon alpha-2a (IFNα-2a; SEQ ID NO: 2162), interferon alpha-2b (IFNα-2b; SEQ ID NO: 2067), interferon gamma (IFN-γ; SEQ ID NO: 661), granulocyte colony stimulating factor (G-CSF; SEQ ID NO: 85), leukemia inhibitory factor (LIF; SEQ ID NO: 1148), growth hormone (GH; SEQ ID NO: 1260), ciliary neurotrophic factor (CNTF; SEQ ID NO: 1), leptin (SEQ ID NO: 1126), oncostatin M (SEQ ID NO: 1181), interleukin-6 (IL-6; SEQ ID NO: 1080), interleukin-12 (IL-12; SEQ ID NO: 860); erythropoietin (EPO; SEQ ID NO: 1886), granulocyte-macrophage colony stimulating factor (GM-CSF; SEQ ID NO: 47), interleukin-2 (IL-2; SEQ ID NO: 946), interleukin-3 (IL-3; SEQ ID NO: 995), interleukin-4 (IL-4; SEQ ID NO: 1018), interleukin-5 (IL-5; SEQ ID NO: 1044), interleukin-13 (IL-13; SEQ ID NO: 917), Flt3 ligand (SEQ ID NO: 118) and stem cell factor (SCE; SEQ ID NO: 1215).

18. The dosage formulation of claim 17, wherein the polypeptide is selected from modified therapeutic polypeptides who sequences are set forth in any of SEQ ID NOS: 2-46, 48-84, 86-117, 119-146, 148-660, 662-808, 810-859, 861-916, 918-945, 947-994, 996-1017, 1019-1043, 1045-1079, 1081-1125, 1127-1147, 1149-1180, 1182-1214, 1216-1259, 1261-1885, 1887-1981, 1983-2066, and 2176-2182.

19. The dosage formulation of claim 1, wherein the modified polypeptide is IFN-α2b or human growth hormone.

20. The dosage formulation of claim 19, wherein the polypeptide is IFN-α2b containing a modification that is or corresponds to E41Q of SEQ ID NO: 2067.

21. The dosage formulation of claim 19, wherein the modified therapeutic polypeptide is a human growth hormone containing a modification that is or corresponds to Y421 of SEQ ID NO: 1260.

22. The dosage formulation of claim 20, wherein the sequence of amino acids of the IFN-α2b is set forth in SEQ ID NO: 1995.

23. The dosage formulation of claim 21, wherein the sequence of the growth hormone is set forth in SEQ ID NO: 1318.

24. A tablet or capsule, comprising 10-100 μg of an interferon-α(IFN-α), wherein the IFN-α is protease resistant by virtue of modifications in its primary sequence of amino acids.

25. The tablet or capsule of claim 24, wherein the IFN-α is IFN-α2a or IFN-α2b.

26. The tablet or capsule of claim 24, comprising a second therapeutic agent for the same indication as the interferon.

27. The tablet or capsule of claim 26, wherein the second therapeutic agent is selected from among a nucleoside analog, an L-nucleoside, a type II interferon receptor antagonist, a tumor necrosis factor (TNF) antagonist, thymosin-α, a SAPK inhibitor, amantidine, an NS3 inhibitor, an NS5B inhibitor, and an alpha-glucosidase inhibitor.

28. The tablet or capsule of claim 27, wherein the nucleoside analog is ribavirin.

29. A tablet or capsule of claim 24, comprising 10-50 μg of an interferon-α protease-resistant polypeptide.

30. The tablet or capsule of claim 24, wherein the IFN-α further comprises additional modifications, whereby it is hyperglycosylated.

31. The tablet or capsule of claim 24 that is enterically coated.

32. The tablet or capsule of claim 25, wherein the IFN-α2b contains a modification that is or corresponds to E41Q of SEQ ID NO: 2067.

33. The tablet or capsule of claim 25, wherein the IFN-α2b comprises the sequence of amino acids set forth in SEQ ID NO: 1995.

34. A tablet or capsule, comprising 1-50 mg, 10-50 mg, 10-40 mg, 15-35 mg, 1-40 mg, 1-45 mg, 5-30 mg, 10-20 mg or 3-30mg of human growth hormone, wherein the human growth hormone is protease resistant.

35. The tablet or capsule of claim 34, comprising a second therapeutic agent for the same indication as the growth hormone.

36. The tablet or capsule of claim 35, wherein the second therapeutic agent is selected from among a gonadotropin releasing hormone (GNRH) analog, insulin-like growth factor 1, (rhIGF-1), a growth hormone-releasing peptide (GRP), a free fatty acid regulation agent, leutenizing hormone releasing hormone, and an anabolic steroid.

37. The tablet or capsule of claim 34, wherein the tablet or capsule contains 3 mg, 12 mg, 24 mg or 30 mg of the protease-resistant growth hormone polypeptide.

38. The tablet or capsule of claim 34 that is enterically coated.

39. The tablet or capsule of claim 34, wherein the human growth hormone further comprises additional modifications, whereby it is hyperglycoslated.

40. The tablet or capsule of claim 34, wherein the human growth hormone contains a modification that is or corresponds to Y42I of SEQ ID NO: 1260.

41. The tablet or capsule of claim 34, wherein the human growth hormone comprises the sequence of amino acids set forth in SEQ ID NO: 1318.

42. A dosage formulation, comprising a plurality of tablets or capsules of claim 24.

43. A dosage formulation, comprising a plurality of tablets or capsules of claim 34.

44. A method of treatment of a disease or condition, comprising administering an oral dosage formulation of a modified therapeutic polypeptide, wherein:
the therapeutic polypeptide is modified in its primary sequence of amino acids, whereby it is more protease resistant than in the absence of the modifications, and the polypeptide can reach therapeutically effective levels in the bloodstream upon oral administration; and
the amount of therapeutic polypeptide in the formulation is 15-40 times the subcutaneous dose for the same indication and for the same therapeutic polypeptide that is not modified to be protease resistant.

45. The method of claim 44, wherein the therapeutic polypeptide is an IFN-α polypeptide.

46. The method of claim 44, wherein the disease or condition is selected from among a viral infection, a fibrotic disorder, condylomata acuminate and cancer.

47. The method of claim 46, wherein the viral infection is a hepatitis C infection or a hepatitis B infection.

48. The method of claim 46, wherein the cancer is selected from among hairy cell leukemia, malignant lymphoma, follicular lymphoma and AIDS-related Karposi sarcoma.

49. The method of claim 46, wherein the fibrotic disorder is selected from among idiopathic pulmonary fibrosis, liver fibrosis and renal fibrosis.

50. The method of claim 44, wherein the therapeutic polypeptide is a growth hormone polypeptide.

51. The method of claim 44, wherein the disease or condition is selected from among a growth deficiency disorder, AIDS wasting, aging, impaired immune function of HIV-infected subjects, a catabolic illness, surgical recovery, a congestive cardiomyopathy, liver transplantation, liver regeneration after hepatectomy, chronic renal failure, renal osteodystrophy, osteoporosis, achondroplasia/hypochondroplasia, skeletal dysplasia, a chronic inflammatory disorder, Crohn's disease, short bowel syndrome, juvenile chronic arthritis, cystic fibrosis, male infertility, X-Iinked hypophosphatemic rickets, Down's syndrome, Spina bifida, Noonan Syndrome, obesity, impaired muscle strength and fibromyalgia.

52. The method of claim 51, wherein the growth deficiency disorder is selected from among Turner's syndrome, intrauterine growth retardation, idiopathic short stature, Prader Willi syndrome, and thalassaenua.

53. The method of claim 44, wherein the dosage formulation contains a plurality of dosage units and all are administered in a single day.

54. The method of claim 44, wherein a dosage formulation is administered one, two, three, four, five, six or seven times a week.

55. The method of claim 44, wherein a dosage formulation is administered one, two or three times a day.

56. A method of treatment of a disease or condition, comprising administering a dosage formulation of claim 1.

57. A method of treatment of a disease or condition, comprising administering the tablet or capsule of claim 24.

58. A method of treatment of a disease or condition, comprising administering the tablet or capsule of claim 34.
